(19) United States
(12) Patent Application Publication
(10) Pub. No.: US 2008/0299204 A1
(43) Pub. Date: Dec. 4, 2008

(54) Dosage forms for movement disorder treatment

(75) Inventors: Avinash Nangia, Sharon, MA (US); Jules Jacob, Taunton, MA (US); James Yeh, Foxboro, MA (US); Peyman Moslemy, Providence, RI (US); Daya D. Verma, Needham, MA (US); Dinesh K. Haswani, Plainville, MA (US); Ze'ev Shaked, San Antonio, TX (US)

Correspondence Address: ROPES & GRAY LLP, PATENT DOCKETING 39/41, ONE INTERNATIONAL PLACE, BOSTON, MA, 02110-2624, US

(73) Assignee: Spherics, Inc., Mansfield, MA (US)

(21) Appl. No.: 11/474,134

(22) Filed: Jun. 23, 2006

Related U.S. Application Data
(60) Provisional application No. 60/693,602, filed on Jun. 23, 2005.

(51) Int. Cl. A61K 9/16; A61K 31/195; A61K 31/428; A61P 25/16; A61K 31/445

(52) U.S. Cl. 424/489; 514/567; 514/367; 514/317

(57) ABSTRACT

The invention relates to the improvement in the treatment of certain neural disorders/diseases, such as Parkinson's disease and other motor disorders. One aspect of the invention relates to drug compositions and dosage forms comprising said drug composition. Another aspect of the invention relates to methods of manufacturing the drug compositions and dosage forms. Another aspect of the invention relates to methods of treatment, comprising administering the drug composition and dosage form to an individual.

### REFERENCE TO RELATED APPLICATION

This application claims the benefit of the filing date of U.S. Provisional Application Ser. No. 60/693,602, entitled “IMPROVED DOSAGE FORMS FOR MOVEMENT DISORDER TREATMENT,” and filed on Jun. 23, 2005. The teachings of the entire referenced application are incorporated herein by reference.

### BACKGROUND OF THE INVENTION

A movement disorder is a neurological disturbance that involves one or more muscles or muscle groups. Movement disorders affect a significant portion of the population, causing disability as well as distress. Movement disorders include Parkinson's disease, Huntington's chorea, progressive supranuclear palsy, Wilson's disease, Tourette's syndrome, epilepsy, tardive dyskinesia, and various chronic tremors, tics and dystonias. Different clinically observed movement disorders can be traced to the same or similar areas of the brain. For example, abnormalities of basal ganglia (a large cluster of cells deep in the hemispheres of the brain) are postulated as a causative factor in diverse movement disorders.

Parkinson's disease is a movement disorder of increasing occurrence in aging populations. It is a progressive neurodegenerative disorder affecting the mobility and control of the skeletal muscular system. The disease is associated with the depletion of dopamine from cells in the corpus striatum. Parkinson's disease is a common disabling disease of old age affecting about one percent of the population over the age of 60 in the United States. The incidence of Parkinson's disease increases with age and the cumulative lifetime risk of an individual developing the disease is about 1 in 40. Symptoms include pronounced tremor of the extremities, bradykinesia, rigidity and postural change. A perceived pathophysiological cause of Parkinson's disease is progressive destruction of dopamine-producing cells in the basal ganglia which comprise the pars compartum of the substantia nigra, basal nuclei located in the brain stem. Loss of dopamineric neurons results in a relative excess of acetylcholine. See Jellinger, Post Mortem Studies in Parkinson's Disease—Is It Possible to Detect Brain Areas For Specific Symptoms?, J. Neural. Transm. 56(Supp): 1-29:1999. Parkinson's disease often begins with mild limb stiffness and infrequent tremors and progresses over a period of ten or more years to frequent tremors and memory impairment, to uncontrollable tremors and dementia.

Tardive Dyskinesia (TD) is a chronic disorder of the nervous system, characterized by involuntary, irregular rhythmic movements of the mouth, tongue, and facial muscles. The upper extremities also may be involved. These movements may be accompanied, to a variable extent, by other involuntary movements and movement disorders. These include rocking, writhing, or twisting movements of the trunk (tardive dystonia), forcible eye closure (tardive blepharospasm), an irresistible impulse to move continually (tardive akathisia), jerking movements of the neck (tardive spasmodic torticollis), and disrupted respiratory movements (respiratory dyskinesia). The vast majority of TD cases are caused by the prolonged use of antipsychotic drugs (neuroleptics). A relatively small number are caused by the use of other medications, such as metoclopramide, that, like neuroleptics, block dopamine receptors. TD often manifests or worsens in severity after neuroleptic drug therapy is discontinued. Resumption of neuroleptic therapy will temporarily suppress the involuntary movements, but may aggravate them in the long run.

TD affects approximately 15-20% of patients treated with neuroleptic drugs (Khot et al., Neuroleptics and Classic Tardive Dyskinesia, in Lang A E, Weiner W J (eds.): Drug Induced Movement Disorders, Futura Publishing Co., 1992, pp 121-166). Therefore, the condition affects hundreds of thousands of people in the United States alone. The cumulative incidence of TD is substantially higher in women, in older people, and in those being treated with neuroleptics for conditions other than schizophrenia, such as bipolar disorder (manic-depressive illness) (see, e.g., Hayashi et al., Clin. Neuropharmacol. 19: 390, 1996; Jeste et al., Arch. Gen. Psychiatry 52: 756, 1995). Unlike the acute motor side effects of neuroleptic drugs, TD does not respond in general to antiparkinson drugs (Decker et al., New Eng. J. Med. Oct. 7, p. 861, 1971).

Focal Dystonias (FD) are a class of related movement disorders involving the intermittent sustained contraction of a group of muscles. The prevalence of focal dystonias in one US county was estimated as 287 per million (Monroe County Study); this suggests that at least 70,000 people are affected in the US alone. The spasms of focal dystonia can last many seconds at a time, causing major disruption of the function of the affected area. Some of the focal dystonias are precipitated by repetitive movements; writer's cramp is the best known example. Focal dystonia can involve the face (e.g., blepharospasm, mandibular dystonia), the neck (torticollis), the limbs (e.g., writer's cramp), or the trunk. Dystonia can occur spontaneously or can be precipitated by exposure to neuroleptic drugs and other dopamine receptor blockers (tardive dystonia). No systemic drug therapy is generally effective, but some drugs give partial relief to some patients. Those most often prescribed are anticholinergics, baclofen, benzodiazepines, and dopamine agonists and antagonists. The most consistently effective treatment is the injection of botulinum toxin into affected muscles.

The various focal dystonias tend to respond to the same drugs (Chen, Clin. Orthop. June 102-6, 1998; Esper et al., Term. Med. 90: 18-20, 1997; De Mattos et al., Arq Neuropsiquiatr 54: 30-6, 1996). This suggests that a new treatment helpful for one focal dystonia would be likely to be helpful for another. Furthermore, the common symptoms, signs, and responses to medication of spontaneous (idiopathic) dystonia and neuroleptic-induced dystonia suggest that an effective treatment for a drug-induced focal dystonia will be effective for the same dystonia occurring spontaneously.

A tic is an abrupt repetitive movement, gesture, or utterance that often mimics a normal type of behavior. Motor tics include movements such as eye blinking, head jerks or shoulder shrugs, but can vary to more complex purposive-appearing behaviors such as facial expressions of emotion or meaningful gestures of the arms and head. In extreme cases, the movement can be obscene (copropraxia) or self-injurious. Phonic or vocal tics range from throat clearing sounds to complex vocalizations and speech, sometimes with coprolalia (obscene speech) (Leckman et al., supra). Tics are irregular in time, though consistent regarding the muscle groups involved. Characteristically, they can be suppressed for a short time by voluntary effort.

Tics are estimated to affect 1% to 13% of boys and 1% to 11% of girls, the male-female ratio being less than 2 to 1. Approximately 5% of children between the ages of 7 and 11 years are affected with tic behavior (Leckman et al., Neuropsychiatry of the Bas. Gang 20(4): 839-861, 1997). The estimated prevalence of multiple tics with vocalization, e.g., Tourette's syndrome, varies among different reports, ranging from 5 per 10,000 to 5 per 1,000.

Gilles de la Tourette syndrome (TS) is the most severe tic disorder. Tourette's syndrome is 34 times more common in boys than girls and 10 times more common in children and adolescents than in adults (Leckman et al., Neuropsychiatry of the Bas. Gang 20(4): 839-861, 1997; Esper et al., Tenn. Med. 90: 18-20, 1997). Patients with TS have multiple tics, including at least one vocal (phonic) tic. TS becomes apparent in early childhood with the presentation of simple motor tics, for example, eye blinking or head jerks. Initially, tics may come and go, but in time tics become persistent and severe, and begin to have adverse effects on the child and the child's family. Phonic tics manifest, on average, 1 to 2 years after the onset of motor tics. By the age of 10, most children have developed an awareness of the premonitory urges that frequently precede a tic. Such premonitions may enable the individual to voluntary suppress the tic, yet premonition unfortunately adds to the discomfort associated with having the disorder. By late adolescence/early adulthood, tic disorders can improve significantly in certain individuals. However, adults who continue to suffer from tics often have particularly severe and debilitating symptoms. (Leckman et al., Neuropsychiatry of the Bas. Gang 20(4): 839-861, 1997).

Although the present day pharmacopeia offers a variety of agents to treat movement disorders, none of these agents can prevent or cure these conditions. Many treatments focus on eliminating or at least alleviating certain symptoms of the disorder. Furthermore, the most effective treatments are often associated with intolerable side effects. There remains a clear-cut need for new treatments for movement disorders that have greater efficacy and fewer side effects than those currently available.

For example, Parkinson's disease (PD) is associated with the depletion of dopamine from cells in the corpus striatum. Since dopamine can't cross the blood brain barrier (BBB), it is ineffective in the treatment of Parkinson's disease. Levodopa, a metabolic precursor of dopamine, readily crosses the BBB, and is metabolically transformed to dopamine by the aromatic L-amino acid decarboxylase enzyme. This enzyme is found throughout the body including gastric juices and the mucosa of the intestine. Thus, treatment with levodopa alone requires administration of large doses of the drug due to extracerebral metabolism by this enzyme. The resulting high concentration of extracerebral dopamine causes nausea in some patients. To overcome this problem, levodopa is usually administered with an inhibitor of the aromatic L-amino acid decarboxylase enzyme such as carbidopa, which cannot itself cross the blood brain barrier and has no effect on the metabolism of levodopa in the brain. The levodopa/carbidopa therapy is considered to be the most effective treatment for symptoms of Parkinson's disease (The Medical Letter 35: 31-34, 1993). Nevertheless, certain limitations become apparent within two to five years of initiating combination therapy. As the disease progresses, the benefit from each dose becomes shorter (“the wearing off effect”), and some patients fluctuate unpredictably between mobility and immobility (“the on-off effect”). “On” periods are usually associated with high plasma levodopa concentrations and often include abnormal involuntary movements, i.e., dyskinesias. “Off” periods have been correlated with low plasma levodopa and bradykinetic episodes.

A second problem for the multiple dose regimen is that the “peak and trough” blood levels produced by multiple daily doses result in fluctuating stimulation of the dopaminergic neurons. These fluctuations may contribute to the pathogenesis of the motor complications in Parkinson disease. For example, commonly occurring adverse effects associated with MIRAPEX® (a marketed PD drug) include nausea, vomiting/emesis, weakness, dizziness, fainting, agitation, confusion, hallucinations, muscle twitching, uncontrollable movements, a tingling sensation, chest pain, insomnia, somnolence, decreased appetite, dry mouth, sweating, headache, constipation and gastric intestinal complications.

Therefore, there is a need to develop new and improved dosage forms to treat various movement disorders, such as using levodopa/carbidopa in alleviating at least one adverse effect associated with the treatment of Parkinson's disease.

### SUMMARY OF THE INVENTION

The present invention is directed to dosage forms that allow drug to be released in a highly controlled, time-dependent manner.

In general, any of the subject dosage forms and/or delivery devices (such as those described in the figures) may be used to deliver any of a large spectrum of compounds (e.g., drugs, prodrugs, metabolic precursors, etc.), especially those with limited absorption windows in upper GI (e.g., stomach).

An exemplary list of compounds that can be delivered using the subject dosage forms and/or delivery devices includes, but not limited to: metformin, acyclovir, ranitidine, riboflavin, chlorthiazide, gabapentin, losartin potassium, ganciclovir, cimetidine, minocycline, fexofenadine, bupropion, orlistat, captopril, diphenhydramine, tripelennamine, chlorpheniramine maleate, promethazine, omeprazole, prostaglandin, carbenoxolane, sucralphate, isosorbide, quinidine, enalapril, nifedipine, verapamil, diltiazem, nadolol, timolol, pindolol, salbutamol, terbutaline, carbuterol, broxaterol, aminophylline, cyclizine, cinnarizine, domperidone, alizapride, vincristine, megestrol acetate, daunorubicin, actinomycin, adriamycin, etoposide, 5-fluorouracil, indomethacin, sulindac, piroxicam, ibuprofen, naproxen, ketoprofen, temazepam, lorazepam, flunitrazepam, amantadine, ampicillin, amoxicillin, erythromycin, tetracyclines, cyanocobalamin, amino acids, iron or calcium salts of essential trace elements, or pharmacologically acceptable salts of the above.

One aspect of the invention relates in general to any drug that may be used to treat Parkinson's disease (or other movement disorders), especially levodopa/carbidopa therapy using the subject dosage forms and delivery devices.

Preferably, the drugs or prodrugs are released at a rate that results in reduction in the frequency or severity of at least one adverse effect associated with levodopa/carbidopa therapy.

In one embodiment, the dosage form releases levodopa and carbidopa at a rate that results in reduction in the frequency or severity of at least one adverse event associated with current levodopa/carbidopa therapies, or allows for a more convenient dosing regimen than current therapies.

As used herein, wherever reference to an effective composition (e.g., “levodopa” or “carbidopa”) is made, it should be understood that the effective composition may include drug and/or prodrug. In other words, any drug may be replaced in whole or in part by its prodrug(s), metabolic precursor(s), or analog(s) that provides the same therapeutic effect.

Thus, generally, one aspect of the invention provides a single dosage formulation of a pharmaceutical composition for treatment of a movement disorder. The single dosage formulation comprises levodopa and/or a metabolic precursor thereof, and optionally a decarboxylase enzyme inhibitor, wherein the dosage formulation produces and maintains a therapeutically effective concentration of levodopa thereof over a period of at least about 6 hours, 7 hours, 8 hours, 10 hours, 12 hours, 14 hours, 16 hours, 18 hours, 20 hours or more.

In one embodiment, the pharmaceutical composition comprises: (1) a first immediate-release (IR) portion comprising levodopa or a metabolic precursor thereof, formulated to provide a therapeutically effective concentration of levodopa or the precursor in the patient within about 2 hours of administration to the patient (e.g., less than about 1 minute, 5 min., 10 min., 15 min., 20 min., 30 min., 45 min., 1 hour, 1.5 hours, 2 hours, etc., or within a range of time bounded by any of these time periods, e.g., 1 min. to 2 hours, 5 min. to 1 hour, 15 to 20 min., etc.) of administration to the patient; (2) a second substantially zero order release portion comprising levodopa or a metabolic precursor thereof, formulated to release levodopa or the precursor at a substantially zero-order release rate over a sustained treatment period to maintain the therapeutically effective concentration of levodopa or the precursor in the patient; wherein at least the first IR portion further comprises a decarboxylase enzyme inhibitor, and the ratio of the inhibitor to levodopa or the precursor in at least the first IR portion is greater than 1:4.

In certain embodiments, the first immediate-release (IR) portion reaches half-maximum dissolution (e.g., when 50% of the effective composition is released) within about 1 hour, 30 minutes, 15 minutes, 10 minutes, or 5 minutes or less.

In certain embodiments, the pharmaceutical composition further comprises: (3) a substantially ascending release portion comprising levodopa or a metabolic precursor thereof, formulated to effect a rapid drop of levodopa concentration in the patient to below the therapeutically effective concentration at the end of the treatment period.

In certain embodiments, the pharmaceutical composition further comprises: (4) a substantially elevating release portion comprising levodopa or a metabolic precursor thereof, formulated to elevate the substantially zero-order release rate to a higher level beginning around a predetermined time point, such as about 3-7, 4-7, or 4-6 hours after administration to the patient.

In certain embodiments, at the end of the treatment period, the substantially elevating release portion is optionally formulated to effect a rapid drop of levodopa concentration in the patient to below the therapeutically effective concentration. This may be effected by, for example, using a similar second immediate-release portion described above.

In certain embodiments, the substantially elevating release portion comprises a decarboxylase enzyme inhibitor.

In certain embodiments, the substantially elevating release portion comprises levodopa or the precursor.

As used herein, “substantially ascending release portion” is an optional portion of the subject single dosage formulation pharmaceutical composition, which release profile resembles a peak, e.g., having sloping ascending and descending slopes with substantially no intervening plateau at which the release is maintained at a substantially constant rate. The ascending and descending slopes of the peak may be, but need not be asymmetrical. In certain embodiments, the ascending slope may be quite steep, e.g., the substantially ascending release portion may be a second immediate release portion, e.g., as a delayed-release immediate release portion. In other embodiments, the substantially ascending release portion may adopt a substantially milder ascending slope than that of the first immediate release portion, e.g., as a delayed-release controlled-release portion.

As used herein, “substantially elevating release portion” is an optional portion of the subject single dosage formulation pharmaceutical composition, which release profile resembles a plateau, e.g., having an ascending slope, a relatively flat plateau, and a descending slope. The plateau is “elevated,” in that the relative constant level of levodopa is higher than the previous substantially zero-order release rate. The ascending and descending slopes of the peak may be, but need not be, asymmetrical. In certain embodiments, the ascending slope may be quite steep, e.g., the substantially ascending release portion may be a second immediate release portion, e.g., as a delayed-release immediate release portion. In other embodiments, the substantially ascending release portion may adopt a substantially milder ascending slope than that of the first immediate release portion, e.g., as a delayed-release controlled-release portion.

As used herein, “rapid” refers to a time period no more than about 3 hours, 2 hours, 1.5 hours, 1 hour, 30 minutes, 20 minutes, 15 minutes, 10 minutes, 5 minutes, or less.

In certain embodiments, the various portions of the composition may include multiple drugs, such as carbidopa and levodopa, and their respective prodrugs. The relative proportions of the different drugs or prodrugs may vary at boundaries between the various portions (i.e., different portions may have uniform drug ratios that differ from neighboring components), or may be formulated to change gradually over one or more portions of the composition.

In certain embodiments, the ratio of drug to prodrug (e.g., carbidopa v. carbidopa prodrug; levodopa v. levodopa prodrug, etc.) may vary, depending on one or more factors such as relative solubility or other pharmacokinetic properties (e.g., absorption, distribution, metabolism and excretion, etc.).

Another aspect of the invention provides a pharmaceutical composition for the treatment of a patient suffering from Parkinson's disease and/or another movement disorder, comprising: (1) a first immediate-release (IR) portion comprising levodopa or a metabolic precursor thereof, formulated to provide a therapeutically effective concentration of levodopa in the patient within about 2 hours of administration to the patient; (2) a second substantially zero order release portion comprising levodopa or a metabolic precursor thereof, formulated to release levodopa or the precursor at a substantially zero-order release rate over a sustained treatment period to maintain the therapeutically effective concentration of levodopa in the patient; and (3) a substantially ascending release portion comprising levodopa or a metabolic precursor thereof, formulated to effect a rapid drop of levodopa concentration in the patient to below the therapeutically effective concentration at the end of the treatment period.

Another aspect of the invention provides a pharmaceutical composition for the treatment of a patient suffering from Parkinson's disease and/or another movement disorder, comprising: (1) a first immediate-release (IR) portion comprising levodopa or a metabolic precursor thereof, formulated to provide a therapeutically effective concentration of levodopa in the patient within about 2 hours of administration to the patient; (2) a second substantially zero order release portion comprising levodopa or a metabolic precursor thereof, formulated to release levodopa or the precursor at a substantially zero-order release rate over a sustained treatment period to maintain the therapeutically effective concentration of levodopa in the patient; and (3) a substantially elevating release portion comprising levodopa or a metabolic precursor thereof, formulated to elevate the substantially zero-order release rate to a higher level beginning at a predetermined time point.

Another aspect of the invention provides a pharmaceutical composition for the treatment of a patient suffering from Parkinson's disease and/or another movement disorder, comprising: (1) a sleep-inducing agent; and, (2) a decarboxylase enzyme inhibitor formulated to provide an effective plasma concentration at a predetermined time after the administration of the pharmaceutical composition to the patient.

In certain embodiments, the sleep-inducing agent is benzodiazepine (e.g., LIBRIUM®, VALIUM®), a prescription sleeping aid medicine (e.g., AMBIEN®, RESTORIL®, DESYREL®, and SONATA®), eszopiclone (e.g., LUNESTA™), or a non-prescription (over-the-counter) sleeping aid medicine (e.g., TYLENOL® PM, EXCEDRIN PM®, UNISOM®/NYTOL®/SLEEPINAL®).

In certain embodiments, the pharmaceutical composition is for administration to a patient before sleeping.

In certain embodiments, the pharmaceutical composition is for administration to a patient before sleeping, and the beginning of the delayed immediate release is calculated to begin just prior to the waking of the patient.

In certain embodiments, the pharmaceutical composition further comprises: (3) a first delayed immediate-release (DIR) portion comprising levodopa or a metabolic precursor thereof, formulated to provide a therapeutically effective concentration of levodopa in the patient within about 2 hours of the predetermined time.

In certain embodiments, the pharmaceutical composition further comprises: (4) a second delayed controlled release (DCR) portion comprising levodopa or a metabolic precursor thereof, formulated to release levodopa or the precursor at a substantially zero-order release rate over a sustained treatment period after the predetermined time, to maintain the therapeutically effective concentration of levodopa in the patient.

In certain embodiments, the predetermined time after administration is 6 to 9 hours after administration.

In certain embodiments, the rapid drop of levodopa takes place in less than two hours.

In certain embodiments, the formulation further comprises one or more of a dopamine precursor, such as L-dopa; a dopaminergic agent, such as Levodopa-carbidopa (SINEMET®, SINEMET CR®) or Levodopa-benserazide (PROLOPA®, MADOPAR®, MADOPAR HBS®); a dopaminergic and anti-cholinergic agent, such as amantadine (SYMMETRYL®, SYMADINE®); an anti-cholinergic agent, such as trihexyphenidyl (ARTANE®), benztropine (COGENTIN®), ethoproprazine (PARSITAN®), or procyclidine (KEMADRIN®); a dopamine agonist, such as apomorphine, bromocriptine (PARLODEL®), cabergoline (DOSTINEX®), lisuride (DOPERGINE®), pergolide (PERMAX®), pramipexole (MIRAPEX®), or ropinirole (REQUIP®); a MAO-B (monoamine oxidase B) inhibitor, such as selegiline or deprenyl (ATAPRYL®, CARBEX®, ELDEPRYL®); a COMT (catechol O-methyltransferase) inhibitor such as CGP-28014, entacapone (COMTAN®), or tolcapone (TASMAR®); a muscle relaxant, such as baclofen (LIORESAL®); a sedative, such as Clonazepam (RIVOTRIL®); an anticonvulsant agent, such as carbamazepine (TEGRETOL®); a dopamine reuptake inhibitor, such as tetrabenazine (NITOMAN®); a dopamine blocker, such as haloperidol (HALDOL®); a β-blocker, such as propranolol (INDERAL®, INDERAL-LA®); a carbonic anhydrase inhibitor, such as acetalzolamide (DIAMOX®) or methazolamide (NEPTAZANE®); a narcotic agent, such as codeine (TYLENOL # 3®); a GABAergic agent, such as gabapentin (NEURONTIN®); or an alpha antagonist, such as clonidine (CATAPRESS®).

In certain embodiments, the formulation further comprises a stool softener selected from: bran or psyllium (e.g., Metamucil, Fiberall), methylcellulose (e.g., Citrucel), polycarbophil, docusate (e.g., Colace, Surfak), docusate sodium and casanthranol combination (e.g., Peri-Colace, Diocto C, Silace-C), magnesium hydroxide (e.g., Phillips' Milk of Magnesia), magnesium citrate, sorbitol, polyethylene glycol solution (e.g., MiraLax), lactulose (e.g., Cephulac, Cholac, Constilac), lubiprostone (e.g., Amitiza) or other osmotic or stimulant laxatives (e.g., Bisacodyl, Cascara, Castor oil, Senna, Tegaserod/Zelnorm), and natural stool softeners.

Alternatively, the stool softener(s) may be separately administered. For example, the stool softener(s) may be administered at a time when the effective compositions of first IR portion, the second substantially zero order release portion, or the substantially ascending release portion (such as the second IR portion) are being released.

Particular such embodiments provide a pharmaceutical composition for the treatment of a patient suffering from Parkinson's disease and/or another movement disorder, comprising: (1) a first immediate-release (IR) portion comprising levodopa or a metabolic precursor thereof, formulated to provide a therapeutically effective concentration of levodopa or the precursor in the patient in less than about 2 hours of administration to the patient; and, (2) a second substantially zero order release portion comprising levodopa or the precursor, formulated to release levodopa or the precursor at a substantially zero-order release rate over a sustained treatment period to maintain the therapeutically effective concentration of levodopa or the precursor in the patient; wherein at least the first IR portion further comprises a decarboxylase enzyme inhibitor, and the ratio of the inhibitor to levodopa or the precursor in at least the first IR portion is greater than 1:4.

In certain embodiments, the ratio of the decarboxylase inhibitor to levodopa or its precursor in the first IR portion is about 1:3, 1:2, 1:1, 2:1, 3:1, 4:1, 5:1, or greater. The ratio may be different in different portions or sub-portions. For example, in the second sustained release portion, two or more sub-portions may be present, each having a different inhibitor/levodopa ratio. In certain embodiments, the ratio for the sub-portions to be released first (earlier) may be higher than that for the sub-portions to be released last (later).

In addition, the carbidopa and levodopa compositions may be released at different rates from different portions or sub-portions. For example, although the carbidopa/levodopa ratio may be consistently 1:4 in all subportions of the second substantially zero order release portion, the earlier sub-portions may release carbidopa faster, such that the release carbidopa/levodopa ratio is more than 1:4. Thus “release ratio” is defined as the ratio of two substances (e.g., carbidopa and levodopa) released at a given time point.

In certain embodiments, the pharmaceutical composition further comprises: (3) a substantially ascending release portion (such as the second IR portion) comprising levodopa or the precursor, formulated to effect a rapid drop of levodopa concentration in the patient to below the therapeutically effective concentration at the end of the treatment period.

In a related embodiment, the invention provides a pharmaceutical composition for the treatment of a patient suffering from Parkinson's disease and/or another movement disorder, comprising: (1) a first immediate-release (IR) portion comprising levodopa or a metabolic precursor thereof, formulated to provide a therapeutically effective concentration of levodopa or the precursor in the patient within about 2 hours of administration to the patient; (2) a second substantially zero order release portion comprising levodopa or the precursor, formulated to release levodopa or the precursor at a substantially zero-order release rate over a sustained treatment period to maintain the therapeutically effective concentration of levodopa or the precursor in the patient; and (3) a substantially ascending release portion comprising levodopa or the precursor, formulated to effect a rapid drop of levodopa concentration in the patient to below the therapeutically effective concentration at the end of the treatment period.

In certain embodiments, the levodopa concentration in the patient drops below the therapeutically effective concentration at the end of the treatment period within about 2 hours, e.g., within about 45 minutes, 1 hour, 1.5 hours, or 2 hours after the start of the substantially ascending release portion.

In certain embodiments, the levodopa precursor may be a methyl, ethyl, or propyl ester of levodopa, or a combination thereof. In certain embodiments, the levodopa precursor may be (−)-L-α-amino-β-(3,4-dihydroxybenzene)propanoic acid, 3-hydroxy-L-tyrosine ethyl ester, phenylglycine, or a mixture thereof.

In certain embodiments, the ratio of the decarboxylase inhibitor to levodopa or the precursor in the first IR portion is about 1:3 or greater.

In certain embodiments, one or more of the various portions may comprise one or more additional drugs, such as the ones listed above.

In certain embodiments, the release ratio of the decarboxylase inhibitor to levodopa and/or its precursor varies between the start and the end of dispensing the second substantially zero order release portion.

In certain embodiments, the ratio changes substantially continuously over the release period of the second substantially zero order release portion.

In certain embodiments, the ratio is substantially constant during all or a part of the release period of the second substantially zero order release portion.

In certain embodiments, the substantially ascending release portion (such as the second IR portion) comprises a decarboxylase enzyme inhibitor.

In certain such embodiments, the ratio of the inhibitor to levodopa and/or the precursor in the second IR portion is less than 1:4, such as 1:6, 1:8, 1:10, 1:15, 1:20, or less.

In certain embodiments, decarboxylase enzyme inhibitor is carbidopa, a carbidopa prodrug, benserazide, methylphenidate, or a combination thereof.

Unlike levodopa, carbidopa and benserazide are not pharmacologically/pharmacodynamically active, and they both have excellent toxicological profiles.

In certain embodiments, the total dose of the decarboxylase enzyme inhibitor per day per human patient is in the range of about 75-600 mg, or in the range of about 100-500 mg, or in the range of about 100-400 mg.

In certain embodiments, the total dose of levodopa and/or metabolic precursor thereof per day per human patient is between about 50 mg and about 300 mg.

In certain embodiments, at least one of the first IR portion, the second substantially zero order release portion, and the substantially ascending release portion (such as the second IR portion) further comprises at least one dopamine transport inhibitor, preferably in sufficient amount to decrease dopamine elimination.

In certain embodiments, the dopamine transport inhibitor is methylphenidate.

In certain embodiments, the dopamine transport inhibitor is present in an amount of about 3 mg to about 60 mg.

In certain embodiments, the dopamine transport inhibitor is released starting after a delay of about 2 hours to about 7 hours.

In certain embodiments, the dopamine transport inhibitor is released over a period of time of about 1 hour to about 6 hours.

In certain embodiments, the first IR portion, the second substantially zero order release portion, and the substantially ascending release portion (such as the second IR portion) (if present), are formulated to provide a sustained dose over at least 4 hours, 6 hours, 7 hours, 8 hours, 12 hours, 16 hours, 20 hours, or 24 hours when administered to the patient.

In certain embodiments, the first IR portion, the second substantially zero order release portion, and the substantially ascending release portion (such as the second IR portion) (if present), are formulated into a stack of compressed inserts encased inside a shell or coating, each portion having an independent dissolution profile, wherein drug is released only from an exposed surface at a predetermined face of the stack, e.g., through an opening at one end of the shell (e.g., the multilayered tablet).

In certain embodiments, some or all of the beads are coated by a dispersion-promoting coating, e.g., exterior to a bioadhesive coating.

In certain embodiments, the beads are less than about 1 mm in diameter.

In certain embodiments, the beads are dispersed in a matrix that disintegrates in less than about 5 minutes, 4 min., 3 min., 2 min., or less than 1 min.

In certain embodiments, the beads are dispersed in an eroding tablet that gradually erodes over the treatment period.

In certain embodiments, the tablet is at least partially coated by a bioadhesive material and/or an immediate release portion.

In certain embodiments, the bioadhesive material, if present, is exposed upon dissolution of the immediate release portion.

In certain embodiments, the shell is fully or partially coated by a bioadhesive polymeric material.

In certain embodiments, the first IR portion, the second substantially zero order release portion, and the substantially ascending release portion (such as the second IR portion) (if present), are each formulated as a one or more, preferably a plurality of, individual beads, each of the portions having an independent dissolution profile (e.g., the multiparticulate capsule).

In certain embodiments, the ratio of beads corresponding to the first IR portion, the second substantially zero order release portion, and the substantially ascending release portion (such as the second IR portion) (if present), are customized for the patient to provide a predetermined release profile, e.g., to provide a predetermined duration of release, a predetermined rate of reaching a therapeutic plasma concentration of the drug or prodrug, or a predetermined maximum release rate (e.g., customized for the size, sensitivity, or clearance rate of the particular patient), etc. For example, using more of the first IR portion may increase the rate at which a therapeutic plasma concentration is reached, using more of the second substantially zero order release portion may increase the maximum release rate and the sustained plasma concentration of the drug or prodrug, and using a different second substantially zero order release portion (or an additional sustained release portion) having additional reserves of drug or additional coatings to delay release can extend the duration of the sustained release phase.

In certain embodiments, some or all of the beads are fully or partially coated by a bioadhesive polymeric material. For example, the beads in the first IR portion may not be coated, but the beads of the second (if present) and third portions (if present), may be so coated to assist delivery of drug over an extended period of time.

In certain embodiments, at least the substantially zero-order release rate second portion is coated or partially covered by a bioadhesive polymeric material.

In certain embodiments, the bioadhesive polymeric material is selected from polyamides, polyalkylene glycols, polyalkylene oxides, polyvinyl alcohols, polyvinylpyrrolidone, polyglycolides, polyurethanes, polymers of acrylic and methacrylic esters, polylactides, poly(butyric acid), polyanhydrides, polyorthoesters, poly(fumaric) anhydride), (need to make sure these are fixed throughout specification), blends, and copolymers thereof.

In certain embodiments, the bioadhesive polymeric material is poly(fumaric-co-sebacic) anhydride.

In certain embodiments, the bioadhesive polymeric material comprises a catechol moiety. For example, the bioadhesive polymeric material may comprise a mixture of a polymeric material and a compound comprising a catechol moiety selected from L-dopa, D-dopa, dopamine, or carbidopa. In addition, the bioadhesive polymeric material may be selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric) anhydride, blends and copolymers thereof.

In certain embodiments, the bioadhesive polymeric material is covalently functionalized with a catechol moiety, such as one derived from L-dopa, D-dopa, dopamine, or carbidopa.

In certain embodiments, the pharmaceutical composition is formulated for oral administration, or for parental administration.

In certain embodiments, the pharmaceutical composition is suitable for human administration, or for veterinary treatment of a non-human mammal.

In certain embodiments, the pharmaceutical composition is provided in solid forms (e.g., powders, beads, etc.). In other embodiments, certain portions or the whole pharmaceutical composition are in liquid forms. For example, the IR portion may be in the form of a liquid, while the CR (second) portion or sub-portions may be suspended as tiny particles or beads in the liquid IR. Alternatively, an inert pharmaceutically acceptable material, carrier, or excipient may be liquid, while both the IR and the CR may be suspended as tiny particles or beads in the liquid.

In certain embodiments, at least one adverse side effect (e.g., the on-off effect or the wearing off effect, etc.) associated with the treatment of a patient suffering from Parkinson's disease and/or another movement disorder is reduced or eliminated.

In certain embodiments, the subject pharmaceutical composition provides a substantially reduced degree of fluctuation in the plasma levels of the effective ingredients (e.g., levodopa or carbidopa) compared to an immediate release pharmaceutical composition of the same dose administered three times daily.

Another aspect of the invention provides a method of making a pharmaceutical composition for the treatment of a patient suffering from Parkinson's disease and/or another movement disorder, comprising combining the first IR portion, the second substantially zero order release portion, the substantially elevating release portion (if present), and the substantially ascending release portion (such as the second IR portion) (if present), of any of the subject pharmaceutical composition into a single dosage form.

Another aspect of the invention provides a method of treating a patient suffering from Parkinson's disease and/or another movement disorder, comprising administering to the patient any of the subject pharmaceutical compositions discussed herein.

In certain embodiments, the method comprises first administering to the patient the subject pharmaceutical composition with the first IR portion and the second zero-order release portion, followed by administering the substantially ascending release portion when the previously administered pharmaceutical composition is or is about to be completed in the patient.

Another aspect of the invention provides a packaged pharmaceutical preparation comprising the subject pharmaceutical composition, in an amount sufficient to treat a patient suffering from Parkinson's disease or another movement disorder, a pharmaceutically acceptable carrier, and a label or instructions (written and/or pictorial) for the use of the formulation for treating Parkinson's disease or another movement disorder, wherein the pharmaceutical composition is formulated to provide a sustained and/or increasing dose over at least about 6, 7, 8, 10, 12, 14, 16, 18, 20, or more hours when administered to the patient.

Another aspect of the invention provides a pharmaceutical preparation comprising the subject pharmaceutical composition, provided in the form of a transdermal patch and formulated for sustained release of the pharmaceutical composition in order to administer an amount sufficient to treat a patient suffering from Parkinson's disease and/or another movement disorder, wherein the pharmaceutical composition is formulated to provide a sustained substantial zero-order release over at least about 6, 7, 8, 9, 10, 12, 14, 16, 18, 20 or more hours when the patch is applied to the patient.

Another aspect of the invention provides a single dosage formulation for treatment of a movement disorder comprising levodopa or a metabolic precursor thereof, wherein the dosage formulation produces and maintains a therapeutically effective concentration of levodopa or precursor thereof over a period of at least about 7, 8, 9, 10, 12, 14, 16, 18, 20 or more hours.

In certain embodiments, the single dosage formulation further comprises a decarboxylase enzyme inhibitor.

In certain embodiments, the single dosage formulation includes a first immediate-release (IR) portion to attain a therapeutically effective concentration of the levodopa or precursor with about 2 hours (e.g., about 1.5 hrs, 1 hour, 45 min., 30 min., 20 min., 15 min., 10 min., 5 min., 2 min., 1 min., etc.) of administration to a patient. In certain embodiments, the single dosage formulation may further comprises: (1) a sustained zero-order release portion to maintain the therapeutically effective concentration of levodopa over a first period of hours; and, (2) a substantially ascending-release portion to maintain the therapeutically effective concentration of levodopa at the end of the sustained zero-order release portion; wherein the single dosage formulation, upon administration to the patient, produces a therapeutically effective concentration of the levodopa or precursor with about 2 hours (e.g., less than about 1 minute, 5 min., 10 min., 15 min., 20 min., 30 min., 45 min., 1 hour, 1.5 hours, 2 hours, etc., or within a range of time bounded by any of these time periods, e.g., 1 min. to 2 hours, 5 min. to 1 hour, 15 to 20 min., etc.) of administration to a patient.

In certain embodiments, the ascending release portion provides for a rate of decrease of levodopa in the patient from a therapeutically effective concentration to a sub-therapeutically effective concentration (e.g., <75%, 50%, 25% or less) in a second period of time less than about 2 hours, e.g., within about 45 minutes, 1 hour, 1.5 hours, or 2 hours, e.g., to reduce sleep side effects.

In certain embodiments, the ratio of the inhibitor to levodopa or the precursor in at least the first IR portion is greater than 1:4.

In certain embodiments, the decarboxylase enzyme inhibitor is present in only the first IR portion and the sustained zero-order release portion.

In certain embodiments, the decarboxylase enzyme inhibitor is present in all the portions, wherein the ratio of decarboxylase enzyme inhibitor to levodopa is different amongst different portions, e.g., the ratio is higher in earlier-released portions than in later-released portions.

In certain embodiments, the sustained zero-order release portion comprises two or more sub-portions differing in the ratio of decarboxylase enzyme inhibitor to levodopa, e.g., the ratio is higher in earlier-released portions than in later-released portions.

In certain embodiments, at least one of the first IR portion, the second substantially zero order release portion, and the substantially ascending release portion further comprise at least one dopamine transport inhibitor, preferably in sufficient amount to decrease dopamine elimination.

In certain embodiments, the second substantially zero order release portion, and/or the substantially ascending release portion further comprise a bioadhesive polymeric material.

In certain embodiments, the bioadhesive polymeric material comprises an additive that stabilizes the polymeric material from erosion, dissolution or both, wherein at least 50% by weight of a 1 mm thick film of the bioadhesive material remains after 12 hours in a buffered pH 4.5 dissolution bath.

In certain embodiments, the bioadhesive polymeric material comprises an additive selected from one or more of a polyanhydride, an acidic component, a metal compound, a stabilizing polymer and a hydrophobic component.

Another aspect of the invention provides a single dosage formulation for treatment (e.g., once-a-day) of a movement disorder comprising levodopa or a metabolic precursor thereof, and a decarboxylase enzyme inhibitor, wherein the single dosage formulation, upon administration to the patient, produces a therapeutically effective concentration of the levodopa or precursor with about 2 hours (e.g., in less than about 1 minute, 5 min., 10 min., 15 min., 20 min., 30 min., 45 min., 1 hour, 1.5 hours, 2 hours, etc., or within a range of time bounded by any of these time periods, e.g., 1 min. to 2 hours, 5 min. to 1 hour, 15 to 20 min., etc.) of administration to a patient, the therapeutically effective concentration being maintained for a period of hours, then at the end of the dosing decreases to a sub-therapeutically effective concentration to, for example, reduce sleep side effects, in a period of time less than about 2 hours, e.g., within about 45 minutes, 1 hour, 1.5 hours, or 2 hours.

Another aspect of the invention provides a therapeutic composition as described above, except that the dosage form is coated by a layer of delayed-release coating, such that the first IR portion will not start to be released until after a pre-determined period of time, such as the normal 6-10 hours of sleep time. According to this embodiment, a dose taken by the patient at night, for example, just before sleep, would start to be released and thus become effective just before or around the time the patient wakes up in the morning. This would allow the patient to have an effective plasma concentration of levodopa or precursor thereof upon waking in the morning, and the patient can immediately participate in normal daily activities without delay.

An additional advantage of the subject formulation relates to tolerance. Specifically, with enteral infusion, patients generally develop tolerance after prolonged period of treatment. However, the subject formulation has the added benefit of having a “break” during the night, so tolerance is generally not developed.

In a related aspect, the invention provides a general method of delivering a pharmaceutical composition, comprising administering to an individual the pharmaceutical composition coated by a delayed release coating, such that the release of the effective components of the pharmaceutical composition is delayed by a predetermined period of time, e.g., at least about 2 hours, 4 hours, 6 hours, 8 hours, 10 hours, or more.

In a related aspect, the invention provide a pharmaceutical composition coated by a delayed release coating, such that upon administering the pharmaceutical composition to an individual, the release of the effective components of the pharmaceutical composition is delayed by a predetermined period of time, e.g., at least about 2 hours, 4 hours, 6 hours, 8 hours, 10 hours, or more.

In certain embodiments, the pharmaceutical composition is administered at night, such that the delayed release starts the next morning.

In certain embodiments, the pharmaceutical composition with the delayed release coating is administered with the same pharmaceutical composition without the delayed release coating, such that the individual needs only take medicine once rather than twice (or multiple times) a day.

Another aspect of the invention provides a packaged pharmaceutical composition for the treatment of a patient suffering from Parkinson's disease and/or another movement disorder, comprising: (1) a first pharmaceutical composition comprising the subject pharmaceutical composition for day time administration (e.g., need not have sleep-inducing agent); (2) a second pharmaceutical composition comprising the subject pharmaceutical composition for night time administration (e.g., comprising sleep-inducing agent).

In certain embodiments, the first and/or the second pharmaceutical composition is packaged separately as individual doses.

In certain embodiments, the package comprises at least one dose each of the first and the second pharmaceutical compositions.

In certain embodiments, the first and the second pharmaceutical compositions are distinctively marked by color, shape, and/or size.

In certain embodiments, the packaged pharmaceutical composition further comprises an instruction that instructs a patient to take the first pharmaceutical composition as a day dose, and to take the second pharmaceutical composition as a night dose.

In certain embodiments, the package comprises sufficient doses for treating a patient over a week, 2 weeks, a month, 3 months, 6 month or more.

Another aspect of the invention provides drug delivery devices, such as those described herein below (e.g., those in FIGS. 1-14 and 18-42, and those described in the Examples. Effective compositions (drugs and/or prodrugs, etc.) in these devices may be formulated to achieve any desired release profiles. In the case of levodopa/carbidopa delivery, for example, these devices may be used to achieve the subject drug release profiles, such as those depicted in FIGS. 15 and 16. Typically, it is desired that the patient's plasma levels of levodopa are within a therapeutic window, e.g., between about 680 and 3400 ng/mL, during periods of activity, e.g., during most or all waking hours. Preferably, administration of a composition of the invention does not provide a plasma level of levodopa above 3400 ng/mL at any time during the course of administration.

Thus, one aspect of the invention provides a multiparticulate pharmaceutical composition, comprising: (1) a plurality of pellets, each said pellets comprising a core comprising one or more effective ingredients; and (2) a matrix material; wherein the pellets are dispersed in the matrix material, and are released upon dissolution of the matrix material.

In certain embodiments, the matrix material disintegrates within about 5 minutes, 4 min., 3 min., 2 min., or less than 1 minute in an aqueous solution.

In certain embodiments, the aqueous solution is gastric acid.

In certain embodiments, the matrix material comprises a cushioning material.

In certain embodiments, the pharmaceutical composition is an eroding tablet and the matrix material gradually erodes over a predetermined period of time.

In certain embodiments, the eroding tablet is at least partially coated by a support material or a bioadhesive material.

In certain embodiments, the plurality of pellets comprise two or more different types of pellets.

In certain embodiments, a first type of pellets further comprises one or more coatings around the core of each pellet.

In certain embodiments, the coatings comprise a bioadhesive polymer, a composition for controlled release, and a dispersion-promoting composition.

In certain embodiments, the coatings comprise a bioadhesive polymer, a composition for controlled release, a composition for delayed release, a dispersion-promoting composition, and/or a functional or non-functional polymer.

In certain embodiments, the different coatings, if present, are in two or more discrete layers.

In certain embodiments, at least two different coatings, e.g., a bioadhesive polymeric material and a controlled-release composition, are combined in the same coating layer.

In certain embodiments, the layers comprise a controlled-release layer disposed around the core, a bioadhesive polymeric material layer disposed around the controlled-release layer, and a dispersion-promoting layer disposed around the bioadhesive polymeric material layer.

In certain embodiments, the effective ingredients comprise about 50-80% (v/v) of the coated pellets.

In certain embodiments, the effective ingredients are at least about 60% (v/v) of the coated pellets, and the effective ingredients are cohesive, plastic, and engage in hydrogen bonding.

In certain embodiments, the pellets are no more than 3 mm, 2 mm, 1 mm, 0.8 mm, 0.7 mm, 0.5 mm, 0.3 mm, or 0.1 mm in size.

In certain embodiments, the pellets are substantially homogeneous in size and/or shape.

In certain embodiments, the core is substantially free of microcrystalline cellulose.

In certain embodiments, the effective ingredient is one or more of: metformin, acyclovir, ranitidine, riboflavin, chlorthiazide, gabapentin, losartin potassium, ganciclovir, cimetidine, minocycline, fexofenadine, bupropion, orlistat, captopril, diphenhydramine, tripelennamine, chlorpheniramine maleate, promethazine, omeprazole, prostaglandin, carbenoxolane, sucralphate, isosorbide, quinidine, enalapril, nifedipine, verapamil, diltiazem, nadolol, timolol, pindolol, salbutamol, terbutaline, carbuterol, broxaterol, aminophylline, cyclizine, cinnarizine, domperidone, alizapride, vincristine, megestrol acetate, daunorubicin, actinomycine, adriamycin, etoposide, 5-fluorouracil, indomethacin, sulindac, piroxicam, ibuprofen, naproxen, ketoprofen, temazepam, lorazepam, flunitrazepam, amantadine, ampicillin, amoxicillin, erythromycin, tetracyclines, cyanocobalamin, amino acids, iron or calcium salts of essential trace elements, or pharmacologically acceptable salts thereof.

Another aspect of the invention provides method to formulate a pharmaceutical composition, comprising: (1) blending the pharmaceutical composition to form a dry mix; (2) granulating the dry mix under low shear condition with a granulation fluid to form a wet granulation; (3) extruding the wet granulation through a screen-type extruder to form extrudate; (4) spheronizing the extrudate to form spheronized pellets; and (5) drying the pellets.

In certain embodiments, the pharmaceutical composition comprises two or more effective ingredients.

Another aspect of the invention provides method to formulate a pharmaceutical composition, comprising: (1) blending the pharmaceutical composition to form a dry mix; (2) granulating the dry mix under

In certain embodiments, the eroding tablet is at least partially coated by a support material or a bioadhesive material.

Another aspect of the invention provides a pharmaceutical composition formulated by any of the subject methods.

Another aspect of the invention provides a method to formulate a pharmaceutical composition, comprising: (1) blending the pharmaceutical composition to form a dry mix; (2) granulating the dry mix under low shear condition with a granulation fluid to form a wet granulation; (3) drying the wet granulation to form dried granulation; (4) grinding the dried granulation, and sieving through a screen of predetermined size to form sieved granules; (5) blending in a lubricant to the sieved granules to form a uniformly lubricated dry mix.

In certain embodiments, the pharmaceutical composition comprises two or more effective ingredients.

In certain embodiments, the effective ingredients comprise levodopa and/or a metabolic precursor thereof, and a decarboxylase enzyme inhibitor.

In certain embodiments, the pharmaceutical composition comprises a bioadhesive polymeric material and/or a pharmaceutically acceptable excipient.

In certain embodiments, the pharmaceutical composition is substantially free of microcrystalline cellulose.

In certain embodiments, in step (1), the pharmaceutical composition is substantially free of lubricants.

In certain embodiments, the granulation fluid is purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, or a solution of a polymeric composition in a chlorinated solvent or in a ketone.

In certain embodiments, the method further comprises: passing the lubricated dry mix through a second screen.

In certain embodiments, the method further comprises: compressing the lubricated dry mix into a tablet.

In certain embodiments, the method further comprises: film-coating the tablet with one or more coating compositions.

In certain embodiments, the coating compositions comprise a bioadhesive polymeric material, a composition for controlled-release, a composition for delayed-release, a dispersion-promoting composition, and/or a functional or non-functional polymer.

In certain embodiments, the different coating compositions, if present, are in discrete layers.

In certain embodiments, at least two different coating compositions are mixed in the same coating layer.

Another aspect of the invention provides a pharmaceutical composition formulated with the subject methods.

Another aspect of the invention provides a multiparticulate pharmaceutical composition for the treatment of a patient suffering from Parkinson's disease and/or another movement disorder, comprising: (1) a first immediate-release (IR) portion comprising: (a) a plurality of pellets comprising levodopa or a metabolic precursor thereof (levodopa pellets), and (b) a plurality of pellets comprising carbidopa or a prodrug thereof (carbidopa pellets), wherein said first IR portion is formulated to provide a therapeutically effective concentration of levodopa in the patient within about 30 minutes of administration to the patient, and (2) a second portion comprising a plurality of pellets (levodopa-carbidopa pellets), each comprising: (a) a first core comprising levodopa (or a metabolic precursor thereof) and carbidopa (or a prodrug thereof); and (b) a bioadhesive polymeric material coating the first core, wherein said second portion is formulated to release levodopa at a substantially zero-order release rate over a sustained treatment period to maintain the therapeutically effective concentration of levodopa in the patient.

In certain embodiments, the w/w ratio of carbidopa:levodopa is about 1:4 in the first and second portions.

In certain embodiments, the second portion comprises about 80-90% of the levodopa in the pharmaceutical composition.

In certain embodiments, the pharmaceutical composition further comprises: (3) a third portion comprising a plurality of pellets (levodopa-bioadhesive pellets), each comprising: (a) a second core comprising levodopa (or a metabolic precursor thereof); and, (b) a bioadhesive polymeric material coating the second core, wherein the second and third portions are formulated to release levodopa at a substantially zero-order release rate over a sustained treatment period to maintain the therapeutically effective concentration of levodopa in the patient.

In certain embodiments, the second and third portions comprise about 80-90% of the levodopa in the pharmaceutical composition.

In certain embodiments, the second portion comprises about 60-70% of the levodopa in the pharmaceutical composition.

In certain embodiments, the levodopa pellets, the carbidopa pellets, the levodopa-carbidopa pellets, and the levodopa-bioadhesive pellets are all disposed in a capsule.

In certain embodiments, the levodopa pellets, the carbidopa pellets, the levodopa-carbidopa pellets, and the levodopa-bioadhesive pellets are all dispersed in a matrix material that disintegrates within about 5 minutes in an aqueous solution.

In certain embodiments, the matrix material comprises a cushioning material, e.g., for absorbing shocks and/or reducing frictions on the surface of the coated pellets.

In certain embodiments, the levodopa pellets, the carbidopa pellets, the levodopa-carbidopa pellets, and the levodopa-bioadhesive pellets are all dispersed in a matrix of an eroding tablet that gradually erodes over a predetermined period of time.

In certain embodiments, the eroding tablet is at least partially coated by a support material or a bioadhesive material.

In certain embodiments, the bioadhesive polymeric material coating the first and the second cores further comprises a dispersion-promoting agent, such as hydroxypropylcellulose.

In certain embodiments, the levodopa pellets, the carbidopa pellets, the levodopa-carbidopa pellets, and the levodopa-bioadhesive pellets (if present) are no more than about 2 mm, 1 mm, 0.8 mm, 0.7 mm, 0.5 mm, 0.3 mm, or 0.1 mm in size.

In certain embodiments, the pellets are substantially homogeneous in size and/or shape.

In certain embodiments, the pharmaceutical composition is substantially free of microcrystalline cellulose.

In certain embodiments, the bioadhesive material comprises an additive that stabilizes the material from erosion, dissolution or both, wherein at least 50% by weight of a 1 mm thick film of the bioadhesive material remains after 12 hours in a buffered pH 4.5 dissolution bath.

In certain embodiments, the bioadhesive material comprises an additive selected from one or more of a polyanhydride, an acidic component, a metal compound, a stabilizing polymer and a hydrophobic component.

Another aspect of the invention provides a multilayer tablet pharmaceutical composition for the treatment of a patient suffering from Parkinson's disease and/or another movement disorder, comprising: (1) a first controlled-release (CR) layer comprising levodopa (or a metabolic precursor thereof) and carbidopa (or a prodrug thereof), wherein the w/w ratio of carbidopa:levodopa is about 1:4 in the CR layer; (2) a second, bioadhesive layer covering at least a portion of the first CR layer; wherein the tablet is formulated to release levodopa at a substantially zero-order release rate over a sustained treatment period to maintain the therapeutically effective concentration of levodopa in the patient.

In certain embodiments, the multilayer tablet pharmaceutical composition further comprises: (3) a third, immediate-release (IR) layer comprising levodopa (or a metabolic precursor thereof) and carbidopa (or a prodrug thereof), said third layer covering at least a portion of the first CR layer and/or the second bioadhesive layer, wherein the w/w ratio of carbidopa:levodopa is about 1:4 in the third IR layer.

In certain embodiments, the CR layer comprises about 75-85%, or about 80% of the total levodopa in the composition.

In certain embodiments, the subject multilayer tablet pharmaceutical composition further comprises: (4) a fourth, pre-compressed immediate-release (IR) portion comprising levodopa (or a metabolic precursor thereof) and carbidopa (or a prodrug thereof), wherein said fourth portion is disposed within the CR layer, and wherein the w/w ratio of carbidopa:levodopa is about 1:4 in the fourth portion.

In certain embodiments, the fourth portion comprises about 15-25% of the total levodopa in the composition, and the CR layer comprises about 50-70% of the total levodopa in the composition.

Embodiments described herein are contemplated to be combined with each other embodiments as appropriate. Embodiments described in detail under one aspect of the invention may be equally applicable for the other aspects of the invention.

### BRIEF DESCRIPTION OF THE DRAWINGS

FIG. 1 is a schematic drawing (not to scale) illustrating a cross-sectional view of one design of the subject delivery device. Layers 11-13 represent the immediate-release composition layer (IR), the substantially zero-order release rate composition layer, and the optional ascending release (e.g., second IR) layer, respectively. Layer 14 is an insoluble plug that seals off one end of the open-ended container/shell 15. In this embodiment, Layers 11, 12, (and optionally 13) are exposed and released in sequential order.

FIG. 2 is a schematic drawing (not to scale) illustrating a cross-sectional view of one design of the subject delivery device. Beads 21-23 represents the immediate-release composition portion (IR), the substantially zero-order release rate composition portion, and the optional ascending release portion (such as the second IR portion), respectively. The container/shell encompassing the beads may be made from any pharmaceutically acceptable material, such as gelatin, starch, HPMC (hydroxypropyl methylcellulose), pullulan, and fast dissolving capsules. The concentric rings on the beads represent different layers of coating, each of which layers may have different compositions and/or result in different release profiles.

FIG. 3 is a schematic drawing (not to scale) illustrating a cross-sectional view of one design of the subject delivery device. The three layers represents the immediate-release composition layer (IR) 31, a bioadhesive layer (hatched lines) 32, and the substantially zero-order release rate composition layer 33. There may be one or more well-defined exit ports 34 on the bioadhesive layer to allow the inner contents to be released, or the bioadhesive layer 32 may be permeable to release of the encapsulated drug. The port size may increase in diameter over time, or when the dissolution progresses.

FIG. 4 is a schematic drawing (not to scale) illustrating a cross-sectional view of one design of the subject delivery device. It is essentially identical to that depicted in FIG. 3 (e.g., having IR layer 41, bioadhesive coating 42, zero-order release core 43, and exit port 44), with an additional (optional) core 45 inside the substantially zero-order release rate composition layer 43, which optional core 45 is the ascending release layer (such as the second IR layer).

FIG. 5 is a schematic drawing (not to scale) illustrating a cross-sectional view of one design of the subject delivery device. The three shown layers represent the immediate-release composition layer (IR) 51, and two substantially zero-order release rate composition layers (CR1 52 and CR2 53). There may be more than two such substantially zero-order release rate composition layers, differing by their specific compositions (for example, the ratio of carbidopa/levodopa in the Parkinson disease therapeutic composition). The inner trilayer core is coated with a semi-permeable coating 54a, which is then coated over by a bioadhesive layer or patch (hatched lines) 54b. The therapeutic compositions are successively released through an orifice 56 close to the IR composition (proximal end) 51. Optionally, the distal end of the shell may comprise a plug 55 that can push the therapeutic compositions towards the orifice 56 at the proximal end. The push mechanism can be any suitable means, such as a water-absorbing gel that swells when in contact with aqueous solution, or a gas-generating unit, or a rigid plate/plunger that can be driven by a micromotor (optionally externally activated).

FIG. 6 is a schematic drawing (not to scale) illustrating a cross-sectional view of one design of the subject delivery device. The three shown cores represent the immediate-release composition core (IR) 61, and two substantially zero-order release rate composition cores (CR1 62 and CR2 63), each coated by its own bioadhesive layer (hatched lines 620 and 630, respectively). There may be more than two such substantially zero-order release rate composition cores, differing by their specific compositions (for example, the ratio of carbidopa/levodopa in the Parkinson's disease therapeutic composition). All such cores are encased inside a shell 64 made from suitable materials such as gelatin.

FIG. 7 is a schematic cross-section view (not to scale) of two embodiments of a portion of the dosage form (e.g., the second zero-order release rate portion). In this specific example, the composition may be formed as a cylinder or a column, or have a trapezoid profile (right panel). The compositions (e.g., levodopa 72 and carbidopa 71) are released starting from the top face and progressing in the order shown by the arrow. The top (beginning) of the dosage form has a different carbidopa/levodopa ratio from the bottom (end) of the dosage form.

FIG. 8 is a schematic drawing (not to scale) illustrating a cross-sectional view of one design of the subject delivery device. The three shown layers represent the immediate-release composition core (IR) 81, one sub-portion of the substantially zero-order release rate composition CR1 82, and a second sub-portion of the substantially zero-order release rate composition CR2 83, in the form of beads with or without bioadhesive coating and/or delayed release coating, and a bioadhesive composition layer 84 adjacent to the zero-order release rate composition CR1 82. There can be more than one sub-portions of the substantially zero-order release rate composition embedded within layer 82 (such as CR3, CR4, etc., not shown). Each sub-portion may differ by their specific compositions (for example, the ratio of carbidopa/levodopa in the Parkinson's disease therapeutic composition). The different sub-portions may be coated with different delayed-release compositions (optionally with different thickness, etc.), and/or beads may adopt a patterned distribution within the CR1 layer, such that the beads of the same sub-portion start to release therapeutic compositions at substantially the same time. Alternatively, beads of the same sub-portion may start to release therapeutic compositions at staggered time points to effect a specific release profile, such as an ascending release profile.

FIG. 9 is a schematic drawing (not to scale) illustrating a cross-sectional view of one design of the subject delivery device. The immediate-release composition layer 91 covers the bioadhesive composition layer 94, which has a hollow core that may adopt any desired geometric shape (regular or irregular, symmetrical or asymmetrical). Once the IR layer 91 is dissolved, it exposes the peripheral ends of CR1 93. The bioadhesive layer 94 covers the inner contents, which are to be gradually released through the peripheral ends. In the shown embodiment, the center of the hollow core is occupied by a sub-portion of the zero-order release rate composition CR2 92 (or the second IR release portion). The rest of the core is filled with other sub-portion(s) of the zero-order release rate composition CR1 93. The geometric shape allows gradually increasing (as shown) or decreasing (not shown) amounts of drugs to be released in unit time periods. Each sub-portion may differ by their specific compositions (for example, the ratio of carbidopa/levodopa in the Parkinson's disease therapeutic composition). The IR layer 91 can also be part of the CR1 93 core, in form of a lip or lid (not shown).

FIG. 10 is a schematic drawing (not to scale) illustrating a cross-sectional view of one design of the subject delivery device. In this shown embodiment, the device is shaped like a torus or donut with a central hole. The immediate-release composition layer 1001 covers the entire surface, or almost the entire surface of the device. Underneath the IR layer 1001 is the bioadhesive composition layer 1004, which covers almost the entire surface except a portion of the inner surface of the donut hole. The inner core covered by the bioadhesive layer is one or more sub-portions of the zero-order release rate composition, for example, CR1 1002 and/or CR2 1003 as shown (or the second IR release portion). When the IR layer is dissolved, the inner surface of the donut hole not covered by the bioadhesive layer is exposed, creating an exit hole to allow the CR sub-portions to be released from the inner core of the device. Although shown as regularly-shaped in the figure, the CR inner cores need not be of regular and/or symmetric shape. Neither does the two or more CR sub-portions need to be horizontally arranged to effect simultaneous release. A vertical arrangement of the CR sub-portions within the inner core may be used to effect sequential release of different CR sub-portions.

FIG. 11 is a schematic drawing (not to scale) illustrating a cross-sectional view of one design of the subject delivery device. The immediate-release composition 1101 covers the two ends of a rod-shaped device (although the IR can cover the entire surface of the device, not shown). Towards the more central parts of the rod are several sub-portions of the zero-order release rate composition CR1 1102, separated from one another by other sub-portions of the zero-order release rate composition CR2 1103. The release of each CR2 1103 is delayed temporarily by a ring of bioadhesive composition 1104, and by the adjacent layers of CR1 1102. Upon the fast release of IR 1101, followed by sustained controlled release of CR1 1102, the rod may break into two or more smaller rods/parts due to the dissolution of CR1 1102 sub-portions. Depending on the spacing between adjacent CR2 1103 sub-portions, one or more CR2 1103 sub-portions may start to release from one side, or both sides of the sub-portion.

FIG. 12 is a schematic drawing (not to scale) illustrating a cross-sectional view of one design of the subject delivery device—a bioadhesive buccal patch or buccal tablet attaching to a mucosa of the mouth 1201. The immediate-release composition layer 1202 covers one sub-portion of the zero-order release rate composition CR1 1203, which covers another sub-portion of the zero-order release rate composition CR2 1204 (or the second IR release portion). The whole device may be formulated as a multilaminate bioadhesive buccal patch or tablet attaching to a mucosa area of the mouth. Either or both CR layers may have their own bioadhesive layer or patch (not shown).

FIG. 13 shows a schematic drawing (not to scale) illustrating a cross-sectional view of one design of the subject delivery device—a dose sipping system. According to this embodiment, the therapeutic compositions are deposited in a straw plugged at one end by a porous plug 1303. By dipping the plug end of the straw into a liquid (e.g., a glass of water), and applying suction through the open end of the straw 1304, the patient will receive the therapeutic composition in the solution taken through the straw. As shown, the immediate-release composition 1301 forms a matrix that contains one or more sub-portions of the substantially zero-order release rate composition CR1 and/or CR2 1302, in the form of beads with or without bioadhesive coating and/or delayed release coating. There can be more than one sub-portion of the substantially zero-order release rate composition embedded within matrix 1301 (such as CR3, CR4, etc., not shown). The sub-portions may differ by their specific compositions (for example, the ratio of carbidopa/levodopa in the Parkinson's disease therapeutic composition). If the sub-portion(s) of the substantially zero-order release rate composition (e.g., CR1 1302) are coated by bioadhesive layers, such sub-portions may adhere to the GI track and release their contents according to the designed release profile. Alternatively, the IR portion may also be formulated as beads embedded with the other CR beads within an inert matrix.

FIG. 14 shows a schematic drawing (not to scale) illustrating a cross-sectional view of one design of the subject delivery device. According to this embodiment, the therapeutic compositions are encompassed within a shell with a cap 1404 and a body 1405. The cap 1404 may be made of gelatin or other equivalent materials, while the body 1405 may be a bioadhesive layer itself, or a part of the gelatin body coated with a bioadhesive composition. Once the device is internalized by a patient and the gelatin cap 1404 is dissolved, the immediate-release composition 1401 will be exposed and quickly released. This in turn allows one or more sub-portions of the substantially zero-order release rate composition CR1 1402 and/or CR2 1403, either in the form of beads embedded within an inert matrix (not shown), with or without bioadhesive coating and/or delayed release coating, or in the form of successive layers. Although the cross-section is shown as a rectangle, it can be in any suitable shape (such as oval), and need not be symmetrical or regularly shaped.

FIG. 15 is a schematic drawing showing plasma concentration profiles of levodopa and carbidopa for an exemplary levodopa-carbidopa dosage formulation: Immediate Release-Controlled Release-Delayed/Extended Release profile.

FIG. 16 is a schematic drawing showing plasma concentration profiles of levodopa and carbidopa for an exemplary levodopa-carbidopa dosage formulation: Immediate Release-Controlled Release-Ascending Release profile.

FIG. 17 illustrates a blister packaging of an exemplary levodopa-carbidopa dosage formulation for day and night administration, e.g., during a period of one week (other packages with different treatment cycles, such as monthly package with multiple such weekly packages, are also contemplated, but not shown). The dosage forms for day and night administration can be differentiated by e.g., color, and optionally by shape, etc. The ratio of levodopa and carbidopa in the exemplary dosage formulations may be different for day and night administrations. In addition, the release rate of levodopa and carbidopa in the exemplary dosage formulations may be different for day and night administrations.

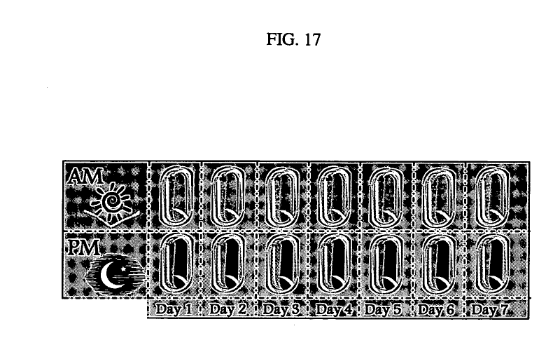

FIG. 18 is a schematic drawing (not to scale) illustrating a cross-sectional view of one design of the subject delivery device.

FIG. 19 is a schematic drawing (not to scale) illustrating a cross-sectional view of one design of the subject delivery device.

FIG. 20 is a schematic drawing (not to scale) illustrating a cross-sectional view of one design of the subject delivery device.

FIG. 21 is a schematic drawing (not to scale) illustrating a cross-sectional view of one design of the subject delivery device.

FIG. 22 is a schematic drawing (not to scale) illustrating a cross-sectional view of one design of the subject delivery device.

FIG. 23 is a schematic drawing (not to scale) illustrating a cross-sectional view of one design of the subject delivery device.

FIG. 24 is a schematic drawing (not to scale) illustrating a cross-sectional view of one design of the subject delivery device.

FIG. 25 is a schematic drawing (not to scale) illustrating a cross-sectional view of one design of the subject delivery device.

FIG. 26 is a schematic drawing (not to scale) illustrating a cross-sectional view of one design of the subject delivery device.

FIG. 27 is a schematic drawing (not to scale) illustrating a cross-sectional view of one design of the subject delivery device.

FIG. 28 is a schematic drawing (not to scale) illustrating a cross-sectional view of one design of the subject delivery device.

FIG. 29 is a schematic drawing (not to scale) illustrating a cross-sectional view of one design of the subject delivery device.

FIG. 30 is a schematic drawing (not to scale) illustrating a cross-sectional view of one design of the subject delivery device.

FIG. 31 is a schematic drawing (not to scale) illustrating a cross-sectional view of one design of the subject delivery device.

FIG. 32 is a schematic drawing (not to scale) illustrating a cross-sectional view of one design of the subject delivery device.

FIG. 33 is a schematic drawing (not to scale) illustrating a cross-sectional view of one design of the subject delivery device.

FIG. 34 is a schematic drawing (not to scale) illustrating a cross-sectional view of one design of the subject delivery device.

FIG. 35 is a schematic drawing (not to scale) illustrating a cross-sectional view of one design of the subject delivery device.

FIG. 36 is a schematic drawing (not to scale) illustrating a cross-sectional view of one design of the subject delivery device.

FIG. 37 is a schematic drawing (not to scale) illustrating a cross-sectional view of one design of the subject delivery device.

FIG. 38 is a schematic drawing (not to scale) illustrating a cross-sectional view of one design of the subject delivery device.

FIG. 39 is a schematic drawing (not to scale) illustrating a cross-sectional view of one design of the subject delivery device.

FIG. 40 is a schematic drawing (not to scale) illustrating a cross-sectional view of one design of the subject delivery device.

FIG. 41 is a schematic drawing (not to scale) illustrating a cross-sectional view of one design of the subject delivery device.

FIG. 42 is a schematic drawing (not to scale) illustrating a cross-sectional view of one design of the subject delivery device.

FIG. 43 shows in vitro dissolution profile of levodopa-carbidopa for SINEMET® 10-100 tablets in 0.1 N HCl.

FIG. 44 shows in vitro dissolution profiles of levodopa and carbidopa for SINEMET® CR 50-200 tablets in 0.1 N HCl.

FIG. 45 shows plasma concentration profiles of levodopa and carbidopa in fed beagle dogs for SINEMET® 10-100 tablets.

FIG. 46 shows plasma concentration profiles of levodopa and carbidopa in fed beagle dogs for SINEMET® CR 50-200 tablets.

FIG. 47 shows plasma concentration profiles of levodopa and carbidopa in fasted beagle dogs for SINEMET® CR 50-200 tablets.

FIG. 48 shows in vitro dissolution profiles of levodopa and carbidopa for bioadhesive levodopa-carbidopa 200 mg/50 mg trilayer tablets in 0.1 N HCl.

FIG. 49 shows plasma concentration profiles of levodopa and carbidopa in fed beagle dogs for bioadhesive levodopa-carbidopa 200 mg/50 mg trilayer tablets.

FIG. 50 shows in vitro dissolution profiles of levodopa and carbidopa for bioadhesive levodopa-carbidopa 200 mg/50 mg trilayer tablets in 0.1 N HCl.

FIG. 51 shows plasma concentration profiles of levodopa and carbidopa in fed Beagle dogs for bioadhesive levodopa-carbidopa 200 mg/50 mg trilayer tablets.

FIG. 52 shows in vitro dissolution profiles of levodopa and carbidopa for bioadhesive levodopa-carbidopa 200 mg/50 mg trilayer tablets in 0.1 N HCl.

FIG. 53 shows plasma concentration profiles of levodopa and carbidopa in fed Beagle dogs for bioadhesive levodopa-carbidopa 200 mg/50 mg trilayer tablets.

FIG. 54 shows in vitro dissolution profiles of levodopa and carbidopa for bioadhesive levodopa-carbidopa 200 mg/50 mg trilayer tablets with pre-compressed insert in 0.1 N HCl.

FIG. 55 shows plasma concentration profiles of levodopa and carbidopa in fed Beagle dogs for bioadhesive levodopa-carbidopa 200 mg/50 mg trilayer tablets with pre-compressed insert.

FIG. 56 shows in vitro dissolution profiles of levodopa and carbidopa for bioadhesive levodopa-carbidopa 200 mg/50 mg trilayer tablets with pre-compressed insert in 0.1 N HCl.

FIG. 57 shows plasma concentration profiles of levodopa and carbidopa in fed beagle dogs for bioadhesive levodopa-carbidopa 200 mg/50 mg trilayer tablets with pre-compressed insert.

FIG. 58 shows in vitro dissolution profiles of levodopa and carbidopa for levodopa-carbidopa 200 mg/50 mg triple pressed tablets in 0.1 N HCl.

FIG. 59 shows plasma concentration profiles of levodopa and carbidopa in fed beagle dogs for levodopa-carbidopa 200 mg/50 mg triple pressed tablets.

FIG. 60 shows in vitro dissolution profiles of levodopa and carbidopa for levodopa-carbidopa 200 mg/50 mg quadrilayer tablets in 0.1 N HCl.

FIG. 61 shows plasma concentration profiles of levodopa and carbidopa in fed beagle dogs for levodopa-carbidopa 200 mg/50 mg quadrilayer tablets.

FIG. 62 shows plasma concentration profiles of levodopa and carbidopa in fasted beagle dogs for levodopa-carbidopa 200 mg/50 mg quadrilayer tablets.

FIG. 63 shows in vitro dissolution profiles of levodopa and carbidopa for levodopa-carbidopa 200 mg/50 mg quadrilayer tablets in 0.1 N HCl.

FIG. 64 shows plasma concentration profiles of levodopa and carbidopa in fed beagle dogs for levodopa-carbidopa 200 mg/50 mg quadrilayer tablets.

FIG. 65 shows plasma concentration profiles of levodopa and carbidopa in fasted beagle dogs for levodopa-carbidopa 200 mg/50 mg quadrilayer tablets.

FIG. 66 shows in vitro dissolution profile of levodopa and carbidopa for levodopa-carbidopa 200 mg/50 mg bioadhesive ER pellets in phosphate buffered saline (pH 4.5).

FIG. 67 shows in vitro dissolution profile of levodopa and carbidopa for levodopa-carbidopa 200 mg/50 mg rapidly disintegrating pelletized ER tablets in phosphate buffer (pH 4.5).

FIG. 68 shows in vitro dissolution profile of levodopa and carbidopa for levodopa-carbidopa 200 mg/50 mg bioadhesive extended release pellets in phosphate buffer (pH 4.5).

FIG. 69 shows in vitro dissolution profile of levodopa and carbidopa for levodopa-carbidopa 200 mg/50 mg slow eroding pelletized ER tablets in phosphate buffer (pH 4.5).

FIG. 70 shows in vitro dissolution profiles of levodopa-cabidopa for SINEMET® 10-100 Tablets in 0.1N HCl.

FIG. 71 shows in vitro dissolution profiles of levodopa-cabidopa for SINEMET® CR 50-200 Tablets in 0.1N HCl.

FIG. 72 shows plasma concentration profiles of levodopa and carbidopa in fed beagle dogs for SINEMET® 10-100 Tablets.

FIG. 73 shows plasma concentration profiles of levodopa and carbidopa in fed beagle dogs for SINEMET® CR 50-200 Tablets.

FIG. 74 shows plasma concentration profiles of levodopa and carbidopa in fasted beagle dogs for SINEMET® CR 50-200 Tablets.

FIG. 75 shows in vitro dissolution profiles of levodopa-cabidopa for levodopa-carbidopa 200 mg/50 mg multiparticulate capsules in 0.1N HCl.

FIG. 76 shows plasma concentration profiles of levodopa and cabidopa in fed beagle dogs for levodopa-carbidopa 200 mg/50 mg capsules.

FIG. 77 shows plasma concentration profiles of levodopa and cabidopa in fasted beagle dogs for levodopa-carbidopa 200 mg/50 mg capsules.

FIG. 78 shows in vitro dissolution profile of levodopa and carbidopa for levodopa-carbidopa 200 mg/50 mg multiparticulate capsules in 0.1N HCl.

FIG. 79 shows plasma concentration profiles of levodopa and cabidopa in fed beagle dogs for levodopa-carbidopa 200 mg/50 mg capsules.

FIG. 80 shows plasma concentration profiles of levodopa and cabidopa in fasted beagle dogs for levodopa-carbidopa 200 mg/50 mg capsules.

FIG. 81 shows in vitro dissolution profile of levodopa-cabidopa for levodopa-carbidopa 200 mg/50 mg pellet capsules in 0.1N HCl.

FIG. 82 shows in vitro dissolution profile of levodopa-cabidopa for levodopa-carbidopa 200 mg/50 mg pellet capsules in 0.1N HCl and PBS-pH 4.5.

FIG. 83 shows in vitro dissolution profile levodopa-cabidopa for levodopa-carbidopa 200 mg/50 mg pellet capsules in 0.1N HCl.

FIG. 84 shows in vitro dissolution profile of levodopa-cabidopa for levodopa-carbidopa 200 mg/20 mg pellets encapsulated in gelatin and PULLULAN capsules.

FIG. 85 shows in vitro dissolution profile of levodopa for levodopa 200 mg pellets encapsulated in gelatin capsules.

FIG. 86 shows in vitro dissolution profile of levodopa for levodopa 200 mg pellets encapsulated in gelatin capsules.

FIG. 87 shows in vitro dissolution profile of levodopa for levodopa 200 mg pellets encapsulated in gelatin capsules.

FIG. 88 shows in vitro dissolution profile of levodopa for levodopa 200 mg pellets encapsulated in gelatin capsules.

FIG. 89 shows in vitro dissolution profile of carbidopa for carbidopa 200 mg pellets encapsulated in gelatin capsules.

FIG. 90 shows in vitro dissolution profile of levodopa for levodopa 200 mg pellets encapsulated in gelatin capsules.

FIG. 91 shows in vitro dissolution profile of levodopa for immediate release levodopa 200 mg pellets encapsulated in gelatin capsules.

FIG. 92 shows in vitro dissolution profile of levodopa for immediate release levodopa 200 mg pellets encapsulated in gelatin capsules.

FIG. 93 shows in vitro dissolution profiles of levodopa and carbidopa for levodopa-carbidopa 200 mg/50 mg multiparticulate capsules in 0.1 N HCl.

FIG. 94 shows plasma concentration profiles of levodopa in fed beagle dogs for SINEMET® CR 50-200 Tablets and levodopa-carbidopa 200 mg/50 mg multiparticulate capsules.

FIG. 95 shows plasma concentration profiles of carbidopa in fed beagle dogs for SINEMET® CR 50-200 Tablets and levodopa-carbidopa 200 mg/50 mg multiparticulate capsules.

FIG. 96 shows in vitro dissolution profiles of levodopa and carbidopa for SINEMET® CR 50-200 Tablets in 0.1 N HCl.

FIG. 97 provides an exemplary scheme or drug cycle regarding the release of different components of the subject single dosage formulation.

### DETAILED DESCRIPTION OF INVENTION

### I. Overview

In general, the present invention relates to the treatment of movement disorders, such as Parkinson's disease and other movement disorders. In certain embodiments, the invention relates to particular dosage forms that provide release profiles of the particular therapeutic compounds that are the most effective for the intended therapeutic use (e.g., treatment of Parkinson's disease).

In one aspect, the present invention provides a dosage form and a method for administering a movement disorder pharmaceutical composition (e.g., levodopa/carbidopa) in a once-a-day or more frequent regimen that ameliorates or overcomes symptoms of a movement disorder (e.g., Parkinson's disease) in a patient.

In one embodiment, the single dosage formulation of the subject invention comprises levodopa or a metabolic precursor thereof, and a decarboxylase enzyme inhibitor, wherein the dosage formulation produces and maintains a therapeutically effective concentration of levodopa or precursor thereof over a period of at least about 6 hours, 7 hours, 8 hours, 10 hours, 12 hours, 14 hours, 16 hours, 18 hours, 20 hours or more. For example, in one embodiment, the pharmaceutical composition of the single dosage formulation may comprise: (1) a first immediate-release (IR) portion that provides a therapeutically effective concentration of a drug in the patient with about 2 hours (e.g., about 1 minute, 5 min., 10 min., 15 min., 20 min., 30 min., 45 min., 1 hour, 1.5 hours, 2 hours, etc.) of administration to the patient; (2) a second substantially zero order release portion comprising the drug, formulated to release the drug at a substantially zero-order release rate over a predetermined sustained treatment period to maintain the therapeutically effective concentration of drug in the patient.

The subject dosage formulation is advantageous for reducing the “wearing off” and the “on off” issues. SINEMET® CR (DuPont Pharma), a controlled release dosage form was designed to provide slow and simultaneous release of levodopa and carbidopa (U.S. Pat. No. 4,900,755). In certain embodiments, the subject dosage formulations provide a longer period of levodopa release within the therapeutically effective concentration than SINEMET® does.

Certain embodiments of the invention overcome the gastric emptying problem, resulting in considerably less fluctuation in levodopa plasma levels, which in turn ameliorates the “on-off” problem.

Prolonged suppression of disease manifestations with many traditional dosage forms is constrained by the mechanism of absorption of levodopa from the gastrointestinal tract. Levodopa is absorbed by the active transport mechanism for amino acids, which is most active in the duodenum region of the small intestine. Sustained release is therefore limited by the transit time of the dosage form through the stomach and duodenum which, though highly variable from individual to individual and dependent upon nutritional state, typically takes only about 3 to 4 hours. Levodopa released after the 3-4 hour therapeutic window has passed is not bioavailable. SINEMET® CR carbidopa-levodopa controlled release tablets have about 75% of the bioavailability of SINEMET® carbidopa-levodopa conventional release tablets. Physicians Desk Reference, p. 979, (54th edition, Medical Economics Co., publisher, 2000). Mean time to peak concentration in healthy elderly subjects was found to be two hours for controlled release carbidopa-levodopa, and only 0.5 hours for the conventional form (Physicians Desk Ref., 47th Ed., p. 976, 1993).

Certain delayed release dosage forms of the invention possess a coating that dissolves slowly in gastrointestinal fluid. Release of the active component is delayed until dissolution of the coating allows gastrointestinal fluid to contact a core of the dosage form containing the drug. In combination with such coatings, the invention further provides certain bioadhesive polymer materials that help retain the pharmaceutical composition, such as one including levodopa, in the stomach of the patient. Thus, the period of release of the composition is timed to capitalize on the window of bioavailability. In other words, certain dosage forms of the invention overcome the gastric emptying problem, resulting in considerably less fluctuation in levodopa plasma levels, which in turn alleviates the “on-off” problem. This is a significant advantage for delivering drugs like levodopa that have a short absorption window.

Another advantage of certain embodiments of the invention is that a high concentration of levodopa in a patient's system, such as a “long tail” of levodopa concentration drop resulting from large doses of controlled release of levodopa/carbidopa at the end of the regimen, may be avoided by using a substantially ascending release portion, such as a second IR portion, in the dosage form released at the end of the treatment window, e.g., as the effects of carbidopa administration are waning, such that at the end of the therapeutic regimen (e.g., at the end of the day), the plasma level of levodopa quickly drops to below the effective level, so that the dosage form will not cause sleeping/resting problems for the patient.

Traditionally, in order to maintain a sufficient levodopa concentration at the end of the traditional release profile, a large dose of controlled release of levodopa/carbidopa has to be used at the end of the traditional regimen, resulting in a “long tail” of levodopa concentration drop long after the ending of the desired treatment period/cycle. This in turn causes sleeping/resting problems for the patient.

The subject release profile replaces the last segment of the traditional release profile with a last IR portion. According to the subject release profile, before the start of this last segment, effective concentration of carbidopa decreases/diminishes, allowing the body to metabolize levodopa faster and clearing it rapidly from the system. The presence of the last IR portion compensates for this more rapid processing, thus maintaining the effective levodopa concentration towards the end of the release profile. However, after the end of the desired treatment period/cycle (e.g., end of the day), levodopa in the last IR portion is quickly consumed, leaving no undesirable long tail to interfere with the sleeping/resting of the patient.

In certain embodiments, the subject pharmaceutical compositions are formulated to deliver rapidly upon administration an immediate-release (IR) dose, followed by a sustained release dose to maintain the effective therapeutic concentration, e.g., over at least 4 hours, and more preferably over at least 5, 8, 10, 12, 14, or even 16 hours after administration.

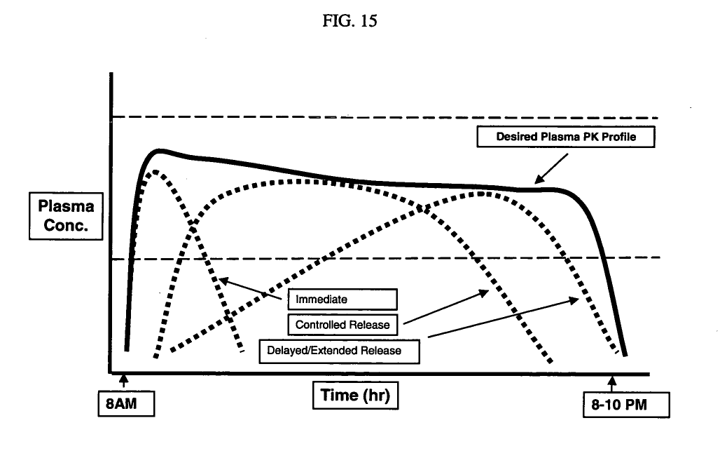

In certain embodiments, an immediate release is followed by a substantially zero-order release rate, which is optionally further followed by a substantially ascending rate of drug release, or additional immediate release. The substantially ascending rate of drug release compensates for the drop off in effective levodopa concentration in the patient's system when the second portion of substantial zero-order release reaches the end of its release profile (see FIG. 15 below; compare the tail-down of the center curve, the corresponding rise of the right-most curve around the same time, and the relative stable plateau represented by the solid curve). For example, if the patient takes the medicine upon arising in the morning, a subsequent ascending release dose (such as the second IR portion) taken separately at mealtime (e.g., dinner) would provide the patient with additional needed therapeutic agent, if necessary, without having to resort to a second full-dose of drug for the same treatment period (e.g., day). The ascending portion (such as the second IR portion) may also be built into the single dosage treatment medicine (dosage form) such that the patient need only administer treatment once a day in the morning.

FIG. 15 shows an illustrative (non-limiting) release profile of the subject dosage form. According to FIG. 15, the first IR portion (left-most sharp curve) allows a quick increase of levodopa concentration in a patient's system to within a therapeutically effective concentration range or window (the two dashed lines). This process should occur in less than about 2 hours (e.g., about 1 minute, 5 min., 10 min., 15 min., 20 min., 30 min., 45 min., 1 hr, 1.5 hrs, 2 hrs, etc., or within a range of time bounded by any of these time periods, e.g., 1 min. to 2 hours, 5 min. to 1 hour, 15 to 20 min., etc.) of administering the dosage form, depending on specific needs. As the concentration reaches its peak, or slightly before or after reaching the peak of the first IR portion release, the second sustained release portion begins to release (the middle dotted-curve in FIG. 15), such that the total levodopa concentration is maintained within the therapeutic window. Certain fluctuation in concentration is tolerated, so long as the total concentration is not too high or too low to fall outside the effective therapeutic window.

The zero-order release from the second portion is expected to maintain the total concentration within the therapeutic window for several hours, such as about 4, 5, 6, 7, 8, 9, 10 or more hours, until the net release into the patient's system is less than the net uptake/metabolism by the system including metabolic processes and other degradation processes. At that point, the total concentration of levodopa may start to drop (the point where the middle dotted-curve touching the plateau region of the thick solid curve). In the absence of an optional ascending release portion (either built in the single dosage treatment regimen or taken separately), the effective concentration will assume a long-tailed drop. But with the optional ascending release portion (either built in the single dosage treatment regimen or taken separately), the release profile can be modified. According to this embodiment, the plateau region of the solid curve is extended to a total period of beyond about 8 hours, 10 hours, 12 hours, 14 hours, 16 hours, 20 hours or more. Again, some concentration fluctuation is tolerable during this extended plateau period, so long as it falls within the effective therapeutic window.

In an alternative embodiment, the substantially ascending release portion may comprise levodopa, formulated to elevate the substantially zero-order release rate to a higher level, beginning around a predetermined time point, such as within 1/2, 1, 1.5, or 2 hours of noon (e.g., about 4-6 hours after administration to the patient).

At the end of the ascending release portion (such as the second IR portion), the (total) concentration of levodopa may optionally be allowed to drop quickly, e.g., within 30-120 minutes, such as within about 45 minutes, 1 hr, 1.5 hrs, or 2 hrs, to below a predetermined sub-therapeutically effective concentration through, for example, controlling the inhibitor/levodopa ratio.

As used herein, “sub-therapeutically effective concentration” refers to a value below the minimal effective concentration, such as less than about 75%, 50%, or about 25% of the minimal therapeutically effective concentration, which may vary depending on individual patients.

It should be noted that, although in FIG. 15 the total concentration represented by the solid line is shown as gradually declining, it need not be the case according to the instant invention. So long as the solid curve is within the therapeutic effective concentration, slight fluctuations may be tolerable. The concentration towards the end of the regimen is not necessarily lower than that towards the beginning of the regimen.

Also note that the size of the plateau represented by the flat portion of the solid curve need not be limited to 4-12 hours as shown in the illustrative FIG. 15.

In an alternative embodiment, the same therapeutic composition as described above may be coated by a layer of delayed-release coating, such that the first IR portion will not start to be released until after a pre-determined period of time, such as the normal 6-10 hours of sleep time. According to this embodiment, the medicine taken by the patient at night, for example, just before sleep, would start to be effective just before the patient wakes up in the morning. This would allow the patient to have an effective therapeutic concentration already in his system when he wakes up in the morning, and immediately participate in his normal daily activities without delay.

Thus the invention provides a pharmaceutical composition for the treatment of a patient suffering from Parkinson's disease and/or another movement disorder, comprising: (1) a sleep-inducing agent; and, (2) a decarboxylase enzyme inhibitor formulated to reach and maintain an optimal plasma concentration at a predetermined time after the administration of the pharmaceutical composition to the patient.

The sleep-inducing agent is advantageous in that it helps to ensure relatively uniform timing between administration and release of the decarboxylase enzyme inhibitor. For example, without the sleep-inducing agent, certain patients may fall sleep quickly after taking the medicine with the decarboxylase enzyme inhibitor, while others may take hours before they finally fall asleep. Assuming the same amount actual sleeping time is needed for both types of patients (e.g., 7-8 hrs), the level of decarboxylase enzyme inhibitor may reach the designed optimal level only in certain patients just before they wake up. In patients who take longer to fall asleep, the optimal level may have already passed when these patients wake up.

Using a formulation with a sleep-inducing agent, patients will awaken with an effective plasma level of decarboxylase inhibitor and can take a morning dose of levodopa and/or carbidopa (as described above) without having to wait for the decarboxylase enzyme inhibitor to reach an effective level before levodopa starts to take effect.

Alternatively, the pharmaceutical composition with sleeping-inducing agent and decarboxylase enzyme inhibitor may additionally comprise delayed-release IR and delayed CR portions. Specifically: (3) a first delayed immediate-release (DIR) portion comprising levodopa or a metabolic precursor thereof, formulated to provide a therapeutically effective concentration of levodopa in the patient within about 2 hours of the predetermined time; and (4) a second delayed controlled release (DCR) portion comprising levodopa and/or its precursor, formulated to release levodopa and/or the precursor at a substantially zero-order release rate over a sustained treatment period after the predetermined time, to maintain the therapeutically effective concentration of levodopa in the patient.

These pharmaceutical compositions, either with or without the levodopa/carbidopa compositions, are suitable for administration to a patient before sleeping. If the sleep-inducing agent and the decarboxylase enzyme inhibitor do not comprise the levodopa/carbidopa composition, the levodopa/carbidopa composition may be separately administered to the patient in the morning upon waking. In this case, the decarboxylase enzyme inhibitor is formulated to reach and maintain an optimal plasma concentration just at or just prior to the wake-up time, e.g., the predetermined time is about the average sleeping time from the administration of the sleep-inducing agent, such as 7 hrs, 8 hrs, 9 hrs, etc. In this case, the sleep-inducing agent and the decarboxylase enzyme inhibitor may be a “night pill,” while the levodopa/carbidopa extended release composition may be a “morning pill.” In PD patients, the sequence of administering the two types of pills (night pill and morning pill) is critical to achieving the optimal therapeutic effect. Both dosage forms may be packaged together, for example, as compliance promoting twin blister packages, making it convenient and clear to the patient about the order and timing of administration of the two types of doses. Optionally, the different dosage forms may be configured differently, e.g., by the appearance of the dosages themselves, such as different colors and/or different shapes, sizes, etc., or by labeling used in the packaging.

If the sleep-inducing agent/decarboxylase enzyme inhibitor formulation further comprises the levodopa/carbidopa composition, the levodopa/carbidopa composition is formulated as delayed release formulation, such that levodopa/carbidopa will start to be released just at or just prior to the patient waking up, obviating the need for a separate morning dosage form.

In certain embodiments, the sleep-inducing agent is benzodiazepine (e.g., LIBRIUM®, VALIUM®, HALCION®), Secobarbital (SECONAL®), a prescription sleeping aid medicine (e.g., AMBIEN®, RESTORIL®, DESYREL®, and SONATA®, eszopiclone (e.g., LUNESTA™), or a non-prescription (over-the-counter) sleeping aid medicine (e.g., TYLENOL® PM, EXCEDRIN PM®, UNISOM®/NYTOL®/SLEEPINAL®).

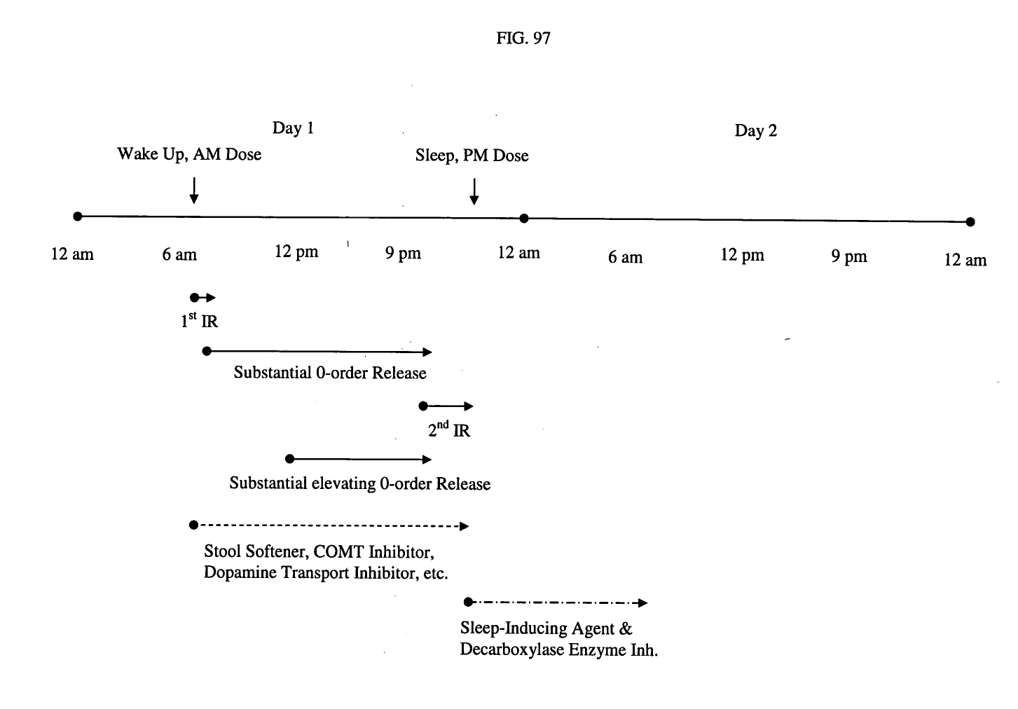

FIG. 97 provides an exemplary scheme or drug cycle regarding the release of different components of the subject single dosage formulation. Depending on the specific embodiments involved, not all components are necessarily present. The timings of release are approximate and for illustration purposes only, and may not be to scale. A typical patient for the purpose of this figure follows a routine of waking up around 7 am in the morning and going to sleep around 10-11 pm at night. Specifically, for each portion (e.g., 1^{st} IR, substantial zero-order release, substantial elevating zero-order release, 2^{nd} IR, sleep-inducing agent & decarboxylase inhibitor, etc.), the closed circle indicates the approximate start of drug release, and the arrowhead indicates the approximate end or tailing off of the release. For components not starting at about the time of administration, a delayed-release coating may be present to effect the delay. The stool softener, COMT inhibitor, and/or dopamine transport inhibitor, and other auxiliary drug components, etc., if present, may be released at any time during the drug cycle, with any effective components (1^{st} IR, substantial zero-order release, 2^{nd} IR, etc.), either simultaneously or sequentially (hence the dashed line).

In one embodiment, a patient can take one AM dose per day, and continue indefinitely if desirable. Alternatively, the patient may take one AM dose when waking up, followed by one PM dose before sleeping, and continue this pattern indefinitely if desirable. The PM dose may contain a sleep-inducing agent & a decarboxylase enzyme inhibitor. In yet another embodiment, the patient may take one PM dose per day, and continue indefinitely if desired. In this embodiment, the PM dose also contains effective components (e.g., carbidopa/levodopa) in delayed-release formulation, such that release profile similar to the left-hand side of FIG. 97 (Day 1) is achieved for Day 2.

It should be understood that such release profiles may also be used to divide the different components into multiple dosage systems (such as a sleep aid, a night-time formulation, and a morning formulation, etc.), so long as the overall formulations are designed to release the drugs at or about the times indicated on the scheme.

In certain embodiments, the subject dosage form allows rapid release of drug (e.g., levodopa) in the morning at a rate that results in rapid and reproducible onset of action, reduced frequency of administration, reduced severity of side effects (motor fluctuations). The onset of action may be effected at about 5 minutes, 10 minutes, 15 minutes after the administration, or about 30 minutes, or about 45 minutes, or about 1 hour after the administration of the pharmaceutical composition comprising an immediate-release composition.

In certain embodiments, the ratio of carbidopa (or other equivalent decarboxylase inhibitors) to levodopa is variable, between different individuals/patients, and/or between the different stages of release (e.g., immediate-release vs. substantially zero-order release vs. the optional substantially ascending/rapid rate of release), and/or within each stage of release (e.g., within the zero-order release stage).

Depending on specific situations, the carbidopa:levodopa ratio may be about 1:20, 1:15, 1:10, 1:5, 1:4, 1:3, 1:2, 1:1, 2:1, 3:1, 4:1, 5:1, or about 6:1 or more.

The usual daily therapeutic dose of carbidopa is approximately 75 mg per day, but carbidopa apparently fails to elicit adverse effects even at doses of 400 mg per day (Ahlskog, Hosp. Form., 27: 146, 1992). Thus the total daily dose of carbidopa may be anywhere below about 600 mg, 500 mg, or 400 mg.

For example, to effect a rapid rise of carbidopa concentration in a patient's system, a greater than 1:4 ratio of carbidopa:levodopa may be used (e.g., greater than 1:3, 1:2, 1:1, 2:1, etc.). This helps ensure that peripheral decarboxylase activity is substantially inhibited in the treated individual, without regard to any individual differences in peripheral decarboxylase level and/or activity. In contrast, compared to the standard 1:4 ratio, the carbidopa:levodopa ratio may be very low in the second IR portion (e.g. less than about 1:10, 1:15, 1:20, etc.), or carbidopa may be omitted from this portion altogether, such that a rapid drop in effective levodopa can be effected towards the end of regimen, allowing the treated patient to sleep or rest normally without being substantially affected by the lingering effects of levodopa therapy that can disrupt normal sleep patterns.

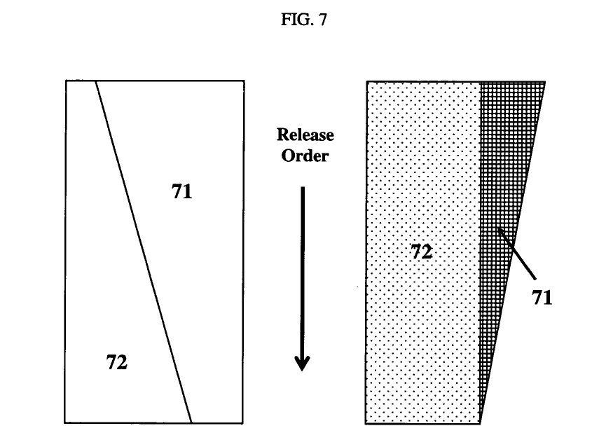

In fact, in certain embodiments, the carbidopa:levodopa ratio may vary even within a single release stage. For example, during the substantially zero-order release stage, the ratio may be closer to 1:4 when entering this stage, and gradually decrease to 1:5, 1:6, 1:8, 1:9, 1:10, etc., such that at the end of this stage, the ratio is substantially smaller than the starting ratio. This effect can be achieved using a number of approaches. For example, carbidopa and levodopa may be mixed together and spun in a container to create a gradient of inhibitor:drug. Alternatively, carbidopa and levodopa may occupy two sides of an imaginal tilted plane dissecting a cylindrical column, such that an increasing or decreasing proportion of the dissolving surface comprises the inhibitor carbidopa. In a related embodiment, the release rate remains constant for the levodopa composition, while the release rate gradually decreases for carbidopa (see FIG. 7). Yet another alternative is to stack many layers, each with a unique carbidopa:levodopa ratio, etc. Obviously, using these methods, the ratio may remain constant for any desired period or periods of time during the stage.

In other embodiments, the invention provides a bioadhesive dosage form that releases the drug at the target absorption site, and is less prone to gastric emptying, thus resulting in a more reproducible and consistent plasma level of levodopa, a drug with a narrow absorption window. In certain embodiments, the bioadhesive layer/patch is used in conjunction with the substantially zero-order release composition.

Another approach to achieving a constant dopamine level in the brain is to work at the level of brain biochemistry. Dopamine in the brain, whether released by a pre-synaptic neuron or supplied by the delivery of levodopa through the brain blood barrier, is removed from the junction by mechanisms of dopamine uptake to stop information transfer. Partial blocking of the dopamine uptake could result in more constant dopamine levels in the brain without the need to modify the levodopa profile in the blood. Methylphenidate, a relatively safe drug used to treat children suffering from Attention Deficit Disorder (ADD) or Attention Deficit Hyperactivity Disorder (ADHD), is a dopamine transport inhibitor. Methylphenidate has been used with levodopa in Parkinson's disease patients, resulting, however, in severe dyskinesia and other motor effects of levodopa on the patients, especially when the two drugs are delivered together. Camicioli, et al. “Methylphenidate Increases the Motor Effects of L-Dopa in Parkinson's Disease: a Pilot Study,” Clin. Neuropharmacol. 24(4): 208-213, 2001.

However, when dosages are properly calibrated, the detrimental results associated with the co-delivery of levodopa and at least one dopamine transport inhibitor can be avoided and beneficial results achieved. Delivery of dopamine transport inhibitors too early in the levodopa-in-blood release profile enhances the adverse motor effects caused by high levels of dopamine in the brain. However, by properly adjusting the levodopa dose and proper timing of the dopamine transport inhibitor to block the dopamine transporter, substantially constant levels of dopamine in the brain can be achieved. Preferably, the dopamine transport inhibitor is administered as the dopamine levels start to decrease.

During normal brain function, dopamine is released in the synapse by neuron cells and eliminated by transport proteins. Co-treatment with a drug that inhibits the transport protein allows the dopamine to reside longer in the brain, thereby making the effective drug troughs shallow. Additionally, the efficient use of the method lowers concentration peaks by lowering levodopa dosing levels. Proper timing of the co-treatment with the two drugs is essential. Administering the dopamine transport inhibitor too early may result in peaks of brain dopamine and problems of dyskinesia. Administering the dopamine transport inhibitor too late may result in too little advantage, because the dopamine levels in the brain will have already been depleted by the normal elimination processes. Proper timing of the transport inhibitor delivery to slightly after the predicted peak in brain dopamine concentration and keeping the inhibitor in place for some time extends the time that effective concentrations of the dopamine are present in the brain. However, extending the time of the transport inhibition too long can have deleterious effects, since it may lead to too high a dopamine concentration as additional levodopa is administered.

Thus, in certain embodiments, the invention provides dosage forms comprising levodopa and at least one dopamine transport inhibitor. The administration of the dopamine transport inhibitor may be delayed such that release coincides with the time the dopamine concentration level starts to decrease. The dopamine transport inhibitor is a compound capable of delaying the dopamine transporter from removing dopamine from the brain. In other words, the dopamine transport inhibitor precludes or diminishes the removal rate of dopamine by the dopamine transporter, thereby prolonging a concentration of dopamine in the brain. Dopamine transporter inhibitors include, but are not limited to, methylphenidate. In the formulation of the invention, methylphenidate may be present in an amount about 1 mg to about 60 mg, preferably from 1 mg to about 15 mg, more preferably, from about 5 mg to about 10 mg, and most preferably methylphenidate may be present in an amount of about 10 mg per dose.

In certain embodiments, a levodopa metabolic precursor like the levodopa ethyl ester of U.S. Pat. No. 5,840,756 (incorporated herein by reference) may be substituted for some or all of the levodopa in the various embodiments of the invention. Typically, levodopa is present in an amount from about 50 mg to about 300 mg, preferably from about 100 mg to about 200 mg and, more preferably, levodopa is present in an amount of about 100 mg to about 150 mg per dose. The amount of levodopa may also be adjusted accordingly if any of the other formulations described below are adapted for use in the instant invention.

As discussed above, the timing of the administration of the individual ingredients of the composition of the invention is important to achieve the desired leveling of peaks and troughs of dopamine concentrations when treating Parkinson's disease. Generally, it is desirable to administer levodopa and, optionally, a decarboxylase enzyme inhibitor, prior to the administration of at least one dopamine transporter inhibitor. Alternatively, the levodopa, decarboxylase enzyme inhibitor, and dopamine transporter inhibitor of the composition may be administered concurrently as a unit dose or co-administered as several doses. Each ingredient, however, may be formulated either as an immediate release formulation or sustained release formulation with or without a time delay. The ratio of each ingredient may also vary between the first (and second, if present) immediate-release and the substantially zero-order release dose.

In certain embodiments, levodopa may be administered as an immediate-release formulation or a sustained-release delivery formulation wherein the levodopa is released over about 1 to about 4 hours. The decarboxylase enzyme inhibitor may be dosed as an immediate-release drug delivery formulation or a sustained-release delivery formulation (with levodopa or independently) wherein the decarboxylase enzyme inhibitor is released over about 1 to about 4 hours. Typically, the dopamine transporter inhibitor is formulated as an immediate-release formulation which releases after about a 2-hour to about 7-hour delay, and preferably after about a 3- to about 5-hour delay. Alternatively, the dopamine transporter inhibitor may be formulated as a sustained-release delivery formulation which releases over one to six hours after about a 1- to about 7-hour delay.

In certain embodiments, the subject pharmaceutical composition is formulated for variable dosing, such as customized dosing for individual patients.

Another aspect of the invention provides a method for making the pharmaceutical compositions with one or more features as described above.

Another aspect of the invention provides a method for using the pharmaceutical compositions with one or more features as described above, in treating a movement disorder, such as Parkinson's disease.

Another aspect of the invention provides the use of a pharmaceutical composition with one or more features as described above in manufacturing medicaments for the treatment of a movement disorder, such as Parkinson's disease.

The subject preparations and methods can be used as part of the treatments for human and/or other animal subjects. In addition to humans, other animal subjects to which the invention is applicable extend to both domestic animals and livestock, raised either as laboratory animals, pets or zoo animals, or for commercial purposes. Examples are rodents such as mice, rats, hamsters, or rabbits; dogs; cats; cattle; horses; sheep; hogs; and goats.

In certain embodiments, the method includes administering, conjointly with the subject pharmaceutical composition, one or more of other therapeutic compositions useful for the treatment of diseases, for which levodopa/carbidopa or pramipexole is indicated for. For example, in the case of treating Parkinson's Disease and certain movement disorders, levodopa/carbidopa or pramipexole may be co-administered with a dopamine precursor, a dopaminergic agent, a dopaminergic and anti-cholinergic agent, an anti-cholinergic agent, a dopamine agonist, a MAO-B (monoamine oxidase B) inhibitor, a COMT (catechol O-methyltransferase) inhibitor, a muscle relaxant, a sedative, an anticonvulsant agent, a dopamine reuptake inhibitor, a dopamine blocker, a β-blocker, a carbonic anhydrase inhibitor, a narcotic agent, a GABAergic agent, or an α antagonist.

In certain embodiments, the method includes administering, conjointly with the pharmaceutical composition, one or more of physical therapy, occupational therapy, or speech/language therapy.

An agent to be administered conjointly with a subject compound may be formulated together with a subject compound as a single pharmaceutical preparation, e.g., as a pill or other medicament including both agents, or may be administered as a separate pharmaceutical preparation.

Another aspect of the invention provides a packaged pharmaceutical composition, comprising the subject pharmaceutical composition in an amount sufficient to treat or prevent a movement disorder in a patient, which may additionally include a pharmaceutically acceptable carrier, and instructions (written and/or pictorial) describing the use of the formulation for treating the patient, wherein the patient suffers from ataxia, corticobasal ganglionic degeneration (CBGD), dyskinesia, dystonia, tremors, hereditary spastic paraplegia, Huntington's disease, multiple system atrophy, myoclonus, Parkinson's disease, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, restless leg syndrome, Wilson's disease, stiff man syndrome, akinetic mutism, psychomotor retardation, painful legs moving toes syndrome, a gait disorder, a drug-induced movement disorder, or other movement disorder.

In certain preferred embodiments, the movement disorder is Parkinson's disease.

Certain general features of the invention are further elaborated in the sections below.

### II. Definitions

For convenience, certain terms employed in the specification, examples, and appended claims are collected here. All other terms have their ordinary meanings as understood by a skilled artisan.

As used herein, “about” means within the pharmaceutically acceptable limits found in the United States Pharmacopia (USP-NF 21), 2003 Annual Edition, or available at the USP website, for amount of active pharmaceutical ingredients. With respect to blood levels, “about” means within FDA acceptable guidelines.

The term “adrenergic” refers to neurotransmitters or neuromodulators chemically related to adrenaline (epinephrine) or to neurons which release such adrenergic mediators. Examples are dopamine, norepinephrine, and epinephrine. Such agents are also referred to as catecholamines, which are derived from the amino acid tyrosine.

As generally used herein “bioadhesives” or “bioadhesive materials” refer to naturally occurring polymers which are bioadhesive or naturally occurring or synthetic polymers, which have been modified or which have been blended with an additive, to have improved bioadhesion. As generally used herein, “modified” refers to monomers or polymers which have undergone a chemical reaction.

As used herein “bioadhesion” generally refers to the ability of a material to adhere to a biological surface for an extended period of time. Bioadhesion requires a contact between the bioadhesive material and a surface, for example, where the bioadhesive material penetrates into the crevice of the surface (e.g. tissue and/or mucus) and chemical bonds form. The amount of bioadhesive force is affected by both the nature of the bioadhesive material, such as a polymer, and the nature of the surrounding medium. Adhesion of materials to tissues may be achieved by (i) physical or mechanical bonds and/or (ii) secondary chemical bonds (e.g., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (e.g., Van der Waals interactions) and stronger specific interactions, which include hydrogen bonds and ionic bonds. The hydrophilic functional groups responsible for forming hydrogen bonds are hydroxyl (—OH) and carboxylic acid groups (—COOH). Bioadhesive forces are measured in units of N/m². These forces are preferably determined by methods defined in U.S. Pat. No. 6,197,346 to Mathiowitz et al. Bioadhesive forces, especially those exhibited by tablets, can also be measured using a Texture Analyser, such as the TA-TX2 Texture Analyser (Stable Micro Systems, Haslemer, Surrey, UK). As described by Michael J. Tobyn et al in Eur. J. Pharm. Biopharm., 41(4):235-241 (1995), a mucoadhesive tablet is attached to a probe on the texture analyzer and lowered until it contacts pig gastric tissue, which is attached to a tissue holder and exposed to liquid at 37° C. to simulate gastric medium. A force is applied for a set period of time and then the probe is lifted at a set rate. Area under the force/distance curve calculations are used to determine the work of adhesion. (See also Michael J. Tobyn et al., Eur. J. Pharm. Biopharm., 42(1):56-61 (1996) and David S. Jones, et al., International J. Pharmaceutics, 151: 223-233 (1997)).

The term “biogenic amines” refers to a class of neurotransmitters which includes catecholamines (e.g., dopamine, norepinephrine, and epinephrine) and serotonin.

As generally used herein “blend” refers to a mixture of two or more polymers or a mixture of one or more polymers with one or more low molecular weight additives containing a catechol functionality. The mixture can be homogeneous or heterogeneous.

As used herein “catechol” refers to a compound with a molecular formula of C₆H₆O₂ and the following structure:

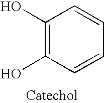

Bioadhesive materials contain a polymer with a catechol functionality or a polymer blended with catechol or a catechol derivative. For materials that contain polymers that have been modified with a catechol functionality, the molecular weight of the bioadhesive materials and percent substitution of the polymer with the aromatic compound may vary greatly. The degree of substitution varies based on the desired adhesive strength, it may be as low as 10%, 20%, 25%, 50%, or up to 100% substitution. On average at least 50% of the monomers in the polymeric backbone are substituted with at least one aromatic group. Preferably, 75-95% of the monomers in the backbone are substituted with at least one aromatic group or a side chain containing an aromatic group. In the preferred embodiment, on average 100% of the monomers in the polymeric backbone are substituted with at least one aromatic group or a side chain containing an aromatic group. The resulting bioadhesive material is a polymer with a molecular weight ranging from about 1 to 2,000 kDa, preferably 1 to 1,000 kDa, more preferably 10 to 1,000 kDa, most preferably 100 to 1,000 kDa. For materials in which a polymer has been blended with catechol or a catechol derivative, the ratio of polymer to catechol can be varied in order to vary the bioadhesive properties of the material. The catechol or catechol derivative can be present in an amount from about 0.5% to about 95% by weight of the polymer, typically about 10% to about 75%, preferably about 10% to about 50% and more preferably about 10% to about 30%.

In certain embodiments of the invention, a polymer may be functionalized by covalently attaching catechol moieties or compounds comprising catechol moieties. Alternatively, a compound comprising a catechol moiety may be blended with a polymer to form a simple mixture with no covalent association between the catechol moieties and the polymer.

The term “catecholamines” refers to neurotransmitters that have a catechol ring (e.g., a 3,4-dihydroxylated benzene ring). Examples are dopamine, norepinephrine, and epinephrine.

The term “cholinergic” refers to neurotransmitters or neuromodulators chemically related to choline or to neurons which release such cholinergic mediators.

The term “Degree of Fluctuation (DFL)” as used herein is expressed as:

DFL=(Cₘₐₓ−Cₘᵢₙ)/C_{avg}

produced by ingestion of the composition of the invention or the t.i.d comparator.

The term “Cₘₐₓ” as used herein means maximum plasma concentration of pramipexole achieved by the ingestion of the composition of the invention or the t.i.d comparator. The term “Cₘᵢₙ” as used herein means minimum plasma concentration of pramipexole achieved by the ingestion of the composition of the invention or the t.i.d comparator. The term “C_{avg}” as used herein means average plasma concentration of pramipexole achieved by the ingestion of the composition of the invention or the t.i.d comparator. C_{avg} is calculated by AUC over a 24 hours intervals divided by 24.

The term “Tₘₐₓ” as used herein means the time to achieve maximum plasma concentrations produced by ingestion of the composition of the invention or the t.i.d comparator. The term “AUC” as used herein means the area under the plasma concentration-time curve, as calculated by the trapezoidal rule over the 24 hour interval for all the formulations.

As used in this application, the term “Cₘᵢₙ” and “trough levels” should be considered synonyms. Likewise, “Cₘₐₓ” and “peak levels” should be considered synonyms.

The term “dopaminergic” refers to neurotransmitters or neuromodulators chemically related to dopamine or to neurons which release such dopaminergic mediators.

The term “dopamine” refers to an adrenergic neurotransmitter, as is known in the art.

The term “ED₅₀” means the dose of a drug which produces 50% of its maximum response or effect.

An “effective amount” of, e.g., a movement disorder pharmaceutical composition, with respect to the subject method of treatment, refers to an amount of the pharmaceutical composition in a preparation which, when applied as part of the subject dosage regimen brings about the desired correction/suppression of the movement disorder (e.g., dyskinesis and/or bradykinesis) according to clinically acceptable standards.

The term “LD₅₀” means the dose of a drug which is lethal in 50% of test subjects.

The term “lethal therapeutic index” refers to the therapeutic index of a drug defined as LD₅₀/ED₅₀.

The term “metabolites” refers to active derivatives produced upon introduction of a compound into a biological milieu, such as a patient.

The term “orally deliverable” herein means suitable for oral, including peroral and intra-oral (e.g., sublingual or buccal) administration, but tablets of the present invention are adapted primarily for peroral administration, i.e., for swallowing, typically whole or broken, with the aid of water or other drinkable fluid.

A “patient,” “individual,” or “subject” to be treated by the subject method can mean either a human or non-human animal.

The term “prevent,” “preventing,” or “prevention” as used herein means reducing the probability/risk of developing a condition in a subject (e.g., a human), or delaying the onset of a condition in the subject, or lessening the severity of one or more symptoms of a condition (e.g., a movement disorder) that may develop in the subject, or any combination thereof.

The term “prodrug” is intended to encompass compounds which, under physiologic conditions, are converted into the therapeutically active agents of the present invention. A common method for making a prodrug is to include selected moieties which are hydrolyzed under physiologic conditions to reveal the desired molecule. In other embodiments, the prodrug is converted by an enzymatic activity of the host animal.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd ed.; Wiley: New York, 1991).

The term “SeD₅₀” means the dose of a drug which is produces a particular side-effect in 50% of test subjects.

The term “side-effect therapeutic index” refers to the therapeutic index of a drug defined as SeD₅₀/ED₅₀.

A “subject” herein is an animal of any species, preferably mammalian, most preferably human. Conditions and disorders in a subject for which a particular agent is said herein to be “indicated” are not restricted to conditions and disorders for which the agent has been expressly approved by a regulatory authority, but also include other conditions and disorders known or believed by a physician to be amenable to treatment with the agent.

“Solid fraction” is the ratio of absolute to apparent density of a compact of the starch. A “compact” herein is a compressed tablet, prepared for example on a tablet press, consisting only of a sample of starch for which it is desired to measure tensile strength. A “solid fraction representative of the tablet” is a solid fraction selected to be similar to the solid fraction of tablets prepared according to the invention. Typically a solid fraction of about 0.75 to about 0.85, illustratively 0.8, will be selected.

The term “statistically significant” as used herein means that the obtained results are not likely to be due to chance fluctuations at the specified level of probability. The two most commonly specified levels of significance are 0.05 (p=0.05) and 0.01 (p=0.01). The level of significance equal to 0.05 and 0.01 means that the probability of error is 5 out of 100 and 1 out of 100, respectively.

By “transdermal patch” is meant a system capable of delivery of a drug to a patient via the skin, or any suitable external surface, including mucosal membranes, such as those found inside the mouth. Such delivery systems generally comprise a flexible backing, an adhesive and a drug retaining matrix, the backing protecting the adhesive and matrix and the adhesive holding the whole on the skin of the patient. On contact with the skin, the drug-retaining matrix delivers drug to the skin, the drug then passing through the skin into the patient's system.

The term “treat,” “treating,” or “treatment” as used herein means to counteract a medical condition (e.g., a movement disorder) to the extent that the medical condition is improved according to clinically acceptable standard(s). For example, “to treat a movement disorder” means to improve the movement disorder or relieve symptoms of the particular movement disorder in a patient, wherein the improvement and relief are evaluated with a clinically acceptable standardized test (e.g., a patient self-assessment scale) and/or an empirical test (e.g., PET scan). “Treatment” herein embraces prophylactic treatment unless the context requires otherwise.

The term “water-soluble” herein means having solubility of at least about 10 mg/ml. Unless otherwise specified, “solubility” herein means solubility in water at 20-25° C. at any physiologically acceptable pH, for example at any pH in the range of about 4 to about 8. In the case of a salt, reference herein to solubility in water pertains to the salt, not to the free base form of pramipexole.

### III. Exemplary Uses of the Dosage Forms

In various embodiments, the present invention contemplates modes of treatment and/or prophylaxis (e.g., treating or preventing the development of symptoms in high-risk populations), which utilize one or more of the subject dosage forms for decreasing or overcoming the defects in a movement disorder patient. The improvement and/or restoration of mental or physical state in an organism has positive behavioral, social, and psychological consequences.

For example, Parkinson's disease is the second most common neurodegenerative disorder, affecting nearly 1 million people in North America. The disease is characterized by symptoms such as muscle rigidity, tremor and bradykinesia. Early studies of Parkinson's disease showed unusual inclusions in the cytoplasm of neurons (i.e., Lewy bodies), occurring predominantly in the substantia nigra, which innervate the striatal region of the forebrain. Although Lewy bodies were also found in other neurodegenerative conditions, the presence of Lewy bodies in Parkinson's disease is accompanied by cell loss in the substantia nigra. This cell loss is considered to be the defining pathological feature of Parkinson's disease.

Epidemiological studies have reported geographic variation in Parkinson's disease incidence, leading to the search for environmental factors (Olanow and Tatton, Ann. Rev. Neurosci. 22: 123-144, 1998). The recent discovery that 1-methyl-4-phenyl-1,2,3,6-tetrahydropyridine (MPTP) toxin causes a Parkinson's-like syndrome indistinguishable from the idiopathic disease suggests that Parkinson's disease may be caused by environmental factors (e.g., toxins and causative agents). (See e.g., Langston, Ann. Neurol. 44: S45-S52, 1998).

Recent research has also identified genes associated with Parkinson's disease (Mizuno et al., Biomed. Pharmacother. 53(3): 109-116, 1999; Dunnett and Bjorklund, Nature 399 (6738 Suppl): A32-A39, 1999); namely, the α-synuclein gene (Polymeropouos et al., Science 276: 2045-2047, 1997), the parkin gene (Kitada et al., Nature 392: 605-608, 1998), and the UCH-L1 thiol protease gene (Leroy et al., Nature 395: 451-452, 1998). Although additional chromosomal loci associated with the disease state have been identified, these chromosomal loci have not been analyzed at the molecular level. At present, the biochemical roles played by these gene products in both normal cells and in diseased neurons remain ambiguous, and no gene therapy protocols involving their use have been developed.

Furthermore, Parkinson's disease is associated with the progressive loss of dopamine neurons in the ventral mesencephalon of the substantia nigra (Shoulson, Science 282: 1072-1074, 1998), which innervates the major motor-control center of the forebrain, the striatum. Although a gradual decline in the number of neurons and dopamine content of the basal ganglia is normally associated with increasing age, progressive dopamine loss is pronounced in people suffering from Parkinson's disease, resulting in the appearance of symptoms when about 70-80% of striatal dopamine and 50% of nigral dopamine neurons are lost (Dunnett and Bjorklund, supra). This loss of dopamine-producing neurons resulting in a dopamine deficiency is believed to be responsible for the motor symptoms of Parkinson's disease.

Although the cause of dopaminergic cell death remains unknown, it is believed that dopaminergic cell death is affected by a combination of necrotic and apoptotic cell death. Mechanisms and signals responsible for the progressive degeneration of nigral dopamine neurons in Parkinson's disease have been proposed (Olanow et al., Ann. Neurol. 44: S1-S196, 1998), and include oxidative stress (from the generation of reactive oxygen species), mitochondrial dysfunction, excitotoxicity, calcium imbalance, inflammatory changes and apoptosis as contributory and interdependent factors in Parkinson's disease neuronal cell death.

Apoptosis (i.e., programmed cell death) plays a fundamental role in the development of the nervous system (Oppenheim, Ann. Rev. Neurosci. 14: 453-501, 1991), and accelerated apoptosis is believed to underlie many neurodegenerative diseases, including Parkinson's disease (Barinaga, Science 281: 1303-1304, 1998; Mochizuki et al., J. Neurol. Sci. 137: 120-123, 1996; and Oo et al., Neuroscience 69: 893-901, 1995). In living systems, apoptotic death can be initiated by a variety of external stimuli, and the biochemical nature of the intracellular apoptosis effectors is at least partially understood.

In a further embodiment, a composition of the invention is administered in combination therapy with one or more additional drugs or prodrugs. The term “combination therapy” or “conjoint therapy” herein means a treatment regimen wherein the agent provided by the composition of the invention and a second agent are administered individually or together, sequentially or simultaneously, in such a way as to provide a beneficial effect from co-action of these therapeutic agents. Such beneficial effect can include, but is not limited to, pharmacokinetic or pharmacodynamic co-action of the therapeutic agents. Combination therapy can, for example, enable administration of a lower dose of one or both agents than would normally be administered during monotherapy, thus decreasing risk or incidence of adverse effects associated with higher doses. Alternatively, combination therapy can result in increased therapeutic effect at the normal dose of each agent in monotherapy.

Compositions of the invention can be especially suited to combination therapies, particularly where the second agent is one that is, or can be, administered once daily. There are significant advantages in patient convenience and compliance where both components of a combination therapy can be administered at the same time and with the same frequency. This is especially true in the case of geriatric patients or those suffering memory impairment.

When administered simultaneously, the two components of the combination therapy can be administered in separate dosage forms or in coformulation, i.e., in a single dosage form, When administered sequentially or in separate dosage forms, the second agent can be administered by any suitable route and in any pharmaceutically acceptable dosage form, for example by a route and/or in a dosage form other than the present composition. In a preferred embodiment, both components of the combination therapy are formulated together in a single dosage form.

The second components of the subject combination therapy, e.g., drugs useful for the treatment Parkinson's disease and other movement disorders, include L-dopa, selegiline, apomorphine and anticholinergics. L-dopa (levo-dihydroxy-phenylalanine) is a dopamine precursor which can cross the blood-brain barrier and be converted to dopamine in the brain. Unfortunately, L-dopa has a short half life in the body and it is typical after long use (i.e., after about 4-5 years) for the effect of L-dopa to become sporadic and unpredictable, resulting in fluctuations in motor function, dyskinesias and psychiatric side effects. Additionally, L-dopa can cause B vitamin deficiencies to arise.

The gastrointestinal absorption of orally administered levodopa depends on the gastrointestinal transit rates as absorption occurs primarily in the proximal third of the intestine (duodenum/jejunum) and not in the stomach (Rivera-Calimlim et al. Europ. J. Clin. Invest. 1, 1313-1320, 1971). Therefore a delayed release dosage form containing levodopa/carbidopa or levodopa/carbidopa/entacapone with pramipexole will allow the levodopa to be released in the target proximal intestine region and release levodopa is a sustained manner similar to enteral infusion of levodopa.

Thus in certain embodiments, the invention provides a pharmaceutical composition comprising pramipexole and levodopa (optionally also carbidopa or a prodrug thereof) for treating PD and other related movement disorders. The invention also provides methods of using such pharmaceutical compositions for treating PD and other related movement disorders. See Examples 86-89.

Selegiline (Deprenyl, Eldepryl) has been used as an alternative to L-dopa, and acts by reducing the breakdown of dopamine in the brain. Unfortunately, selegiline becomes ineffective after about nine months of use. Apomorphine, a dopamine receptor agonist, has been used to treat Parkinson's disease, although is causes severe vomiting when used on its own, as well as skin reactions, infection, drowsiness and some psychiatric side effects.

Systemically administered anticholinergic drugs (such as benzhexyl and orphenedrine) have also been used to treat Parkinson's disease and act by reducing the amount of acetylcholine produced in the brain and thereby redress the dopamine/acetylcholine imbalance present in Parkinson's disease. Unfortunately, about 70% of patients taking systemically administered anticholinergics develop serious neuropsychiatric side effects, including hallucinations, as well as dyskinetic movements, and other effects resulting from wide anticholinergic distribution, including vision effects, difficulty swallowing, dry mouth, and urine retention. See e.g. Playfer, Parkinson's Disease, Postgrad Med J 73: 257-264, 1997 and Nadeau, Parkinson's Disease, J Am Ger Soc 45: 233-240, 1997.

Newer drug refinements and developments include direct-acting dopamine agonists, slow-release L-dopa formulations, inhibitors of the dopamine degrading enzymes catechol-O-methyltransferase (COMT) and monoamine oxidase B (MAO-B), and dopamine transport blockers. These treatments enhance central dopaminergic neurotransmission during the early stages of Parkinson's disease, ameliorate symptoms associated with Parkinson's disease, and temporarily improve the quality of life. However, despite improvements in the use of L-dopa for treating Parkinson's disease, the benefits accorded by these dopaminergic therapies are temporary, and their efficacy declines with disease progression. In addition, these treatments are accompanied by severe adverse motor and mental effects, most notably dyskinesias at peak dose and “on-off” fluctuations in drug effectiveness (Poewe and Granata, in Movement Disorders. Neurological Principles and Practice (Watts and Koller, eds) McGraw-Hill, New York, 1997; and Marsden and Parkes, Lancet 1: 345-349, 1977). No drug treatments are currently available that lessen the progressive pace of nigrostriatal degeneration, postpone the onset of illness, or that substantively slow disability (Shoulson, supra).

Other methods for the treatment of Parkinson's disease involve neurosurgical intervention, such as thalamotomy, pallidotomy, and deep brain stimulation. The thalamic outputs of the basal ganglia are an effective lesion target for the control of tremor (i.e., thalamotomy). Thalamotomy destroys part of the thalamus, a brain region involved in movement control. Unilateral stereotactic thalamotomy has proven to be effective for controlling contralateral tremor and rigidity, but carries a risk of hemiparesis. Bilateral thalamotomy carries an increased risk of speech and swallowing disorders resulting.

Stereotactic pallidotomy, surgical ablation of part of the globus pallidus (a basal ganglia), has also be used with some success. Pallidotomy is performed by inserting a wire probe into the globus pallidus and heating the probe to destroy nearby tissue. Pallidotomy is most useful for the treatment of peak-dose diskinesias and for dystonia that occurs at the end of a dose.

Aside from surgical resection, deep brain stimulation, high frequency stimulating electrodes placed in the ventral intermedialis nucleus, has been found to suppress abnormal movements in some cases. A variety of techniques exist to permit precise location of a probe, including computed tomography and magnetic resonance imaging. Unfortunately, the akinesia, speech and gait disorder symptoms of Parkinson's disease are little helped by these surgical procedures, all of which result in destructive brain lesions. Despite the development of modern imaging and surgical techniques to improve the effectiveness of these neurosurgical interventions for the treatment of Parkinson's disease tremor symptoms, the use of neurosurgical therapies is not widely applicable. For example, thalamotomy does not alleviate the akinetic symptoms which are the major functional disability for many people suffering from Parkinson's disease (Marsden et al., Adv. Neurol. 74: 143-147, 1997).

Therapeutic methods aimed at controlling suspected causative factors associated with Parkinson's disease (e.g., therapies which control oxidative stress and excitotoxicity) have also been developed. Clinical trials have shown that administration of antioxidative agents vitamin E and deprenyl provided little or no neuroprotective function (Shoulson et al., Ann. Neurol. 43: 318-325, 1998). Glutamate-receptor blockers and neuronal nitric oxide synthase (NOS) inhibitors have been proposed as therapies for Parkinson's disease; however, no experimental results from human studies have yet been published (Rodriguez, Ann. Neurol. 44: S175-S188, 1998).

The use of neurotrophic factors to stimulate neuronal repair, survival, and growth in Parkinson's disease has also been studied, particularly the use of glial cell line-derived neurotrophic factor (GDNF). Although GDNF protein protects some dopamine neurons from death, it is difficult to supply GDNF protein to the brain. Furthermore, the use of such protein therapies in general is problematic, since protein molecules show rapid in vivo degradation, are unable to penetrate the blood-brain barrier, and must be directly injected into the ventricles of the patient's brain (Palfi et al., Soc. Neurosci. Abstr. 24: 41, 1998; Hagg, Exp. Neurol. 149: 183-192, 1998; and Dunnett and Bjorklund, supra). Other neurotrophic factors which may have therapeutic value have been proposed based on in vitro and animal model systems, including neurturin, basic fibroblast growth factor (bFGF), brain-derived neurotrophic factor (BDNF), neurotrophins 3 and 4/5, ciliary neurotrophic factor and transforming growth factor β (TGF-β). However, the effectiveness of these therapies in humans remains unknown. At present, no single chemical compound or peptide has been reported to completely protect dopamine neurons from death by tropic factor withdrawal or neurotoxin exposure.

Cell replacement therapies have also received much attention as potential methods for treating Parkinson's disease (Freed et al., Arch. Neurol. 47: 505-512, 1990; Freed et al., N. Engl. J. Med. 327: 1549-1555, 1992; Lindvall et al., Science 247: 574-577, 1990; Spencer et al., N. Engl. J. Med. 327: 1541-1548, 1992; Widner et al., N. Engl. J. Med. 327: 1556-1563, 1992; Lindvall, NeuroReport 8: iii-x, 1997; Olanow et al., Adv. Neurol. 74: 249-269, 1997; and Lindvall, Nature Biotechn. 17: 635-636, 1999). These neural grafting therapies use dopamine supplied from cells implanted into the striatum as a substitute for nigrostriatal dopaminergic neurons that have been lost due to neurodegeneration. Although animal models and preliminary human clinical studies have shown that cell replacement therapies may be useful in the treatment of Parkinson's disease, the failure of the transplanted neurons to survive in the striatum is a major impediment in the development of cell replacement therapies.

Various sources of dopaminergic neurons for use in the transplantation process have been tried in animal experiments, including the use of mesencephalic dopamine neurons obtained from human embryo cadavers, immature neuronal precursor cells (i.e., neuronal stem cells), dopamine secreting non-neuronal cells, terminally differentiated teratocarcinoma-derived neuronal cell lines (Dunnett and Bjorkland, supra), genetically modified cells (Raymon et al., Exp. Neurol. 144: 82-91, 1997; and Kang, Mov. Dis. 13: 59-72, 1998), cells from cloned embryos (Zawada et al., Nature Medicine 4: 569-573, 1998) and xenogenic cells (Bjorklund et al., Nature 298: 652-654, 1982; Huffaker et al., Exp. Brain Res. 77: 329-336, 1989; Galpem et al., Exp. Neurol. 140:1-13, 1996; Deacon et al., Nature Med. 3: 350-353, 1997; and Zawada et al., Nature Med. 4: 569-573, 1998). Nonetheless, in current grafting protocols, no more than 5-20% of the transplanted dopamine neurons survive.

Additional therapies are also available, such as physical therapy, occupational therapy, or speech/language therapy. Exercise, diet, nutrition, patient/caregiver education, and psychosocial interventions have also been shown to have a positive effect on the mental and/or physical state of a person suffering from Parkinson's disease.

Various methods of evaluating Parkinson's disease in a patient include Hoehn and Yahr Staging of Parkinson's Disease, Unified Parkinson Disease Rating Scale (UPDRS), and Schwab and England Activities of Daily Living Scale.

A person suffering from Parkinson's disease should avoid contraindicated and potentially contraindicated drugs such as antipsychotic drugs, Haloperidol (Haldol), Perphenazine (Trilafon), Chlorpromazine (Thorazine), Trifluoperazine (Stelazine), Flufenazine (Prolixin, Permitil) Thiothixene (Navane), Thioridazine (Mellaril); antidepressant drug, combination of Perphenazine and Amitriptyline (Triavil); anti-vomiting drugs, Prochlorperazine (Compazine), Metoclopramide (Reglan, Maxeran), Thiethylperazine (Torecan), Reserpine (Serpasil), Tetrabenazine (Nitoman); blood pressure drug, Alpha-methyldopa (Aldomet); anti-seizure drug, Phenyloin (Dilantin); mood stabilizing drug, lithium; and anti-anxiety drug, Buspirone (Buspar).

### IV. Exemplary Levodopa/Carbidopa Pharmaceutical Compositions for First Immediate-Release Portion, Second Substantial Zero-Order Release Portion, and Second IR or Substantially Ascending Release Portion

Certain embodiments of the invention provides a pharmaceutical preparation comprising an oral dosage formulation in a therapeutically effective amount sufficient to treat movement disorder (e.g., Parkinson's disease or another movement disorder) in a patient, wherein the dosage formulation, when administered to the patient, provides a treatment regimen characterized by a rapid (immediate) release portion that quickly (e.g., in less than about 2 hours, e.g., in less than about 1 minute, 5 min., 10 min., 15 min., 20 min., 30 min., 45 min., 1 hour, 1.5 hours, 2 hours, etc., or within a range of time bounded by any of these time periods, e.g., 1 min. to 2 hours, 5 min. to 1 hour, 15 to 20 min., etc., after administration) boosts effective levodopa concentration to a therapeutically effective level, followed by a substantially sustained dose (zero-order release dose) over at least about 2 hours, 4 hours, 6 hours, 8, hours, 12 hours, 16 hours, 20 hours, or at least about 24 hours. Optionally, a substantially ascending portion, such as a second IR portion, is also provided subsequent to the second zero-order release portion to ensure a rapid drop at the end of the therapeutic regimen cycle (e.g., at day end, or before the patient goes to bed).

Certain embodiments of the invention provide a pharmaceutical preparation/dosage formulation provided in the form of a transdermal patch and formulated for sustained release formulation, in a therapeutically effective amount sufficient to treat a movement disorder (e.g., Parkinson's disease and related movement disorders) in a patient, wherein the dosage formulation, when administered (provided as a patch) to the patient, provides a substantially sustained dose over at least about 2 hours, 4 hours, 6 hours, 8, hours, 12 hours, 20 hours, or at least about 24 hours.

For the treatment of Parkinson's disease, the first IR portion preferably contains a relatively high ratio of decarboxylase inhibitor (e.g., carbidopa)/levodopa. In case of carbidopa/levodopa, the ratio is preferably >1:4, or 1:3, 1:2, 1:1, 2:1, 3:1, 4:1, 5:1, 6:1 or higher. The first IR is formulated to quickly release the compositions such that an effective therapeutic concentration of levodopa is reached in less than about 2 hours (e.g., in less than about 1 minute, 5 min., 10 min., 15 min., 20 min., 30 min., 45 min., 1 hour, 1.5 hours, 2 hours, etc., or within a range of time bounded by any of these time periods, e.g., 1 min. to 2 hours, 5 min. to 1 hour, 15 to 20 min., etc.) of administration.

In certain embodiments, carbidopa may be administered before release of the first IR portion of levodopa, thereby more effectively inhibiting peripheral decarboxylase activity and maximizing the efficacy of the levodopa in the first IR portion. For example, there may be a layer comprising carbidopa that is released prior to the first IR portion, carbidopa in the IR portion may be formulated to release faster than the levodopa in the IR portion (e.g., higher release ratio of carbidopa/levodopa), or a bioadhesive layer comprising a high proportion of carbidopa that at least partially undergoes immediate release may be present. Alternatively, the carbidopa may be administered as a separate formulation, e.g., together with a levodopa composition coated with a delayed release coating.

The second sustained release (zero-order release) portion may contain a single uniform composition (e.g., with a uniform ratio of carbidopa/levodopa throughout). Alternatively, the second substantially zero order release portion may have a gradient of carbidopa/levodopa ratio from start to finish. For example, the ratio may approach 1:4 at the beginning of the second portion, but drop continuously or discontinuously to, for example, 1:5, 1:6, 1:7, 1:8, 1:9, 1:10, 1:15, or 1:20, etc. If there is a discontinuous drop, the second portion may comprise several sub-portions, each possibly having a unique carbidopa/levodopa ratio.

The rapidly ascending release portion (such as the second IR portion) may contain no carbidopa, or fairly low ratio of carbidopa/levodopa, such that a rapid drop in effective levodopa concentration may be achieved, thus avoiding the long-tail effect that interferes with patient sleeping or rest.

In addition to carbidopa/levodopa, the portions of the subject dosage forms may additionally comprise compositions other than pharmaceutically acceptable carriers, excipients, or diluents, etc. (see details below). Such additional compositions may comprise a dopamine transporter inhibitor to be released with a delay. Such additional compositions may also comprise other pharmaceutical compositions useful for treating Parkinson's disease (e.g., in conjoint therapy).

In either oral or patch form, the above-described dosage preparation can be one wherein the pharmaceutical composition is formulated in a multiplicity of (sub-)portions or polymeric layers. For example, in certain embodiments, the second sustained release portion may comprise a multiplicity of layers such that the preparation optionally delivers to the patient a sustained release portion with varying ratios of decarboxylase/levodopa over time, even when the amount of released levodopa remains largely constant. Thus, the subject pharmaceutical composition can be provided in an initial portion (e.g., for immediate-release or IR), followed by a second portion (e.g., substantially zero-order sustained release or SR, optionally with more than one sub-portions or a continuously changing ratio of inhibitor/levodopa), and a final portion (e.g., an additional immediate-release portion), whereby the preparation delivers the initial dose, the second dose, then a final dose over time.

In other embodiments, the dose preparation can also be a plurality of beads, each bead including a subject pharmaceutical composition independently having a dissolution profile, which plurality of beads is a variegated population with respect to ratios of the pharmaceutical composition and/or dissolution profile, so as deliver, upon administration, the immediate, sustained, and increasing dose of the subject pharmaceutical composition. Several exemplary embodiments of the dosage forms are described in more details below.

In still other embodiments, the dose preparation is generated such that the subject pharmaceutical composition is (i) contained within a nonabsorbable shell that releases the drug at a controlled rate, and (ii) formulated in at least two different dissolution profiles.

In certain embodiments, the dosage formulations of the present invention have a side-effect therapeutic index, (SeD₅₀/ED₅₀), such as with respect to the movement disorder, that is at least 2 times greater than the same amount of drug provided in immediate release form, and more preferably at least 5, 10 or even 100 times greater.

In certain embodiments, the subject packages, preparations, pharmaceutical compositions, and methods for the treatment of movement disorders further comprise one or more therapeutic agents for treating Parkinson's disease selected from a dopamine precursor, such as L-dopa; a dopaminergic agent, such as Levodopa-carbidopa (SINEMET®, SINEMET CR®) or Levodopa-benserazide (PROLOPA®, MADOPAR®, MADOPAR HBS®); a dopaminergic and anti-cholinergic agent, such as amantadine (SYMMETRYL®, SYMADINE®); an anti-cholinergic agent, such as trihexyphenidyl (ARTANE®), benztropine (COGENTIN®), ethoproprazine (PARSITAN®), or procyclidine (KEMADRIN®); a dopamine agonist, such as apomorphine, bromocriptine (PARLODEL®), cabergoline (DOSTINEX®), lisuride (DOPERGINE®, pergolide (PERMAX®), pramipexole (MIRAPEX®), or ropinirole (REQUIP®); a MAO-B (monoamine oxidase B) inhibitor, such as selegiline or deprenyl (ATAPRYL®, CARBEX®, ELDEPRYL®); a COMT (catechol O-methyltransferase) inhibitor, such as CGP-28014, tolcapone (TASMAR®) or entacapone (COMTAN®); or other therapeutic agents, such as baclofen (LIORESAL®), domperidone (MOTILIUM®), fludrocortisone (FLORINEF®), midodrine (AMATINE®), oxybutynin (DITROPAN®), propranolol (INDERAL®, INDERAL-LA®), clonazepam (RIVOTRIL®), or yohimbine.

The subject treatment may also be used either in conjoint therapy with, or additionally include one or more other pharmaceutical compositions, such as the ones described below.

For example, US20030045539 (incorporated herein by reference) discloses a combination treatment of cabergoline and pramipexole provided concurrently to a patient suffering from various central nervous system diseases, and in particular for the treatment of Parkinson's Disease (PD). The initial dose of cabergoline is administered to the patient at a dose of 0.5 to 1 mg/patient/day and is adjusted upward at weekly intervals to a therapeutic dosage of 2, 4, 6, 8 or 10 mg/patient/day and where the initial dose of pramipexole is started at 0.375 mg/patient/day and is adjusted upward every 5 to 7 days to a therapeutic dosage of 3, 4, 5, 6, or 7 mg/patient/day. At least one portion of the subject pharmaceutical composition may additional comprise cabergoline and pramipexole for treating Parkinson's disease.

US20040166159 (incorporated herein by reference) discloses a pharmaceutical dosage forms having immediate and controlled release properties that contain an aromatic amino acid decarboxylase (AAAD) inhibitor (such as carbidopa), levodopa, and optionally a catechol-O-methyltransferase (COMT) inhibitor, for the treatment of medical conditions associated with reduced dopamine levels in a patient's brain. The dosage form may comprise up to about 1000 mg, or about 20-500 mg, about 50-500 mg, or about 100-200 mg of COMT inhibitor. The COMT inhibitor may be contained only within the immediate release component, or only within the sustained release component, or both. The COMT inhibitor may be CGP-28014, entacapone, or tolcapone. The dosage form may further comprise one or more drugs such as anti-cholinergics, beta 2-agonists, cyclooxygenase-2 (COX-2) inhibitors, dopamine receptor agonists, monoamine oxidase (MAO) inhibitors, opiate delta receptor agonists, opiate delta receptor antagonists, and N-methyl-D-aspartate (NMDA) antagonists. The dosage form may further comprise one or more drugs selected from albuterol, alpha-lipoic acid, amantadine, andropinirole, apomorphine, baclofen, biperiden, benztropine, bromocriptine, budipine, cabergoline, clozapine, deprenyl, dextromethorphan, dihydroergokryptine, dihydrolipoic acid, eliprodil, eptastigmine, ergoline, formoterol, galanthamine, lazabemide, lysuride, mazindol, memantine, mofegiline, orphenadrine, pergolide, pirbuterol, pramipexole, propentofylline, procyclidine, rasagiline, remacemide, riluzole, rimantadine, ropinirole, salmeterol, selegiline, spheramine, terguride, and trihexyphenidyl.

Similarly, other movement disorders may also be treated with similar methods and suitable pharmaceutical compositions, such as the ones described below.

For example, in certain embodiments of the packages, preparations, compositions, and methods for the treatment of a movement disorder, the invention further comprises one or more therapeutic agents for treating dystonia selected from an anti-cholinergic agent, such as trihexyphenidyl (ARTANE®), benztropine (COGENTIN®), ethoproprazine (PARSITAN®), or procyclidine (KEMADRIN®); a dopaminergic agent, such as Levodopa-carbidopa (SINEMET®, SINEMET CR®) or Levodopa-benserazide (PROLOPA®, MADOPAR®, MADOPAR HBS®); a muscle relaxant, such as baclofen (LIORESAL®); a sedative, such as Clonazepam (RIVOTRIL®); an anticonvulsant agent, such as carbamazepine (TEGRETOL®); a dopamine reuptake inhibitor, such as tetrabenazine (NITOMAN®); or a dopamine blocker, such as haloperidol (HALDOL®).

In certain embodiments of the packages, preparations, compositions, and methods for the treatment of a movement disorder, the invention further comprises one or more therapeutic agents for treating tremor selected from a O-blocker, such as propranolol (INDERAL®, INDERAL-LA®); an anticonvulsant agent, such as primidone (MYSOLINE®); or a carbonic anhydrase inhibitor, such as acetalzolamide (DIAMOX®) or methazolamide (NEPTAZANE®).

In certain embodiments of the packages, preparations, compositions, and methods for the treatment of a movement disorder, the invention further comprises one or more therapeutic agents for treating myoclonus selected from a sedative, such as clonazepam (RIVOTRIL®); or an anticonvulsant agent, such as valproic acid (EPIVAL®).

In certain embodiments of the packages, preparations, compositions, and methods for the treatment of a movement disorder, the invention further comprises one or more therapeutic agents for treating chorea selected from a dopamine blocker, such as haloperidol (HALDOL®); or a dopamine reuptake inhibitor, such as tetrabenazine (NITOMAN®).

In certain embodiments of the packages, preparations, compositions, and methods for the treatment of a movement disorder, the invention further comprises one or more therapeutic agents for treating restless leg syndrome selected from a dopaminergic, such as Levodopa-carbidopa (SINEMET®, SINEMET CR®) or Levodopa-benserazide (PROLOPA®, MADOPAR®, MADOPAR HBS®); a sedative, such as clonazepam (RIVOTRIL®); a dopamine agonists, such as bromocriptine (PARLODEL®), pergolide (PERMAX®), pramipexole (MIRAPEX®), or ropinirole (REQUIP®); a narcotic agent, such as codeine (TYLENOL # 3®); or a GABAergic agent, such as gabapentin (NEURONTIN®).

In certain embodiments of the subject packages, preparations, compositions, and methods for the treatment of movement disorders, the invention further comprises one or more therapeutic agents for treating tics selected from a sedative, such as clonazepam (RIVOTRIL®); an alpha antagonist, such as clonidine (CATAPRESS®); a dopamine reuptake inhibitor, such as tetrabenazine (NITOMAN®); or a dopamine blocker, such as haloperidol (HALDOL®) or perphenazine.

In certain embodiments, the present invention provides pharmaceutical preparations comprising, as an active ingredient, an enantiomerically enriched preparation of R-(−) amphetamine or a derivative thereof. The subject amphetamine compound is formulated in an amount sufficient to treat or prevent a movement disorder in an animal.

Still another embodiment of the invention relates to the use of enantiomerically enriched preparations of amphetamine compounds for lessening the severity or prophylactically preventing the occurrence of movement disorders in an animal, and thus, altering the mental or physical state of the animal. The compounds of the present invention may also be useful for treating and/or preventing memory impairment, e.g., due to a movement disorder.

It should be noted that levodopa and carbidopa of the subject pharmaceutical composition can be replaced in whole or in part in this invention with appropriate prodrugs, stereoisomers, acceptable salts, hydrates, solvates, etc. Levodopa prodrugs include any pharmaceutically suitable ester of levodopa such as, but not limited to, the methyl, ethyl, or propyl esters of levodopa, or combinations thereof. Levodopa may be in the form of (−)-L-α-amino-β-(3,4-dihydroxybenzene) propanoic acid, 3-hydroxy-L-tyrosine ethyl ester, phenylglycine, or a mixture thereof. The following specific examples describe levodopa prodrugs and carbidopa prodrugs, as well as additional compositions (such as fillers, organic acids, metals, metal chelators, etc.) that might constitute useful supplements to the backbone levodopa/carbidopa composition. These compositions may be used as the subject pharmaceutical composition.

For example, US20020151589A1 (incorporated herein by reference) describes a dispersible pharmaceutical composition comprising a therapeutically effective amount of L-DOPA ethyl ester, a therapeutically effective amount of a decarboxylase inhibitor, a filler, a disintegrant, and a lubricant, and a method of preparing the pharmaceutical composition described herein. The filler may be corn starch, glucose, various natural gums, methylcellulose, carboxymethylcellulose, microcrystalline cellulose, calcium phosphate, calcium carbonate, calcium sulfate kaolin, sodium chloride, powdered cellulose, sucrose, mannitol and starch, preferably microcrystalline cellulose (with a moisture content of up to about 1.5%, or up to about 5.0%). The decarboxylase inhibitor may be carbidopa (with a moisture content of, for example, between 5.0-10.0%, preferably 7.5%) or benserazide. The disintegrant may be kaolin, starch, powdered sugar, sodium starch glycolate, crosscarmelose sodium, carboxymethyl cellulose, microcrystalline cellulose and sodium alginate, preferably pregelatinized starch (with a moisture content of up to about 5, 7, 12, or 14%). The lubricant may be talc, sodium stearyl fumarate, magnesium stearate, calcium stearate, hydrogenated castor oil, hydrogenated soybean oil, and polyethylene glycol, preferably magnesium stearate. The excipient may be a binding agent such as sorbitol, glucose, xylitol, and mannitol. The composition may further comprise an antioxidant such as tocopherol, sodium metabisulphite, butylated hydroxytoluene, butylated hydroxyanisole, ascorbic acid and sodium ascorbate, preferably sodium metabisulphite. Various weight percentages and amounts per dose are also disclosed, and incorporated herein by reference. Such levodopa ethyl ester and the other described components may be used as the levodopa composition of the invention.

In another example, US20020192290A1 (incorporated herein by reference) discloses a pharmaceutical composition comprising a therapeutically effective amount of levodopa and of carbidopa, dispersed in a hydrophilic matrix, the composition further comprising an organic acid. The process for preparing the composition, comprising granulation, in particular in a fluidized bed, of the various components and compression of the granules obtained, is also disclosed. The organic acid may be fumaric acid, citric acid, ascorbic acid, maleic acid, glutamic acid, malonic acid and oxalic acid. The organic acid may represent from 0.2% to 20% by weight relative to the weight of the composition. The hydrophilic matrix (such as hydroxypropylmethyl cellulose) may represent from 10% to 80% by weight relative to the weight of the composition. The hydrophilic matrix may also comprise an insoluble substance, such as microcrystalline cellulose.

US20040028613A1 (incorporated herein by reference) discloses formulation useful for enhancing peak concentrations in CNS tissues or fluids and for treating, for example, Parkinson's disease, comprises dopamine agonist and at least one delivery enhancing agent. The dopamine receptor agonist may be apomorphine or a pharmaceutically acceptable salt or derivative thereof, and is administered to the subject in an effective dose of between about 0.25 and 2.0 mg. The delivery-enhancing agent(s) is/are selected from: (a) an aggregation inhibitory agent; (b) a charge modifying agent; (c) a pH control agent; (d) a degradative enzyme inhibitory agent; (e) a mucolytic or mucus clearing agent; (f) a ciliostatic agent; (g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, or (x) an inhibitor of cholesterol synthesis; or (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, support or complex-forming species with which the dopamine receptor agonist is effectively combined, associated, contained, encapsulated or bound resulting in stabilization of the dopamine receptor agonist for enhanced mucosal delivery, wherein the formulation of the dopamine receptor agonist with the one or more delivery-enhancing agents provides for increased bioavailability of the dopamine receptor agonist in a central nervous system tissue or fluid of the subject. The delivery-enhancing agent(s) may also be selected from citric acid, sodium citrate, propylene glycol, glycerin, L-ascorbic acid, sodium metabisulfite, edetate disodium, benzalkonium chloride, sodium hydroxide and mixtures thereof.

US20050070608 (incorporated herein by reference) discloses a composition useful for treating dopamine disorders, e.g., Parkinson's disease, comprises levodopa, carbidopa, acid and optionally metal chelator or thioether compound. The metal chelator may be EDTA, or deferoxamine mesylate. The EDTA may be in the form of a salt of a free base, and/or at a concentration of at least about 0.01 mg/ml. The acid may be a carboxylic acid, a mineral acid, citric acid, tartaric acid, ascorbic acid, dehydroascorbic acid, acetic acid (ethanoic acid), formic acid (methanoic acid), butyric acid (butanoic acid), benzoic acid, malic acid, propionic acid, epoxysuccinic acid, muconic acid, furanacrylic acid, citramalic acid, capric acid, stearic acid, caproic acid, malonic acid, succinic acid, diethylacetic acid, methylbutryic acid, hydrochloric acid, hydrobromic acid, phosphoric acid, nitric acid, and sulfuric acid, but preferably not ascorbic acid. The composition does not contain sugar. The composition may be a liquid. Preferably, less than 10%, or 5% of the carbidopa has degraded at 25° C. after 7 days, or less than 10% of the carbidopa has degraded at 25° C. after 30 days, or less than 5% of the carbidopa has degraded at 25° C. after 4 days. The composition may further comprise an artificial sweetener, such as aspartame. The composition may further comprise a preservative, such as sodium benzoate. The composition may be clear or translucent.

US20040167216 and its PCT counterpart WO04/052841A1 (both incorporated herein by reference) discloses prodrugs of carbidopa, derivatives of carbidopa prodrugs, methods of making and using such prodrugs and derivatives thereof, and compositions of such prodrugs and derivatives thereof. All such prodrugs may be used as carbidopa substitutes in the instant invention.

For example, in one embodiment, the prodrug is compound of Formula (I):

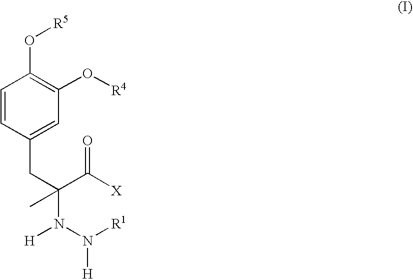

a stereoisomer thereof, an enantiomer thereof, a pharmaceutically acceptable salt thereof, a hydrate thereof, or a solvate of any of the foregoing, wherein:

X is selected from —OR¹⁰ and moieties of Formulae (II) and (M):

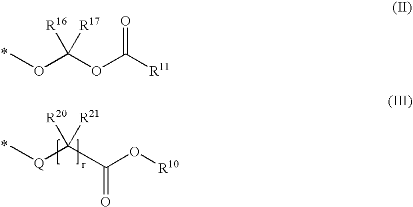

where: r is an integer from 1 to 6; Q is O or —NR¹⁵; —R¹ is selected from hydrogen and a moiety comprising Formula (IX):

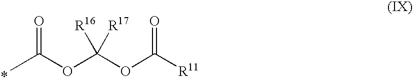

R⁴ and R⁵ are independently selected from hydrogen, alkyl, substituted alkyl, aryl, substituted aryl, heteroalkyl, substituted heteroalkyl, arylalkyl, substituted arylalkyl, heteroaryl, substituted heteroaryl, heteroarylalkyl, substituted heteroarylalkyl, cycloalkyl, substituted cycloalkyl, cycloheteroalkyl, substituted cycloheteroalkyl, —C(O)OR²⁷, —C(O)R²⁷, —(CR¹⁶R¹⁷)OC(O)R¹¹ and moieties of Formulae (XVII) and (XVIII):

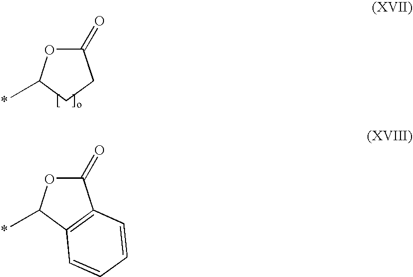

wherein o is 1-3, and the cycloheteroalkyl rings in (XVII) and (XVTII) are optionally substituted with one or more groups selected from halo, CN, NO₂, OH, C₁₋₆ alkyl, and C₁₋₆ alkoxy; or R⁴ and R⁵ together form a structure selected from Formulae (XII) to (XVI):

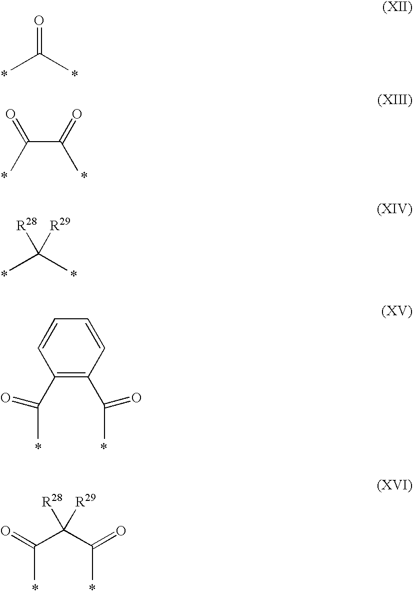

wherein the aryl ring in Formula (XV) is optionally substituted with one or more groups selected from halo, CN, OH, C₁₋₆ alkyl, C₁₋₆ alkoxy, and —CO₂R³¹;

R¹⁰ is selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R¹¹ is selected from hydrogen, alkyl, substituted alkyl, alkoxy, substituted alkoxy, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or optionally, R¹¹ and either R¹⁶ or R¹⁷, together with the atoms to which R¹¹, and either R¹⁶ or R¹⁷ are attached, form a cycloheteroalkyl or substituted cycloheteroalkyl ring, optionally to which is fused an aryl, substituted aryl, heteroaryl, substituted heteroaryl, cycloalkyl, substituted cycloalkyl, cycloheteroalkyl or substituted cycloheteroalkyl ring;

R¹⁵ is selected from hydrogen, alkyl, substituted alkyl, aryl, substituted aryl, arylalkyl, and substituted arylalkyl;

R¹⁶ and R¹⁷ are independently selected from hydrogen, alkyl, substituted alkyl, alkoxycarbonyl, substituted alkoxycarbonyl, aryl, substituted aryl, arylalkyl, substituted arylalkyl, carbamoyl, substituted carbomoyl, cycloalkyl, substituted cycloalkyl, cycloalkoxycarbonyl, substituted cycloalkoxycarbonyl, cycloheteroalkyl, substituted cycloheteroalkyl, heteroaryl, substituted heteroaryl, heteroarylalkyl, and substituted heteroarylalkyl or optionally, R¹⁶ and R¹⁷ together with the carbon atom to which R¹⁶ and R¹⁷ are attached form a cycloalkyl, substituted cycloalkyl, cycloheteroalkyl or substituted cycloheteroalkyl ring; each R²⁰ and R²¹ is independently selected from hydrogen, alkyl, substituted alkyl, alkoxy, substituted alkoxy, acyl, substituted acyl, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxy, or optionally, when r is 1, then R²⁰ and R²¹ together with the carbon atom to which R²⁰ and R²¹ are attached form a cycloalkyl, substituted cycloalkyl, cycloheteroalkyl or substituted cycloheteroalkyl ring, or optionally when R²⁰ and R¹⁵ are present and are attached to adjacent atoms then R¹⁵ and R²⁰ together with the atoms to which R¹⁵ and R²⁰ are attached form a cycloheteroalkyl or substituted cycloheteroalkyl ring;

R²⁷ is selected from alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R²⁸ and R²⁹ are independently selected from hydrogen, alkyl, substituted alkyl, alkoxy, substituted alkoxy, alkoxycarbonyl, substituted alkoxycarbonyl, aryl, substituted aryl, cycloalkyl, substituted cycloalkyl, heteroalkyl, and substituted heteroalkyl;

and R³¹ is selected from hydrogen, alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

with the provisos that
when X is —OR¹⁰, R¹ is hydrogen, and R⁴ and R⁵ are independently selected from hydrogen and C₁₋₁₉ alkyl, C₁₋₁₉ aryl or C₁₋₁₉ arylalkyl, then R¹⁰ is not hydrogen or C₁₋₆ alkyl; and
none of R¹, R⁴, R⁵, R¹⁰, R¹¹, R¹⁵, R¹⁶, R¹⁷, R²⁰, R²¹, R²⁷, R²⁸, R²⁹, and R³¹ comprise a bile acid moiety.

In another embodiment, the prodrug is a compound of Formula (Ia):

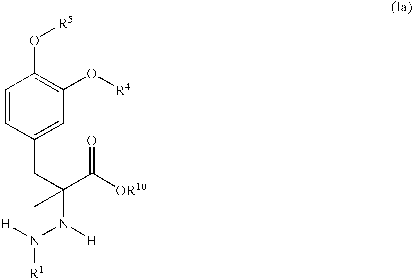

a stereoisomer thereof, an enantiomer thereof, a pharmaceutically acceptable salt thereof, a hydrate thereof, or a solvate of any of the foregoing, wherein:

R¹ is selected from hydrogen and the structure of Formula (IX): see above;

R⁴ and R⁵ are independently selected from hydrogen, alkyl, substituted alkyl, aryl, substituted aryl, heteroalkyl, substituted heteroalkyl, arylalkyl, substituted arylalkyl, heteroaryl, substituted heteroaryl, heteroarylalkyl, substituted heteroarylalkyl, cycloalkyl, substituted cycloalkyl, cycloheteroalkyl, substituted cycloheteroalkyl, —C(O)OR²⁷, —C(O)R²⁷, —(CR¹⁶R¹⁷)OC(O)R and moieties of Formulae (XVII) and (XVIII): see above;

wherein o is 1-3, and the cycloheteroalkyl rings in (XVII) and (XVIII) are optionally substituted with one or more groups selected from halo, CN, NO₂, OH, C₁₋₆ alkyl, and C₁₋₆ alkoxy;

or R⁴ and R⁵ together form a structure selected from Formulae (XII) to (XVI) (see above);

wherein the aryl ring in Formula (XV) is optionally substituted with one or more groups selected from halo, CN, OH, C₁₋₆ alkyl, C₁₋₆ alkoxy, and —CO₂R³¹;

R¹⁰ is selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R¹¹ is selected from hydrogen, alkyl, substituted alkyl, alkoxy, substituted alkoxy, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or optionally, R¹¹ and either R¹⁶ or R¹⁷, together with the atoms to which R¹¹, and either R¹⁶ or R¹⁷ are attached, form a first cycloheteroalkyl or substituted cycloheteroalkyl ring, to which an aryl, substituted aryl, heteroaryl, substituted heteroaryl, cycloalkyl, substituted cycloalkyl, cycloheteroalkyl or substituted cycloheteroalkyl ring is optionally fused to said first cycloheteroalkyl or substituted cycloheteroalkyl ring;

R¹⁶ or R¹⁷ are independently selected from hydrogen, alkyl, substituted alkyd, aryl, substituted aryl, arylalkyl, substituted arylalkyl, cycloalkyl, substituted cycloalkyl, cycloheteroalkyl, substituted cycloheteroalkyl, heteroarylalkyl, and substituted heteroarylalkyl or optionally, R¹⁶ or R¹⁷ together with the carbon atoms to which R¹⁶ or R¹⁷ are attached form a cycloalkyl, substituted cycloalkyl, cycloheteroalkyl or substituted cycloheteroalkyl ring;

R²⁷ is selected from hydrogen, alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R²⁸ and R²⁹ are independently selected from hydrogen, alkyl, substituted alkyl, alkoxy, substituted alkoxy, alkoxycarbonyl, substituted alkoxycarbonyl, aryl, substituted aryl, cycloalkyl, substituted cycloalkyl, heteroalkyl, and substituted heteroalkyl; and

R³¹ is selected from hydrogen, alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

with the provisos that
when R¹ hydrogen, and R⁴ and R⁵ are independently selected front hydrogen, Cl₁₋₁₉ alkyl, C₁₋₁₉ aryl, or Cl₁₋₁₉ arylalkyl, then R¹⁰ is not hydrogen or C₁₋₆ alkyl; and
none of R¹, R⁴, R⁵, R¹⁰, R¹¹, R¹⁵, R¹⁶, R¹⁷, R²⁷, R²⁸, R²⁹, and R³¹ comprise a bile acid moiety.

In yet another embodiment, the prodrug is a compound of Formulae (Ib) or (Ic):

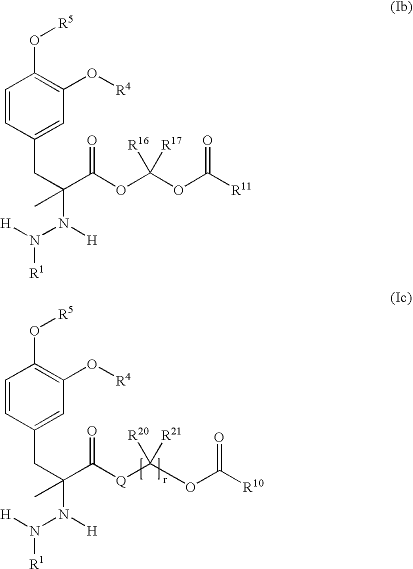

a stereoisomer thereof, an enantiomer thereof, a pharmaceutically acceptable salt thereof, a hydrate thereof, or a solvate of any of the foregoing, wherein:

Q is O or —NR¹⁵;

r is an integer from 1 to 6;

R is selected from hydrogen and a moiety comprising Formula (IX) (see above);

R⁴ and R⁵ are independently selected from hydrogen, alkyl, substituted alkyl, aryl, substituted aryl, heteroalkyl, substituted heteroalkyl, arylalkyl, substituted arylalkyl, heteroaryl, substituted heteroaryl, heteroarylalkyl, substituted heteroarylalkyl, cycloalkyl, substituted cycloalkyl, cycloheteroalkyl, substituted cycloheteroalkyl, —C(O)OR²⁷, —C(O)R²⁷, —(CR¹⁶R¹⁷)OC(O)R¹¹, and moieties of Formulae (XVII) and (XVIII) (see above);

wherein o is 1-3, and the cycloheteroalkyl rings in (XVII) and (XVIII) are optionally substituted with one or more groups selected from halo, CN, NO₂, OH, C₁₋₆ alkyl, and C₁₋₆ alkoxy;

or R⁴ and R⁵ together form a structure selected from Formulae (XII) to (XVI) (see above);

wherein the aryl ring in Formula (XV) is optionally substituted with one or more groups selected from halo, CN, OH, C₁₋₆ alkyl, C₁₋₆ alkoxy, and —CO₂R³¹;

R¹⁰ is selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R¹¹ is selected from hydrogen, alkyl, substituted alkyl, alkoxy, substituted alkoxy, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or optionally, R¹¹ and either R¹⁶ or R¹⁷, together with the atoms to which R¹¹, R¹⁶ and R¹⁷ are attached, form a cycloheteroalkyl or substituted cycloheteroalkyl ring, to which an aryl, substituted aryl, heteroaryl, substituted heteroaryl, cycloalkyl, substituted cycloalkyl, cycloheteroalkyl or substituted cycloheteroalkyl ring is optionally fused to said cycloheteroalkyl or substituted cycloheteroalkyl ring;

R¹⁵ is selected from hydrogen, alkyl, substituted alkyl, aryl, substituted aryl, arylalkyl, and substituted arylalkyl;

R¹⁶ and R¹⁷ are independently selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroarylalkyl, and substituted heteroarylalkyl or optionally, R¹⁶ and R¹⁷ together with the carbon atoms to which R¹⁶ and R¹⁷ are attached form a cycloalkyl, substituted cycloalkyl, cycloheteroalkyl or substituted cycloheteroalkyl ring;

each R²⁰ and R²¹ is independently selected from hydrogen, alkyl, substituted alkyl, alkoxy, substituted alkoxy, acyl, substituted acyl, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxy, or optionally, when r is 1, then R²⁰ and R²¹ together with the carbon atom to which R²⁰ and R²¹ are attached form a cycloalkyl, substituted cycloalkyl, cycloheteroalkyl or substituted cycloheteroalkyl ring, or optionally when R²⁰ and R¹⁵ are present and are attached to adjacent atoms then R²⁰ and R¹⁵ together with the atoms to which R²⁰ and R¹⁵ are attached form a cycloheteroalkyl or substituted cycloheteroalkyl ring;

R²⁷ is selected from hydrogen, alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R²⁸ and R²⁹ are independently selected from hydrogen, alkyl, substituted alkyl, alkoxy, substituted alkoxy, alkoxycarbonyl, substituted alkoxycarbonyl, aryl, substituted aryl, cycloalkyl, substituted cycloalkyl, heteroalkyl, and substituted heteroalkyl;

and R³¹ is selected from hydrogen, alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

with the proviso that none of R¹, R⁴, R⁵, R¹⁰, R¹¹, R¹⁵, R¹⁶, R¹⁷, R²⁰, R²¹, R²⁷, R²⁸, R²⁹, and R³¹ comprise a bile acid moiety.

### V. Other Exemplary Levodopa/Carbidopa Dosage Forms

Many dosage forms in the art may be readily adapted for use in the instant invention according to the general teaching of the invention. The following describe certain levodopa ethyl esters or derivatives thereof, which may be used in the instant invention.

US20030152628 (incorporated herein by reference) discloses a tablet which comprises an inner core formulated for controlled release consisting essentially of a mixture of levodopa ethyl ester or a derivative or a pharmaceutically acceptable salt thereof, a carrier and an inner core excipient component; and an outer layer encapsulating the inner core and formulated for immediate release comprising a mixture of a decarboxylase inhibitor and levodopa ethyl ester or a derivative or a pharmaceutically acceptable salt thereof.

US20030147957 (incorporated herein by reference) discloses a tablet which comprises: an inner core formulated for controlled release comprising a mixture of (a) a granulated admixture of a decarboxylase inhibitor and a surfactant, and (b) levodopa ethyl ester or a derivative or a pharmaceutically acceptable salt thereof; and an outer layer encapsulating the inner core and formulated for immediate release comprising a mixture of a granulated decarboxylase inhibitor and levodopa ethyl ester or a derivative or a pharmaceutically acceptable salt thereof. It also provides methods of manufacturing such tablets.

US20040234608 (incorporated herein by reference) discloses a pharmaceutical composition for use in a dosage form for oral administration to a patient. The composition expands upon contact with gastric fluid and promotes retention of the dosage form in the patient's stomach for a prolonged period of time. The application further provides pharmaceutical dosage forms containing an active ingredient, and the pharmaceutical composition. The forms are adapted for immediate or controlled release of the active ingredient. The dosage forms may be used advantageously in the treatment of Parkinson's disease with levodopa and hyperactivity and attention deficit disorder with methylphenidate. The composition comprises a hydrogel, a superdisintegrant and tannic acid wherein the volume of the composition increases about three fold within about 15 minutes of contacting gastric fluid. Its volume increases about 5-8 fold within about fifteen minutes of contacting gastric fluid, or increases about 3 fold within about five minutes of contacting gastric fluid. The hydrogel may comprise hydroxypropyl methylcellulose, and may further comprise hydroxypropyl cellulose, preferably in a weight ratio of from about 1:3 to about 5:3. The hydrogel may further comprise a cross-linked polyacrylate, such as a polyacrylic acid polymer crosslinked with allyl sucrose. The superdisintegrant may be selected from cross-linked carboxymethylcellulose sodium, sodium starch glycolate and cross-linked polyvinylpyrrolidone, preferably cross-linked carboxymethylcellulose sodium or sodium starch glycolate. The tannic acid may be present in an amount of from about 2 weight percent to about 12 weight percent of the total weight of hydrogel, superdisintegrant and tannic acid, exclusive of other excipients that may be present.

US20040180086 (incorporated herein by reference) discloses gastro-retentive dosage forms for prolonged delivery of levodopa and carbidopa/levodopa combinations. The dosage forms comprise a tablet containing the active ingredient and a gas-generating agent sealed within an expandable, hydrophilic, water-permeable and substantially gas-impermeable membrane. Upon contact with gastric fluid, the membrane expands as a result of the release of gas from the gas-generating agent in the tablet. The expanded membrane is retained in the stomach for a prolonged period of time up to 24 hours or more during which period the active ingredient is released from the tablet providing delivery of levodopa to the site of optimum absorption in the upper small intestine. For example, the application discloses a gastro-retentive dosage form of levodopa for oral administration to a patient in need thereof, the dosage form comprising (a) a tablet comprising a therapeutically effective amount of levodopa, a binder, and a pharmaceutically-acceptable gas-generating agent capable of releasing carbon dioxide upon contact with gastric juice, and (b) an expandable, hydrophilic, water-permeable and substantially gas-impermeable, membrane surrounding the tablet, wherein the membrane expands as a result of the release of carbon dioxide from the gas-generating agent upon contact with the gastric juice, whereby the dosage form becomes too large to pass into the patient's pyloric sphincter. The dosage form may further comprise a covering (e.g., a dry-fill capsule) for containing the dosage form, wherein the covering disintegrates upon contact with gastric fluid. The membrane may comprise polyvinyl alcohol. The gas-generating agent may be sodium bicarbonate, sodium carbonate, sodium glycine carbonate, potassium carbonate, calcium carbonate, magnesium carbonate or mixtures thereof. The binder may be a polyoxyethylene stearate, a poloxamer, a polyethylene glycol, a glycerol palmitostearate, a glyceryl monostearate, a methylcellulose or a polyvinyl pyrrolidone, such as Myrj 52, Lutrol F68, PEG 3350, a methylcellulose or a polyvinyl pyrrolidone.

Other exemplary dosage forms that may be adapted according to the teachings of the instant invention include US20030228360A1 and US20040052843.

### VI. Exemplary Delivery Devices

Various amounts of the subject drugs or pharmaceutical compositions can be included in tablets and drug eluting devices of the invention. Such tablets and drug eluting devices typically contain at least 1 mg of a drug/pharmaceutical composition. These tablets and drug eluting devices can also contain at least 2 mg, at least 5 mg, at least 10 mg, at least 25 mg, at least 50 mg, at least 100 mg, at least 500 mg or at least 1000 mg of a drug/pharmaceutical composition.

Any of the devices discussed below can be used to administer carbidopa, levodopa, or combinations of these drugs as discussed above, or they can be used to deliver any other drug desired to be administered, e.g., to treat any medical condition or disease state, or for any therapeutic or diagnostic purpose. In general, drugs/pharmaceutical compositions suitable for use herein can be small organic molecules (e.g., non-polymeric molecules having a molecular weight of 2000 amu or less, such as 1000 amu or less), peptides or polypeptides and nucleic acids.

More than one type of drug can be present in a tablet or a drug eluting device of the invention. The drugs can be evenly distributed throughout a medicament or can be heterogeneously distributed in a medicament, such that one drug is fully or partially released before a second drug. See different embodiments of the drug devices and/or layering in other parts of this specification.

Dosage forms of the invention typically weigh at least 5 mg. Dosage forms (such as the various shell designs of the invention) can also weigh at least 10 mg, at least 15 mg, at least 25 mg, at least 50 mg, at least 100 mg, at least 500 mg or at least 1000 mg.

Dosage forms of the invention typically measure at least 2 mm in one direction. For example, dosage forms can measure at least 5 mm, at least 10 mm, at least 15 mm or at least 20 mm in one direction. Typically, the diameter of the dosage forms is 2 to 40 mm, preferably 10 to 30 mm such as 20 to 26 mm. Mini-tablets have a diameter of 2 mm to about 5 mm. Such dosage forms can measure at least 2 mm, at least 5 mm, at least 10 mm, at least 15 mm or least 20 mm in a second direction and, optionally, a third direction. Preferably, the dosage form is of a size that facilitates swallowing by a subject.

The volume of a typical dosage form of the invention is at least 0.008 m/L, at least 0.01 mL, at least 0.05 mL, at least 0.1 mL, at least 0.125 m/L, at least 0.2 mL, at least 0.3 mL, at least 0.4 mL or at least 0.5 mL.

To produce a dosage form that can release at least two or three drugs at two or three different rates, and with preprogrammed delays, special dosage forms are used. For example, in the embodiments of the invention wherein levodopa, carbidopa, and the transport inhibitors are designed to be released concomitantly, the drugs may be formulated as bilayer (or other multilayer) tablets or shells (e.g., stacked layer of cakes, each may represent an independent formulation). Alternatively, levodopa and carbidopa may be formulated as a tablet within a tablet or bead (not limited to two nested layers). The outer tablet may contain a levodopa/carbidopa combination designed to be released together either as immediate release delivery patterns or as a sustained release delivery. The inner tablet/bead may be formulated to release after the outer tablet/bead has released the formulations. Optionally, the inner tablet(s)/bead(s) may be formulated with a coating layer to help achieve the desired delay in time.

In certain embodiments, the drugs may be formulated into a core tablet held in a recessed fashion within an annular ring of drug material. Such a dosage form is described in U.S. patent application Ser. No. 10/419,536 entitled “Dosage Form with a Core Tablet of Active Ingredient Sheathed in a Compressed Angular Body of Powder or Granular Material, and Process and Tooling for Producing It,” filed on Apr. 21, 2003 and Ser. No. 10/379,338 entitled “Controlled Release Dosage Forms,” filed on Mar. 3, 2003 and are incorporated herein by reference. The outer annular ring is formulated with the levodopa and decarboxylase enzyme inhibitor and formulated for either immediate release or sustained release delivery for the desired time. The inner core(s) of the dosage form contain the dopamine transport inhibitor to be released after a delay which may be formulated for the desired release profile.

Other embodiments of the invention use the dosage form described in U.S. patent application Ser. No. 10/191,298 entitled “Drug Delivery System for Zero-order, Zero-Order Biphasic, Ascending or Descending Drug Delivery,” filed on Jul. 10, 2002, incorporated herein by reference. The dopamine transport inhibitor may be formulated in the tablet mantle and released at the desired rate after a delay. The levodopa and decarboxylase enzyme inhibitor may be formulated in the expanding plug and released at the desired rate upon entry into the stomach.

Another embodiment of this invention may be achieved by formulating each of the drugs as pellets/beads, each with its own release profile and delay where applicable, and delivering the mixture of the three pellets in a shell using methods commonly known in the art. Furthermore, the proportions of the different types of pellets/beads may be altered or customized by a skilled artisan (e.g., qualified physician or pharmacologist), based on an individual patient's conditions, such as weight, age, gender, ethnicity, and/or specific genetic backgrounds. Such customization may be effected with the aid of, or automatically executed by a computer program based on relevant parameters such as those described above.

Embodiments of the invention wherein each drug may be released at a different rate can be formulated as tri-layer (or multilayer if necessary) tablets. Each layer of the tablet may have a distinct release profile. For example, a tablet within a tablet with an immediate release coating wherein the innermost tablet would be formulated with the dopamine transport inhibitor, the outer portion of the tablet formulated with levodopa, and the outer coating formulated with decarboxylase enzyme inhibitor, in an appropriate ratio according to the teachings of the instant invention. In another preferred embodiment, the drugs may be formulated into tablets held in a recessed fashion within an annular ring of drug material, as described above. The recessed core may be formulated as a delayed release of dopamine transport inhibitor at the desired release profile; the annular ring may be formulated to give the desired release profile of levodopa (immediate release and sustained release delivery); and an outermost coating layer may give an immediate release of decarboxylase enzyme inhibitor.

Another embodiment uses the delivery system as described in U.S. patent application Ser. No. 10/191,298, wherein the dopamine transport inhibitor is formulated in the mantle and the expanding plug is a bilayer tablet. One layer of the bilayer tablet comprising levodopa formulated for sustained release delivery and the other layer comprising decarboxylase enzyme inhibitor formulated to release at the desired rate. Yet another embodiment of the invention could be achieved by formulating each of the drugs as pellets each with its own release profile and delay where applicable and delivering the mixture of the three pellets in a shell as commonly understood by one of ordinary skill in the art.

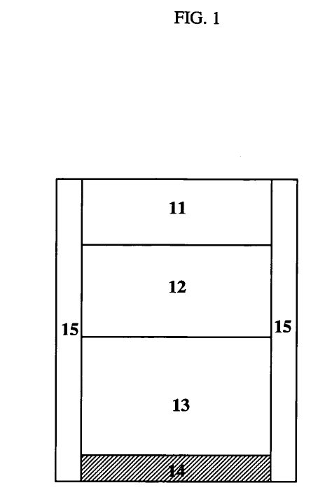

In yet another example, the tablet is a longitudinally compressed tablet containing a plurality of precompressed inserts of the various compositions of the invention, mixtures thereof, excipients, and optionally a permeation enhancer. The precompressed inserts may each have different compositions (e.g., the top insert may constitute the first IR portion, the next one or more inserts may constitute the substantially zero-order release rate second portion, etc. Drug is only released at the edge or surface of this tablet, which can result in zero-order kinetics in, for example, the second portion. In certain embodiments, the tablet may be encased in a sheath or shell, which has an insoluble, impermeable plug at one end to seal off the end, and has an opening (e.g., orifice) at the opposite end to allow drug release from successive layers of inserts (see FIG. 1). The thickness of each insert may be adjusted to accommodate different dosages. The overall shape of the device is not necessarily cylindrical, cubic column, etc., but can be any desired shape or size.

In certain embodiments, the tablet is a trilayer tablet having an inner core that includes one or more drugs in an appropriate matrix of excipients (e.g., HPMC, MCC, lactose) and is surrounded on two sides by a bioadhesive polymeric coating. Preferred bioadhesive polymeric coatings are a DOPA-BMA polymer and a mixture of poly(fumaric-co-sebacic) anhydride and EUDRAGIT™ RS PO. Other bioadhesive polymers are described in the section below.

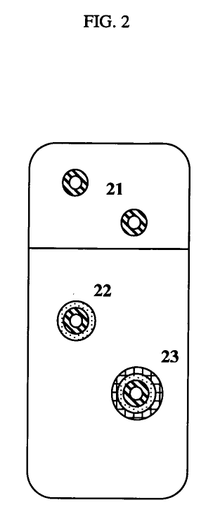

In another example, the tablet is comprised of a multiplicity of bioadhesive-coated microspheres or beads that have been compressed into a tablet core and subsequently coated with a bioadhesive coating and one or more additional coatings (e.g., enteric coatings). For example, in an illustrative embodiment as shown in FIG. 2, different types of beads, each type with separate types and/or thickness of coatings, may be mixed together in desired or customized proportions to deliver varying amounts of first IR, second portion of zero-order release, and optionally second portion of IR, etc. The coatings on different types of beads may control the release timing of each type of beads.

Various drug-eluting devices are described in U.S. Pat. Nos. 4,290,426, 5,256,440, 5,378,475, 5,773,019 and 6,797,283, the contents of which are incorporated herein by reference.

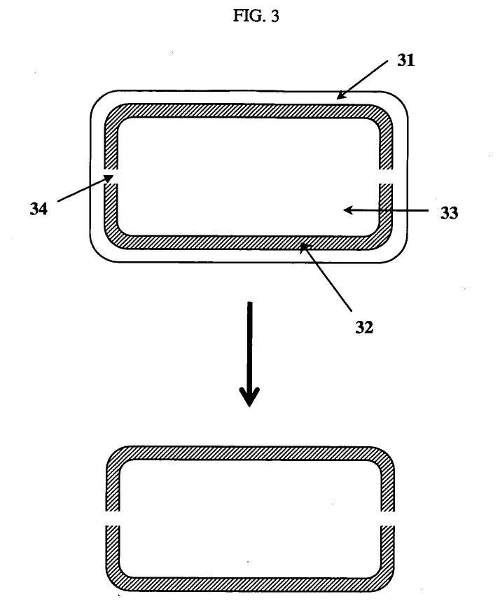

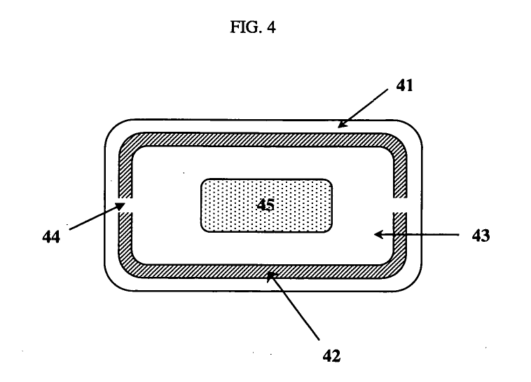

In one example, the drug-eluting device includes an inner reservoir comprising the effective agent; a first coating layer, which is essentially impermeable to the passage of the effective agent; and a second coating layer, which is permeable to the passage of the effective agent. The first coating layer covers at least a portion of the inner reservoir; however, at least a small portion of the inner reservoir is not coated with the first coating layer (e.g., there are one or more pores in the first coating layer). The second coating layer essentially completely covers the first coating layer and the uncoated portion of the inner reservoir. Typically, the first coating layer is a non-bioerodable or a slowly bioerodable polymer (e.g., a polymer having a polymethylene backbone). For the present invention, one illustrative embodiment is shown in FIG. 3, where the first coating is the bioadhesive coating, and the second coating is the first IR portion. The inner reservoir contains the second zero-order release portion, which may comprise one or a few layers to effect, for example, changing ratios of levodopa/carbidopa. One of these layers may also be the 3^{rd} IR portion or the dopamine transporter inhibitor (see FIG. 4).

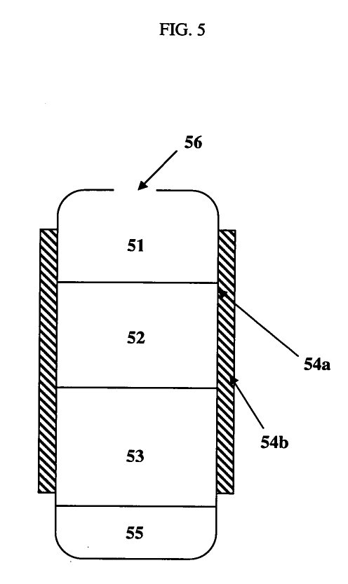

In another example, the drug eluting device includes a multilayer core, often bilayer or more layers, formed of polymer matrices that swell upon contact with the fluids of the stomach or other GI fluids. At least one layer of the multilayer core includes a drug. A portion of the polymer matrices are surrounded by a band of insoluble material that prevents the covered portion of the polymer matrices from swelling and provides a segment of the dosage form that is of sufficient rigidity to withstand the contractions of the stomach. The core and the band of soluble material are coated with a bioadhesive polymeric coating. FIG. 5 provides an illustrative embodiment of this configuration. As shown, the three depicted layers represent the immediate-release composition layer (IR), and two substantially zero-order release rate composition layers (CR1 and CR2). There may be more than two such substantially zero-order release rate composition layers, differing by their specific compositions (for example, the ratio of carbidopa/levodopa in the Parkinson disease therapeutic composition). A bioadhesive layer or patch (hatched lines) is shown to be on the outside wall of the shell encompassing the therapeutic compositions, which are successively released through an orifice close to the IR composition (proximal end).

In a further example, the drug eluting device is an osmotic delivery system. Typically, the reservoir of such devices contains osmotic agents to draw water across a semi-permeable membrane and a swelling polymer to push drug out of the device at a controlled rate. For example, FIG. 5 shows that the distal end of the shell may comprise a plug that can push the therapeutic compositions towards the orifice at the proximal end. The push mechanism can be any suitable means, such as a water-absorbing gel that swells when in contact with aqueous solution, or a rigid plate/plunger that can be driven by a micromotor (optionally externally activated).

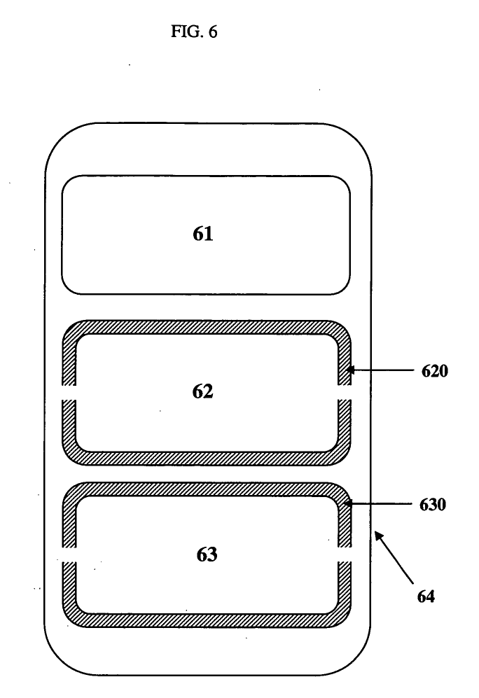

In yet another embodiment, there may be two or more reservoirs within a shell, each reservoir coated by a bioadhesive layer as described above, and each reservoir contains a different composition, such as different second doses designed to be released at successive times, or second zero-order release portion and the second IR portion (FIG. 6).

In another embodiment (FIG. 7), the composition may be formed as a cylinder or a column, or have a trapezoid profile (right panel). The compositions (e.g., levodopa 72 and carbidopa 71) are released starting from the top face and progressing in the order shown by the arrow. The top (beginning) of the dosage form has a different carbidopa/levodopa ratio from the bottom (end) of the dosage form.

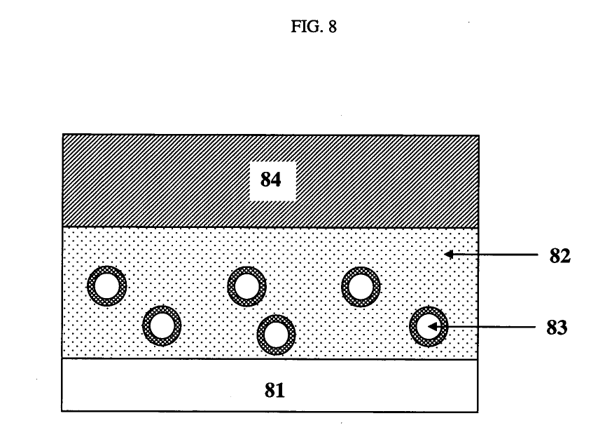

In another embodiment (FIG. 8), the first IR portion is formed as a layer, while one or more second portions (sustained zero-order release portion, e.g., CR2) may be formulated as coated beads embedded in a layer of another sustained zero-order release sub-portion (e.g., CR1). After the first IR portion is released, one sub-portion of the sustained zero-order release portion (e.g., CR1) starts to release, while release of the other sub-portions (e.g., CR2) is delayed due to the coating on the beads. There can be more than one layer and/or more than one types of beads according to this embodiment of the invention.

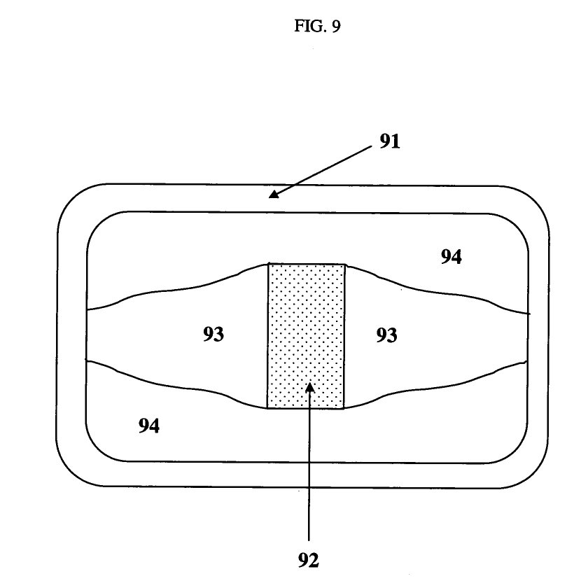

In another embodiment (FIG. 9), the first IR portion 91 covers the bioadhesive layer 94, which in turn covers the inner compositions, such as various sustained release (zero-order release) sub-portions (e.g., CR1 93), and/or an optional ascending release portion (92), which may also be occupied by another sub-portion of sustained release (zero-order release). The first sub-portion of the zero-order release (CR1 93) may have a geometric shape (regular or irregular, symmetrical or asymmetrical) such that increasing or decreasing amounts of drugs may be released in unit time periods.

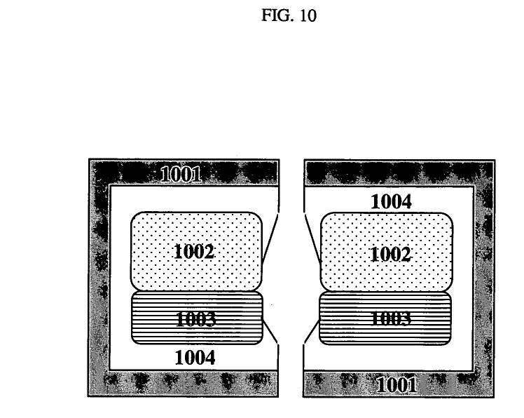

In another embodiment (FIG. 10), drug release from the two sub-portions of zero-order release (CR1 1002 and CR2 1003) is initiated from the inner core of a donut-shaped delivery device. After the outer IR layer 1001 is dissolved, holes on the bioadhesive layer 1004 encompassing the sub-portion(s) of zero-order release composition(s) may be release sequentially or simultaneously.

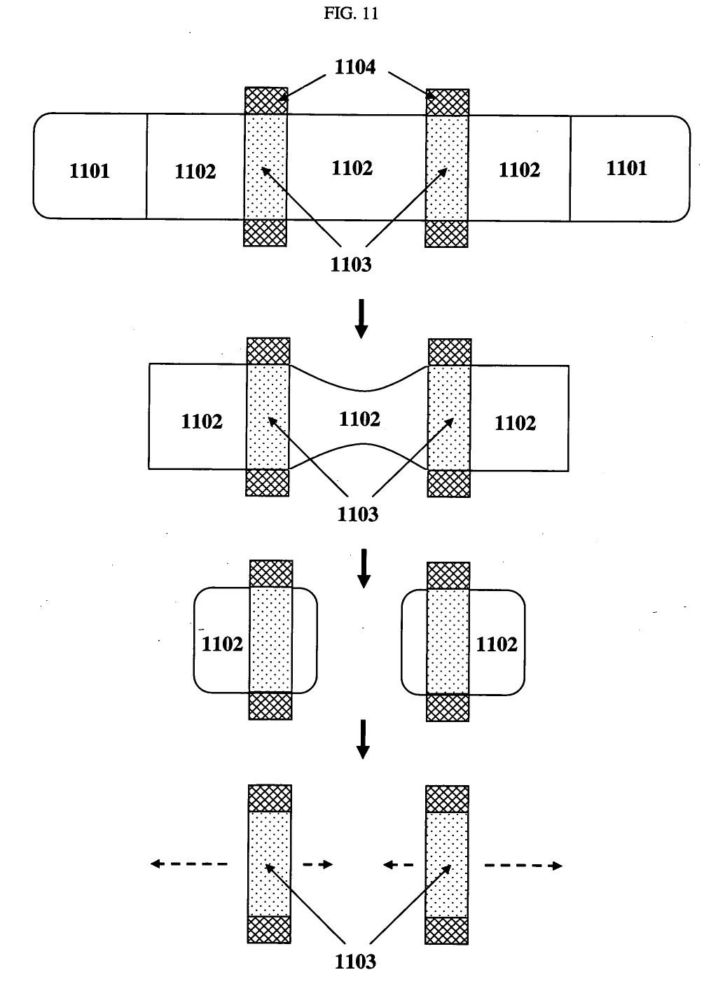

In another embodiment (FIG. 11), the dosage form takes the shape of a rod, with the first IR portion 1101 situated at one or both ends (or on the surface (not shown)) of the rod. If there is one or more controlled release (zero-order release) sub-portion (such as CR2 1103), they are each sealed off by a substantially impermeable bioadhesive band 1104, such that their release is delayed, until the other controlled release sub-portions (such as CR1 1102) are substantially completely released (see FIG. 11).

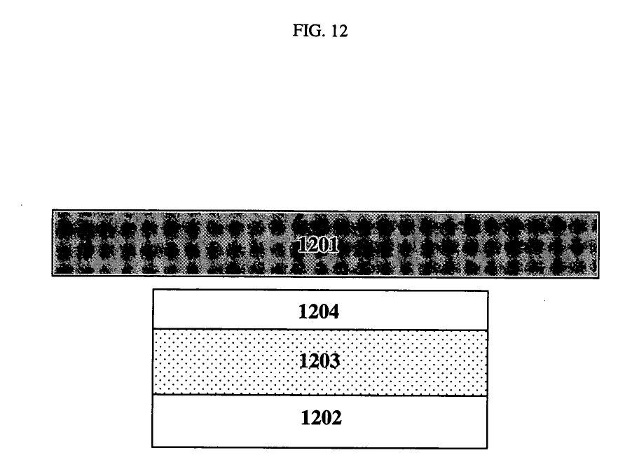

FIG. 12 shows yet another embodiment depicting a multilaminate bioadhesive buccal patch or tablet. The dosage form attaches to the mucosa surface (preferably through a bioadhesive layer attached to the CR2 layer, optionally also the CR1 layer, as described above), and sequentially releases the first IR 1202, and one or more sub-portions (e.g., CR1 1203 and CR2 1204) of controlled release sub-portions of the zero-order release portion (second portion).

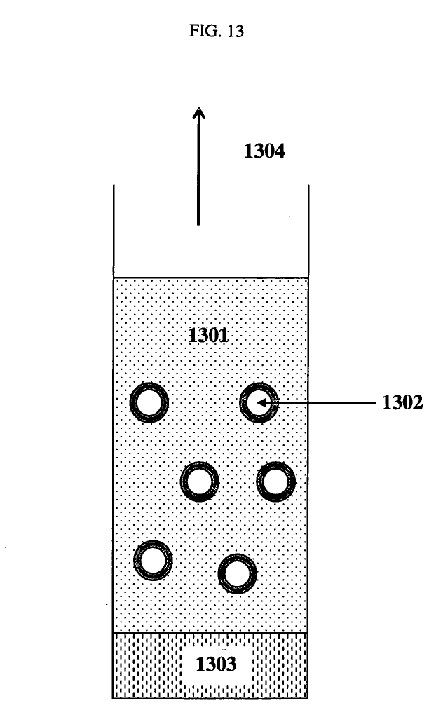

FIG. 13 features a dose sipping system, where various types of beads corresponding to various sub-portions of controlled release (only two sub-portions, CR1 and CR2, are shown as 1302) are embedded in a matrix of the first IR portion 1301, which in turn is deposited in a straw/tube. One end of the straw is sealed off by a porous plug 1303 to allow aqueous bodily fluid to seep in upon applying suction from the other end of the straw.

This embodiment also relates to a general concept for drug delivery, wherein a first portion of a multi-portion dosage form is formulated as a matrix for embedding one or more other portions of the same dosage form. In certain embodiments, the first portion is an immediate release (IR) formulation, and the other embedded portions are controlled release (CR) portions, each CR portion is optionally coated by a bioadhesive coat and/or a delayed release coat. Each CR portion may be formed as microparticles (e.g., beads) suspended in the first portion (e.g., IR portion) matrix. The disintegration of the matrix leads to the release of the embedded microparticles, which may re-adhere to the gut or other tissues (if coated by bioadhesive layer), and provided for sustained release.

This embodiment also relates to a general system for drug delivery, wherein therapeutic compositions are deposited at the end of a hollow tube sealed off by a porous plug. The plug holds the therapeutic compositions inside the tube, but is also porous enough to allow liquid to come into the tube through the plug if a vacuum is generated inside the tube (such as by sipping or applying suction). The dissolved therapeutic compositions can then exit through the opposite end of the tube, e.g., into the patient's mouth.

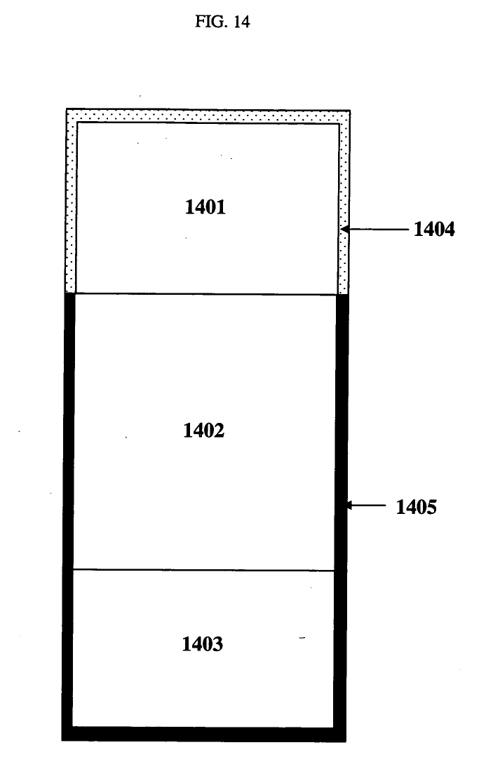

FIG. 14 features a delivery device with a shell comprising a cap and a bioadhesive body. The cap is made of gelatin-type of material that is readily dissolved once the shell is internalized by a patient. The body of the shell comprises a bioadhesive material of the subject invention. Upon dissolution of the cap, the IR portion, and the substantially zero-order release portion (or sub-portions thereof) may be sequentially released as shown. Alternatively, the one or more CR sub-portions may be embedded within the IR matrix as described in other embodiments of the invention.

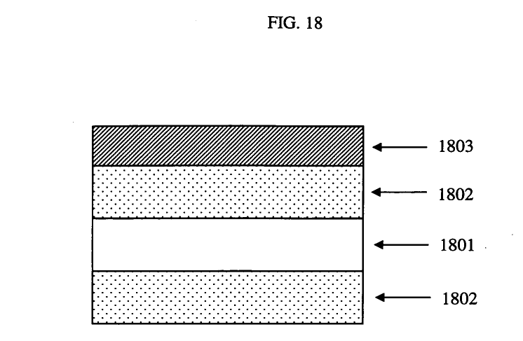

FIG. 18 features yet another configuration of the delivery device, in which a CR portion 1801 is sandwiched between two adhesive layers 1802 (e.g., a layered cross section) or inside one continuous adhesive layer 1802 (e.g., configured as a filled tube). SPHEROMER™ I [p(FASA)] and SPHEROMER™ III are exemplary such bioadhesive layers (see, e.g., Example 5). The portion/layer can (but need not) be substantially flat. In one embodiment, there are two substantially flat adhesive layers 1802 sandwiching one CR layer 1801. Components of the CR can be either released from surfaces not in contact with the adhesive parts 1802, and/or through the adhesive materials if such materials are at least partially permeable. An immediate release portion IR 1803 is coated over all or a part of the adhesive layer 1802. In one embodiment, the rapid dissolution of the IR portion exposes a CR surface not in contact with the adhesive material. In another embodiment, the dissolution of the IR portion does not substantially change the release rate of the CR portion. The tablet may be produced using methods such as those described in Examples 6-12.

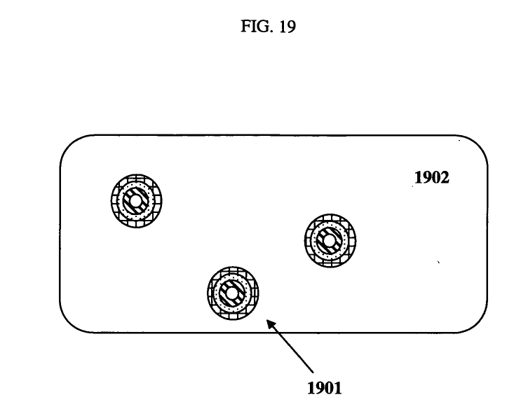

FIG. 19 features yet another configuration of the subject delivery device, which may be used in general to deliver any kind of drugs (or prodrugs, metabolic precursors thereof, etc.). Although levodopa and/or carbidopa were used in the Examples to illustrate the delivery method, device and dosage form, it should be understood that the subject delivery devices (such as the one described in FIG. 19), dosage form, and methods of making and using are not limited to these specific exemplary drug compositions described herein.

Thus according to this aspect of the invention, any drug to be delivered (e.g., levodopa and/or carbidopa), optionally including a bioadhesive polymer composition, and/or pharmaceutically acceptable excipients, may be formulated using the subject granulation-extrusion-spheronization process into multiparticulate pellets, which in turn may be dispersed in certain matrix materials, or simply encapsulated in capsules.

Specifically, appropriate amounts of the different ingredients are first weighed and mixed.

Suitable excipients for use in the subject granulation-extrusion-spheronization process include: Starcap-1500, starch-1500, and glycerine monostearate. In certain embodiments, the mixture is substantially free of microcrystalline cellulose.

In an exemplary embodiment, about 30-90%, about 40-85%, or about 50-80% (v/v) of the mixture (and the pellets formed therefrom) is effective ingredient (e.g., drug composition), rather than excipients or polymers. Such loadings can be achieved using any drug or combination of drugs that are suitably cohesive, plastic, and engage in hydrogen bonding. Levodopa and carbidopa are examples of such drugs, though others will be known to or can be easily identified by those of skill in the art.

These different ingredients can then be blended together in any suitable device, such as a planetary type mixer (e.g., Hobart Mixer with a 5-qt mixing bowl, operating at the speed setting #1, for about 5-15 min.). Optionally, the blending process is done in small volume to reduce any possible loss of the ingredients due to their non-specific adherence to the blending device. The blending step is typically done to ensure the formation of a uniform dry mix of the ingredients, typically over a period of, e.g., 5-15 min.

The dry mix is then granulated, e.g., under low shear with a granulation fluid, so as to form a wet granulation. Granulation fluids may be purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone, etc.

In certain embodiments, the granulation process is conducted in a small volume, such as in a 500-mL cylindrical vessel.

In certain embodiments, the granulation process is conducted with manual mixing, or conducted mechanically, e.g., in a planetary type mixer (such as a Hobart Mixer with a 5-qt mixing bowl). If the Hobart Mixer is used, it can be operated at its speed setting #1, depending on the batch size. Other types of mechanical mixers may also be used, with their respective appropriate settings, to achieve substantially the same result.

Once the wet granulation is formed, it may be extruded through the screen of a screen-type extruder. In certain embodiments, a Caleva Model 20 (or Model 25) Extruder may be used, operating at 10-20 rpm, and forming breakable wet strands (“the extrudate”). The screen aperture may be set at 0.8, 1, or 1.5 mm. Other types of extruders may be used to achieve substantially the same result.

The extrudate may then be spheronized in a spheronizer. For example, a Caleva Model 250 spheronizer equipped with a 2.5-mm spheronization plate may be used, which may be operated at a speed of about 1000-2000 rpm, typically for 5-10 min., in order to form spheronized pellets. Other types of spheronizer may be used to achieve substantially the same result.

For certain effective ingredients, such as carbidopa, the extruding step and the spheronization step may be omitted.

The spheronized pellets may then be dried. The drying may be conducted in a fluidized bed drier, such as a Vector MFL.01 Micro Batch Fluid Bed System. If the Vector drier is used, it may be operated at an inlet air flow rate of 100-300 lpm (liters per minute) and an inlet air temperature of about 50° C. Alternatively, the pellets may be dried in an ACT (Applied Chemical Technology) fluidized bed drier, operating at an inlet air flow rate of 140-150 fpm (foot per minute) and an inlet air temperature of 104° F. Other types of driers may also be used to achieve substantially the same result. Depending on the specific type of drugs/compositions, the drying temperature for a drier similar to the Vector drier may be between 35-70° C., or 40-65° C., or 45-60° C., or 45-55° C., etc. The drying temperature for a drier similar to the ACT drier may be between 70-140° F., or 80-130° F., or 90-120° F., or 100-110° F., etc.

In yet another embodiment, the spheronized pellets may be dried in an oven, such as a Precision gravity oven, operating at about 50° C., for 4-48 hrs, or 8-24 hrs. Depending on the specific type of drugs/compositions, the oven drying temperature for a drier similar to the Precision gravity oven may be between 35-70° C., or 40-65° C., or 45-60° C., or 45-55° C., etc.

The dried pellets may then be screened and/or classified. This can be done by using a stack of sieves, such as stainless steel sieves U.S. standard mesh sizes 8, 10, 12, 14, 16, 18, 20, 25, 30, 40, 45, or 60, etc., and using a mechanical sieve shaker (e.g., W.S. Tyler Sieve Shaker Ro-Tap Rx-29, operated for 5 min.). Particle size and distribution of pellet formulations can then be analyzed, and the classified pellets ranging from 0.25 mm (mesh # 60) to 2 mm (mesh # 10) may be selected for use or future formulation, such as additional film coating or other experimentation.

In certain embodiments, the formed pellets may be film-coated, e.g., with a delayed-release coating (such as an enteric coating), a controlled-release (CR) coating, a bioadhesive polymeric composition, and/or a dispersion-promoting coating, etc.

For example, the pellet core may be optionally surrounded by a CR coating, such as polymeric substance based on acrylates and/or methacrylates, e.g., a EUDRAGIT™ polymer (sold by Rohm America, Inc.). Specific EUDRAGIT™ polymers can be selected having various permeability and water solubility, which properties can be pH dependent or pH independent. For example, EUDRAGIT™ RL, EUDRAGIT™ NE, and EUDRAGIT™ RS are acrylic resins comprising copolymers of acrylic and methacrylic acid esters with a low content of quaternary ammonium groups, which are present as salts and give rise to the permeability of the lacquer films. EUDRAGIT™ RL is freely permeable and EUDRAGIT™ RS is slightly permeable, independent of pH. In contrast, the permeability of EUDRAGIT™ L is pH dependent. EUDRAGIT™ L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable. If desired, two or more types of polymeric substances may be mixed for use as the CR coating. Other polymers suitable for CR coatings, such as ethyl cellulose and cellulose acetate, can also be used in the CR coating. In certain embodiments, the CR coating may comprise one or more suitable polymers, such as a combination of two or more of the polymers discussed above.

Optionally, the pellets may also be coated by a bioadhesive polymeric composition. The adhesive material may facilitate the adhesion of the pellets to a desired surface, such as a preferred GI tract surface. For example, the pellets/beads may be coated by a top-layer of a bioadhesive polymer such as SPHEROMER™ I [p(FASA)], SPHEROMER™ II, SPHEROMER™ III, SPHEROMER™ IV, or mixtures thereof (see, e.g., Example 14).

In certain embodiments, the functions of a CR coating and bioadhesive coating can be combined in a single layer by using a mixture of polymers including a bioadhesive polymeric material and a polymer suitable for controlled release, i.e., a single layer may be both the CR layer and the bioadhesive layer of a particle.

Optionally, the pellets can also be film-coated with an additional layer of a so-called “non-functional polymer,” such as OPADRY™ II, EUDRAGIT™ E, ACRYL-EZE™, hydroxypropylmethyl cellulose, hydroxypropyl cellulose, polyvinyl alcohol, polyvinylacetate, polyanhydride, etc. (see, e.g., Example 14). This layer may serve as a dispersion-promoting coating that inhibits clumping and aggregation of the particles during dispersion. In embodiments wherein the pellets are further compressed with excipients to form tablets, this layer is preferably sufficiently strong or resilient to remain substantially intact during the compression process. This layer may also be protected by including a cushioning material among the excipients of the tablet matrix such as spray dried lactose, various grades of microcrystalline cellulose, glyceryl monostearate, pregelatinized starch, compressible sugar, PEG 8000, dicalcium phosphate (Di-Tab), calciumhydrogenphosphate (Bekapress D2) and cellactose.

The coating material (such as bioadhesive polymeric materials and/or functional/nonfunctional polymers) may be dissolved in an appropriate solvent, such as methylene chloride (e.g., for SPHEROMER™ I), methanol (e.g., for SPHEROMER™ III), a binary mixture of methanol and methylene chloride (e.g., for SPHEROMER™ I and SPHEROMER™ III), methanol or a binary mixture of ethanol and water (3:1 v/v) (e.g., for SPHEROMER™ IV), or methanol, ethanol, or isopropanol, or their binary mixture with acetone (e.g., functional or non-functional polymer).

The film coating may be performed in a fluidized bed coater, such as a Vector MFL.01 Micro Batch Fluid Bed System, equipped with a Wurster insert, operating at an inlet air flow rate of 100-300 lpm (liters per minute), and an inlet air temperature of about 25-45° C., or about 30-40° C., depending on the specific drugs and coatings (e.g., 25-30° C. for SPHEROMER™ I-coated levodopa-carbidopa; about 35° C. for SPHEROMER™ III-coated levodopa-carbidopa, etc.). If the Vector System is used, the pellets may be pre-warmed at 35° C. for 2-5 min., and after film-coating, post-dried at about 30° C. for about 15-30 min.

Alternatively, pellets may be coated in a fluid bed processor, such as a Fluid Air Model 5 fluid bed processor equipped with a Wurster insert, operating at an inlet air flow rate of about 70 cfm (cubic foot per minute) and an inlet air temperature of about 35° C. For this type of fluid bed processor, the pellets may be pre-warmed at 40° C. for 5-7 min., and after film-coating, post-dried at about 35° C. for about 30 min.

Other types of coaters may also be used to achieve substantially the same result.

Different lots or even different types of the same pellets produced using the subject method may optionally be mixed, e.g., by using a blender (such as a GlobePharma Maxiblend Blender equipped with an 8-qt stainless steel V-shell).

In certain embodiments, different types of pellets may be mixed. For example, some pellets may have no coating and are simply a core comprising the effective ingredients. Other pellets, even if identically in core structure, may further be coated by one or more types of coatings, e.g., bioadhesive coating, delayed-release coating, controlled-release coating, and/or dispersion-promoting coating, etc.

In certain embodiments, pellets produced using the methods of the invention may be encapsulated in capsules, such as hard gelatin capsules or pullulan capsules (NPcaps™), each with a predetermined amount of effective ingredients. For example, if the effective ingredients are carbidopa and levodopa, 50 mg carbidopa and 200 mg of the levodopa may be encapsulated.

In certain embodiments, pellets produced using the methods of the invention may be dispersed in a matrix material to assist the delivery of the effective ingredients of the pellets. There are at least two preferred configurations according to this embodiment of the invention.

FIG. 19 shows a schematic drawing (not to scale) of one such configuration. In FIG. 19, the active components 1901 (such as the pellets produced using the subject method, which are not necessarily round in shape) are embedded/dispersed within an inactive material or carrier matrix 1902. The carrier matrix 1902 can rapidly disintegrate, e.g., dissolve substantially completely (superdisintegrant) within about 15 minutes, 10 minutes, 8 minutes, 7 minutes, 6 minutes, 5 minutes, 3 minutes, 2 minutes, or about 1 minute or less. See, e.g., Example 15.

The inactive material 1902 may additionally comprise one or more cushioning materials dispersed throughout, e.g., sufficient to protect the active components 1901 when preparing the delivery device, by substantially absorbing the impact of compacting, and/or reducing friction on the surface of the particles 1901 (to prevent damaging the substructure of the particles, see below).

In certain embodiments, in order to incorporate these particles into a tablet matrix, a filler/binder must be used in the tableting process that will not allow the destruction of the pellets during the tableting process. Materials that are suitable for this purpose include, but are not limited to, microcrystalline cellulose (AVICEL®), soy polysaccharide (EMCOSOY®), pre-gelatinized starches (STARCH® 1500, NATIONAL® 1551), and polyethylene glycols (CARBOWAX®). These materials may be present in the range of about 5%-75% (w/w), and preferably between about 25%-50% (w/w).

In addition, disintegrants are added to the tablets in order to disperse the beads once the tablet is ingested. Suitable disintegrants include, but are not limited to: crosslinked sodium carboxymethyl cellulose (AC-DI-SOL®), sodium starch glycolate (EXPLOTAB®, PRIMOJEL®), and crosslinked polyvinylpolypyrrolidone (Plasone-XL). These materials may be present in the range of about 3%-15% (w/w), with a preferred range of about 5%-10% (w/w).

Lubricants are also added to assure proper tableting, and these can include, but are not limited to: magnesium stearate, calcium stearate, stearic acid, polyethylene glycol, leucine, glyceryl behenate, and hydrogenated vegetable oil. These lubricants should be present in amounts from about 0.1%-10% (w/w), with a preferred range of about 0.3%-3.0% (w/w).

The particles 1901 may be in any suitable size and shape (rods, beads, or other regular or irregular shapes). In one embodiment, the particles are beads with a diameter of less than about 2 mm, about 1.5 mm, about 1 mm, about 0.8 mm, about 0.7 mm, about 0.5 mm, about 0.3 mm, or about 0.1 mm. In certain embodiments, the pellets are substantially homogeneous in size and/or shape. In certain embodiments, for pellets with levodopa and/or carbidopa as effective ingredient, the pellet size is about 0.8-1 mm. Particles are formulated to these sizes in order to enable high drug loading when needed.

As described above, particles 1901 may have substructures, such as various coating layers surrounding a drug/prodrug core. Although the following describes the substructures using a bead with levodopa and/or carbidopa as effective ingredient, it is an illustrative example only, and the description also applies to other shapes of particles with other effective ingredients.

The core by itself may be an immediate release portion, or may have release-controlling components (e.g., CR portion), and preferably, the core is made by extrusion, such as the granulation-extrusion-spheronization process described in, e.g., Example 13. The core is optionally surrounded by a CR coating, such as polymeric substance based on acrylates and/or methacrylates, e.g., a EUDRAGIT™ polymer (sold by Rohm America, Inc.). Specific EUDRAGIT™ polymers can be selected having various permeability and water solubility, which properties can be pH dependent or pH independent. For example, EUDRAGIT™ RL, EUDRAGIT™ NE, and EUDRAGIT™ RS are acrylic resins comprising copolymers of acrylic and methacrylic acid esters with a low content of quaternary ammonium groups, which are present as salts and give rise to the permeability of the lacquer films. EUDRAGIT™ RL is freely permeable and EUDRAGIT™ RS is slightly permeable, independent of pH. In contrast, the permeability of EUDRAGIT™ L is pH dependent. EUDRAGIT™ L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable. If desired, two or more types of polymeric substances may be mixed for use as the CR coating. Other polymers suitable for CR coatings, such as ethyl cellulose and cellulose acetate, can be used in the CR coating. The CR coating may comprise one or more suitable polymers, such as a combination of two or more of the polymers discussed above.

Optionally, the CR coating is itself coated by a layer of adhesive material that facilitates the adhesion of the particles/beads to a desired surface, such as a preferred GI tract surface. Various suitable adhesive materials are described herein above. For example, the pellets/beads may be coated by a top-layer of a bioadhesive polymer such as SPHEROMER™ I [p(FASA)], SPHEROMER™ III, SPHEROMER™ IV, or mixtures thereof (see, e.g., Example 14). In certain embodiments, the functions of a CR coating and bioadhesive coating can be combined in a single layer by using a mixture of polymers including a bioadhesive polymeric material and a polymer suitable for controlled release, i.e., a single layer may be both the CR layer and the bioadhesive layer of a particle.

Optionally, pellets can be further film-coated with an additional layer of a so-called “non-functional polymer” such as OPADRY™ II, EUDRAGIT™ E, ACRYL-EZE™, hydroxypropylmethyl cellulose, hydroxypropyl cellulose, polyvinyl alcohol, polyvinylacetate, polyanhydride, etc. (see, e.g., Example 14). This layer may serve as a dispersion-promoting coating that inhibits clumping and aggregation of the particles during dispersion. In embodiments wherein the pellets are further compressed with excipients to form tablets, this layer is preferably sufficiently strong or resilient to remain substantially intact during the compression process. This layer may also be protected by including a cushioning material among the excipients of the tablet matrix such as spray dried lactose, various grades of microcrystalline cellulose, glyceryl monostearate, pregelatinized starch, compressible sugar, PEG 8000, dicalcium phosphate (Di-Tab), calciumhydrogenphosphate (Bekapress D2) and cellactose, e.g., so that the outer layer is not significantly scratched or gouged during the compression process, and/or retains its dispersion-promoting properties.

Optionally, an IR portion is included in the particle, such as over the dispersion-promoting coating, or between the dispersion-promoting coating and the adhesive layer, etc.

In an alternative embodiment, particles 1901 are not embedded within the inactive material 1902, but are instead disposed loose in a capsule that dissolves and releases the particles in the GI tract.

It should be understood that any other embodiments of the invention, such as those utilizing beads/pellets (see e.g., FIGS. 2, 8, 13, etc.), may additionally or alternatively use the coated pellets shown in FIG. 19.

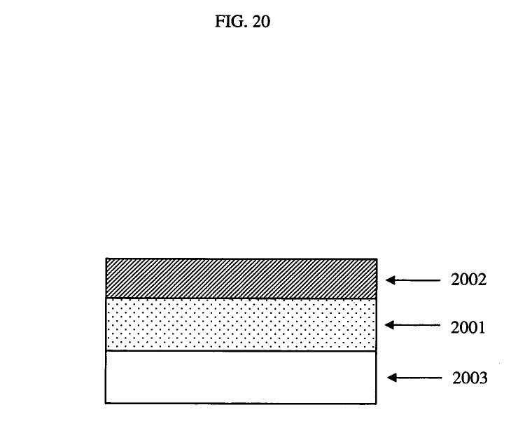

FIG. 20 features yet another embodiment of the delivery device, in which particles described herein above (e.g., with respect to FIG. 19) are embedded within a slow eroding material 2001 (e.g., that gradually erodes over 30 minutes, 45 minutes, 1 hr, 2 hrs, 4 hrs, 6 hrs, or longer). At least a portion of the eroding material 2001 is covered by an IR portion 2002, which disintegrates relatively rapidly to expose a surface of eroding material 2001. A portion of the slow eroding material 2001 is also optionally covered by a passive polymer support layer and/or an adhesive material 2003 as described herein above. In certain embodiments, the IR portion 2002 may be disposed on the adhesive layer 2003 instead of the eroding material 2001 as depicted. See, e.g., Example 16.

According to a related aspect of the invention, any drug to be delivered (e.g., levodopa and/or carbidopa), optionally including a bioadhesive polymer composition, and/or pharmaceutically acceptable excipients, may also be formulated as a multilayer tablet.

Specifically, different ingredients (such as those described above) are weighed and mixed. These ingredients, possibly with the exception of any lubricants, can then be blended together in any suitable device, such as an end-over-end ATR rotator (e.g., model RKVS), or a planetary type mixer (e.g., Hobart Mixer). Optionally, the blending process is done in small volume to reduce any possible loss of the ingredients due to their non-specific adherence to the blending device. The blending step is typically done to ensure the formation of a uniform dry mix of the ingredients, typically over a period of, e.g., 5-15 min.

The dry mix is then granulated, e.g., under low shear with a granulation fluid, so as to form a wet granulation. Granulation fluids may be purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone, etc.

In certain embodiments, the granulation process is conducted in a small volume, such as in a 500-mL cylindrical vessel.

In certain embodiments, the granulation process is conducted with manual mixing, or conducted mechanically, e.g., in a planetary type mixer (such as a Hobart Mixer with a 5-qt mixing bowl). If the Hobart Mixer is used, it can be operated at its speed setting #1, depending on the batch size. Other types of mechanical mixers may also be used, with their respective appropriate settings, to achieve substantially the same result.

Once the wet granulation is formed, it is dried. In certain embodiments, the wet granulation is dried in an oven (e.g., a Precision gravity oven, operating at about 50° C., for 8-24 hrs; or similar appropriate conditions for other types of ovens). Alternatively, the granulation may be dried in a fluidized bed drier, such as a Vector MFL.01 Micro Batch Fluid Bed System, operating at an inlet air flow rate of 100-300 lpm (liters per minute) and an inlet air temperature of about 50° C. The drying temperature is generally around 50° C. However, depending on different types of drugs/compositions, the temperature may be 35-70° C., or 40-65° C., or 45-60° C., or 45-55° C., etc.

The dried granulation is then ground, e.g., by using a pestle in a mortar, optionally followed by sieving the ground material, e.g., through an appropriate-sized screen (such as a U.S. Std. mesh # 60 screen), depending on the desired size of the granules.

At this point, the sieved granulation may be blended with a lubricant. In certain embodiments, the blending is conducted using an end-over-end ATR rotator (e.g., model RKVS). In certain embodiments, the blending is conducted using a planetary type mixer (e.g., Hobart Mixer, operating at the speed setting #1, for 5-15 min.). As a result, a uniformly lubricated dry mix is formed, which is then ready for compression.

Optionally, before compression, the lubricated dry mix may be passed through a sieve or screen, e.g., a U.S. Std. mesh # 60 screen.

Different components of the pharmaceutical composition (e.g., the effective ingredients, any bioadhesive polymeric materials, or other coatings, etc.) may be prepared as a mixture or separately using the subject methods. Once the dry mixes are formed, they can be compressed into single layer or multilayer tablets. For example, the lubricated dry mix may be pressed into tablets, such as by using a single-station manual tablet press (e.g., GlobePharma Manual Tablet Compaction Machine MTCM-I, equipped with adequate die and punch set). If the GlobePharma machine is used, tablets may be prepared, e.g., at a pressure ranging from 250 to 4000 pounds per square inch (psi), and a compression time of, e.g., 1 to 4 seconds. Other machines may also be used to achieve substantially the same result.

Alternatively, in certain embodiments, tablets may be produced with wet granulation of active ingredients followed by direct compression (see, e.g., Example 6).

In certain embodiments, multilayer tablets may be produced, with each layer comprising a different ingredient. In these embodiments, a single-station manual tablet press (e.g., GlobePharma Manual Tablet Compaction Machine MTCM-I, equipped with adequate die and punch set) may be used in several steps to produce the multilayer tablets. For example, for a bilayer tablet, the compression process may include:

(1) adding the first layer blend into the die cavity, optionally followed by manually tapping it, e.g., using a stainless steel spatula;

(2) adding the second layer blend into the die cavity;

(3) pre-compressing the two layers together, e.g., at a pressure ranging from 250 to 500 pounds per square inch (psi) and a compression time of, e.g., 1 to 5 seconds.

(4) compressing the pre-compacted layers together, e.g., at a pressure ranging from 1000 to 4000 pounds per square inch (psi) and a compression time of, e.g., 1 to 4 seconds.

The process can be repeated or modified if more than two layers of ingredients are to be used.

In certain embodiments, the tablet can be made with a pre-compressed insert with effective ingredients. Such pre-compressed inserts may be produced with direct compression (see, e.g., Example 10). The same press machine may be used for this process. For example, if using the GlobePharma Manual Tablet Compaction Machine MTCM-I machine, tablet inserts may be prepared, e.g., at a pressure ranging from 500 to 1000 pounds per square inch (psi), and a compression time of, e.g., 1 to 2 seconds. Other machines may also be used to achieve substantially the same result. The pre-compressed insert may be used as one of the layers (e.g., the second layer) in the tablet, or embedded in the middle of another layer (e.g., the second layer). See, for example, Example 10.

Optionally, the tablets may be coated with one or more coating compositions, such as in the form of successive layers. The coating compositions may include bioadhesive layers, delayed release layers, controlled-release layers, and/or other functional/non-functional polymers etc. (supra). For example, tablets may be film-coated for this purpose, using a pan coater (e.g., O'Hara Labcoat, operating at an inlet air flow rate of about 60 cfm (cubic foot per minute) and an inlet air temperature of about 35° C.). The tablets may be pre-warmed at 35° C. for 5-10 min., and after film coating, may be post-dried at about 30° C. for about 15-30 min. Other coaters may also be used to achieve substantially the same result.

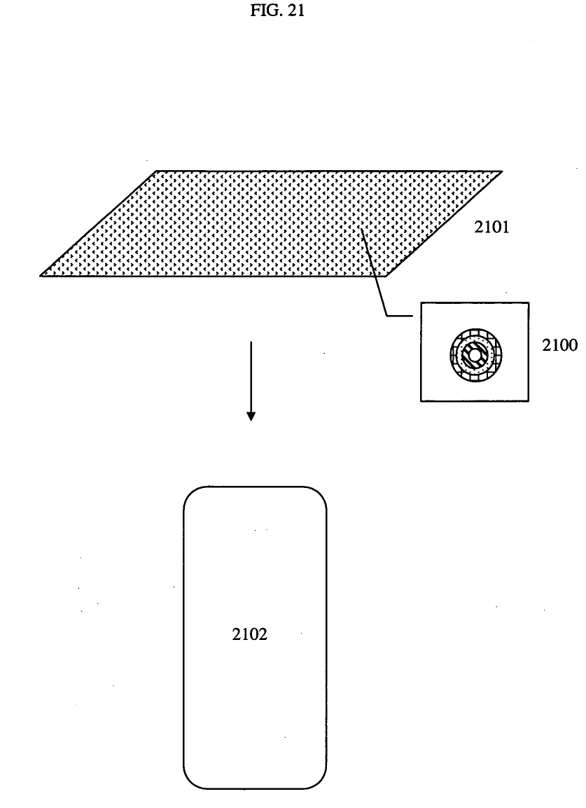

FIG. 21 features yet another embodiment of the delivery device, in which particles 2100 described herein above (e.g., with respect to FIG. 19) are dispersed on the surface of a bioadhesive film 2101. The film may optionally be dried or cured, e.g., without disrupting the particle adhesion. The film may then be placed in a capsule 2102 for administration to a patient. If needed, the film may first be folded or cut to a suitable shape or size. Once administered to a patient, the capsule releases the film, which then rehydrates (if necessary) and adheres to a mucosal surface, allowing the particles spreaded and adhered thereto to release the active components.

In certain embodiments, the subject composition is formulated for variable dosing, such as customized dosing for individual patients.

In addition, more than one type of drugs can be present in a tablet or a drug eluting device of the invention, e.g., for combination therapy with other pharmaceutical compositions effective for treating PD or other movement disorders (see below). The drugs can be evenly distributed throughout a medicament or can be heterogeneously distributed in a medicament, such that one drug is fully or partially released before a second drug. See different embodiments of the drug devices and/or layering in other parts of this specification.

Dosage forms of the invention typically weigh at least about 50 mg. Dosage forms (such as the various shell designs of the invention) can also weigh at least 100 mg, at least 150 mg, at least 250 mg, at least 500 mg, or at least 1000 mg, etc.

Dosage forms (e.g, capsule or tablet) of the invention typically measure at least 2 mm in one direction. For example, dosage forms can measure at least 5 mm, at least 10 mm, at least 15 mm or at least 20 mm in one direction. Typically, the diameter of the dosage forms is 2 to 40 mm, preferably 10 to 30 mm such as 20 to 26 mm. Mini-tablets have a diameter of 2 mm to about 5 mm. Such dosage forms can measure at least 2 mm, at least 5 mm, at least 10 mm, at least 15 mm or least 20 mm in a second direction and, optionally, a third direction. Preferably, the dosage form is of a size that facilitates swallowing by a subject.

The volume of a typical dosage form of the invention is at least 0.008 mL, at least 0.01 mL, at least 0.05 mL, at least 0.1 mL, at least 0.125 mL, at least 0.2 mL, at least 0.3 mL, at least 0.4 mL or at least 0.5 mL.

Dosage forms of the invention may be a tablet that can be of any suitable size and shape, for example, round, oval polygonal or pillow-shaped, and optionally bears nonfunctional surface markings. Especially in the case of coated tablets, they are preferably designed to be swallowed whole and are therefore typically not provided with a breaking score. Tablets of the invention can be packaged in a container, e.g., accompanied by a package insert providing pertinent information such as, for example, dosage and administration information, contraindications, precautions, drug interactions and adverse reactions.

To produce a dosage form that can release at least two or three drugs at two or three different rates, and with preprogrammed delays, special dosage forms are used. For example, in the embodiments of the invention wherein different dosage forms of levodopa/carbidopa are designed to be released concomitantly, the drugs may be formulated as bilayer (or other multilayer) tablets or shells (e.g., stacked layer of cakes, each may represent an independent formulation). Alternatively, the drugs may be formulated as a tablet within a tablet or bead (not limited to two nested layers). Optionally, a bioadhesive layer may be coated over part or all of a gel capsule (or other forms of delivery device) to enhance the stay of the device within a certain area of the GI tract, such as the intestine.

The various embodiments described herein are only a sample of numerous possible configurations to deliver the subject dosage forms. Other variations may be readily envisioned based on the principals and teachings of the instant specification. For example, various other drug-eluting devices are described in U.S. Pat. Nos. 4,290,426, 5,256,440, 5,378,475, 5,773,019 and 6,797,283, the contents of which are incorporated herein by reference.

In these and other embodiments of the invention, the various bioadhesive coatings that can be used are described in detail in the section below.

Many of the different embodiments described above may be implemented by using rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of a subject pharmaceutical composition at a particular target site. The biodegradable polymers undergo chemical decomposition to form soluble monomers or soluble polymer units. The biodegradation of polymers usually involves chemically or enzymatically catalyzed hydrolysis. Representative biodegradable polymers comprise a member selected from biodegradable poly(amides), poly(amino acids), poly(esters), poly(lactic acid), poly(glycolic acid), poly(orthoesters), poly(anhydrides), biodegradable poly(dehydropyrans), and poly(dioxinones). The polymers are known to the art in Controlled Release of Drugs, by Rosoff, Ch. 2, pp. 53-95 (1989); and in U.S. Pat. Nos. 3,811,444; 3,962,414; 4,066,747; 4,070,347; 4,079,038; and 4,093,709.

In certain embodiments, representative dosage forms include hydrogel matrix containing a plurality of tiny pills or other particles. The hydrogel matrix comprises a hydrophilic polymer, such as selected from a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin and a hydrophilic colloid. The hydrogel matrix comprises a plurality of tiny pills or particles (such as 4 to 50), each tiny pill or particle may comprise a different portion of the subject compositions (e.g., IR, etc.). Representative of wall-forming materials include a triglyceryl ester selected from glyceryl tristearate, glyceryl monostearate, glyceryl dipalmitate, glyceryl laureate, glyceryl didecenoate and glyceryl tridecenoate. Other wall forming materials comprise polyvinyl acetate phthalate, methylcellulose phthalate, and microporous vinyl olefins. Procedures for manufacturing tiny pills are disclosed in U.S. Pat. Nos. 4,434,153; 4,721,613; 4,853,229; 2,996,431; 3,139,383 and 4,752,470, which are incorporated by reference herein.

In still other embodiments, the invention employs a dosage form comprising a polymer that releases a drug by diffusion, flux through pores, or by rupture of a polymer matrix. The dosage form matrix can be made by procedures known to the polymer art. An example of providing a dosage form comprises blending a pharmaceutically acceptable carrier, like polyethylene glycol, with a known dose of the subject pharmaceutical composition, and adding it to a silastic medical grade elastomer with a cross-linking agent, like stannous octanoate, followed by casting in a mold. The step is repeated for each successive layer. The system is allowed to set, e.g., for 1 hour, to provide the dosage form. Representative polymers suitable for manufacturing the dosage form include olefin and vinyl polymers, condensation polymers, carbohydrate polymers, and silicon polymers as represented by poly(ethylene), poly(propylene), poly(vinyl acetate), poly(methyl acrylate), poly(isobutyl methacrylate), poly(alginate), poly(amide), and poly(silicone). The polymers and manufacturing procedures are known in Polymers, by Coleman et al., Vol. 31, pp. 1187-1230 (1990); Drug Carrier Systems, by Roerdink et al., Vol. 9, pp. 57-109 (1989); Adv. Drug Delivery Rev., by Leong et al., Vol. 1, pp. 199-233 (1987); Handbook of Common Polymers, Compiled by Roff et al., (1971) published by CRC Press; and U.S. Pat. No. 3,992,518.

Other exemplary embodiments of the delivery devices are described in the subsection below, and in the Example section of the application.

Other Exemplary Delivery Devices and Systems

This subsection describes additional exemplary delivery systems that can be used to deliver any of a large spectrum of compounds (e.g., drugs, prodrugs, metabolic precursors, etc.), especially those with limited absorption windows in upper GI (e.g., stomach).

An exemplary list of compounds that can be delivered using the subject dosage forms and/or delivery devices includes, but not limited to: metformin, acyclovir, ranitidine, riboflavin, chlorthiazide, gabapentin, losartin potassium, ganciclovir, cimetidine, minocycline, fexofenadine, bupropion, orlistat, captopril, diphenhydramine, tripelennamine, chlorpheniramine maleate, promethazine, omeprazole, prostaglandin, carbenoxolane, sucralphate, isosorbide, quinidine, enalapril, nifedipine, verapamil, diltiazem, nadolol, timolol, pindolol, salbutamol, terbutaline, carbuterol, broxaterol, aminophylline, cyclizine, cinnarizine, domperidone, alizapride, vincristine, megestrol acetate, daunorubicin, actinomycin, adriamycin, etoposide, 5-fluorouracil, indomethacin, sulindac, piroxicam, ibuprofen, naproxen, ketoprofen, temazepam, lorazepam, flunitrazepam, amantadine, ampicillin, amoxicillin, erythromycin, tetracyclines, cyanocobalamin, amino acids, iron or calcium salts of essential trace elements, or pharmacologically acceptable salts of the above.

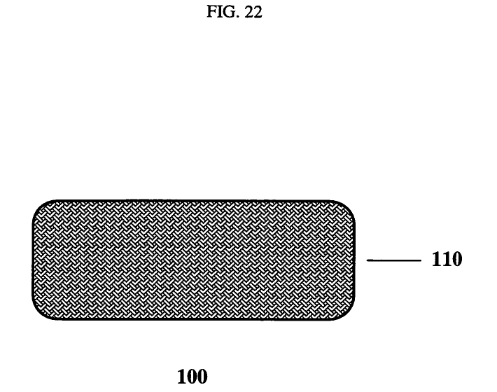

In certain embodiments of the invention, illustrated in FIG. 22, the oral solid dosage form is a monolayer matrix tablet 100, containing one or more drug(s), pharmaceutically acceptable excipients or salts thereof, optionally one or more permeation and/or dissolution enhancers, one or more bioadhesive polymer compositions, formulated in a single monolithic layer 110. Various drug release profiles can be achieved by tailoring the composition and/or configuration of each layer. In this embodiment, the tablet is designed to provide immediate release (IR) or controlled release (CR) of one or more soluble, poorly soluble or insoluble drugs from all sides. The cross-section of this dosage form is illustrated in FIG. 22.

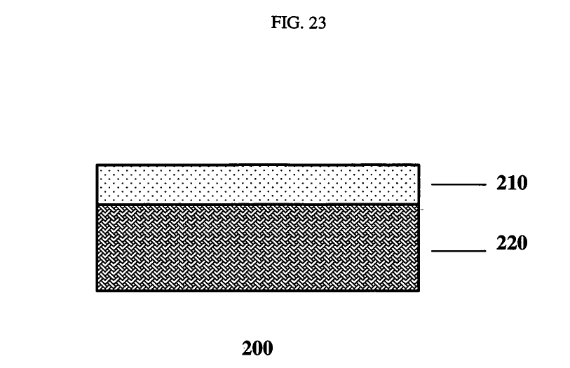

In certain embodiments of the invention, illustrated in FIG. 23, the oral solid dosage form is a bilayer tablet 200, containing one or more drugs, pharmaceutically acceptable excipients or salts thereof, optionally permeation and/or dissolution enhancers, one or more bioadhesive polymer compositions, formulated in two monolithic layers 210 and 220. Various drug release profiles can be achieved by tailoring the composition and/or configuration of each layer. In these embodiments, the tablet is designed to provide immediate release (IR) or controlled release (CR) of one or more soluble, poorly soluble or insoluble drugs from layer 210, and optionally an extended release (ER) of one or more soluble drugs from the other layer 220. One or more bioadhesive polymer compositions are incorporated into layer 220. This layer may optionally contain release rate controlling polymer(s), pore former(s), and/or other polymer(s) to regulate its rigidity and permeability. The cross-section of this dosage form is illustrated in FIG. 23.

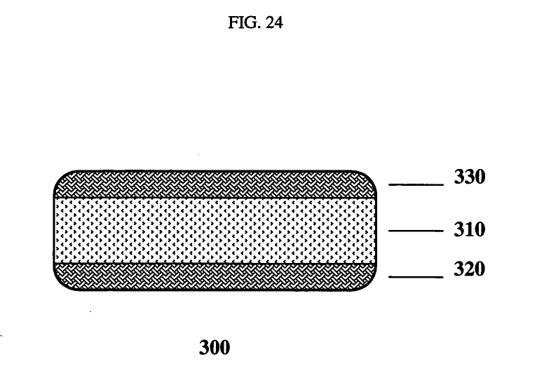

In certain embodiments of the invention, illustrated in FIG. 24, the oral solid dosage form is a trilayer tablet 300, containing one or more drugs, pharmaceutically acceptable excipients or salts thereof, optionally permeation and/or dissolution enhancers, one or more bioadhesive polymer compositions, formulated in three monolithic layers 310, 320 and 330. Various drug release profiles can be achieved by tailoring the composition and/or configuration of each layer. In these embodiments, the tablet is designed to provide controlled release (CR) of one or more soluble, poorly soluble or insoluble drug from layer 310, and optionally an extended release (ER) of one or more soluble drugs from the other layers 320 and 330. One or more bioadhesive polymer compositions may be incorporated into layers 320 and 330. These layers may have same or different compositions, and may optionally contain release rate controlling polymer(s), pore former(s), and other polymer(s) to regulate their rigidity and permeability. The cross-section of this dosage form is illustrated in FIG. 24.

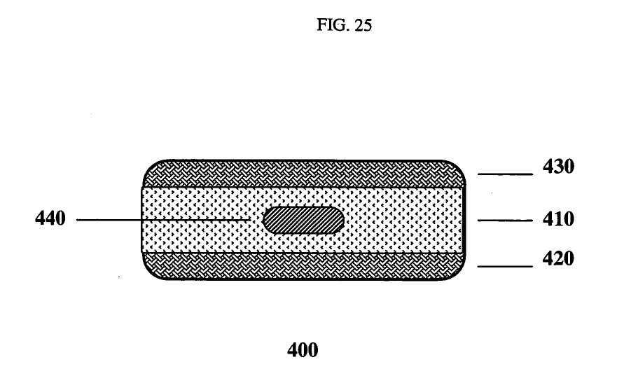

In certain embodiments of the invention, illustrated in FIG. 25, the oral solid dosage form is a trilayer tablet with a pre-compressed insert 400, containing one or more drugs, pharmaceutically acceptable excipients or salts thereof, optionally permeation and/or dissolution enhancers, one or more bioadhesive polymer compositions, formulated in three layers 410, 420 and 430, and a pre-compressed tablet 440, inserted in layer 410. Various drug release profiles can be achieved by tailoring the composition and/or configuration of each layer and the pre-compressed insert. In this embodiment, the tablet is designed to provide ascending controlled release (aCR) of one or more poorly soluble or insoluble drugs from layer 410 and pre-compressed insert 440, and optionally an extended release (ER) of one or more soluble drugs from layers 420 and 430. One or more bioadhesive polymer compositions may be incorporated into layers 420 and 430. These layers may have same or different compositions, and may optionally contain release rate controlling polymer(s), pore former(s), and other polymer(s) to regulate their rigidity and permeability. The cross-section of this dosage form is illustrated in FIG. 25.

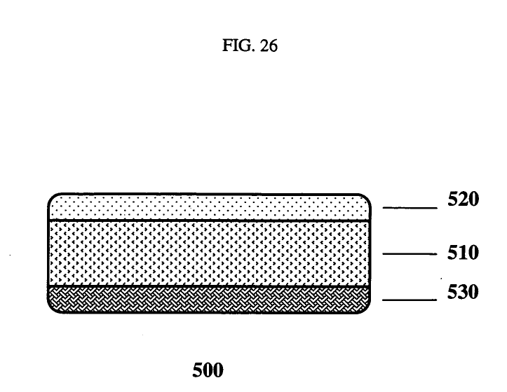

In certain embodiments of the invention, illustrated in FIG. 26, the oral solid dosage form is a trilayer tablet 500, containing one or more drugs, pharmaceutically acceptable excipients or salts thereof, optionally permeation and/or dissolution enhancers, one or more bioadhesive polymer compositions, formulated in three monolithic layers 510, 520 and 530. Various drug release profiles can be achieved by tailoring the composition and/or configuration of each layer. In these embodiments, the tablet layers are designed to provide immediate release (IR) layer 520, and/or controlled release (CR) layer 510, of one or more soluble, poorly soluble or insoluble drugs, and optionally an extended release (ER) layer 530, of one or more soluble drugs. One or more bioadhesive polymer compositions are incorporated into layer 530. This layer may optionally contain release rate controlling polymer(s), pore former(s), and other polymer(s) to regulate its rigidity and permeability. The cross-section of this dosage form is illustrated in FIG. 26.

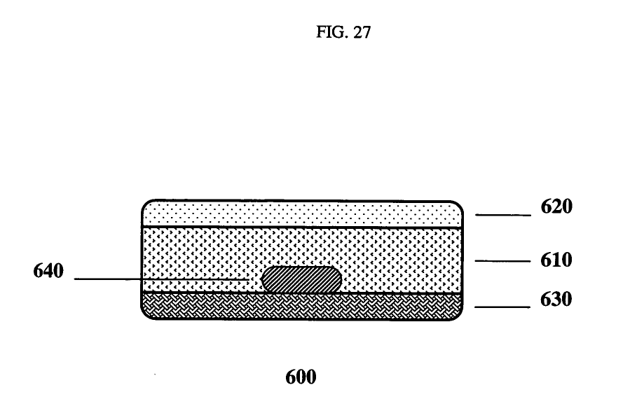

In certain embodiments of the invention, illustrated in FIG. 27, the oral solid dosage form is a trilayer tablet with a pre-compressed insert 600, containing one or more drugs, pharmaceutically acceptable excipients or salts thereof, optionally permeation and/or dissolution enhancers, and one or more bioadhesive polymer compositions, formulated in three layers 610, 620 and 630, and a pre-compressed tablet 640, inserted in layer 610, laying approximately on the middle of the bottom layer 630. Various drug release profiles can be achieved by tailoring the composition and/or configuration of each layer and the pre-compressed insert. In this embodiment, the tablet layers and the pre-compressed insert are designed to provide immediate release (IR) from layer 620, and ascending controlled release (aCR) from layer 610 and pre-compressed insert 640, of one or more soluble, poorly soluble or insoluble drug, and optionally an extended release (ER), from layer 630, of one or more soluble drugs. One or more bioadhesive polymer compositions is incorporated into layer 630. This layer may contain release rate controlling polymer(s), pore former(s), and other polymer(s) to regulate its rigidity and permeability. The cross-section of this dosage form is illustrated in FIG. 27.

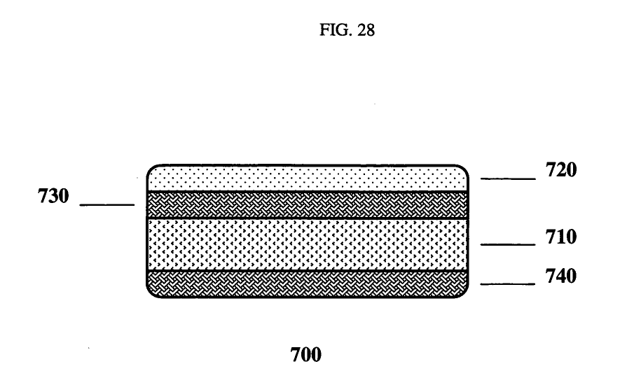

In certain embodiments of the invention, illustrated in FIG. 28, the oral solid dosage form is a quadrilayer tablet 700, containing one or more drugs, pharmaceutically acceptable excipients or salts thereof, optionally permeation and/or dissolution enhancers, and one or more bioadhesive polymer compositions, formulated in four monolithic layers 710, 720, 730 and 740. Various drug release profiles can be achieved by tailoring the composition and/or configuration of each layer. In this embodiment, the tablet layers are designed to provide immediate release (IR), from layer 720, and controlled release (CR), from layer 710, of one or more soluble, poorly soluble or insoluble drugs, and optionally an extended release (ER), from layers 730 and/or 740, of one or more soluble drugs. One or more bioadhesive polymer compositions is incorporated into layers 730 and 740. These layers may optionally contain release rate controlling polymer(s), pore former(s), and other polymer(s) to regulate their rigidity and permeability. The cross-section of this dosage form is illustrated in FIG. 28.

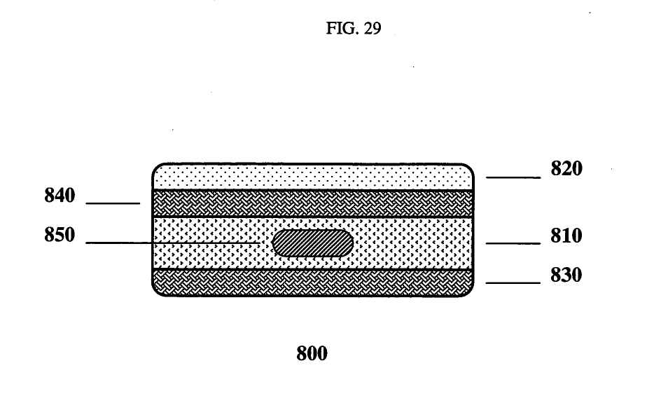

In certain embodiments of the invention, illustrated in FIG. 29, the oral solid dosage form is a quadrilayer tablet with a pre-compressed insert 800, containing one or more drugs, pharmaceutically acceptable excipients or salts thereof, optionally permeation and/or dissolution enhancers, and one or more bioadhesive polymer compositions, formulated in four monolithic layers, 810, 820, 830 and 840, and a pre-compressed tablet 850, inserted in the center of layer 810. Various drug release profiles can be achieved by tailoring the composition and/or configuration of each layer and the pre-compressed insert. In this embodiment, the tablet layers are designed to provide immediate release (IR), from layer 820, and ascending controlled release (aCR), from layer 810 and pre-compressed insert 850, of one or more soluble, poorly soluble or insoluble drug, and optionally an extended release (ER), layers 830 and/or 840, of one or more soluble drug. One or more bioadhesive polymer compositions is incorporated into layers 830 and 840. These layers may optionally contain release rate controlling polymer(s), pore former(s), and other polymer(s) to regulate their rigidity and permeability. The cross-section of this dosage form is illustrated in FIG. 29.

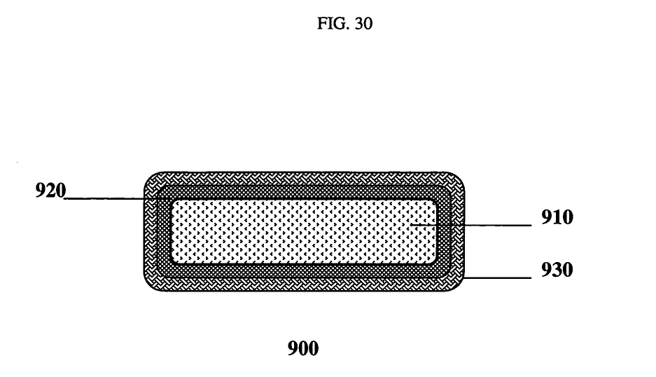

In certain embodiments of the invention, illustrated in FIG. 30, the oral solid dosage form is a monolayer matrix tablet, 900, containing one or more drugs, pharmaceutically acceptable excipients or salts thereof, optionally permeation and/or dissolution enhancers, and optionally one or more bioadhesive polymer compositions, formulated in a single monolithic layer 910. The tablet may be optionally coated with a release rate-controlling membrane 920, before applying the bioadhesive coating membrane 930. Optionally the release rate-controlling and bioadhesive membranes, 920 and 930, may have plasticizer(s), pore former(s), and other polymer(s) to regulate their rigidity and permeability. Various drug release profiles can be achieved by tailoring the composition and/or configuration of the core tablet and the coating membrane(s). In this embodiment, the tablet is designed to provide controlled release (CR) of one or more soluble, poorly soluble or insoluble drug from all sides. The cross-section of this dosage form is illustrated in FIG. 30.

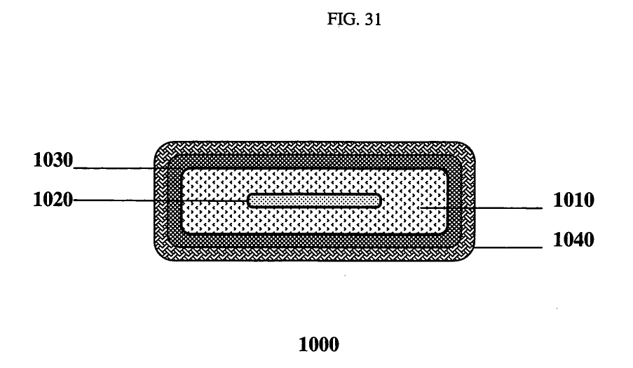

In certain embodiments of the invention, illustrated in FIG. 31, the oral solid dosage form is a monolayer tablet with a pre-compressed insert 1000, containing one or more drugs, pharmaceutically acceptable excipients or salts thereof, optionally permeation and/or dissolution enhancers, and optionally one or more bioadhesive polymer compositions, formulated in a single layer 1010, and a pre-compressed tablet 1020, inserted in the center of that layer. The tablet may be optionally coated with a release rate-controlling membrane 1030, before applying the bioadhesive coating membrane 1040. Optionally the release rate-controlling and bioadhesive membranes 1030 and 1040, may have plasticizer(s), pore former(s), and other polymer(s) to regulate their rigidity and permeability. Various drug release profiles can be achieved by tailoring the composition and/or configuration of the core tablet and the coating membrane(s). In this embodiment, the tablet is designed to provide controlled release (CR) or ascending controlled release (aCR) of one or more soluble, poorly soluble or insoluble drug from all sides. The cross-section of this dosage form is illustrated in FIG. 31.

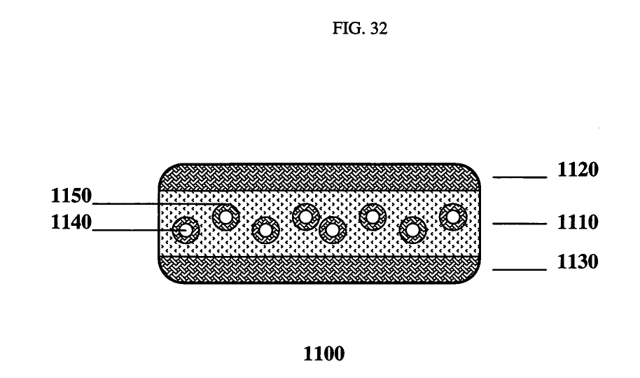

In certain embodiments of the invention, illustrated in FIG. 32, the oral solid dosage form is a trilayer tablet with granulated, spheronized, pelletized, or mini-tableted multiparticulates 1100, containing one or more drugs, pharmaceutically acceptable excipients or salts thereof, optionally permeation and/or dissolution enhancers, and one or more bioadhesive polymer compositions, formulated in three layers, 1110, 1120 and 1130, and granulated, spheronized, pelletized, or mini-tableted multiparticulates 1140, dispersed in layer 1110. The multiparticulates are film-coated with one or more bioadhesive polymer compositions 1150. A release rate-controlling membrane may be optionally applied onto the multiparticulates as a sub-coat before applying the bioadhesive layer. A rapidly dissolving non-functional membrane may be applied as a top-coat onto the bioadhesive pellets. The top-coat membrane may serve different purposes, including functioning as a moisture and/or oxygen barrier, and isolating the bioadhesive multiparticulates from each other immediately upon the release from the carrying layer 1110. Various drug release profiles can be achieved by tailoring the composition and/or configuration of each layer. In this embodiment, the tablet is designed to provide controlled release (CR) of one or more soluble, poorly soluble or insoluble drug from bioadhesive multiparticulates 1140/1150 and possibly layer 1110, and optionally an extended release (ER) of one or more soluble drug from layers 1120 and/or 1130. One or more bioadhesive polymer compositions is incorporated into layers 1120 and 1130. These layers may have same or different compositions, and may optionally contain release rate controlling polymer(s), pore former(s), and other polymer(s) to regulate their rigidity and permeability. The cross-section of this dosage form is illustrated in FIG. 32.

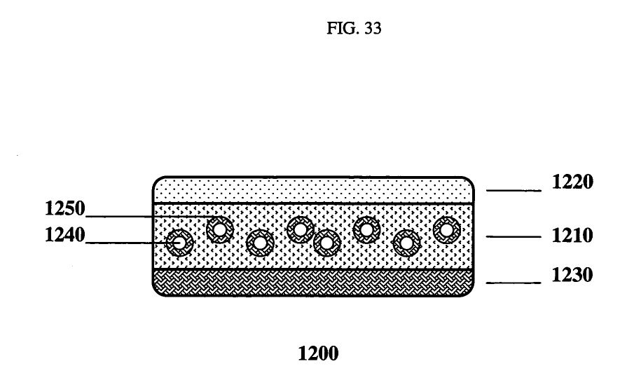

In certain embodiments of the invention, illustrated in FIG. 33, the oral solid dosage form is a trilayer tablet with granulated, spheronized, pelletized, or mini-tableted multiparticulates 1200, containing one or more drugs, pharmaceutically acceptable excipients or salts thereof, optionally permeation and/or dissolution enhancers, and one or more bioadhesive polymer compositions, formulated in three layers, 1210, 1220 and 1230, and granulated, spheronized, pelletized, or mini-tableted multiparticulates 1240, dispersed in layer 1210. The multiparticulates are film-coated with one or more bioadhesive polymer compositions 1250. A release rate-controlling membrane may be optionally applied onto the multiparticulates as a sub-coat before applying the bioadhesive layer. A rapidly dissolving non-functional membrane may be applied as a top-coat onto the bioadhesive pellets. The top-coat membrane may serve different purposes, including functioning as a moisture and/or oxygen barrier, and isolating the bioadhesive multiparticulates from each other immediately upon the release from the carrying layer 1210. Various drug release profiles can be achieved by tailoring the composition and/or configuration of each layer. In this embodiment, the tablet is designed to provide immediate release (IR), from layer 1220, and controlled release (CR), from bioadhesive multiparticulates 1240/1250 and possibly layer 1210, of one or more soluble, poorly soluble or insoluble drug, and optionally an extended release (ER) of one or more soluble drug from layers 1240 and/or 1250. One or more bioadhesive polymer compositions is incorporated into layers 1240 and 1250. These layers may have same or different compositions, and may optionally contain release rate controlling polymer(s), pore former(s), and other polymer(s) to regulate their rigidity and permeability. The cross-section of this dosage form is illustrated in FIG. 33.

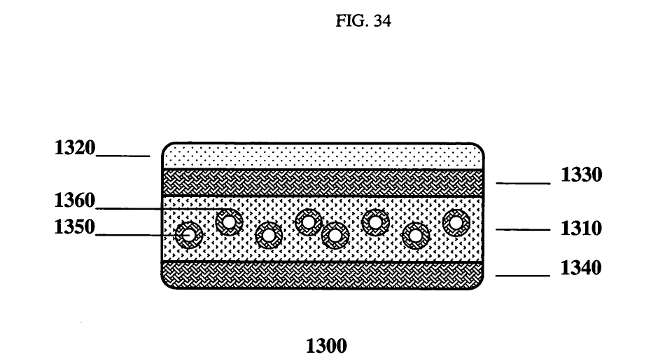

In certain embodiments of the invention, illustrated in FIG. 34, the oral solid dosage form is a quadrilayer tablet with granulated, spheronized, pelletized, or mini-tableted multiparticulates 1300, containing one or more drugs, pharmaceutically acceptable excipients or salts thereof, optionally permeation and/or dissolution enhancers, and one or more bioadhesive polymer compositions, formulated in four layers, 1310, 1320, 1330, and 1340, and granulated, spheronized, pelletized, or mini-tableted multiparticulates 1350, dispersed in layer 1310. The multiparticulates are film-coated with one or more bioadhesive polymer compositions 1360. A release rate-controlling membrane may be optionally applied onto the multiparticulates as a sub-coat before applying the bioadhesive layer. A rapidly dissolving non-functional membrane may be applied as a top-coat onto the bioadhesive multiparticulates. The top-coat membrane may serve different purposes, including functioning as a moisture and/or oxygen barrier, and isolating the bioadhesive multiparticulates from each other immediately upon the release from the carrying layer 1310. Various drug release profiles can be achieved by tailoring the composition and/or configuration of each layer and the multiparticulates. In this embodiment, the tablet is designed to provide immediate release (IR), from layer 1320, and controlled release (CR), from bioadhesive multiparticulates 1350/1360 and possibly layer 1310, of one or more soluble, poorly soluble or insoluble drug, and optionally an extended release (ER) of one or more soluble drug from layers 1330 and/or 1340. One or more bioadhesive polymer compositions is incorporated into layers 1330 and 1340. These layers may have same or different compositions, and may optionally contain release rate controlling polymer(s), pore former(s), and other polymer(s) to regulate their rigidity and permeability. The cross-section of this dosage form is illustrated in FIG. 34.

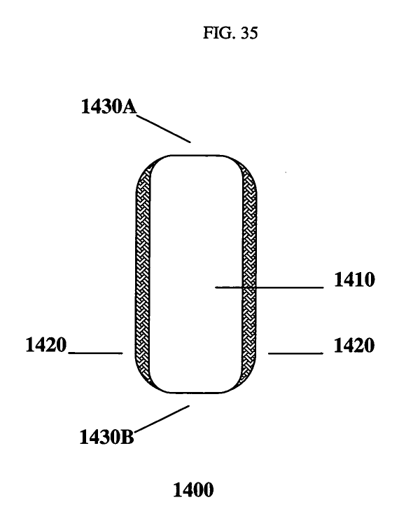

In certain embodiments of the invention, illustrated in FIG. 35, the solid oral dosage form is a longitudinally compressed tablet 1400, containing one or more drugs, pharmaceutically acceptable excipients or salts thereof, optionally permeation and/or dissolution enhancers, and optionally one or more bioadhesive polymer compositions, formulated in a single monolithic layer 1410. The tablet is sealed peripherally with a layer of bioadhesive composition 1420, leaving the upper and lower sides, 1430A and 1430B, of the tablet available for drug release. Optionally, the tablet may be coated with a release rate-controlling layer before applying the bioadhesive coating. Optionally, the release rate-controlling and bioadhesive coatings may have plasticizer(s), pore former(s), and other polymer(s) to regulate their rigidity and permeability. Various drug release profiles, and preferably and more advantageously, zero-order release profiles, can be achieved by tailoring the composition of the core matrix. The cross-section of this dosage form is illustrated in FIG. 35.

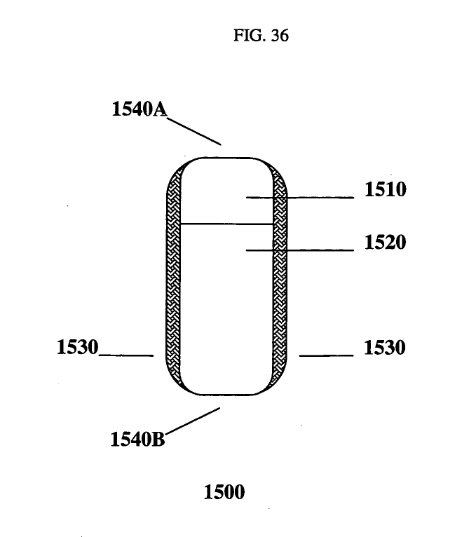

In certain embodiments of the invention, illustrated in FIG. 36, the solid oral dosage form is a longitudinally compressed tablet 1500, containing one or more drugs, pharmaceutically acceptable excipients or salts thereof, optionally permeation and/or dissolution enhancers, and optionally one or more bioadhesive polymer compositions, formulated in two monolithic layers, 1510 and 1520. The tablet is sealed peripherally with a layer of bioadhesive composition 1530, leaving the upper and lower sides, 1540A and 1540B, of the tablet available for drug release. Optionally, the bioadhesive coating may have plasticizer(s), pore former(s), and other polymer(s) to regulate its rigidity and permeability. Various drug release profiles can be achieved by tailoring the composition and/or configuration of each layer. In this embodiment, the tablet is designed to provide immediate release (IR) of one or more soluble, poorly soluble or insoluble drugs from layer 1510, and controlled release (CR) of one or more soluble drugs from the other layer 1520. The cross-section of this dosage form is illustrated in FIG. 36.

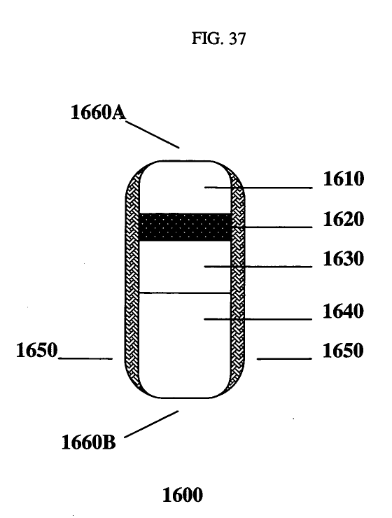

In certain embodiments of the invention, illustrated in FIG. 37, the solid oral dosage form is a longitudinally compressed tablet 1600, containing one or more drugs, pharmaceutically acceptable excipients or salts thereof, optionally permeation and/or dissolution enhancers, and optionally one or more bioadhesive polymer compositions, formulated in four monolithic layers, 1610, 1620, 1630 and 1640. The tablet is sealed peripherally with a layer of bioadhesive composition 1650, leaving the upper and lower sides, 1660A and 1660B, of the tablet available for drug release. Optionally, the bioadhesive coating may have plasticizer(s), pore former(s), and other polymer(s) to regulate its rigidity and permeability. Various drug release profiles can be achieved by tailoring the composition and/or configuration of each layer. In this embodiment, the tablet is designed to provide immediate release (IR) of one or more soluble, poorly soluble or insoluble drugs from layer 1610, and controlled release (CR) of one or more soluble, poorly soluble or insoluble drugs from layer 1640, followed by fast release of one or more soluble, poorly soluble or insoluble drugs from layer 1640. Layers 1610 and 1630 are separated by a slow dissolving passive matrix 1620, which may completely dissolve following the depletion of drug(s) from layer 1640. The cross-section of this dosage form is illustrated in FIG. 37.

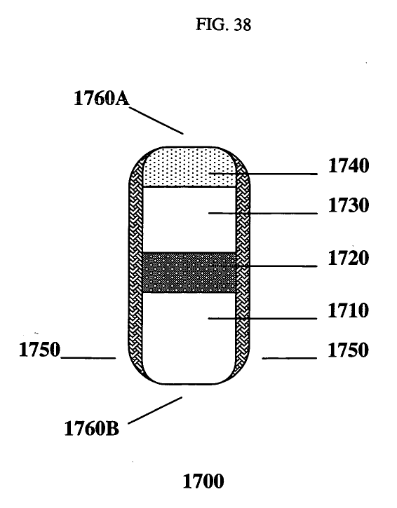

In certain embodiments of the invention, illustrated in FIG. 38, the solid oral dosage form is a longitudinally compressed tablet 1700, containing one or more drugs, pharmaceutically acceptable excipients or salts thereof, optionally permeation and/or dissolution enhancers, and optionally one or more bioadhesive polymer compositions, formulated in four monolithic layers, 1710, 1720, 1730 and 1740. The tablet is sealed peripherally with a layer of bioadhesive composition 1750, leaving the upper and lower sides, 1760A and 1760B, of the tablet available for drug release. Optionally, the bioadhesive coating may have plasticizer(s), pore former(s), and other polymer(s) to regulate its rigidity and permeability. Various drug release profiles can be achieved by tailoring the composition and/or configuration of each layer. In this embodiment, the tablet is designed to provide immediate release (IR) or fast controlled release (CR) of one or more soluble, poorly soluble or insoluble drugs from layer 1710, followed by delayed release of one or more soluble, poorly soluble or insoluble drugs from layer 1730 in an immediate release (IR) or fast controlled release (CR) fashion. Layer 1740 is a slow dissolving passive matrix 1720, which completely dissolves following the depletion of drug(s) from layer 1710. Layers 1710 and 1730 are separated by a slow dissolving passive matrix 1720, which may completely dissolve following the depletion of drug(s) from layers 1710 and 1730. The cross-section of this dosage form is illustrated in FIG. 38.

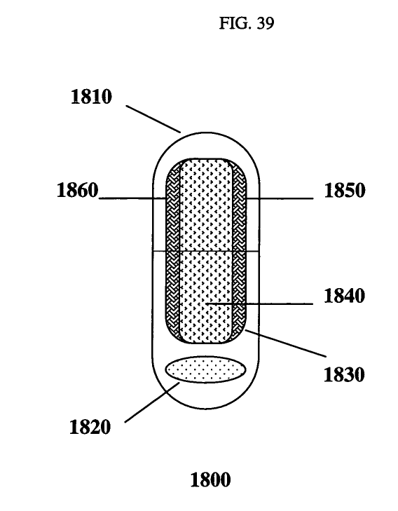

In certain embodiments of the invention, illustrated in FIG. 39, the oral solid dosage form 1800, is a hard shell two-piece capsule 1810, containing a monolayer matrix tablet 1820, and a trilayer tablet 1830. The monolayer tablet 1820, containing one or more drugs, pharmaceutically acceptable excipients or salts thereof, optionally permeation and/or dissolution enhancers, and optionally one or more bioadhesive polymer compositions, formulated in a single layer matrix. The trilayer tablet 1830, contains one or more drug, pharmaceutically acceptable excipients, optionally permeation and/or dissolution enhancers, and one or more bioadhesive polymer compositions, formulated in three monolithic layers, 1840, 1850 and 1860. Various drug release profiles can be achieved by tailoring the composition and/or configuration of each tablet. In this embodiment, the tablets are designed to provide immediate release (IR) of one or more soluble, poorly soluble or insoluble drugs from monolayer tablet 1820, and controlled release (CR) of one or more soluble, poorly soluble or insoluble drugs from trilayer tablet 1830. One or more bioadhesive polymer compositions is incorporated into layers 1850 and 1860. These layers may have identical or different compositions, and may optionally contain release rate controlling polymer(s), pore former(s), and other polymer(s) to regulate their rigidity and permeability. The cross-section of this dosage form is illustrated in FIG. 39.

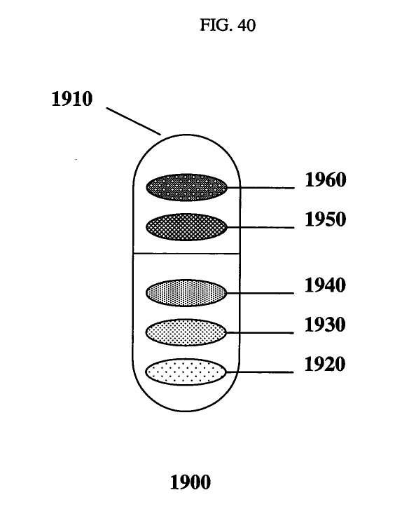

In certain embodiments of the invention, illustrated in FIG. 40, the oral solid dosage form 1900, is a hard shell two-piece capsule 1910, containing a multiplicity of monolayer matrix tablets, 1920, 1930, 1940, 1950, and 1960. The monolayer tablets, contains one or more drug, pharmaceutically acceptable excipients, optionally permeation and/or dissolution enhancers, and optionally one or more bioadhesive polymer compositions, formulated in a single layer matrix. Various drug release profiles can be achieved by tailoring the composition and/or configuration of each tablet. In this embodiment, the tablets are designed to provide immediate release (IR) and controlled release (CR) of one or more soluble, poorly soluble or insoluble drugs. The cross-section of this dosage form is illustrated in FIG. 40.

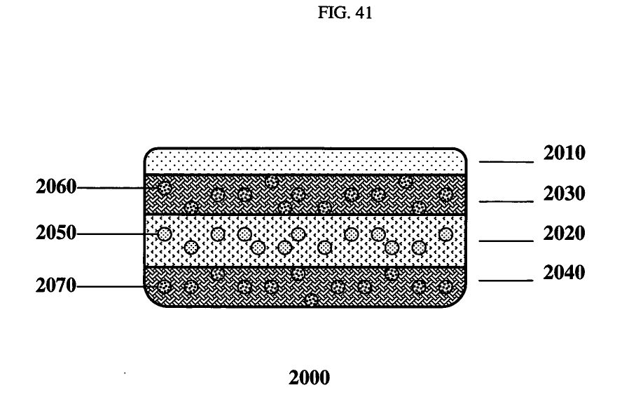

In certain embodiments of the invention, illustrated in FIG. 41, the oral solid dosage form is a quadrilayer tablet with granulated, spheronized, pelletized, or mini-tableted multiparticulates 2000, containing one or more drugs, pharmaceutically acceptable excipients or salts thereof, optionally permeation and/or dissolution enhancers, and one or more bioadhesive polymer compositions, formulated in four layers, 2010, 2020, 2030, and 2040, and granulated, spheronized, pelletized, or mini-tableted multiparticulates, 2050, 2060 and 2070, respectively dispersed in layers 2020, 2030, and 2040. The multiparticulates may be optionally film-coated with one or more bioadhesive polymer compositions. A release rate-controlling membrane may be optionally applied onto the multiparticulates as a sub-coat before applying the bioadhesive layer. A rapidly dissolving non-functional membrane may be applied as a top-coat onto the bioadhesive pellets. The top-coat membrane may serve different purposes, including functioning as a moisture and/or oxygen barrier, and isolating the bioadhesive multiparticulates from each other immediately upon the release from the carrying layers. Various drug release profiles can be achieved by tailoring the composition and/or configuration of each layer and the multiparticulates. In this embodiment, the tablet is designed to provide immediate release (IR), from layer 2010, and controlled release (CR), from multiparticulates 2050 and possibly layer 2020, of one or more soluble, poorly soluble or insoluble drug. Optionally one or more soluble, poorly soluble or insoluble drug is released from the multiparticulates 2060 and 2070, and their respective layers, 2030 and 2040, in an extended release (ER) fashion. One or more bioadhesive polymer compositions is incorporated into layers 2060 and 2070. These layers may have same or different compositions, and may optionally contain release rate controlling polymer(s), pore former(s), and other polymer(s) to regulate their rigidity and permeability. The cross-section of this dosage form is illustrated in FIG. 41.

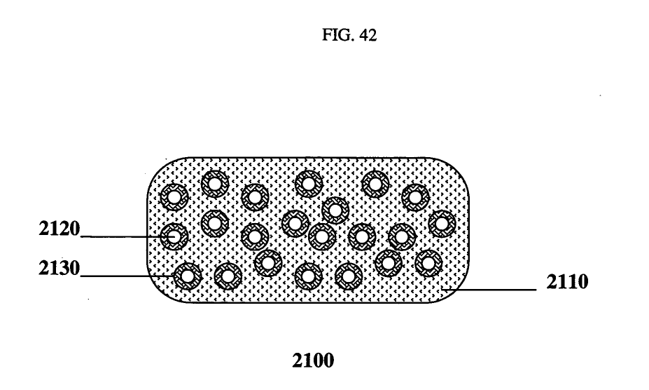

In certain embodiments of the invention, illustrated in FIG. 42, the oral solid dosage form is a monolayer tablet with granulated, spheronized, pelletized, or mini-tableted multiparticulates 2100, containing one or more drugs, pharmaceutically acceptable excipients or salts thereof, optionally permeation and/or dissolution enhancers, and one or more bioadhesive polymer compositions. The tablet is formulated as a single rapidly disintegrating matrix 2110, and contains a solid dispersion of granulated, spheronized, pelletized, or mini-tableted multiparticulates 2120. The multiparticulates are film-coated with one or more bioadhesive polymer compositions 2130. A release rate-controlling membrane may be optionally applied onto the multiparticulates as a sub-coat before applying the bioadhesive layer. A rapidly dissolving non-functional membrane may be applied as a top-coat onto the bioadhesive multiparticulates. The top-coat membrane may serve different purposes, including functioning as a moisture and/or oxygen barrier, and isolating the bioadhesive multiparticulates from each other immediately upon the release from the carrying matrix 2110. Various drug release profiles can be achieved by tailoring the composition and/or configuration of the matrix and the multiparticulates. In this embodiment, the tablet is designed to provide immediate release (IR), from matrix 2110, and controlled release (CR), from bioadhesive multiparticulates 2120/2130, of one or more soluble, poorly soluble or insoluble drug. The cross-section of this dosage form is illustrated in FIG. 42.

These various embodiments are only a sample of numerous possible configurations to deliver the subject dosage forms. Other variations may be readily envisioned based on the principals and teachings of the instant specification.

In these and other embodiments of the invention, the various bioadhesive coatings that can be used are described in detail in the section below. The terms “bioadhesive polymer composition” and “bioadhesive polymer material” is intended to encompass both compositions where the polymer itself is bioadhesive, as well as compositions where a non- or poorly bioadhesive polymer is combined with a compound that imparts bioadhesive properties to the composition as a whole, as described in detail herein.

Preferably, other than the desired immediate release doses, drug eluting devices of the invention release the drug or prodrug contained therein with zero-order kinetics.

Many of the different embodiments described above may be implemented by using rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of a subject pharmaceutical composition at a particular target site.

In certain embodiments, representative dosage forms include hydrogel matrix containing a plurality of tiny pills or other particles (see FIG. 2). The hydrogel matrix comprises a hydrophilic polymer, such as selected from a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin and a hydrophilic colloid. The hydrogel matrix comprises a plurality of tiny pills or particles (such as 4 to 50), each tiny pill or particle may comprise a different ratio of decarboxylase inhibitor/levodopa, either as first or second IR or the second zero-order release portion, etc. The tiny pills or particles may comprise a release rate controlling wall of 0.01 mm to 10 mm thickness to provide for the timed release of drug in different portions. Representative of wall-forming materials include a triglyceryl ester selected from glyceryl tristearate, glyceryl monostearate, glyceryl dipalmitate, glyceryl laureate, glyceryl didecenoate and glyceryl tridecenoate. Other wall forming materials comprise polyvinyl acetate phthalate, methylcellulose phthalate, and microporous vinyl olefins. Procedures for manufacturing tiny pills are disclosed in U.S. Pat. Nos. 4,434,153; 4,721,613; 4,853,229; 2,996,431; 3,139,383 and 4,752,470, which are incorporated by reference herein.

In certain embodiments, the drug-releasing beads are characterized by a dissolution profile wherein 0 to 20% of the beads undergo dissolution and release the drug in 0 to 2 hours, 20 to 40% undergo dissolution and release the drug in 2 to 4 hours, 40 to 60% exhibit dissolution and release in 4 to 6 hours, 60 to 80% in 6 to 8 hours, and 80 to 100% in 8 to 10 hours or longer. The drug-releasing beads can include a central composition or core comprising a drug and pharmaceutically acceptable composition forming ingredients including a lubricant, antioxidant, and buffer. The beads comprise increasing doses of drug, for example, 0.1 mg, 0.2 mg, 0.5 mg, and so forth to a high dose. The beads are coated with a release rate-controlling polymer that can be selected utilizing the dissolution profile disclosed above. The manufacture of the beads can be adapted from, for example, Liu et al., Inter. J. of Pharm. 112: 105-116, 1994; Liu et al., Inter. J. of Pharm. 112: 117-124, 1994; Pharm. Sci., by Remington, 14th Ed. pp. 1626-1628 (1970); Fincher et al., J. Pharm. Sci. 57: 1825-1835, 1968; and U.S. Pat. No. 4,083,949.

Another dosage form provided by the invention comprises a multiplicity of layers (see FIGS. 1, 3, 4, etc.). The phrase “multiplicity of layers” typically denotes 2 to 6 layers in contacting lamination (can be more layers if necessary). The multiplicity of layers are positioned consecutively, that is, one layer after another in order, with a first exposed layer, the sixth layer in contact with the fifth layer and its exposed surface coated with a drug impermeable polymer. The sixth layer is coated with a drug impermeable polymer to insure release of the subject pharmaceutical composition from the first layer to the sixth layer. The biodegradable polymers undergo chemical decomposition to form soluble monomers or soluble polymer units. The biodegradation of polymers usually involves chemically or enzymatically catalyzed hydrolysis. Representative of biodegradable polymers acceptable for an increase drug loading in each layer of from 5 to 50 wt % over the first and successive layers wherein the first layer comprises 100 ng. Representative biodegradable polymers comprise a member selected from biodegradable poly(amides), poly(amino acids), poly(esters), poly(lactic acid), poly(glycolic acid), poly(orthoesters), poly(anhydrides), biodegradable poly(dehydropyrans), and poly(dioxinones). The polymers are known to the art in Controlled Release of Drugs, by Rosoff, Ch. 2, pp. 53-95 (1989); and in U.S. Pat. Nos. 3,811,444; 3,962,414; 4,066,747; 4,070,347; 4,079,038; and 4,093,709.

In still other embodiments, the invention employs a dosage form comprising a polymer that releases a drug by diffusion, flux through pores, or by rupture of a polymer matrix. The drug delivery polymeric system delivers a substantially zero-order release portion of the pharmaceutical composition may optionally comprise an inhibitor/levodopa ratio gradient, wherein the gradient is, for example, a descent in inhibitor/levodopa ratio from a beginning or initial ratio to a final, or lower ratio (comparably less inhibitor). The dosage form comprises an exposed surface at the beginning dose and a distant nonexposed surface at the final dose. The nonexposed surface is coated with a pharmaceutically acceptable material impermeable to the passage of drug. The dosage form structure provides for a delivery of drug at a relatively sustained level, with an optionally changing (e.g., decreasing) inhibitor/levodopa ratio from the beginning to the final delivered dose of the second portion of the dosage form. The ratio may also be different in the first and second (if present) IR (or other substantially ascending dose portion) according to the instant invention.

The dosage form matrix can be made by procedures known to the polymer art. In one manufacture, 3 to 5 or more casting compositions are independently prepared wherein each casting composition comprises a portion of the dosage form, with each portion overlayered from, for example, a high to low inhibitor/levodopa ratio in the zero-order release dosage portion (second portion). This provides a series of layers that come together to provide a unit polymer matrix with an optionally varying inhibitor/levodopa ratio gradient. In another manufacture, the lower ratio portion is cast first followed by laminating with layers of ascending ratio portions to provide a polymer matrix with an inhibitor/levodopa ratio gradient. An example of providing a dosage form comprises blending a pharmaceutically acceptable carrier, like polyethylene glycol, with a known dose of the subject pharmaceutical composition, and adding it to a silastic medical grade elastomer with a cross-linking agent, like stannous octanoate, followed by casting in a mold. The step is repeated for each successive layer. The system is allowed to set, e.g., for 1 hour, to provide the dosage form. Representative polymers suitable for manufacturing the dosage form include olefin and vinyl polymers, condensation polymers, carbohydrate polymers, and silicon polymers as represented by poly(ethylene), poly(propylene), poly(vinyl acetate), poly(methyl acrylate), poly(isobutyl methacrylate), poly(alginate), poly(amide), and poly(silicone). The polymers and manufacturing procedures are known in Polymers, by Coleman et al., Vol. 31, pp. 1187-1230 (1990); Drug Carrier Systems, by Roerdink et al., Vol. 9, pp. 57-109 (1989); Adv. Drug Delivery Rev., by Leong et al., Vol. 1, pp. 199-233 (1987); Handbook of Common Polymers, Compiled by Roff et al., (1971) published by CRC Press; and U.S. Pat. No. 3,992,518.

In yet other embodiments, the subject pharmaceutical compositions are delivered by way of a transdermal patch, a buccal patch, or a buccal tablet. A patch is generally a flat hollow device with a permeable membrane on one side and also some form of adhesive to maintain the patch in place on the patient's skin, with the membrane in contact with the skin so that the medication can diffuse out of the patch reservoir and into and through the skin. The outer side of the patch is formed of an impermeable layer of material, and the membrane side and the outer side are joined around the perimeter of the patch, forming a reservoir for the medication and carrier between the two layers.

Patch technology is based on the ability to hold an active ingredient in constant contact with the epidermis. Over substantial periods of time, drug molecules, held in such a state, will eventually find their way into the bloodstream. Thus, patch technology relies on the ability of the human body to pick up drug molecules through the skin. Transdermal drug delivery using patch technology has recently been applied for delivery of nicotine, in an effort to assist smokers in quitting, the delivery of nitroglycerine to angina sufferers, the delivery of replacement hormones in post-menopausal women, etc. These conventional drug delivery systems comprise a patch with an active ingredient such as a drug incorporated therein, the patch also including an adhesive for attachment to the skin so as to place the active ingredient in close proximity to the skin. Exemplary patch technologies are available from Ciba-Geigy Corporation and Alza Corporation. Such transdermal delivery devices can be readily adapted for use with the subject pharmaceutical compositions.

The flux of the subject pharmaceutical compositions across the skin can be modulated by changing either (a) the resistance (the diffusion coefficient), or (b) the driving force (the solubility of the drug in the stratum corneum and consequently the gradient for diffusion). Various methods can be used to increase skin permeation by the subject reuptake inhibitors, including penetration enhancers, use of pro-drug versions, superfluous vehicles, iontophoresis, phonophoresis, macroflux with micro projections, and thermophoresis. Many enhancer compositions have been developed to change one or both of these factors. See, for example, U.S. Pat. Nos. 4,006,218; 3,551,154; and 3,472,931, for example, respectively describe the use of dimethylsulfoxide (DMSO), dimethyl formamide (DMF), and N,N-dimethylacetamide (DMA) for enhancing the absorption of topically applied drugs through the stratum corneum. Combinations of enhancers comprising diethylene glycol monoethyl or monomethyl ether with propylene glycol monolaurate and methyl laurate are disclosed in U.S. Pat. No. 4,973,468. A dual enhancer comprising glycerol monolaurate and ethanol for the transdermal delivery of drugs is shown in U.S. Pat. No. 4,820,720. U.S. Pat. No. 5,006,342 lists numerous enhancers for transdermal drug administration comprising fatty acid esters or fatty alcohol ethers of C2 to C4 alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C2 or C3 alkanol; and an inert diluent such as water. Other examples are included in the teachings of U.S. Pat. No. 4,933,184 which discloses the use of menthol as a penetration enhancer; U.S. Pat. No. 5,229,130 discloses the use of vegetable oil (soybean and/or coconut oil) as a penetration enhancer; and U.S. Pat. No. 4,440,777 discloses the use of eucalyptol as a penetration enhancer.

The patch preferably comprises a drug-impermeable backing layer. Suitable examples of drug-impermeable backing layers which may be used for transdermal or medicated patches include films or sheets of polyolefins, polyesters, polyurethanes, polyvinyl alcohols, polyvinyl chlorides, polyvinylidene chloride, polyamides, ethylene-vinyl acetate copolymer (EVA), ethylene-ethylacrylate copolymer (EEA), vinyl acetate-vinyl chloride copolymer, cellulose acetate, ethyl cellulose, metal vapor deposited films or sheets thereof, rubber sheets or films, expanded synthetic resin sheets or films, non-woven fabrics, fabrics, knitted fabrics, paper and foils. Preferred drug-impermeable, elastic backing materials are selected from polyethylene terephthalate (PET), polyurethane, ethylene-vinyl acetate copolymer (EVA), plasticized polyvinylchloride, woven and non-woven fabric. Especially preferred is non-woven polyethyleneterephthalate (PET). Other backings will be readily apparent to those skilled artisan.

The dosage formulations described above, in the forms of cores of tablets and drug eluting devices of the invention, contain one or more excipients, carriers or diluents. These excipients, carriers or diluents can be selected, for example, to control the disintegration rate of a tablet or drug eluting device to fit the desired release profile according to the instant invention. In addition, the one or more carriers (additives) and/or diluents may be pharmaceutically acceptable.

The phrase “pharmaceutically acceptable carrier” as used herein means a pharmaceutically acceptable material, composition or vehicle, such as a liquid or solid filter, diluent, excipient, solvent or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Typical excipients to be added to a capsule formulation include, but are not limited to: fillers such as microcrystalline cellulose, soy polysaccharides, calcium phosphate dihydrate, calcium sulfate, lactose, sucrose, sorbitol, or any other inert filler. In addition, there can be flow aids such as fumed silicon dioxide, silica gel, magnesium stearate, calcium stearate or any other materials that impart good flow properties. A lubricant can also be added if desired, such as polyethylene glycol, leucine, glyceryl behenate, magnesium stearate or calcium stearate.

In certain embodiments, the disintegration time of a particular composition (such as the immediate release composition) may be less than the gastric (or small/large intestinal) retention time. In one embodiment, the disintegration time of a tablet is at least 25% of the gastric retention time, at least 50% of the gastric retention time or at least 75% of the gastric retention time. In other embodiments, the disintegration time of a composition may be formulated to effect a substantially zero-order release, over a period of 2, 4, 6, 8, 12, or 24 hours, for instance.

The formulations can conveniently be presented in unit dosage form and can be prepared by any of the methods well known in the art of pharmacy. All methods include bringing into association the drug with the carrier or diluent which constitutes one or more accessory ingredients. In general, the formulations are prepared by uniformly and intimately bringing into association the agent with the carriers and then, if necessary, dividing the product into unit dosages thereof. It will be understood by those skilled in the art that any vehicle or carrier conventionally employed and which is inert with respect to the active agent, and preferably does not interfere with bioadhesiveness, may be utilized for preparing and administering the pharmaceutical compositions of the present invention. Illustrative of such vehicles and carriers are those described, for example, in Remington's Pharmaceutical Sciences, 18th ed. (1990), the disclosure of which is incorporated herein by reference.

Examples of carriers and diluents include pharmaceutically accepted hydrogels such as alginate, chitosan, methylmethacrylates, cellulose and derivatives thereof (microcrystalline cellulose, hydroxypropyl cellulose, hydroxypropyl methyl cellulose, carboxymethylcellulose, ethylcellulose), agarose and Povidone™, kaolin, magnesium stearate, starch, lactose, sucrose, density-controlling agents such as barium sulfate and oils, dissolution enhancers such as aspartic acid, citric acid, glutamic acid, tartartic acid, sodium bicarbonate, sodium carbonate, sodium phosphate, glycine, tricine, Tromethamine, and TRIS.

The excipients, carriers or diluents can also be selected to control the time until a dosage form detaches from a mucosal membrane. In particular, the addition of one or more disintegrating agents will reduce the time until a tablet or drug eluting device detaches. Alternatively or in combination with the disintegrating agents, an agent that interferes with the mucosa-tablet/device adhesion can be used to control the time until detachment occurs.

As set out above, certain embodiments of the present pharmaceutical compositions may contain a basic functional group, such as amino or alkylamino, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include but are not limited to following: 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, fumarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphate/diphosphate, picrate, pivalate, polygalacturonate, propionate, p-toluenesulfonate, salicylate, stearate, subacetate, succinate, sulfate, sulfosaliculate, suramate, tannate, tartrate, teoclate, thiocyanate, tosylate, triethiodide, undecanoate, and valerate salts, and the like. (See, for example, Berge et al., “Pharmaceutical Salts”, J. Pharm. Sci. 66: 1-19, 1977).

In certain embodiments, the pharmaceutically acceptable salts of the subject compounds include the conventional non-toxic salts of the compounds, e.g., from non-toxic organic or inorganic acids. Particularly suitable are salts of weak acids. For example, such conventional non-toxic salts include those derived from inorganic acids such as hydrochloric, hydrobromic, hydriodic, cinnamic, gluconic, sulfuric, sulfamic, phosphoric, nitric, and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, and magnesium salts and the like. Representative organic amines useful for the formation of base addition salts include ethylamine, diethylamine, ethylenediamine, tromethamin, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Pharmaceutically acceptable antioxidants may also be included. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

### VII. Controlled Release Bioadhesive Layer

In certain embodiments of the invention, the subject dosage form is administered orally to the gastrointestinal (GI) tract. In such embodiments, it is desirable that the drug not be delivered substantially beyond the desired site of action and eliminated before it has had a chance to exert a topical effect or to pass into the bloodstream, particularly in the context of avoiding the gastric emptying and its adverse contribution to the On-Off effect. Thus, it is desirable that the subject drug delivery system adhere to the lining of the appropriate viscus, such that its contents can be delivered as a function of proximity and duration of contact.

An orally ingested product can adhere to either the epithelial surface or the mucus lining of the GI tract. For the delivery of bioactive substances, it can be advantageous to have a polymeric drug delivery device adhere to the epithelium or to the mucous layer. Bioadhesion in the GI tract may proceed in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (e.g., ionic). Physical or mechanical bonds can result from deposition and inclusion of the adhesive material in the crevices of the mucus or the folds of the mucosa. Secondary chemical bonds, contributing to bioadhesive properties, include dispersive interactions (e.g., van der Waals interactions) and stronger specific interactions, which include hydrogen bonds. The hydrophilic functional groups primarily responsible for forming hydrogen bonds are the hydroxyl and the carboxylic groups.

In certain embodiments, the subject dosage forms having increased gastrointestinal retention time. For purposes of this invention, gastric residence time is the time required for a dosage form to transit through the stomach to the pyloric sphincter. For example, a dosage form of the invention has a gastric residence time of at least 3 hours, at least 4 hours, at least 6 hours, at least 8 hours or at least 12 hours. The dosage forms of the invention may also have an increased retention time in the small and/or large intestine, or in the area of the gastrointestinal tract that absorbs the drug contained in the dosage form. For example, dosage forms of the invention can be retained in the small intestine (or one or two portions thereof, selected from the duodenum, the jejunum and the ileum) for at least 6 hours, at least 8 hours or at least 12 hours, such as from 16 to 18 hours. For dosage forms having an enteric coating or an equivalent, the increased gastric residence time may not be applicable. These dosage forms, as a whole, include a bioadhesive polymeric coating that is applied to at least one surface of the tablet or drug eluting device.

Certain polymers for use in the subject invention are described in more details below.

### Polymers

### I. Bioadhesives

a. Polymers

Suitable bioadhesive polymeric coatings are disclosed in U.S. Pat. Nos. 6,197,346, 6,217,908 and 6,365,187 (the contents of which are incorporated herein by reference), and include soluble and insoluble, biodegradable and nonbiodegradable polymers. These can be hydrogels or thermoplastics, homopolymers, copolymers or blends, and/or natural or synthetic polymers. The preferred polymers are synthetic polymers, with controlled synthesis and degradation characteristics. Particularly preferred polymers are anhydride copolymers of fumaric acid and sebacic acid (P(FA:SA)), which have exceptionally good bioadhesive properties when administered to the GI tract. Examples of P(FA:SA) copolymers include those having a 1:99 to 99:1 ratio of fumaric acid to sebacic acid, such as 5:95 to 75:25, for example, 10:90 to 60:40 or at least 15:85 to 25:75. Specific examples of such copolymers have a 20:80 or a 50:50 ratio of fumaric acid to sebacic acid.

Polymers used in dosage forms of the invention produce a bioadhesive interaction (fracture strength) of at least 100 N/m² (10 mN/cm²) when applied to the mucosal surface of rat intestine. The fracture strength of the dosage forms is advantageously at least 250 N/m², at least 500 N/m² or at least 1000 N/m². For example, the fracture strength of a polymer-containing dosage form can be from 100 to 500 N/m². The forces described herein refer to measurements made upon rat intestinal mucosa, unless otherwise stated. The same adhesive measurements made on a different species of animal will differ from those obtained using rats. This difference is attributed to both compositional and geometrical variations in the mucous layers of different animal species as well as cellular variations in the mucosal epithelium. However, the data shows that the same general trends prevail no matter what animal is studied (i.e., P(FA:SA) produces stronger adhesions than polylactic acid (PLA) in rats, sheep, pigs, etc.). For example, the fracture strength of dosage forms of the invention on rat intestine is generally at least 125 N/m², such as at least 150 N/m², at least 250 N/m², at least 500 N/m² or at least 1000 N/m².

The fracture strength of a dosage form can be measured according to the methods disclosed by Duchene et al. Briefly, the dosage form is attached on one side to a tensile tester and is contacted with a testing surface (e.g., a mucosal membrane) on the opposite surface. The tensile tester measures the force required to displace the dosage form from the testing surface. Common tensile testers include a Texture Analyzer and the Instron tensile tester.

In the preferred method for mucoadhesive testing, dosage forms are pressed using flat-faced tooling, 0.3750″ (9.525 mm) in diameter. Dosage form weight will depend on composition; in most cases, the dosage forms have a final weight of 200 mg. These dosage forms are then glued to a plastic 10 mm diameter probe using a common, fast-drying cyanoacrylate adhesive. Once the dosage forms are firmly adhered to the probe, the probe is attached to the Texture Analyzer. The Texture Analyzer is fitted with a 1 kg load cell for maximum sensitivity. The following settings are used:

| Pre-Test Speed | 0.4 | mm/sec | Stop Plot At | Final Position |
|---|---|---|---|---|
| Test Speed | 0.1 | mm/sec | Tare Mode | Auto |
| Post-Test Speed | 0.1 | mm/sec | Delay Acquisition | Off |
| Applied Force | 20.0 | g | Advanced | On |
| | | | Options | |
| Return Distance | 0 | mm | Proportional Gain | 0 |
| Contact Time | 420 | s | Integral Gain | 0 |
| Trigger Type | Auto | | Differential Gain | 0 |
| Trigger Force | 0.5 | g | Max. Tracking | 0 mm/sec |
| | | | Speed | |

The Test and Post-Test Speeds are as low as the instrument will allow, to ensure a maximum number of data points captured. The Pre-Test speed is used only until the probe encounters the Trigger Force; i.e., prior to contacting the tissue.

The Proportional, Integral, and Differential Gain are set to 0. These settings, when optimized, maintain the system at the Applied Force for the duration of the Contact Time. With soft tissue as a substrate, however, the probe and dosage form are constantly driven into the deformable surface. This results in visible damage to the tissue. Thus, the probe and dosage form are allowed to relax gradually from the Applied Force by setting these parameters to 0. The tracking speed, which is a measure of how rapidly the feedback is adjusted, is also set to 0.

The tissue on which the dosage forms are tested is secured in the Mucoadhesive Rig; the rig is then completely immersed in a 600 mL Pyrex beaker containing 375 mL of PBS. The tissue is maintained at approximately 37° C. for the duration of the test; no stirring is used as the machine can detect the oscillations from the stir bar.

In the past, two classes of polymers have shown useful bioadhesive properties, hydrophilic polymers and hydrogels. In the large class of hydrophilic polymers, those containing carboxylic groups (e.g., poly[acrylic acid]) exhibit the best bioadhesive properties. It is thus expected that polymers with the highest concentrations of carboxylic groups are preferred materials for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are particularly suitable for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers suitable for the present invention include proteins. (e.g., hydrophilic proteins), such as zein, modified zein, chitin, chitosan, casein, gelatin, gluten, serum albumin, or collagen, and polysaccharides such as cellulose, dextrans, polyhyaluronic acid, polymers of acrylic and methacrylic esters and alginic acid. These are generally less suitable for use in bioadhesive coatings due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers for use in bioadhesive coatings include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers suitable for use in the invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, and polyvinylphenol. Representative bioerodible polymers for use in bioadhesive coatings include polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Polyanhydrides are particularly suitable for use in bioadhesive delivery systems because, as hydrolysis proceeds, causing surface erosion, more and more carboxylic groups are exposed to the external surface. However, polylactides erode more slowly by bulk erosion, which is advantageous in applications where it is desirable to retain the bioadhesive coating for longer durations. In designing bioadhesive polymeric systems based on polylactides, polymers that have high concentrations of carboxylic acid are preferred. The high concentrations of carboxylic acids can be attained by using low molecular weight polymers (MW of 2000 or less), because low molecular weight polymers contain a high concentration of carboxylic acids at the end groups.

The polymers listed above can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif., or can alternatively be synthesized from monomers obtained from these suppliers using standard techniques.

When the bioadhesive polymeric coating is a synthetic polymer coating, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric) anhydride, blends, and copolymers of thereof. Preferably, the synthetic polymer is poly(fumaric-co-sebacic) anhydride.

Another group of polymers suitable for use as bioadhesive polymeric coatings are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymeric coatings are polymers having a hydrophobic backbone with at least one hydrophilic group pendant from the backbone. Suitable hydrophilic groups are groups that are capable of hydrogen bonding to another functional group. Example of such hydrophilic groups include negatively charged groups such as carboxylic acids, sulfonic acids and phosphonic acids, positively charged groups such as (protonated) amines and neutral, polar groups such as amides and imines. Preferably, the hydrophilic groups are selected to not interfere and instead to enhance the bioadhesiveness of the polymers. The hydrophilic groups can be either directly attached to a hydrophobic polymer backbone or attached through a spacer group. Typically, a spacer group is an alkylene group, particularly a C₁-C₈ alkyl group such as a C₂-C₆ alkyl group. Preferred compounds containing one or more hydrophilic groups include amino acids (e.g., phenyalanine, tyrosine and derivatives thereof) and amine-containing carbohydrates (sugars) such as glucosamine.

Polymers can be modified by increasing the number of carboxylic groups accessible during biodegradation, or on the polymer surface. The polymers can also be modified by binding amino groups to the polymer. The polymers can be modified using any of a number of different coupling chemistries available in the art to covalently attach ligand molecules with bioadhesive properties to the surface-exposed molecules of the polymeric microspheres.

The attachment of any positively charged ligand, such as polyethyleneimine or polylysine, to a polymer may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most polymers with the appropriate chemistry, such as with carbodiimidazole (CDI), and be expected to influence the binding to the gut. For example, polyclonal antibodies raised against components of mucin or else intact mucin, when covalently coupled to a polymer, would provide for increased bioadhesion. Similarly, antibodies directed against specific cell surface receptors exposed on the lumenal surface of the intestinal tract would increase the residence time when coupled to polymers using the appropriate chemistry. The ligand affinity need not be based only on electrostatic charge, but other useful physical parameters such as solubility in mucin or specific affinity to carbohydrate groups.

The covalent attachment of any of the natural components of mucin in either pure or partially purified form to the polymers would increase the solubility of the polymer in the mucin layer. The list of useful ligands would include but not be limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, such as polyaspartic acid and polyglutamic acid, may also increase bioadhesiveness. The polyamino chains would increase bioadhesion by means of chain entanglement in mucin strands as well as by increased carboxylic charge.

In certain embodiments, certain polymers suitable for the subject invention may be blended with catechol or a catechol derivative. Such polymers may be any non-biodegradable or biodegradable polymer. The polymers can be homopolymers or copolymers. The polymers that are copolymers can be block, alternating or random copolymers. The backbone of the bioadhesive polymer is preferably flexible in order to penetrate mucus and/or epithelial tissue. In the preferred embodiment, the polymer is a hydrophobic polymer. In one embodiment, the polymer is a biodegradable polymer and is used to form an oral dosage formulation.

Examples of biodegradable polymers suitable for use in the invention include synthetic polymers such as poly hydroxy acids, such as polymers of lactic acid and glycolic acid, polyanhydrides, poly(ortho)esters, polyesters, polyurethanes, poly(butyric acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein, modified zein, chitin, chitosan, and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In one aspect of the invention, a bioadhesive polymer is formed by first coupling a compound to a monomer and then polymerizing the coupled monomer. In this embodiment, the monomers are polymerized to form a polymer, including biodegradable and non-biodegradable polymers. Suitable polymers include, but are not limited to: polyanhydrides, polyamides, polycarbonates, polyalkylenes, polyalkylene oxides such as polyethylene glycol and poloxamers, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polyhydroxy acids, polysiloxanes, polyurethanes and copolymers thereof, modified celluloses, alkyl cellulose, hydroxyalkyl celluloses, cellulose ethers, cellulose esters, nitro celluloses, chitosan, chitin, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methacrylate) poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate) and poly(octadecyl acrylate).

In some embodiments, one can use non-biodegradable polymers, especially hydrophobic polymers. Examples of preferred non-biodegradable polymers include ethylene vinyl acetate, poly(meth)acrylic acid, copolymers of maleic anhydride with other unsaturated polymerizable monomers, poly(butadiene maleic anhydride), polyamides, copolymers and mixtures thereof, and dextran, cellulose and derivatives thereof.

Hydrophobic polymers include polyanhydrides, poly(ortho)esters, and polyesters such as polycaprolactone. In the preferred embodiment, the polymer is sufficiently hydrophobic that it is not readily water soluble, for example, the polymer should be soluble up to less than about 1% w/w in water, preferably about 0.1% w/w in water, at room temperature or body temperature. In the most preferred embodiment, the polymer is a polyanhydride, such as a poly(butadiene maleic anhydride) and other copolymers of maleic anhydrides.

Polyanhydrides may be formed from dicarboxylic acids as described in U.S. Pat. No. 4,757,128 to Domb et al. Suitable diacids include: aliphatic dicarboxylic acids, aromatic dicarboxylic acids, aromatic-aliphatic dicarboxylic acids, combinations of aromatic, aliphatic and aromatic-aliphatic dicarboxylic acids, aromatic and aliphatic heterocyclic dicarboxylic acids, and aromatic and aliphatic heterocyclic dicarboxylic acids in combination with aliphatic dicarboxylic acids, aromatic-aliphatic dicarboxylic acids, and aromatic dicarboxylic acids of more than one phenyl group. Suitable monomers include sebacic acid (SA), fumaric acid (FA), bis(p-carboxyphenoxy)propane (CPP), isophthalic acid (IPh), and dodecanedioic acid (DD).

For materials in which the monomer or polymer has been modified, a wide range of molecular weights are suitable for the polymer that forms the backbone of the bioadhesive material. The molecular weight may be as low as about 200 Da (for oligomers) up to about 2,000 kDa. Preferably the polymer has a molecular weight of at least 1,000 Da, more preferably at least 2,000 Da, most preferably the polymer has a molecular weight of up to 20 kDa or up to 200 kDa. The molecular weight of the polymer may be up to 2,000 kDa. For polymers that are blended with catechol or a catechol derivative, the molecular weight is in the range of 20,000 to 1,000,000 Daltons, preferably 20,000 to 200,000 Daltons.

For materials in which the monomer or polymer has been modified, the range of substitution on the polymer varies greatly and depends on the polymer used and the desired bioadhesive strength. For example, a butadiene maleic anhydride copolymer that is 100% substituted with DOPA will have the same number of DOPA molecules per chain length as a 67% substituted ethylene maleic anhydride copolymer. Typically, the polymer has a percent substitution ranging from 10% to 100%, preferably greater than 50%, ranging up to 100%.

The polymers and copolymers that form the backbone of the bioadhesive material contain reactive functional groups which interact with the functional groups on the aromatic compound. Where the aromatic compound is blended with one or more polymers, the polymers preferably have functional groups that do not react with the functional groups of the aromatic compound.

b. Reactive Functional Groups

For the polymers modified with a catechol functionality, it is important that the polymer or monomer that forms the polymeric backbone contains accessible functional groups that readily react with functional groups contained in the aromatic compounds, such as amines and thiols. In a preferred embodiment, the polymer contains amino reactive moieties (i.e., moieties that react with an amine, preferably to form a covalent linkage), such as aldehydes, ketones, carboxylic acid derivatives, anhydrides (e.g. cyclic anhydrides), alkyl halides, acyl azides, isocyanates, isothiocyanates, and succinimidyl esters.

c. Sidechains Containing Aromatic Groups with One or More Hydroxyl Groups

Aromatic groups containing one or more hydroxyl groups are attached to the polymeric backbone. The aromatic groups may be part of a compound that is grafted to the polymer backbone or the aromatic groups may be part of larger sidechains which are grafted to the polymer backbone. In the preferred embodiment, the aromatic group containing one or more hydroxyl groups is catechol or a derivative thereof. Optionally the aromatic compound is a polyhydroxy aromatic compound, such as a trihydroxy aromatic compound (e.g., phloroglucinol) or a multihydroxy aromatic compound (e.g., tannin). The aromatic moiety can also be an aromatic moiety that includes two or more (e.g., three or more) hydroxyl substituents, methoxy substituents, substituents hydrolyzable to hydroxyl substituents, or a combination thereof, more typically, hydroxyl substituents or substituents hydrolyzable to hydroxyl substituents. A substituent hydrolyzable to a hydroxyl substituent is a substituent, which when cleaved by water (optionally mediated by an enzyme), that leaves a hydroxyl substituent attached to the phenyl ring. Common examples of such substituents include esters (—O—C(O)—R), carbamates (—O—C(O)—NRR′) and carbonates (—O—C(O)—OR).

The catechol derivative may also contain a reactive group, such as an amino, thiol, or halide group. Suitable sidechains, which can be grafted to the polymer backbone, include poly (amino acids), peptides, or proteins, having a molecular weight of 20 kDa or less, where at least 10% of the amino acids contain catechol or catechol-like residues. Preferably greater than 50%, more preferably 75%, and most preferably 100% of the amino acids contain catechol residues. Common amino acids with catechol-like residues are phenylalanine, tyrosine and tryptophan. Additionally, synthetic amino acids that contain catechol residues may be prepared.

A preferred catechol derivative is 3,4-dihydroxyphenylalanine (DOPA), which contains a primary amine. L-DOPA is known to be pharmaceutically active and is used as a treatment for Parkinson's disease. Tyrosine, the immediate precursor of DOPA, which differs only by the absence of one hydroxyl group in the aromatic ring, can also be used. Tyrosine is capable of conversion (e.g. by hydroxylation) to DOPA.

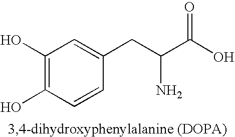

In a preferred embodiment, the aromatic group is an amine-containing aromatic compound, such as an amine-containing catechol derivative. Other suitable compounds for forming blends include 3,4-dimethoxyphenyl-2-hydrazino-2-methyl propanoic acid, 2-aminocarbonyl-amino-3-(3,4-dimethoxyphenyl)-2-methylpropanoic acid, 2-amino-3-(3,4-dimethoxyphenyl)-2-methyl hydrochloride, 2-amino-3-(3,4-dimethoxyphenyl)-2-methyl propane nitrile, methyl-DOPA, 3-O-methylcarbidopa and 4-O-methylcarbidopa, and enantiomers and mixtures thereof.

In another preferred embodiment, the aromatic group is a compound comprising:
a) an aromatic moiety comprising two or more hydroxyl substituents, methoxy substituents, substituents hydrolyzable to hydroxyl substituents, or a combination thereof, and
b) a primary or secondary amino moiety,

where the cumulative amount of the compound (i.e., compound not functionalized to a polymer backbone) that is converted to dopamine when infused into rat striatum is at least 65% less than for an equimolar amount of L-3,4-dihydroxyphenylalanine or where the blood-brain barrier is substantially impermeable to the compound. In certain embodiments, the compounds used to form residues are selected such that the cumulative amount of the compound converted to dopamine when infused into rat striatum is at least 70%, 75%, 80%, 85%, 90%, 95% or 100% (i.e., the compound is not converted to dopamine) less than an equimolar amount of L-3,4-dihydroxyphenylalanine. The cumulative amount of a compound converted to dopamine when infused into rat striatum can be measured according to the method described in Brannan, et al., Brain Res. 718:165-168 (1996), the contents of which are incorporated herein by reference. Briefly, a microdialysis probe is lowered into the corpus striatum of anesthetized rats. The probe generally has a tip length of 3 mm and is perfused with an artificial cerebrospinal fluid solution. Concentrations of dopamine in the microdialysis samples are monitored at regular intervals by HPLC or another suitable analytical method. Once the dopamine concentration reaches a basal level, a 1 mM solution of a sidechain residue compound is perfused into the striatum via the probe, with continued monitoring of the dopamine concentration.

Separately or in addition to selection of aromatic compounds based upon their ability to be converted into dopamine, aromatic compounds can be selected such that the blood-brain barrier is substantially impermeable to these compounds when present as free molecules (i.e., not covalently attached to a polymer). Typically, less than 10%, such as less than 5%, 4%, 3%, 2% or 1%, of a substantially impermeable compound is able to cross the blood-brain barrier. A suitable assay for determining permeability of the blood-brain barrier to a compound is described by Gomes and Soeares-da-Silva in Brain Res. 829:143-150 (1999), the contents of which are incorporated herein by reference. Briefly, the assay measures the uptake of a compound by immortalized rat capillary cerebral endothelial cells (RBE 4), which represent the blood-brain barrier. The endothelial cells are seeded in collagen-treated 24-well plastic culture clusters (16 mm internal diameter) at a density of 40,000 cells per well (20,000 cells/cm²). For 24 hours prior to an experiment, the cell medium is free of fetal bovine serum and basic fibroblast growth factor. Uptake experiments are typically performed 6 days after seeding. On the day of the experiment, the growth medium is aspirated and the cells are washed with Hanks' medium at 4° C., followed by incubating the cells in Hanks' medium at 37° C. for 30 minutes. The cells are incubated for 6 minutes with 2 mL of 1 μM substrate (e.g., sidechain residue compound) in Hanks' medium. Uptake is terminated by rapid removal of uptake solution with a vacuum pump connected to a Pasteur pipette, followed by a rapid wash with cold Hanks' medium and the addition of 250 μL of 0.2 mM perchloric acid. The acidified samples are stored under appropriate conditions until the substrate concentration is measured (e.g., via HPLC).

In another embodiment, the aromatic compounds include all sidechain residue compounds having the moieties discussed above, except L-DOPA and/or DL-DOPA.

Typically, the aromatic moiety is a monocyclic aromatic moiety that includes two or more hydroxyl substituents, methoxy substituents, substituents hydrolyzable to hydroxyl substituents, or a combination thereof, more typically, hydroxyl substituents or substituents hydrolyzable to hydroxyl substituents. Preferably, the aromatic moiety is a phenyl moiety that includes two or more (e.g., three or more) hydroxyl substituents, methoxy substituents, substituents hydrolyzable to hydroxyl substituents, or a combination thereof, more typically, hydroxyl substituents or substituents hydrolyzable to hydroxyl substituents. An exemplary aromatic moiety is catechol. The aromatic moiety can include other substituents in addition to those indicated, but typically does not include additional substituents.

A substituent hydrolyzable to a hydroxyl substituent is a substituent, which when cleaved by water (optionally mediated by an enzyme), that leaves a hydroxyl substituent attached to the phenyl ring. Common examples of such substituents include esters (—O—C(O)—R) and carbonates (—O—C(O)—OR).

The primary or secondary amino moiety can be directly attached to a carbon atom or can be part of a hydrazinyl moiety (—NH—NHR).

Suitable compounds for forming residues include D-3,4-dihydroxyphenylalanine (D-DOPA), (D-, L- or a mixture thereof) carbidopa and (D-, L-, or a mixture thereof) benserazide, which have the following structures, respectively:

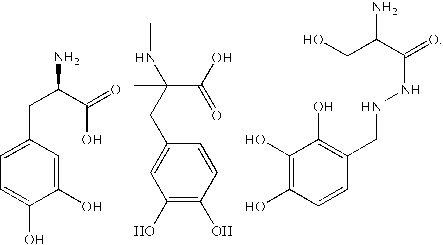

Other suitable compounds for forming residues include 3,4-dimethoxyphenyl-2-hydrazino-2-methyl propanoic acid, 2-aminocarbonyl-amino-3-(3,4-dimethoxyphenyl)-2-methylpropanoic acid, 2-amino-3-(3,4-dimethoxyphenyl)-2-methyl hydrochloride, 2-amino-3-(3,4-dimethoxyphenyl)-2-methyl propane nitrile, methyl-DOPA, 3-O-methylcarbidopa and 4-O-methylcarbidopa, including enantiomers and mixtures thereof.

d. Blends Containing a Catechol or a Catechol Derivative

In one embodiment, the catechol or catechol derivative is blended with a biodegradable or non-biodegradable polymer to form a bioadhesive composition. The polymer is preferably a hydrophobic polymer. Suitable hydrophobic polymers include ethyl cellulose, poly(anhydrides), and polyesters. The preferred catechol derivatives are 3,4-dihydroxyphenylalanine (DOPA), which contains a primary amine, or carbidopa. The catechol derivative can be present in an amount from about 0.5% to about 95% by weight of the polymer. For example, blending polycaprolactone with L-DOPA in a ratio of 2:1 w/w results in a bioadhesive material with an adhesive force of 491 mN/cm² compared to 50 mN/cm² for polycaprolactone alone.

### II. Method of Forming Bioadhesives

Three general methods are used to form the bioadhesive materials. In one embodiment, a compound containing an aromatic group which contains one or more hydroxyl groups is grafted onto a polymer. In this embodiment, the polymeric backbone is a biodegradable polymer. In a second embodiment, the aromatic compound may be coupled to individual monomers and then polymerized. In a third embodiment, the polymer is blended with a compound containing an aromatic group which contains one or more hydroxyl groups.

Any chemistry which allows for the conjugation of a polymer or monomer to an aromatic compound containing one or more hydroxyl groups may be used. For example, if the aromatic compound contains an amino group and the monomer or polymer contains an amino reactive group, this modification to the polymer or monomer is performed through a nucleophilic addition or a nucleophilic substitution reaction, such as a Michael-type addition reaction, between the amino group in the aromatic compound and the polymer or monomer. Additionally, other procedures can be used in the coupling reaction. For example, carbodiimide and mixed anhydride based procedures form stable amide bonds between carboxylic acids or phosphates and amino groups, bifunctional aldehydes react with primary amino groups, bifunctional active esters react with primary amino groups, and divinylsulfone facilitates reactions with amino, thiol, or hydroxy groups.

a. Polymer Grafting

The aromatic compounds are grafted onto the polymer using standard techniques to form the bioadhesive material. An example of the grafting procedure is schematically depicted in Reaction 1, which depicts a nucleophilic substitution reaction between the amino group in the aromatic compound and the polymer. L-DOPA is grafted to maleic anhydride copolymers by reacting the free amine in L-DOPA with the maleic anhydride bond in the copolymer.

A variety of different polymers can be used as the backbone of the bioadhesive material. Representative polymers include 1:1 random copolymers of maleic anhydride with ethylene, vinyl acetate, styrene, or butadiene. The variable portions of the backbone structures are designated as the R groups at the bottom of Reaction 1. In addition, a number of other compounds containing aromatic rings with hydroxy substituents, such as tyrosine or derivatives of catechol, can be used in reaction 1.

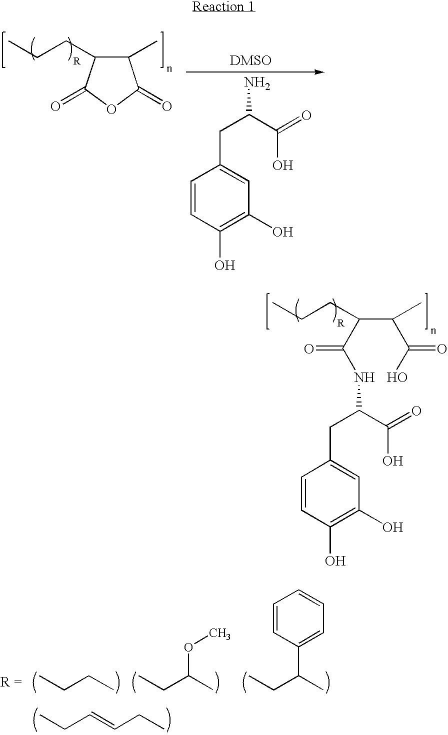

b. Polymer Synthesis

In another embodiment, the polymers are prepared by conjugate addition of a compound containing an aromatic group and an amine functionality to one or more monomers containing an amino reactive group. In the preferred method the monomer is an acrylate or a polymer acrylate. In the most preferred method the monomer is a diacrylate such as 1,4-butanediol diacrylate; 1,3-propanediol diacrylate; 1,2-ethanediol diacrylate; 1,6-hexanediol diacrylate; 2,5-hexanediol diacrylate; or 1,3-propanediol diacrylate. In the coupling reaction, the monomer and the compound containing an aromatic group are each dissolved in an organic solvent (e.g., THF, CH2Cl2, methanol, ethanol, CHCl3, hexanes, toluene, benzene, CCl4, glyme, diethyl ether, etc.) to form two solutions. The resulting solutions are combined, and the reaction mixture is heated to yield the desired polymer. The molecular weight of the synthesized polymer may be determined by the reaction conditions (e.g., temperature, starting materials, concentration, solvent, etc) used in the synthesis.

For example, a monomer, such as 1,4 phenylene diacrylate or 1,4 butanediol diacrylate having a concentration of 1.6 M, and DOPA or another primary amine containing aromatic molecule are each dissolved in an aprotic solvent such as DMF or DMSO to form two solutions, the solutions are mixed in a 1:1 molar ratio between the diacrylate and the amine group and heated to 56° C. to form a bioadhesive material.

c. Blending a Polymer with a Catechol or Catechol Derivative

Blends of a biodegradable or non-degradable polymer with a catechol or catechol derivative can be prepared by mixing, such as by dissolving the polymer and the catechol or catechol derivative in a suitable solvent and then removing the solvent under controlled conditions of temperature and rate of solvent removal. The resulting blends can be spray dried or dried at room temperature. Alternatively, the blend can be prepared by melt blending the polymer and the catechol or catechol derivative at a temperature corresponding to the melting point of the polymer. For example, polycaprolactone can be melt-blended with L-DOPA (m.p. 295° C.) at a temperature of 58-60° C., which corresponds to the meting point of polycaprolactone. The blends can be also coated onto a substrate using melt extrusion, a fluidized bed, or any method of coating known in the art. The catechol or catechol derivative is present in amount from about 0.5% to about 95% by weight of the polymer.

### III. Method for Stabilizing Bioadhesives

The invention includes a bioadhesive material comprising (1) a polymeric component selected from (a) a polymeric backbone and a side chain or side group containing an aromatic group substituted with one or more hydroxyl groups and (b) a polymer blended with an aromatic compound substituted with one or more hydroxyl groups and (2) an additive that stabilizes the polymeric component from erosion, dissolution or both, where at least 50% by weight of a 1 mm thick film of the bioadhesive material remains after 12 hours in a buffered pH 4.5 dissolution bath.

In certain embodiments, the bioadhesive material film is tested in a dissolution bath for 6 hours, 8 hours, 10 hours, 12 hours, 14 hours, 16 hours, 18 hours, 20 hours, 24 hours or longer. In certain such embodiments, the amount of bioadhesive material film remaining after testing in the dissolution bath is at least 50% by weight, at least 60% by weight, at least 70% by weight, at least 80% by weight, at least 90% by weight, at least 95% by weight, at least 97% by weight, at least 98% by weight or even at least 99% by weight. A suitable dissolution bath, a USP II apparatus, is described below in the Examples. In certain embodiments, the dissolution bath is stirred at 50 rpm and the temperature is 37° C.

In certain embodiments, the bioadhesive polymers are stabilized against erosion by incorporating one or more additives selected from (1) polyanhydrides, such as those having a molecular weight average in excess of 20.000, (2) acidic components (including precursors thereof), (3) metal compounds, (4) stabilizing polymers, and (5) hydrophobic components.

a. Polyanhydrides

Suitable polyanhydrides for stabilizing the bioadhesive polymers discussed above are described in U.S. Pat. No. 4,757,128 to Domb et al. and U.S. Pat. No. 5,955,096 to Mathiowitz et al., the contents of which are incorporated herein by reference. Polymers may be synthesized from highly pure isolated prepolymers formed from: aliphatic dicarboxylic acids, aromatic dicarboxylic acids, aromatic-aliphatic dicarboxylic acids, combinations of aromatic, aliphatic and aromatic-aliphatic dicarboxylic acids, aromatic and aliphatic heterocyclic dicarboxylic acids and aromatic and aliphatic heterocyclic dicarboxylic acids in combination with aliphatic dicarboxylic acids, aromatic-aliphatic dicarboxylic acids, and aromatic dicarboxylic acids of more than one phenyl group. For example, the following monomers are suitable for synthesizing bioadhesive copolymers: bis(p-carboxyphenoxy)alkanes; hydroquinone-O,O′ diacetic acid; 1,4-bis-carboxymethyl benzene; 2,2-bis(4-hydroxphenyl)propane-O,O′-diacetic acid; 2,2-bis(4-carboxyphenyl)propane; terephthalic acid; bis(4-carboxyphenyl)alkanes; 1,4-phenylene dipropionic acid; cyclohexane dicarboxylic acids, adipic acid, sebacic acid (SA), bis(p-carboxyphenoxy)propane (CPP), isophthalic acid (IPh), and dodecanedioic acid (DD). A particular polyanhydride is poly(fumaric acid-co-sebacic acid) (pFA:SA) (e.g. a 20:80 copolymer of p(FA:SA)). Another particular polyanhydride is polyadipic anhydride.

Anhydride monomers or oligomers can be incorporated into the polyanhydrides described above to enhance their bioadhesiveness. As used herein, the term “anhydride oligomer” refers to a diacid or polydiacid linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. The anhydride oligomer is hydrolytically labile. As analyzed by gel permeation chromatography, the molecular weight may be, for example, on the order of 200400 for fumaric acid oligomer (FAPP) and 2000-4000 for sebacic acid oligomer (SAPP). In one embodiment, the diacids are those normally found in the Krebs glycolysis cycle. The anhydride oligomer compounds preferably have high chemical reactivity. The anhydride oligomers may be combined with metal oxide particles to improve bioadhesion even more than with the organic additives alone.

Anhydride oligomers can be incorporated into a polyanhydride by combining a finely ground dispersion of particles of oligomer in a solution or dispersion with the polyanhydride. Alternatively, the oligomer compound can be incorporated into the polymer by dispersing the polyanhydride in a solution or dispersion of the oligomer compound and then removing the solvent by evaporation or filtration.

While Applicants do not wish to be bound by theory, it is believed that free carboxylic acid groups of the polyanhydrides form hydrogen bonds with hydroxyl group in the polymers functionalized or blended with catechol and derivatives thereof and/or create a local acidic environment, thereby stabilizing the latter polymers. It is also believed that the erosion of polyanhydrides is less affected by pH than the polymers functionalized or blended with catechol or a derivative, such that a polyanhydride selected for use herein advantageously erodes at a largely pH-independent rate and/or erodes slowly upon hydration.

Typically, the amount of polyanhydride added to a bioadhesive polymer is from about 0.5% to about 75% by weight, preferably about 5% to about 50% and more preferably about 10% to about 25%.

b. Acidic Components

The bioadhesive polymers can additionally be stabilized by the incorporation of a small molecule (i.e., non-polymeric or oligomeric) acidic component, preferably a slow release acidic component. Typically, the acid is a weak organic acid, for example, an acid having a pKa of about 1 to about 7, such as about 1 to about 5.5, typically about 1.2 to 4.5. Preferably, the acid is poorly soluble in water as defined in the USP, but miscible with the bioadhesive polymer. The acid may contain one or more carboxylic, phosphonic, phosphoric, sulfonic, sulfinic or sulfenic acid moieties, preferably two or more acid moieties. Typically, the acid contains two or more carboxylic acid moieties. Exemplary acids include succinic acid, fumaric acid, citric acid, sebacic acid, adipic acid, lactic acid, malic acid, ascorbic acid, tartaric acid and sorbic acid. In certain embodiments, the acid is not citric acid. In certain such embodiments, the acid is not citric acid, fumaric acid, sebacic acid or lactic acid. In other embodiments, the acid is not a sugar. A combination of two or more such acids may be incorporated into a polymer.

The acid may be an acid precursor, particularly an anhydride. An acid precursor is a molecule that is hydrolyzed or metabolized into an acid. Suitable anhydrides includes symmetrical anhydrides (e.g., acetic anhydride, cyclohexanecarboxylic anhydride, hexanoic anhydride, chloroacetic anhydride, thiobenzoic anhydride, thiopropionic anhydride, 2-chloroethanesulfinic anhydride, benzenesulfonic anhydride and cyclic anhydrides formed from two acid groups attached to the same molecule such as succinic anhydride, cyclohexane-1,2,3,4-tetracarboxylic acid 3,4-anhydride and phthalic anhydride), unsymmetric (mixed anhydrides (e.g., acetic propionic anhydride, benzoic thioacetic anhydride, acetic chloroacetic anhydride, benzenesulfinic ethanesulfonic anhydride, chloroacetic-4-nitrobenzenesulfonic anhydride) and chalcogen analogues of anhydrides (e.g., benzoic thioanhydride, 4-chlorocyclohexane-1-carbothioic thioanhydride, acetic propionic thioanhydride, acetic thiopropionic anhydride, propionic thioacetic anhydride, acetic thiopropionic thioanhydride, propionic thioacetic thioanhydride, thioacetic thiopropionic anhydride). Preferably, the anhydride is succinic anhydride, phthalic anhydride, maleic anhydride, adipic anhydride, butyric anhydride, isobutyric anhydride, propionic anhydride or another carboxylic acid anhydride. More preferably, the anhydride is succinic anhydride.

The acids advantageously are present in a bioadhesive polymer for an extended period of time (e.g., not washed away in an aqueous environment), which is typically achieved either by virtue of low water solubility or by virtue of coating the acids with an appropriate coating. Such acids are collectively referred to herein as slow-release acid components. Acids selected on the basis of solubility typically have a solubility in water of less than 10 mg/mL at pH 4.5 and below. Coatings for an acid are selected such that they do not appreciably dissolve at pH 4.5 or below or such that they coat the acid until the formulation (i.e., polymer) into which the coated acid is incorporated has passed through the stomach (e.g., an enteric coating).

Typically, the amount of an acidic component (including acid precursors) added to a bioadhesive polymer is from about 0.5% to about 75% by weight, such as about 1% to about 65%, preferably about 5% to about 50% (about 5% to about 45%, about 10% to about 30%) and more preferably about 10% to about 25%.

c. Metal Compounds

The bioadhesive polymers described above can also be stabilized by the incorporation of a metal compound, as described in U.S. Pat. No. 5,985,312 to Jacob et al.

The metal compounds preferably are water-insoluble metal compounds, such as water-insoluble metal oxides and hydroxides, including oxides of calcium, iron, copper and zinc. The metal compounds can be combined with a wide range of hydrophilic and hydrophobic polymers including proteins, polysaccharides and synthetic biocompatible polymers.

Metal compounds which can be incorporated into polymers preferably are water-insoluble metal compounds, such as water-insoluble metal oxides and metal hydroxides, which are capable of becoming combined with a polymer to thereby improve the bioadhesiveness of the polymer. As defined herein, a water-insoluble metal compound is defined as a metal compound with little or no solubility in water, for example, less than about 0.0 to 0.9 mg/ml.

The water-insoluble metal compounds can be derived from a wide variety of metals, including, but not limited to, calcium, iron, copper, zinc, cadmium, zirconium and titanium. The water insoluble metal compound preferably is a metal oxide or hydroxide. Water insoluble metal compounds of multivalent metals are preferred. Representative metal oxides suitable for use in the compositions described herein include cobalt oxide (I) (CoO), cobalt oxide (II)(Co₂O₃), selenium oxide (SeO₂), chromium double oxide (CrO₂), manganese oxide (MnO₂), titanium oxide (TiO₂), lanthanum oxide (La₂O₃), zirconium oxide (ZrO₂), silicon oxide (SiO₂), scandium oxide (Sc₂O₃), beryllium oxide (BeO), tantalum oxide (Ta₂O₅), cerium oxide (CeO₂), neodymium oxide (Nd₂O₃), vanadium oxide (V₂O₅), molybdenum oxide (MO₂O₃), tungsten oxide (WO), tungsten trioxide (WO₃), samarium oxide (Sm₂O₃), europium oxide (Eu₂O₃), gadolinium oxide (Gd₂O₃), terbium oxide (Tb₄O₇), dysprosium oxide (Dy₂O₃), holmium oxide (Ho₂O₃), erbium oxide (Er₂O₃), thulium oxide (Tm₂O₃), ytterbium oxide (Yb₂O₃), lutetium oxide (Lu₂O₃), aluminum oxide (Al₂O₃), indium oxide (InO₃), germanium oxide (GeO₂), antimony oxide (Sb₂O₃), tellurium oxide (TeO₂), nickel oxide (NiO), and zinc oxide (ZnO). Other oxides include barium oxide (BaO), calcium oxide (CaO), nickel oxide (III) (Ni₂O₃), magnesium oxide (MgO), iron oxide (11) (FeO), iron oxide (III) (Fe₂O₃), copper oxide (II) (CuO), cadmium oxide (CdO), and zirconium oxide (ZrO₂). In certain embodiments, the metal compound is ferric oxide, copper oxide or zinc oxide or a combination thereof. In other embodiments, the metal compound is a zirconate, such as magnesium zirconate or calcium zirconate. In yet other embodiments, the metal compound is a silicate, such as magnesium silicate (e.g., a hydrated magnesium silicate such as talc) or calcium silicate. Advantageously, metal compounds which are incorporated into polymers are metal compounds which are already approved by the FDA or an equivalent agency as either food or pharmaceutical additives, such as zinc oxide or talc.

The water-insoluble metal compounds can be incorporated into a polymer by, for example, one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bed, pan coating, or any similar methods known to those skilled in the art, which produce a metal compound enriched layer on the surface of the device. In one embodiment, nanoparticles or microparticles of the water-insoluble metal compound are incorporated into the polymer, preferably as a uniform dispersion.

Fine metal oxide particles can be produced, for example, by micronizing a metal oxide by mortar and pestle treatment to produce particles ranging in size, for example from 10.0 to 300 nm. The metal oxide particles can be incorporated into a polymer, for example, by dissolving or dispersing the particles into a solution or dispersion of the polymer.

Metal compounds are optionally coated with a protective coating, such as an enteric coating or a rate controlling coating. Such coatings are selected in order to release the metal compound only when the system is exposed to gastric fluid or another targeted environment.

Typically, the amount of a metal compound added to a bioadhesive polymer is from about 1% to about 65% by weight, preferably about 5% to about 45% and more preferably about 10% to about 30%.

d. Stabilizing Polymers

The bioadhesive polymers described above can also be stabilized by the incorporation of certain polymers, particularly a hydrophilic polymer (hydrogel) that forms a rigid gel at pH 4.5 and higher or a hydrophobic polymer. Preferably, a hydrogel has little or no swelling at pH 4.5 or less. One group of suitable polymers includes polymers with pendant hydroxyl, carboxylic acid, amine, amide and/or urea moieties (or, more generally, hydrogen bond donors and/or acceptors). Specific examples of stabilizing polymers include polyvinyl alcohol, polyacrylamide, polyacrylonitrile, polymethacrylic acid, polyacrylic acid (e.g., Carbomer), alginate (e.g., sodium alginate), chitin, chitosan, zein and shellac. Typically, the hydrogel is Carbomer or an alginate. In certain embodiments, the stabilizing polymer is not an alginate. In certain embodiments, the stabilizing polymer is not ethyl cellulose, cellulose acetate, zein, modified zein, chitin, and/or chitosan.

Stabilizing polymers can be combined with a bioadhesive polymer by combining a finely ground dispersion of particles in a solution or dispersion with the bioadhesive polymer. Alternatively, the stabilizing polymer can be combined with the bioadhesive polymer by dispersing the bioadhesive polymer in a solution or dispersion of the hydrogel and then removing the solvent by evaporation or filtration.

Typically, the amount of a stabilizing polymer added to a bioadhesive polymer is from about 1% to about 90% by weight, preferably about 5% to about 70% and more preferably about 10% to about 50%.

e. Hydrophobic Components

The bioadhesive polymers described above can also be stabilized by combination with one or more hydrophobic components. Examples of hydrophobic small molecules include waxy materials (e.g., carnauba wax, beeswax, Chinese wax, spermaceti, lanolin, bayberry wax, Candelilla wax, castor wax, esparto wax, Japan wax, jojoba oil, ouricury wax, rice bran wax, ceresin waxes, montan wax, ozocerite, peat waxes, paraffin wax, polyethylene waxes) and polyglycerol fatty acid esters.

Typically, the amount of a hydrophobic component added to a bioadhesive polymer is from about 1% to about 25% by weight, preferably about 2% to about 10%.

f. Combinations of Additives

The stability of bioadhesive polymers can also be enhanced by incorporating materials from two or more of the classes of materials described above. Thus, the invention includes combinations including: (1) a polyanhydride and an acidic component, (2) a polyanhydride and a metal compound, (3) a polyanhydride and a stabilizing polymer, (4) a polyanhydride and a hydrophobic component, (5) an acidic component and a metal compound, (6) an acidic component and a stabilizing polymer, (7) an acidic component and a hydrophobic component, (8) a metal compound and a stabilizing polymer, (9) a metal compound and a hydrophobic component, (10) a stabilizing polymer and a hydrophobic component, (11) a polyanhydride and an acidic component and a metal compound, (12) a polyanhydride and an acidic component and a stabilizing polymer, (13) a polyanhydride and an acidic component and a hydrophobic component, (14) a polyanhydride and a metal compound and a stabilizing polymer, (15) a polyanhydride and a metal compound and a hydrophobic component, (16) a polyanhydride and a stabilizing polymer and a hydrophobic component, (17) an acidic component and a metal compound and a stabilizing polymer, (18) an acidic component and a metal compound and a hydrophobic component, (19) an acidic component and a stabilizing polymer and a hydrophobic component, (20) a metal compound and a stabilizing polymer and a hydrophobic component, (21) a polyanhydride and an acidic component and a metal compound and a stabilizing polymer, (22) a polyanhydride and an acidic component and a metal compound and a hydrophobic component, (23) a polyanhydride and a metal compound and a stabilizing polymer and a hydrophobic component, (24) an acidic component and a metal compound and a stabilizing polymer and a hydrophobic component and (25) at least one material from each of the five categories. In a one embodiment, a combination of an acidic component and a hydrophobic component are incorporated into a bioadhesive polymer, particularly citric acid and ethylcellulose.

The proportion of additives, when there is a combination of additives, typically falls within the ranges for the individual classes of additives disclosed above.

Bioadhesive materials described herein may be used in a wide variety of drug delivery, tissue engineering, and other medical and diagnostic applications. Bioadhesive materials may be formed into the subject microparticles, such as microspheres or microcapsules, or may be a coating on such microparticles. In the preferred embodiment, the material is applied as a coating to a solid oral dosage formulation, such as a tablet or gel-capsule or to multiparticulates. The coating may be applied by direct compression or by applying a solution containing the material to the tablets or gel-capsules. In one embodiment, the bioadhesive material is in the matrix of a tablet or other drug delivery device. Optionally, the tablet or drug delivery device contains a coating, such as a coating containing the bioadhesive material, another bioadhesive polymer, a rate-controlling coating or an enteric coating.

Bioadhesive materials used as coatings preferably do not appreciably swell upon hydration, such that they do not substantially inhibit or block movement (e.g., of ingested food) through the gastrointestinal tract, as compared to the polymers disclosed by Duchene et al. Generally, polymers that do not appreciably swell upon hydration include one or more hydrophobic regions, such as a polymethylene region (e.g., (CH₂)ₙ, where n is 4 or greater). The swelling of a polymer can be assessed by measuring the change in volume when the polymer is exposed to an aqueous solution. Polymers that do not appreciably swell upon hydration expand in volume by 50% or less when fully hydrated. Preferably, such polymers expand in volume by less than 25%, less than 20%, less than 15%, less than 10% or less than 5%. A polymer that does not appreciably swell upon hydration can be mixed with a polymer that does swell (e.g., Carbopol™, poly(acrylic acid), provided that the amount of swelling in the polymer does not substantially interfere with bioadhesiveness.

In one embodiment, the bioadhesive coating consists of two layers, an inner bioadhesive layer that does not substantially swell upon hydration and an outer bioadhesive layer that is readily hydratable and optionally bioerodable, such as one comprised of Carbopol™.

A tablet or a drug eluting device can have one or more coatings in addition to the bioadhesive coating. These coatings and their thickness can, for example, be used to control where in the gastrointestinal tract the bioadhesive coating becomes exposed. In one example, the additional coating prevents the bioadhesive coating from contacting the mouth or esophagus. In another example, the additional coating remains intact until reaching the small intestine.

Examples of coatings include methylmethacrylates, zein, modified zein, chitin, chitosan, cellulose acetate, cellulose phthalate, HPMC, sugars, enteric polymers, gelatin and shellac. Premature dissolution of a tablet in the mouth can be prevented with hydrophilic polymers such as HPMC or gelatin.

Coatings used in tablets of the invention, typically include a pore former, such that the coating is permeable to the drug.

Tablets, capsules and drug eluting devices of the invention can be coated by a wide variety of methods. Suitable methods include compression coating, coating in a fluidized bed or a pan, hot melt (extrusion) coating and enrobing. Such methods are well known to those skilled in the art.

The bioadhesive coating adheres to the mucosa in the aqueous environment of the gastrointestinal tract. As a result, the bioavailability of therapeutic agents is enhanced through increased residence time at the target absorption rate. In a preferred embodiment, the solid oral dosage form contains rate controlling agents, such as hydroxypropylmethyl cellulose (HPMC) and microcrystalline cellulose (MCC). Optionally, the drug may be in the form or microparticles or nanoparticles. In one embodiment, a tablet contains a core containing a nanoparticulate drug and enhancers in a central matrix of rate controlling agents, such as hydroxypropylmethyl cellulose (HPMC) and microcrystalline cellulose (MCC). The core is surrounded on its circumference by bioadhesive polymer (preferably DOPA-BMA polymer). Optionally, the final tablet is coated with an enteric coating, such as Eudragit L100-55, to prevent release of the drug until the tablet has moved to the small intestine.

The bioadhesive materials may be used in or as a coating on prosthetics, such as dental prosthetics. The materials may be used as dental adhesives, or bone cements and glues. The materials are suitable for use in wound healing applications, such as synthetic skins, wound dressings, and skin plasters and films.

In order to alter the physical properties of bioadhesive materials, additional components can be added to a composition. Such components include bioadhesive modifiers, solvents, thermoplastic polymers and plasticizers.

Bioadhesive materials can be mixed with one or more plasticizers or thermoplastic polymers. Such agents typically increase the strength and/or reduce the brittleness of polymeric coatings. Plasticizers can be hydrophobic or hydrophilic. Examples of plasticizers include dibutyl sebacate, polyethylene glycol, triethyl citrate, dibutyl adipate, dibutyl fumarate, diethyl phthalate, ethylene oxide-propylene oxide block copolymers such as Pluronic™ F68 and di(sec-butyl) fumarate. Example of thermoplastic polymers include polyesters, poly(caprolactone), polylactide, poly(lactide-co-glycolide), methyl methacrylate (e.g., EUDRAGIT™), cellulose and derivatives thereof such as ethyl cellulose, cellulose acetate and hydroxypropyl methyl cellulose (HPMC) and large molecular weight polyanhydrides. The plasticizers and/or thermoplastic polymers are mixed with a bioadhesive polymer to achieve the desired properties. Typically, the proportion of plasticizers and thermoplastic polymers, when present, is from 0.5% to 50% by weight.

Bioadhesive modifiers include both natural and synthetic bioadhesive modifiers, which can be swellable or non-swellable and gellable or non-gellable. Swellable modifiers include fluid-imbibing displacement polymers (osmopolymers), such as poly(alkylene oxide), hydrogels (CARBOPOL®), polyacrylamide, crosslinked poly(indene-co-maleic anhydride), poly(acrylic acid), polysaccharides and polyglucan.

Gellable or non-gellable modifiers include karaya gum, guar gum, okra gum, gum arabic, acacia gum, pectina gum, ghatti gum, tragacanth gum, xanthan gum, locust bean gum, psyllium seed gum, tamarind gum, destria gum, casein gum and other gums.

Natural bioadhesive modifiers include cellulose compounds (cellulose, ethylcellulose, methylcellulose, nitrocellulose, propylcellulose, hydroxypropyl cellulose, hydroxyethylcellulose, carboxymethylcellulose and hydroxypropylmethylcellulose, including alkyl and hydroxyalkyl derivatives), karaya gum, prolamines (zein, modified zein, chitin, chitosan), L-DOPA, benserazide, carbidopa, dopamine, 3-O-methyldopa and other L-DOPA metabolites. In certain embodiments, the natural bioadhesive modifiers exclude L-DOPA and/or its metabolites.

The bioadhesive modifiers can, for example, be blended with the bioadhesive materials of the invention during the preparation of a pharmaceutical composition. For tablets, a bioadhesive modifier is generally blended with a bioadhesive material though dry or wet mixing prior to tablet preparation.

As disclosed in U.S. Pat. Nos. 5,985,312, 6,123,965 and 6,368,586, the contents of which are incorporated herein by reference, bioadhesive polymers and compositions, such as those named above, having a metal compound combined therewith have a further improved ability to adhere to tissue surfaces, such as mucosal membranes. The metal compound combined with the polymer can be, for example, a water-insoluble metal oxide. The combination of metal compounds with a wide range of different polymers, even those that are not normally bioadhesive, improves their ability to adhere to tissue surfaces such as mucosal membranes.

Control of the rate that an active drug (e.g., a sustained release or controlled delivery form of a drug) is introduced to a targeted delivery site and its residence time at the targeted delivery site (e.g., site of absorption) is achieved, at least in part, by using excipients, such as polymeric excipients. The exact mechanism by which a polymer interacts with the mucosa or controls the delivery of the drug is at least partially dependent on the rate of polymer hydration and swelling, which is related to its molecular weight. Therefore, any process that significantly reduces the molecular weight of the polymer is likely to affect its ability to control the drug delivery. Oxidative degradation can lead to a loss in molecular weight for several polymers commonly used in controlled release applications (Waterman, K. C., et. al., Pharm. Dev. Technol., 2002, 1-32). In addition to a loss in molecular weight, such degradation in polymers can produce reactive impurities and end groups to compromise the chemical stability of drugs and also their effectiveness as a bioadhesive polymer or release controlling agent. An example of class of controlled release polymers that can degrade to compromise the drug release rate is the polyoxyethylenes, including poly(ethylene oxides) (Polyox™), poly(ethylene glycols), and poly(oxyethylene) alkyl ethers. The polyethylene oxide is usually treated by the manufacturer (Dow chemicals) with 100-1000 ppm of butylated hydroxy toluene (BHT) to reduce such degradation. While this antioxidant is quite effective, it is volatile and can be lost during any heating steps and therefore it is advisable to include an additional antioxidants to the formulation matrix to retain the polymer behavior intact (Waterman, K. C., et. al., Pharm. Dev. Technol., 2002, 1-32).

Hence, it is advisable to incorporate some stabilizers, preferably antioxidants or chelating agents, to inhibit any impurity-related degradation of drugs. Antioxidants can reduce formation of peroxides, but may be less effective in eliminating of peroxides already present in a dosage form. Currently, the marketed form of bupropion hydrochloride is stabilized with an antioxidant like L-cysteine hydrochloride. In contrast, chelating agents such as citric acid, edetic acid, fumaric acid and malic acid are recommended for inhibition of any metal induced oxidation. Chelating agents are generally more effective when added during a granulation step or by coating particles using fluid bed technology, rather than simply during physical mixing. Suitable antioxidants and chelating agents are disclosed in U.S. Pat. No. 6,423,351, the contents of which are incorporated herein by reference, which discloses prevention of drug oxidation using a ferrous ion source. Other suitable antioxidants include vitamin E, vitamin C, butylated hydroxytoluene, and butylated hydroxyanisole.

The pH to which a polymer is exposed can play a significant role in the stabilization of the polymer to oxidation. It is in general more difficult to remove an electron from a polymer when it is positively charged. For this reason, stability against oxidation is often greater under low pH conditions, which promote protonation of polymers if protonation is possible. In the converse, higher pH conditions, which deprotonate a polymer, generally make a drug more susceptible to oxidation.

U.S. Pat. Nos. 5,358,970; 5,541,231; 5,731,000 and 5,763,493 to Ruff et al, the contents of which are incorporated herein by reference, describe a stabilized bupropion hydrochloride formulation having a stabilizer selected from group consisting of L-cysteine hydrochloride, glycine hydrochloride, malic acid, sodium metabisulfite, citric acid, tartaric acid, L-cystine dihydrochloride, ascorbic acid, and isoascorbic (erythorbic) acid. Such stabilizers are useful herein as antioxidants and/or chelating agents. U.S. Pat. No. 6,652,882 to Odidi et. al describes stabilization of drug by a saturated polyglycolised glyceride like Gelucire™, and such compounds are suitable for use in the present invention.

Other oxidation stabilization strategies for bupropion formulations, which are suitable for use herein, include the addition of inorganic acids like hydrochloric acid, phosphoric acid, nitric acid and sulfuric acid (U.S. Pat. No. 5,968,553, the contents of which are incorporated herein by reference); dicarboxylic acids like oxalic acid, succinic acid, adipic acid, fumaric acid, benzoic acid and phthalic acid (U.S. Pat. Nos. 6,194,002; 6,221,917; 6,242,496; 6,482,987 and 6,652,882, the contents of which are incorporated herein by reference); sulfites like potassium metabisulfite and sodium bisulfite (U.S. Pat. No. 6,238,697, the contents of which are incorporated herein by reference); organic esters like L-ascorbic acid palmitate, tocopherol solution in alcohol, butylated hydroxy anisole, tocopherol or tocopherol, vitamin E succinate, vitamin E 700 acetate, and L-ascorbic acid G palmitate (U.S. Pat. No. 6,312,716, the contents of which are incorporated herein by reference). The use of acidified granules of microcrystalline cellulose (U.S. Pat. No. 6,153,223, the contents of which are incorporated herein by reference); salts of organic bases like creatinine hydrochloride, pyridoxine hydrochloride and thiamine hydrochloride and inorganic acid like potassium phosphate monobasic (U.S. Pat. No. 6,333,332, the contents of which are incorporated herein by reference) is also suitable for the present invention.

Typically, antioxidants used in the present invention are selected from ascorbyl palmitate, butylated hydroxyanisole, butylated hydroxytoluene, malic acid, propyl gallate, sodium bisulfite, sodium sulfite, sodium metabisulfite, potassium metabisulfite, potassium bisulfite, sodium thiosulfate, sodium formaldehyde sulfoxylate, L-ascorbic acid, D-ascorbic acid, acetylcysteine, cysteine, thioglycerol, thioglycollic acid, thiolactic acid, thiourea, dithiothreitol, dithioerythreitol, glutathione, nordihydroguaiaretic acid, tocopherol, fumaric acid and succinic acid.

The term “acidification” refers to any method of lowering the pH of the bioadhesive polymers either before or after combination with a compatible pharmaceutical drug. Preferably, acidification employs a pharmaceutically acceptable acid to lower pH. Suitable pharmaceutically acceptable acids are well known in the art and include, by way of example only, hydrochloric acid, phosphoric acid, acetic acid, citric acid, fumaric acid, succinic acid, lactic acid, and the like.

Preferably, an antioxidant or a chelating agent is added to a bioadhesive polymer prior to formulating it with a drug. The antioxidant or chelating agent can be added as a dry material or during wet granulation or following the extrusion or annealing process.

Antioxidants (also sometimes referred to as free radical absorbers) self-sacrificially stabilize materials against free radicals (for example, free radicals generated from photooxidation as a result of exposure to sunlight). The antioxidant and the bioadhesive polymer are preferably maintained in sufficiently close proximity such that a synergistic effect on stability of polymer is achieved. In that regard, a a bioadhesive polymer (e.g., a carbidopa-BMA polymer) can be maintained in sufficiently close proximity to the antioxidant moiety to enhance the stability of the polymer in an environment in which photo-oxidation can occur. Such close proximity is not typically obtained upon mere physical mixing of antioxidant and UV-absorber.

In order to further protect a drug formulation, an antioxidant can be present in combination with a UV-absorber such as PABA or BHT. These components can be localized such that the UV-absorber is within a single molecule (for example, within a single oligomeric or polymer chain). For example, the antioxidant and the UV-absorber can be localized through covalent bonding by reacting (for example, copolymerizing) at least one monomer including or incorporating the antioxidant with at least one monomer including or incorporating the UV-absorber. Antioxidants and UV-absorbers can also be conjugated to a suitably reactive polymer.

Antioxidants, chelating agents and UV-absorbers should be selected such that they do not react with a drug planned to be delivered with the polymer.

Typically, about 0.1% to about 20% by weight, such as about 0.5% to about 10% or about 1% to about 5%, of antioxidant and/or chelating agent is added to a bioadhesive polymer.

In general, there is no specific limitation on the material that can be encapsulated within the bioadhesive materials. Any kind of therapeutic, prophylactic or diagnostic agent, including organic compounds, inorganic compounds, proteins, polysaccharides, nucleic acids, or other materials can be incorporated using standard techniques. Flavorants, nutraceuticals, and dietary supplements are among the materials that can be incorporated in the bioadhesive material. In one embodiment, L-3,4-dihydroxyphenylalanine (“levodopa” or “L-dopa”) is incorporated into the bioadhesive material for delivery to a patient. The bioadhesive material may contain carbidopa. In one embodiment, levodopa and carbidopa are both incorporated in the bioadhesive material. In a preferred embodiment, the bioadhesive material is a coating on an oral dosage formulation which contains levodopa and carbidopa in separate drug layers.

The bioadhesive polymer may also be used as one or more layers in a subject bioadhesive drug delivery tablet formulation.

Polymer-Metal Complexes

As described above, metal can be used to stabilize certain polymers. In addition, as disclosed in U.S. Pat. Nos. 5,985,312, 6,123,965 and 6,368,586 (the contents of which are incorporated herein by reference), polymers, such as those named above, having a metal compound incorporated therein have a further improved ability to adhere to tissue surfaces, such as mucosal membranes.

The metal compound incorporated into the polymer can be, for example, a water-insoluble metal oxide. The incorporation of metal compounds into a wide range of different polymers, even those that are not normally bioadhesive, improves their ability to adhere to tissue surfaces such as mucosal membranes.

The metal compounds preferably are water-insoluble metal compounds, such as water-insoluble metal oxides and hydroxides, including oxides of calcium, iron, copper and zinc. The metal compounds can be combined with a wide range of hydrophilic and hydrophobic polymers including proteins, polysaccharides and synthetic biocompatible polymers.

Metal compounds which can be incorporated into polymers preferably are water-insoluble metal compounds, such as water-insoluble metal oxides and metal hydroxides, which are capable of becoming combined with a polymer to thereby improve the bioadhesiveness of the polymer. As defined herein, a water-insoluble metal compound is defined as a metal compound with little or no solubility in water, for example, less than about 0.0 to 0.9 mg/ml.

The water-insoluble metal compounds can be derived from a wide variety of metals, including, but not limited to, calcium, iron, copper, zinc, cadmium, zirconium and titanium. The water insoluble metal compound preferably is a metal oxide or hydroxide. Water insoluble metal compounds of multivalent metals are preferred. Representative metal oxides suitable for use in the compositions described herein include cobalt oxide (I) (CoO), cobalt oxide (II)(Co₂O₃), selenium oxide (SeO₂), chromium double oxide (CrO₂), manganese oxide (MnO₂), titanium oxide (TiO₂), lanthanum oxide (La₂O₃), zirconium oxide (ZrO₂), silicon oxide (SiO₂), scandium oxide (Sc₂O₃), beryllium oxide (BeO), tantalum oxide (Ta₂O₅), cerium oxide (CeO₂), neodymium oxide (Nd₂O₃), vanadium oxide (V₂O₅), molybdenum oxide (Mo₂O₃), tungsten oxide (WO), tungsten trioxide (WO3), samarium oxide (Sm₂O₃), europium oxide (Eu₂O₃), gadolinium oxide (Gd₂O₃), terbium oxide (Tb₄O₇), dysprosium oxide (Dy₂O₃), holmium oxide (Ho₂O₃), erbium oxide (Er₂O₃), thulium oxide (Tm₂O₃), ytterbium oxide (Yb₂O₃), lutetium oxide (Lu₂O₃), aluminum oxide (Al₂O₃), indium oxide (InO₃), germanium oxide (GeO₂), antimony oxide (Sb₂O₃), tellurium oxide (TeO₂), nickel oxide (NiO), and zinc oxide (ZnO). Other oxides include barium oxide (BaO), calcium oxide (CaO), nickel oxide (III) (Ni₂O₃), magnesium oxide (MgO), iron oxide (II) (FeO), iron oxide (III) (Fe₂O₃), copper oxide (II) (CuO), cadmium oxide (CdO), and zirconium oxide (ZrO₂).

In certain embodiments, the metal compound is ferric oxide, copper oxide or zinc oxide or a combination thereof. In other embodiments, the metal compound is a zirconate, such as magnesium zirconate or calcium zirconate. In yet other embodiments, the metal compound is a silicate, such as magnesium silicate (e.g., a hydrated magnesium silicate such as talc) or calcium silicate. Advantageously, metal compounds which are incorporated into polymers are metal compounds which are already approved by the FDA or an equivalent agency as either food or pharmaceutical additives, such as zinc oxide or talc.

Preferred properties defining the metal compound include: (a) substantial insolubility in aqueous environments, such as acidic or basic aqueous environments (such as those present in the gastric lumen); and (b) ionizable surface charge at the pH of the aqueous environment.

The water-insoluble metal compounds can be incorporated into a polymer by, for example, one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bed, pan coating, or any similar methods known to those skilled in the art, which produce a metal compound enriched layer on the surface of the device. In one embodiment, nanoparticles or microparticles of the water-insoluble metal compound are incorporated into the polymer, preferably as a uniform dispersion.

In one embodiment, the metal compound is provided as a fine particulate dispersion of a water-insoluble metal oxide which is incorporated throughout the polymer or at least on the surface of the polymer which is to be adhered to a tissue surface. The metal compound also can be incorporated in an inner layer of the polymer and exposed only after degradation or else dissolution of a “protective” outer layer. For example, a tablet core containing a polymer and metal may be covered with an enteric coating designed to dissolve when exposed to gastric fluid. The metal compound-enriched core then is exposed and become available for binding to GI mucosa.

Fine metal oxide particles can be produced, for example, by micronizing a metal oxide by mortar and pestle treatment to produce particles ranging in size, for example from 10.0 to 300 nm. The metal oxide particles can be incorporated into a polymer, for example, by dissolving or dispersing the particles into a solution or dispersion of the polymer.

Metal compounds are optionally coated with a protective coating, such as an enteric coating or a rate controlling coating. Such coatings are selected in order to release the metal compound only when the system is exposed to gastric fluid or another targeted environment.

Typically, the amount of a metal compound added to a bioadhesive polymer is from about 1% to about 65% by weight, preferably about 5% to about 45% and more preferably about 10% to about 30%.

Advantageously, metal compounds which are incorporated into polymers to improve their bioadhesive properties can be metal compounds which are already approved by the FDA as either food or pharmaceutical additives, such as zinc oxide.

Suitable polymers which can be used and into which the metal compounds can be incorporated include soluble and water-insoluble, and biodegradable and nonbiodegradable polymers, including hydrogels, thermoplastics, and homopolymers, copolymers and blends of natural and synthetic polymers, provided that they have the requisite fracture strength when mixed with a metal compound. In additional to those listed above, representative polymers which can be used in conjunction with a metal compound include hydrophilic polymers, such as those containing carboxylic groups, including polyacrylic acid. Bioerodible polymers including polyanhydrides, poly(hydroxy acids) and polyesters, as well as blends and copolymers thereof also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene also can be used.

Representative natural polymers which also can be used include proteins, such as zein, modified zein, chitin, chitosan, casein, gelatin, gluten, serum albumin, or collagen, and polysaccharides such as dextrans, polyhyaluronic acid and alginic acid. Representative synthetic polymers include polyphosphazenes, polyamides, polycarbonates, polyacrylamides, polysiloxanes, polyurethanes and copolymers thereof. Celluloses also can be used. As defined herein the term “celluloses” includes naturally occurring and synthetic celluloses, such as alkyl celluloses, cellulose ethers, cellulose esters, hydroxyalkyl celluloses and nitrocelluloses. Exemplary celluloses include ethyl cellulose, methyl cellulose, carboxymethyl cellulose, hydroxymethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, cellulose triacetate and cellulose sulfate sodium salt.

Polymers of acrylic and methacrylic acids or esters and copolymers thereof can be used. Representative polymers which can be used include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other polymers which can be used include polyalkylenes such as polyethylene and polypropylene; polyarylalkylenes such as polystyrene; poly(alkylene glycols), such as poly(ethylene glycol); poly(alkylene oxides), such as poly(ethylene oxide); and poly(alkylene terephthalates), such as poly(ethylene terephthalate). Additionally, polyvinyl polymers can be used, which, as defined herein includes polyvinyl alcohols, polyvinyl ethers, polyvinyl esters and polyvinyl halides. Exemplary polyvinyl polymers include poly(vinyl acetate), polyvinyl phenol and polyvinylpyrrolidone.

Water soluble polymers can also be used. Representative examples of suitable water soluble polymers include polyvinyl alcohol, polyvinylpyrrolidone, methyl cellulose, hydroxypropyl cellulose, hydroxypropylmethyl cellulose and polyethylene glycol, copolymers of acrylic and methacrylic acid esters, and mixtures thereof. Water insoluble polymers also can be used. Representative examples of suitable water insoluble polymers include ethylcellulose, cellulose acetate, cellulose propionate (lower, medium or -higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride), polyurethanes, and mixtures thereof. In one embodiment, a water insoluble polymer and a water soluble polymer are used together, such as in a mixture. Such mixtures are useful in controlled drug release formulations, wherein the release rate can be controlled by varying the ratio of water soluble polymer to water insoluble polymer.

Polymers varying in viscosity as a function of temperature or shear or other physical forces also may be used. Poly(oxyalkylene) polymers and copolymers such as poly(ethylene oxide)-poly(propylene oxide) (PEO-PPO) or poly(ethylene oxide)-poly(butylene oxide) (PEO-PBO) copolymers, and copolymers and blends of these polymers with polymers such as poly(alpha-hydroxy acids), including but not limited to lactic, glycolic and hydroxybutylc acids, polycaprolactones, and polyvalerolactones, can be synthesized or commercially obtained. For example, polyoxyalkylene copolymers are described in U.S. Pat. Nos. 3,829,506, 3,535,307, 3,036,118, 2,979,578, 2,677,700 and 2,675,619. Polyoxyalkylene copolymers are sold, for example, by BASF under the trade name PLURONICS™. These materials are applied as viscous solutions at room temperature or lower which solidify at the higher body temperature. Other materials with this behavior are known in the art, and can be utilized as described herein. These include KLUCEL™ (hydroxypropyl cellulose), and purified konjac glucomannan gum.

Other suitable polymers are polymeric lacquer substances based on acrylates and/or methacrylates, commonly called EUDRAGIT™ polymers (sold by Rohm America, Inc.). Specific EUDRAGIT™ polymers can be selected having various permeability and water solubility, which properties can be pH dependent or pH independent. For example, EUDRAGIT™ RL and EUDRAGIT™ RS are acrylic resins comprising copolymers of acrylic and methacrylic acid esters with a low content of quaternary ammonium groups, which are present as salts and give rise to the permeability of the lacquer films, whereas EUDRAGIT™ RL is freely permeable and EUDRAGIT™ RS is slightly permeable, independent of pH. In contrast, the permeability of EUDRAGIT™ L is pH dependent. EUDRAGIT™ L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable.

Polymer solutions that are liquid at an elevated temperature but solid or gelled at body temperature can also be utilized. A variety of thermoreversible polymers are known, including natural gel-forming materials such as agarose, agar, furcellaran, beta-carrageenan, beta-1,3-glucans such as curdlan, gelatin, or polyoxyalkylene containing compounds, as described above. Specific examples include thermosetting biodegradable polymers for in vivo use described in U.S. Pat. No. 4,938,763, the contents of which are incorporated herein by reference.

Polymer Blends with Monomers and/or Oligomers

Polymers with enhanced bioadhesive properties are provided by incorporating anhydride monomers or oligomers into one of the polymers listed above by dissolving, dispersing, or blending, as taught by U.S. Pat. Nos. 5,955,096 and 6,156,348, the contents of which are incorporated herein by reference. The polymers may be used to form drug delivery systems which have improved ability to adhere to tissue surfaces, such as mucosal membranes. The anhydride oligomers are formed from organic diacid monomers, preferably the diacids normally found in the Krebs glycolysis cycle. Anhydride oligomers which enhance the bioadhesive properties of a polymer have a molecular weight of about 5000 or less, typically between about 100 and 5000 daltons, or include 20 or fewer diacid units linked by anhydride linkages and terminating in an anhydride linkage with a carboxylic acid monomer.

The oligomer excipients can be blended or incorporated into a wide range of hydrophilic and hydrophobic polymers including proteins, polysaccharides and synthetic biocompatible polymers, including those described above. In one embodiment, anhydride oligomers may be combined with metal oxide particles, such as those described above, to improve bioadhesion even more than with the organic additives alone. Organic dyes, because of their electronic charge and hydrophobicity or hydrophilicity, can either increase or decrease the bioadhesive properties of polymers when incorporated into the polymers.

As used herein, the term “anhydride oligomer” refers to a diacid or polydiacid linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. In one embodiment, the diacids are those normally found in the Krebs glycolysis cycle. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example, from toluene or other organic solvents. The oligomer is collected by filtration, and washed, for example, in ethers. The reaction produces anhydride oligomers of mono and poly acids with terminal carboxylic acid groups linked to each other by anhydride linkages.

The anhydride oligomer is hydrolytically labile. As analyzed by gel permeation chromatography, the molecular weight may be, for example, on the order of 200400 for fumaric acid oligomer (FAPP) and 2000-4000 for sebacic acid oligomer (SAPP). The anhydride bonds can be detected by Fourier transform infrared spectroscopy by the characteristic double peak at 1750 cm⁻¹ and 1820 cm⁻¹, with a corresponding disappearance of the carboxylic acid peak normally at 1700 cm⁻¹.

In one embodiment, the oligomers may be made from diacids described for example in U.S. Pat. Nos. 4,757,128, 4,997,904 and 5,175,235, the disclosures of which are incorporated herein by reference. For example, monomers such as sebacic acid, bis(p-carboxy-phenoxy)propane, isophathalic acid, fumaric acid, maleic acid, adipic acid or dodecanedioic acid may be used.

Organic dyes, because of their electronic charge and hydrophilicity or hydrophobicity, may alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2, 3, 13 and 86, rose bengal, safranin, Sudan III and IV, Sudan black B and toluidine blue.

Polymers Functionalized with Hydroxy-Substituted Aromatic Groups

Polymers having an aromatic group which contains one or more hydroxyl groups grafted onto them or coupled to individual monomers are also suitable for use in the bioadhesive coatings of the invention. Such polymers can be biodegradable or non-biodegradable polymers. The polymer can be hydrophobic. Preferably, the aromatic group is catechol or a derivative thereof and the polymer contains reactive functional groups. Typically, the polymer is a polyanhydride and the aromatic compound is the catechol derivative DOPA. These materials display bioadhesive properties superior to conventional bioadhesives used in therapeutic and diagnostic applications.

The molecular weight of the suitable polymers and percent substitution of the polymer with the aromatic group may vary greatly. The degree of substitution varies based on the desired adhesive strength, it may be as low as 10%, 25% or 50%, or up to 100% substitution. Generally, at least 50% of the monomers in the polymeric backbone are substituted with at least one aromatic group. Preferably, about 100% of the monomers in the polymeric backbone are substituted with at least one aromatic group. The resulting polymer has a molecular weight ranging from about 1 to 2,000 kDa.

The polymer that forms that backbone of the bioadhesive material can be a biodegradable polymer. Examples of preferred biodegradable polymers include synthetic polymers such as poly hydroxy acids, such as polymers of lactic acid and glycolic acid, polyanhydrides, poly(ortho)esters, polyesters, polyurethanes, poly(butyric acid), poly(valeric acid), poly(caprolactone), poly(hydroxybutyrate), poly(lactide-co-glycolide) and poly(lactide-cocaprolactone), and natural polymers such as alginate and other polysaccharides, collagen and chemical derivatives thereof (substitutions, additions of chemical groups, for example, alkyl, alkylene, hydroxylations, oxidations, and other modifications routinely made by those skilled in the art), albumin and other hydrophilic proteins, zein, modified zein, chitin, chitosan, and other prolamines and hydrophobic proteins, copolymers and mixtures thereof. In general, these materials degrade either by enzymatic hydrolysis or exposure to water in vivo and by surface or bulk erosion. The foregoing materials may be used alone, as physical mixtures (blends), or as co-polymers.

Suitable polymers can formed by first coupling the aromatic compound to the monomer and then polymerizing. In this example, the monomers may be polymerized to form a polymer backbone, including biodegradable and non-biodegradable polymers. Suitable polymer backbones include, but are not limited to, polyanhydrides, polyamides, polycarbonates, polyalkylenes, polyalkylene oxides such as polyethylene glycol, polyalkylene terephthalates such as poly(ethylene terephthalate), polyvinyl alcohols, polyvinyl ethers, polyvinyl esters, polyethylene, polypropylene, poly(vinyl acetate), poly(vinyl chloride), polystyrene, polyvinyl halides, polyvinylpyrrolidone, polyhydroxy acids, polysiloxanes, polyurethanes and copolymers thereof, alkyl cellulose, hydroxyalkyl celluloses, cellulose ethers, cellulose esters, nitrocellulloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate).

A suitable polymer backbone can be a known bioadhesive polymer that is hydrophilic or hydrophobic. Hydrophilic polymers include CARBOPOL™, polycarbophil, cellulose esters, and dextran.

Non-biodegradable polymers, especially hydrophobic polymers are also suitable as polymer backbones. Examples of preferred non-biodegradable polymers include ethylene vinyl acetate, poly(methacrylic acid), copolymers of maleic anhydride with other unsaturated polymerizable monomers, poly(butadiene maleic anhydride), polyamides, copolymers and mixtures thereof and dextran, cellulose and derivatives thereof.

Hydrophobic polymer backbones include polyanhydrides, poly(ortho)esters, and polyesters such as polycaprolactone. Preferably, the polymer is sufficiently hydrophobic that it is not readily water soluble, for example the polymer should be soluble up to less than about 1% w/w in water, preferably about 0.1% w/w in water at room temperature or body temperature. In the most preferred embodiment, the polymer is a polyanhydride, such as a poly(butadiene maleic anhydride) or another copolymer of maleic anhydride. Polyanhydrides may be formed from dicarboxylic acids as described in U.S. Pat. No. 4,757,128 to Domb et al., incorporated herein by reference. Suitable diacids include aliphatic dicarboxylic acids, aromatic dicarboxylic acids, aromatic-aliphatic dicarboxylic acid, combinations of aromatic, aliphatic and aromatic-aliphatic dicarboxylic acids, aromatic and aliphatic heterocyclic dicarboxylic acids, and aromatic and aliphatic heterocyclic dicarboxylic acids in combination with aliphatic dicarboxylic acids, aromatic-aliphatic dicarboxylic acids, and aromatic dicarboxylic acids of more than one phenyl group. Suitable monomers include sebacic acid (SA), fumaric acid (FA), bis(p-carboxyphenoxy)propane (UP), isophthalic acid (IPh), and dodecanedioic acid (DD).

A wide range of molecular weights are suitable for the polymer that forms the backbone of the bioadhesive material. The molecular weight may be as low as about 200 Da (for oligomers) up to about 2,000 kDa. Preferably the polymer has a molecular weight of at least 1,000 Da, more preferably at least 2,000 Da, most preferably the polymer has a molecular weight of up to 20 kDa or up to 200 kDa. The molecular weight of the polymer may be up to 2,000 kDa.

The range of substitution on the polymer varies greatly and depends on the polymer used and the desired bioadhesive strength. For example, a butadiene maleic anhydride copolymer that is 100% substituted with DOPA will have the same number of DOPA molecules per chain length as a 67% substituted ethylene maleic anhydride copolymer. Typically, the polymer has a percentage substitution ranging from 10% to 100%, preferably ranging from 50% to 100%.

The polymers and copolymers that form the backbone of the bioadhesive material include reactive functional groups that interact with the functional groups on the aromatic compound.

It is desirable that the polymer or monomer that forms the polymeric backbone contains accessible functional groups that easily react with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, aryl azides, isocyanates, isothiocyanates, succinimidyl esters or a combination thereof.

Preferably, the aromatic compound containing one or more hydroxyl groups is catechol or a derivative thereof. Optionally, the aromatic compound is a polyhydroxy aromatic compound, such as a trihydroxy aromatic compound (e.g., phloroglucinol) or a multihydroxy aromatic compound (e.g., tannin). The catechol derivative may contain a reactive group, such as an amino, thiol, or halide group. The preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g., by hydroxylation) to the DOPA form. A particularly preferred aromatic compound is an amine-containing aromatic compound, such as an amine-containing catechol derivative (e.g., dopamine).

Two general methods are used to form the polymer product. In one example, a compound containing an aromatic group which contains one or more hydroxyl groups is grafted onto a polymer. In this example, the polymeric backbone is a biodegradable polymer. In a second example, the aromatic compound is coupled to individual monomers and then polymerized.

Any chemistry which allows for the conjugation of a polymer or monomer to an aromatic compound containing one or more hydroxyl groups can be used, for example, if the aromatic compound contains an amino group and the monomer or polymer contains an amino reactive group, this modification to the polymer or monomer is performed through a nucleophilic addition or a nucleophilic substitution reaction, such as a Michael-type addition reaction, between the amino group in the aromatic compound and the polymer or monomer. Additionally, other procedures can be used in the coupling reaction. For example, carbodiimide and mixed anhydride based procedures form stable amide bonds between carboxylic acids or phosphates and amino groups, bifunctional aldehydes react with primary amino groups, bifunctional active esters react with primary amino groups, and divinylsulfone facilitates reactions with amino, thiol, or hydroxy groups.

The aromatic compounds are grafted onto the polymer using standard techniques to form the bioadhesive material. In one example, L-DOPA is grafted to maleic anhydride copolymers by reacting the free amine in L-DOPA with the maleic anhydride bond in the copolymer.

A variety of different polymers can be used as the backbone of the bioadhesive material, as described above. Additional representative polymers include 1:1 random copolymers of maleic anhydride with ethylene, vinyl acetate, styrene, or butadiene. In addition, a number of other compounds containing aromatic rings with hydroxy substituents, such as tyrosine or derivatives of catechol, can be used in this reaction.

In another embodiment, the polymers are prepared by conjugate addition of a compound containing an aromatic group that is attached to an amine to one or more monomers containing an amino reactive group. In a preferred method, the monomer is an acrylate or the polymer is acrylate. For example, the monomer can be a diacrylate such as 1,4-butanediol diacrylate, 1,3-propanediol diacrylate, 1,2-ethanediol diacrylate, 1,6-hexanediol diacrylate, 2,5-hexanediol diacrylate or 1,3-propanediol diacrylate. In an example of the coupling reaction, the monomer and the compound containing an aromatic group are each dissolved in an organic solvent (e.g., THF, CH₂Cl₂, methanol, ethanol, CHCl₃, hexanes, toluene, benzene, CCl₄, glyme, diethyl ether, etc.) to form two solutions. The resulting solutions are combined, and the reaction mixture is heated to yield the desired polymer. The molecular weight of the synthesized polymer can be controlled by the reaction conditions (e.g., temperature, starting materials, concentration, solvent, etc.) used in the synthesis.

For example, a monomer, such as 1,4-phenylene diacrylate or 1,4-butanediol diacrylate having a concentration of 1.6 M, and DOPA or another primary amine containing aromatic molecule are each dissolved in an aprotic solvent such as DMF or DMSO to form two solutions. The solutions are mixed to obtain a 1:1 molar ratio between the diacrylate and the amine group and heated to 56° C. to form a bioadhesive material.

Bioadhesive Polymer Blends

Hydrophobic polymers, such as polyesters, poly(anhydrides), ethyl cellulose, even if possibly non-adhesive on their own, may nevertheless be made bioadhesive simply by physically mixing the hydrophobic polymers with one or more suitable compounds (such as catechols or derivatives L-DOPA, D-DOPA, dopamine, or carbidopa, etc.) to create “bioadhesive compositions.” Similarly, metal oxides may also be used for this purpose.

The molecular weight of the bioadhesive polymers and percent substitution of the polymers with residues of the compounds disclosed may vary greatly. The degree of substitution varies based on the desired adhesive strength, it may be as low as 10%, 20%, 25%, 50%, or up to 100% substitution. On average, at least 50% of the repeat units in the polymeric backbone are substituted with at least one residue. In one particular embodiment, 75-95% of the residues in the backbone are substituted with at least one residue. In another particular embodiment, on average 100% of the repeat units in the polymeric backbone are substituted with at least one residue. The resulting bioadhesive polymer typically has a molecular weight ranging from about 1 to 2,000 kDa, such as 1 to 1,000 kDa, 10 to 1,000 kDa or 100 to 1,000 kDa. Polymers used in bioadhesive compositions typically have the same range of molecular weights.

Unlike the bioadhesive polymers described above, there is typically no covalent bond formed between the compounds and the polymer in the bioadhesive compositions (i.e., the polymer does not chemically react with the compound, although hydrogen bonds, ionic bonds and/or van der Waals interactions can occur).

Suitable polymers for use in bioadhesive compositions are described above. Typically, the polymer itself may not be bioadhesive, but the polymer can be bioadhesive (e.g., a polymer with hydrogen bond-forming pendant groups). Preferably, the polymer is a hydrophobic polymer such as a poly(lactone), e.g., poly(caprolactone).

To form the bioadhesive compositions of the invention, typically a polymer and a suitable compound are dissolved in a compatible solvent and mixed together. The solvent is then evaporated, preferably at a controlled temperature and rate of removal. Alternatively or in combination with general evaporation, the bioadhesive composition can be spray dried or dried at room temperature.

In another example, a mixture of a polymer and a suitable compound are melted at or slightly above the melting point of the polymer, typically while being mixed. Both the polymer and the suitable compound should be selected such that they are chemically stable (e.g., do not decompose, do not become oxidized) at the melting point temperature. After the composition has re-solidified, it can be milled in order to obtain particles of the desired size.

The subject bioadhesive compositions can also be prepared by dry mixing of a polymer and a suitable compound, provided that the suitable compound is sufficiently distributed throughout the composition.

In each of the above methods, additional components can be added to the mixture prior to dissolution, melting and/or mixing. The additional components are preferably stable under the conditions the mixture is exposed to. In particular, active agents should be stable at the melting point temperature if that method is employed.

The weight ratio of polymer to the suitable compound in a bioadhesive composition can be selected to give the desired amount of bioadhesion. Typically, the weight ratio of polymer to compound is 9:1 to 1:9, such as 3:1 to 1:3 or 2:1 to 1:2. For example, when the polymer is predominant component, the weight ratio is 9:1 to 1:1, 3:1 to 1:1 or 2:1 to 1:1.

In the subject methods and pharmaceutical compositions, the suitable compounds (such as L-DOPA, D-DOPA, dopamine, or carbidopa, etc.) may be used as agents to render the hydrophobic polymers bioadhesive, and/or be used as active ingredients in the pharmaceutical composition to be delivered to the patient. Thus, in certain embodiments, if carbidopa is used as part of the bioadhesive layer (for example, as the bioadhesive material on the shell of FIG. 5, or as the layer to coat the core comprising the second zero-order release portion), the total carbidopa dosage may be adjusted to account for the release of carbidopa from the bioadhesive material.

Similarly, in certain embodiments, when L- or D-dopa is used as the suitable compound to render the hydrophobic polymer bioadhesive, the dosage of total levodopa or precursor thereof may be adjusted elsewhere in, for example, the relevant portion or sub-portions of the IR or CR (controlled release, e.g., zero-order release rate portion).

In certain embodiments, a higher proportion of L-dopa (or D-Dopa) may be used to achieve a significant amount of release (e.g., more or less immediate release) from the polymers. In other embodiments, less L- or D-Dopa may be used such that the polymer is still adhesive, but the release of L- or D-Dopa from the bioadhesive polymer is less significant compared to the levodopa or precursors thereof in IR, and/or one or more other portions or sub-portions of the subject dosage form.

### Coatings

Preferred bioadhesive coatings do not appreciably swell upon hydration, such that they do not substantially inhibit or block movement (e.g., of ingested food) through the gastrointestinal tract, as compared to the polymers disclosed by Duchene et al. Generally, polymers that do not appreciably swell upon hydration include one or more hydrophobic regions, such as a polymethylene region (e.g., (CH₂)ₙ, where n is 4 or greater). The swelling of a polymer can be assessed by measuring the change in volume when the polymer is exposed to an aqueous solution. Polymers that do not appreciably swell upon hydration expand in volume by 50% or less when fully hydrated. Preferably, such polymers expand in volume by less than 25%, less than 20%, less than 15%, less than 10% or less than 5%. Even more preferably, the bioadhesive coatings are mucophilic. A polymer that does not appreciably swell upon hydration can be mixed with a polymer that does swell (e.g., Carbopol™, poly(acrylic acid), provided that the amount of swelling in the polymer does not substantially interfere with bioadhesiveness.

In one embodiment, the bioadhesive polymeric coating has two layers, an inner bioadhesive layer that does not substantially swell upon hydration and an outer bioadhesive layer that is readily hydratable and optionally bioerodable, such as one comprised of Carbopol™.

The bioadhesive polymers discussed above can be mixed with one or more plasticizers or thermoplastic polymers. Such agents typically increase the strength and/or reduce the brittleness of polymeric coatings. Examples of plasticizers include dibutyl sebacate, polyethylene glycol, triethyl citrate, dibutyl adipate, dibutyl fumarate, diethyl phthalate, ethylene oxide-propylene oxide block copolymers such as Pluronic™ F68 and di(sec-butyl)fumarate. Example of thermoplastic polymers include polyesters, poly(caprolactone), polylactide, poly(lactide-co-glycolide), methyl methacrylate (e.g., EUDRAGIT™), cellulose and derivatives thereof such as ethyl cellulose, cellulose acetate and hydroxypropyl methyl cellulose (HPMC) and large molecular weight polyanhydrides. The plasticizers and/or thermoplastic polymers are mixed with a bioadhesive polymer to achieve the desired properties. Typically, the proportion of plasticizers and thermoplastic polymers, when present, is from 0.5% to 40% by weight.

In one embodiment, the bioadhesive polymer coating, in a dry packaged form of a tablet, is a hardened shell.

A tablet or a drug eluting device can have one or more coatings in addition to the bioadhesive polymeric coating. These coatings and their thickness can, for example, be used to control where in the gastrointestinal tract the bioadhesive coating becomes exposed. In one example, the additional coating prevents the bioadhesive coating from contacting the mouth or esophagus. In another example, the additional coating remains intact until reaching the small intestine (e.g., an enteric coating).

Examples of coatings include methylmethacrylates, zein, modified zein, chitin, chitosan, cellulose acetate, cellulose phthalate, HMPC, sugars, enteric polymers, gelatin and shellac. Premature dissolution of a tablet in the mouth can be prevented with hydrophilic polymers such as HPMC or gelatin.

Coatings used in tablets of the invention typically include a pore former, such that the coating is permeable to the drug. Exemplary pore formers include: sugar, mannitol, HPC (hydroxypropyl cellulose), HPMC, dendrites, NaCl, etc.

Tablets and drug eluting devices of the invention can be coated by a wide variety of methods. Suitable methods include compression coating, coating in a fluidized bed or a pan, enrobing, and hot melt (extrusion) coating, etc. Such methods are well known to those skilled in the art.

All the above compositions, derivatives, precursors, additional components that can be used with levodopa/carbidopa, dosage forms, methods of making and using, etc., are adaptable or directly useable with the instant invention, and are thus expressly incorporated herein by reference.

### EXAMPLES

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Example 1

### Release Profile of a Dosage Form with IR+CR+IR Components

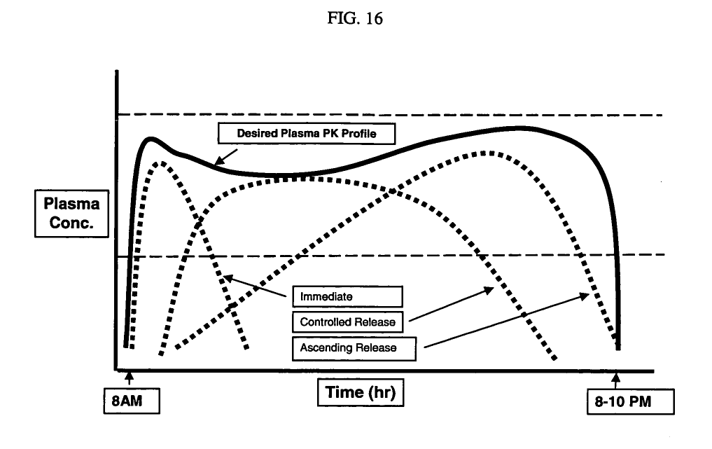

FIGS. 15 and 16 show two representative release profiles of a subject dosage formulation comprising a first immediate release portion (IR), a second substantially zero-order release portion (CR), and a third portion comprising a second immediate release portion (IR).

Specifically, FIG. 15 shows a representative release profile of an exemplary dosage formulation comprising a first immediate release portion (IR), a second substantially zero-order release portion (CR), and a third delayed immediate release portion (IR). For instance, each of the three portions may amount to about ⅓ of the total dosage form.

The percentage of carbidopa and levodopa released over time clearly shows a three-stage release profile. In the first stage of release, the first IR portion is rapidly dissolved, such that about 30% of the total carbidopa and about 30% of the total levodopa are released within about 30 minutes. Then both carbidopa and levodopa are released at a substantially zero-order release rate, such that another about 30-40% of the total carbidopa and another about 30-40% of the total levodopa are released over the next 34 hours. At the end of the second portion release, the third release stage (IR portion) began. During the third portion of release a final 30% of the total carbidopa and levodopa are dissolved within about 24 hours.

The ratio of levodopa-carbidopa in this exemplary dosage formulation is roughly the same (constant) in all three portions. But it needs not be the case in other embodiments of the invention. In addition, the release rate of carbidopa and levodopa from this exemplary dosage formulation is roughly the same. Again, this needs not be the case in other embodiments of the invention. Finally, the exemplary dosage formulation delivers carbidopa and levodopa at roughly the same time. This needs not be the case in other embodiments of the invention.

FIG. 16 shows a representative release profile of an exemplary dosage formulation comprising a first immediate release portion (IR), a second substantially zero-order release portion (CR), and a third delayed immediate release portion (IR). For instance, each of the three portions may amount to about ⅓ of the total dosage form.

The percentage of carbidopa and levodopa released over time clearly shows a three-stage release profile. In the first stage of release, the first IR portion is rapidly dissolved, such that about 30% of the total carbidopa and about 30% of the total levodopa are released within about 30 minutes. Then both carbidopa and levodopa are released at a substantially zero-order release rate, such that another about 30-40% of the total carbidopa and another about 30-40% of the total levodopa are released over the next 34 hours. At the end of the second portion release, the third release stage (IR portion) began. Again, the third portion release generates relatively steep lines representing carbidopa and levodopa dissolution, such that a final 30% of the total levodopa and carbidopa are dissolved within about 1-2 hours.

The ratio of levodopa-carbidopa in this exemplary dosage formulation is roughly the same (constant) in all three portions. But it needs not be the case in other embodiments of the invention. In addition, the release rate of levodopa and carbidopa from this exemplary dosage formulation is roughly the same. Again, this needs not be the case in other embodiments of the invention. Finally, the exemplary dosage formulation delivers levodopa and carbidopa at roughly the same time. This needs not be the case in other embodiments of the invention.

### Example 2

### In Vivo Release of Levodopa and Carbidopa

The following experiment was designed to determine if effective levodopa concentration in vivo is increased at the presence of a higher ratio of carbidopa to levodopa (as compared to that used in conventional therapy).

SINEMET® CR tablets (50 mg carbidopa/200 mg levodopa) were administered to fed beagle dogs either alone or after pre-dosing with 12.5 mg of carbidopa, and plasma concentrations of carbidopa and levodopa were measured over time (data not shown). The AUC (Area Under the Concentration-time curve) for each set of measurements were also summarized in Table 1 below.

TABLE I
AUC₀₋₂₄ (ng/mL × hr) of Carbidopa and Levodopa

| | SINEMET ® CR | Carbidopa + SINEMET ® CR |
|---|---|---|
| Levodopa | 3903 ± 298 | 8640 ± 2064 |
| Carbidopa | 215 ± 43 | 592 ± 303 |

Table I clearly shows a significant (almost 100%) increase in both peak concentrations for carbidopa and levodopa, and AUC₀₋₂₄, despite the fact that the total amount of levodopa in all experiments remained the same (e.g., 200 mg). This demonstrates that higher ratio of carbidopa/levodopa in the immediate release portion, or pre-dosing using carbidopa can lead to a higher effective levodopa concentration or AUC in animal models.

### Example 3

### Exemplary Multilayer Tablet and Multiparticulate Capsule Formulations

Given any specific release profiles, the effective components of the subject pharmaceutical compositions may be formulated in a number of ways to achieve the given release profile. The following examples provide two specific formulations—a multilayer tablet form and a multiparticulate capsule form—that both may be formulated to achieve substantially the same release profile.

For both the tablet and the capsule forms, the subject pharmaceutical compositions (e.g., Levodopa and/or Carbidopa) may be formulated as extended release formulations. Applicants have provided two different formulations, the multilayer tablet and the multiparticulate capsule forms. The multilayer extended release tablet approach is identical to the multiparticulate extended release capsule formulations with respect to the active pharmaceutical ingredients and the achieved dose level.

The SPHEROMER™ III or IV bioadhesive polymers, citric acid, and hydroxypropyl cellulose components are common to both the tablet formulation and the multiparticulate extended release capsule formulations. Additional excipients used in the multilayer extended release tablet formulations include magnesium stearate, succinic acid, hypromellose, corn starch, Ludipress®, butylated hydroxytoluene and p[FA:SA] or (SPHEROMER™ I). Poly[fumaric-co-sebacic acid anhydride] or p[FA:SA] (SPHEROMER™ I), is a bioadhesive polymer developed by Applicants that is similar to SPHEROMER™ III. All other excipients utilized in the multilayer extended release tablet formulations meet USP/NF specifications.

One exemplary multilayer extended release formulation approach provides a four-layer tablet. In this system, an immediate release (IR) active layer comprises levodopa/carbidopa, Ludipress®, citric acid, butylated hydroxytoluene and magnesium stearate. An inner controlled release (CR) layer is composed of levodopa, carbidopa, corn starch, succinic acid, butylated hydroxytoluene, magnesium stearate and hypromellose polymers. The CR layer is sandwiched between two bioadhesive layers containing poly[fumaric-co-sebacic acid] or p[FA:SA], citric acid, and levodopa-(graft) butadiene maleic anhydride polymer (SPHEROMER™ III). Formulations A and B differ in the levels of rate-controlling hypromellose polymers in the inner CR layer and consequently have different dissolution profiles for levodopa and carbidopa.

Unit dose composition of Levodopa-Carbidopa Multilayer Extended Release Tablet Type A and Type B formulations are listed below in Table A and Table B, respectively.

TABLE A
Unit dose composition of Levodopa-Carbidopa Multilayer
Extended Release Tablet, 200 mg/50 mg Type A

| | | Wt per |
|---|---|---|
| Components | % w/w | Tablet (mg) |
| Levodopa, USP | 20.25 | 200.0 |
| Carbidopa, monohydrate, USP | 5.46 | 53.9 |
| Citric acid, anhydrous, USP | 2.49 | 24.6 |
| Succinic Acid, FCC | 6.58 | 65.0 |
| Butylated hydroxytoluene, NF | 0.04 | 0.4 |
| Hypromellose 2208, 100 cps, USP | 3.58 | 35.3 |
| Corn Starch, NF | 0.98 | 9.7 |
| Hypromellose 2910, 5 cps, USP | 5.32 | 52.5 |
| Ludipress ® | 6.00 | 59.2 |
| Magnesium stearate, NF | 0.27 | 2.7 |
| SPHEROMER ™ III | 31.53 | 311.3 |
| p[FA:SA] 1:4, (SPHEROMER ™ I) | 10.93 | 107.9 |
| Hydroxypropylcellulose, NF | 6.57 | 64.9 |
| Dehydrated alcohol, USP | * | * |
| Methylene Chloride, NF | * | * |
| Methyl alcohol NF | * | * |
| Total | 100.00 | 987.4 |

* Evaporated during drying of granulation.

TABLE B
Unit dose composition of Levodopa-Carbidopa Multilayer Extended
Release Tablet, 200 mg/50 mg Type B

| | | Wt per |
|---|---|---|
| Components | % w/w | Tablet (mg) |
| Levodopa, USP | 20.25 | 200.0 |
| Carbidopa, monohydrate, USP | 5.46 | 53.9 |
| Citric acid, anhydrous, USP | 2.49 | 24.6 |
| Succinic Acid, FCC | 6.58 | 65.0 |
| Butylated hydroxytoluene, NF | 0.04 | 0.40 |
| Hypromellose 2208, 100 cps, USP | 7.13 | 70.3 |
| Hypromellose 2208, 4000 cps, USP | 0.35 | 3.5 |
| Corn Starch, NF | 0.63 | 6.2 |
| Hypromellose 2910, 5 cps, USP | 1.77 | 17.5 |
| Ludipress ® | 6.00 | 59.2 |
| Magnesium stearate, NF | 0.27 | 2.7 |
| SPHEROMER ™ III | 31.53 | 311.3 |
| p[FA:SA] 1:4 (SPHEROMER ™ I) | 10.93 | 107.9 |
| Hydroxypropylcellulose, NF | 6.57 | 64.9 |
| Dehydrated alcohol, USP | * | * |
| Methylene Chloride, NF | * | * |
| Methyl alcohol, NF | * | * |
| Total | 100.00 | 987.4 |

*Evaporated during drying of granulation.

With the exception of SPHEROMER™ I, and SPHEROMER™ III, all excipients utilized in both formulations are within or below the listed levels for orally administered products.

The subject bioadhesive multilayer extended releases tablets using p[FA:SA] as the bioadhesive polymer typically result in an improved bioavailability and reduced variability compared to SINEMET® tablets. Such formulations and dosage forms can be generally used for a broad spectrum of compounds (e.g., drugs, prodrugs, metabolic precursors, etc.), especially those with limited absorption windows in upper GI (e.g., stomach), such as levodopa and carbidopa.

Examples below provide details of making the various component pellets in the subject pharmaceutical compositions (such as levodopa, carbidopa, or levodopa-carbidopa pellets), the in vitro and/or in vivo dissolution profiles of the subject pharmaceutical compositions, and the comparison with those of the SINEMET® tablets. It is apparent that the subject compositions consistently deliver steady levels of the exemplary pharmaceutic composition (levodopa and carbidopa in these cases) within the intended effective range (see Cₘₐₓ), over a longer period of time while avoiding the large/sharp peaks and valleys typically seens in the release profile of SINEMET® tablets (compare AUC and Tₘₐₓ).

### Example 4

### Low-shear Wet Granulation of Levodopa, Carbidopa, and Levodopa-Carbidopa

This example provides exemplary levodopa, carbidopa, and levodopa-carbidopa granules, which were produced with low-shear wet granulation method. The following steps (or minor variations thereof) may be followed to make such granules:
(1) Weighing levodopa or carbidopa, or both levodopa and carbidopa, optionally a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer, operating at the speed setting #1, for 5-15 min, forming a uniform dry mix.
(3) Granulating the dry mix from step (2) under low shear with a granulation fluid, forming a wet granulation. Granulation fluids were mainly selected from purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone. The granulation was conducted in a small 500-mL cylindrical vessel with manual mixing or in a planetary type mixer, Hobart Mixer with a 5-qt mixing bowl, operating at the speed setting #1, depending on the batch size.
(4) Drying the wet granulation from step (3), e.g., in a Precision gravity oven, operating at 50° C., for 8-24 h. Alternatively, the granulation was dried in a fluidized bed drier, Vector MFL.01 Micro Batch Fluid Bed System, operating at an inlet air flow rate of 100-300 lpm (liters per minute) and an inlet air temperature of 50° C.
(5) Grinding the dried granulation from step (4), e.g., by using a pestle in a mortar, followed by sieving the ground material, e.g., through a U.S. Std. mesh # 60 screen.
(6) Blending the sieved granulation from step (5) with a lubricant, e.g., using an end-over-end ATR rotator, model RKVS, or in a planetary type mixer, Hobart Mixer, operating at the speed setting #1, for 5-15 min, forming a uniformly lubricated dry mix ready for compression. and
(7) Optionally, passing the lubricated dry mix from step (6) through a sieve or screen, e.g., a U.S. Std. mesh # 60 screen.

### Example 5

### Low-Shear Wet Granulation of a Bioadhesive Polymer, SPHEROMER™ I [p(FASA)] or SPHEROMER™ III

This example provides exemplary Bioadhesive Polymer, SPHEROMER™ I [p(FASA)] or SPHEROMER™ III granules, which were produced with low-shear wet granulation method. The following steps (or minor variations thereof) may be followed to make such granules:
(1) Weighing the bioadhesive polymer and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Granulating the dry mix from step (2) under low shear with a granulation fluid, forming a wet granulation. Granulation fluids were mainly selected from a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone. The granulation was conducted in a small cylindrical vessel with manual mixing or in a planetary type mixer, Hobart Mixer with a 5-qt mixing bowl, operating at the speed setting #1, depending on the batch size.
(4) Drying the wet granulation from step (3), e.g., in a Precision gravity oven, operating at 50° C., for 8-24 h. Alternatively, the granulation was dried in a fluidized bed drier, Vector MFL.01 Micro Batch Fluid Bed System, operating at an inlet air flow rate of 100-300 lpm (liters per minute) and an inlet air temperature of 55° C.
(5) Grinding the dried granulation from step (4), e.g., by using a pestle in a mortar, followed by sieving the ground material through a U.S. Std. mesh # 40 screen.
(6) Blending the sieved granulation from step (5) with a lubricant, e.g., using an end-over-end ATR rotator, model RKVS, or in a planetary type mixer, Hobart Mixer, operating at the speed setting #1, for 5-15 min, forming a uniformly lubricated dry mix ready for compression. and
(7) Optionally, passing the lubricated dry mix from step (6) through a sieve or screen, such as a U.S. Std. mesh # 40 screen.

### Example 6

### Production of Levodopa, Carbidopa, and Levodopa-Carbidopa Tablets

This example provides exemplary levodopa, carbidopa, and levodopa-carbidopa tablets, which were produced with direct compression. The following steps (or minor variations thereof) may be followed to make such tablets:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, or in a GlobePharma Maxiblend V-shell blended equipped with a 0.5-qt V-shell for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.
(4) Compressing the lubricated dry mix from step (3), e.g., into tablets, such as by using a single-station manual tablet press, GlobePharma Manual Tablet Compaction Machine MTCM-I, equipped with adequate die and punch set. Tablets were prepared at a pressure ranging from 250 to 4000 pounds per square inch (psi) and a compression time of 1 to 4 seconds.

Alternatively, tablets were produced with wet granulation of active ingredients followed by direct compression. The production processes may include the following:
(1) Granulating levodopa or carbidopa, or both levodopa and carbidopa, optionally a bioadhesive polymer composition, and pharmaceutically acceptable excipients in accordance with the method explained in Example 4.
(2) Compressing the lubricated dry granulation mix from step (1) into tablets using a single-station manual tablet press, e.g., GlobePharma Manual Tablet Compaction Machine MTCM-I, equipped with adequate die and punch set. Tablets were prepared at a pressure ranging from 250 to 4000 pounds per square inch (psi) and a compression time of 1 to 4 seconds.

### Example 7

### Production of Levodopa-Carbidopa Bilayer Tablets

This example provides exemplary levodopa-carbidopa bilayer tablets, which were produced with direct compression. The following steps (or minor variations thereof) may be followed to make each tablet layer:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

Bilayer tablets were produced using a single-station manual tablet press, GlobePharma Manual Tablet Compaction Machine MTCM-I, equipped with adequate die and punch set. The compression process included:
(4) Adding the first lubricated layer blend into the die cavity, optionally followed by manually tapping it using a stainless steel spatula.
(5) Adding the second lubricated layer blend into the die cavity.
(6) Pre-compressing the two layers together, e.g., at a pressure ranging from 250 to 500 pounds per square inch (psi) and a compression time of 1 to 5 seconds.
(7) Compressing the pre-compacted layers together, e.g., at a pressure ranging from 1000 to 4000 pounds per square inch (psi) and a compression time of 1 to 4 seconds.

Alternatively, bilayer tablets were prepared by first granulating the layer blends followed by blending the granulations with a lubricant in accordance with the method of Example 4, and finally compressing the lubricated layer granulations together into a tablet.

### Example 8

### Production of Levodopa-Carbidopa Trilayer Tablets

This example provides exemplary levodopa-carbidopa trilayer tablets, which were produced with direct compression. The following steps (or minor variations thereof) may be followed to make each tablet layer:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

Trilayer tablets were produced using a single-station manual tablet press, GlobePharma Manual Tablet Compaction Machine MTCM-I, equipped with adequate die and punch set. The compression process included:
(4) Adding the first lubricated layer blend into the die cavity, optionally followed by manually tapping it using a stainless steel spatula.
(5) Adding the second lubricated layer blend into the die cavity, optionally followed by manually tapping it together with the first layer using a stainless steel spatula.
(6) Adding the third lubricated layer blend into the die cavity.
(7) Pre-compressing the three layers together, e.g., at a pressure ranging from 200 to 500 pounds per square inch (psi) and a compression time of 1 to 5 seconds.
(8) Compressing the pre-compacted layers together, e.g., at a pressure ranging from 1000 to 4000 pounds per square inch (psi) and a compression time of 1 to 4 seconds.

Alternatively, trilayer tablets were prepared by first granulating the layer blends followed by blending the granulations with a lubricant in accordance with the method of Example 4, and finally compressing the lubricated layer granulations together into a tablet.

### Example 9

### Production of Levodopa-Carbidopa Quadrilayer Tablets

This example provides exemplary levodopa-carbidopa quadrilayer tablets, which were produced with direct compression. The following steps (or minor variations thereof) may be followed to make each tablet layer:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5 qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

Quadrilayer tablets were produced using a single-station manual tablet press, GlobePharma Manual Tablet Compaction Machine MTCM-I, equipped with adequate die and punch set. The compression process included:
(4) Adding the first lubricated layer blend into the die cavity, optionally followed by manually tapping it using a stainless steel spatula.
(5) Adding the second lubricated layer blend into the die cavity, optionally followed by manually tapping it together with the first layer using a stainless steel spatula.
(6) Adding the third lubricated layer blend into the die cavity, optionally followed by manually tapping it together with the first and second layers using a stainless steel spatula.
(7) Adding the fourth lubricated layer blend into the die cavity.
(8) Pre-compressing the four layers together, e.g., at a pressure ranging from 250 to 500 pounds per square inch (psi) and a compression time of 1 to 5 seconds.
(9) Compressing the pre-compacted layers together, e.g., at a pressure ranging from 1000 to 4000 pounds per square inch (psi) and a compression time of 1 to 4 seconds.

Alternatively, quadrilayer tablets were prepared by first granulating the layer blends followed by blending the granulations with a lubricant in accordance with the method of Example 4, and finally compressing the lubricated layer granulations together into a tablet.

### Example 10

### Production of Levodopa-Carbidopa Trilayer Tablets with a Pre-Compressed Insert

This example provides exemplary levodopa-carbidopa trilayer tablets with a pre-compressed insert, which were produced with direct compression. The following steps (or minor variations thereof) may be followed to make each tablet layer:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

The pre-compressed insert was produced with direct compression and the production processes included:
(4) Weighing levodopa or carbidopa, or both levodopa and carbidopa, and or a bioadhesive polymer composition, and pharmaceutically acceptable excipients.
(5) Blending the weighed ingredients from step (4) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(6) Blending the dry mix from step (5) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.
(7) Compressing the lubricated mix from step (6) using a single-station manual tablet press, GlobePharma Manual Tablet Compaction Machine MTCM-I, equipped with adequate die and punch set. Tablets were prepared, e.g., at a pressure ranging from 500 to 1000 pounds per square inch (psi) and a compression time of, e.g., 1 to 2 seconds.

The trilayer tablets with pre-compressed insert were produced using a single-station manual tablet press, GlobePharma Manual Tablet Compaction Machine MTCM-I, equipped with adequate die and punch set. The compression process included:
(8) Adding the first lubricated layer blend into the die cavity, optionally followed by manually tapping it using a stainless steel spatula.
(9) Placing the pre-compressed tablet on the first layer in the center of the die.
(10) Adding the second lubricated layer blend into the die cavity, optionally followed by manually tapping it together with the first layer and the pre-compressed tablet using a stainless steel spatula.
(11) Adding the third lubricated layer blend into the die cavity.
(12) Compressing the three layers together with the pre-compressed insert, e.g., at a pressure ranging from 2000 to 4000 pounds per square inch (psi) and a compression time of 1 to 4 seconds.

The pre-compressed tablet was alternatively placed in the middle of the second layer in the center of the die.

Alternatively, the tablets were prepared by first granulating the layer blends and the pre-compressed tablet ingredients blend followed by mixing the granulations with a lubricant in accordance with the method of Example 4, preparing the pre-compressed insert, and finally compressing the lubricated layer granulations together with the pre-compressed insert into a tablet.

### Example 11

### Production of Levodopa-Carbidopa Longitudinally Compressed Bioadhesive Multilayer Tablets

This example provides exemplary levodopa-carbidopa longitudinally compressed bioadhesive multilayer tablets, which were produced with direct compression. Each tablet had at least three layers, longitudinally compressed together, having a cylindrical shape. The trilayer tablet was sealed peripherally with a bioadhesive polymeric composition by compression or heat-sealing technique. The following steps (or minor variations thereof) may be followed to make each tablet layer, including the coating layer:
(1) Weighing levodopa or carbidopa, or both levodopa and carbidopa, or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

Trilayer tablets were produced using, e.g., a single-station manual tablet press, GlobePharma Manual Tablet Compaction Machine MTCM-I, equipped with adequate die and punch set. The compression process included:
(4) Adding the first lubricated layer blend into the die cavity, optionally followed by manually tapping it using a stainless steel spatula.
(5) Adding the second lubricated layer blend into the die cavity, optionally followed by manually tapping it together with the first layer using a stainless steel spatula.
(6) Adding the third lubricated layer blend into the die cavity.
(7) Pre-compressing the three layers together longitudinally into a trilayer tablet at a pressure ranging from 250 to 500 pounds per square inch (psi) and a compression time of 1 to 5 seconds.
(8) Compression-coating of the trilayer tablet from step (7) with the coating layer, e.g., at a pressure ranging from 2000 to 4000 pounds per square inch (psi) and a compression time of, e.g., 1 to 4 seconds.

Alternatively, trilayer tablets were prepared by first granulating the layer blends followed by blending the granulations with a lubricant in accordance with the method of Example 4, and finally compressing the lubricated layer granulations together longitudinally into a tablet.

### Example 12

### Production of Levodopa-Carbidopa Triple Pressed Tablets

This example provides exemplary levodopa-carbidopa triple pressed tablets produced with compression of a pre-compressed core tablet with two layers of coating materials. The following steps (or minor variations thereof) may be followed to make each coating layer:
(1) Weighing levodopa or carbidopa, or both levodopa and carbidopa, and or a bioadhesive polymer composition, and pharmaceutically acceptable excipients.
(2) Blending the weighed ingredients from step (1) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(3) Blending the dry mix from step (2) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.

The pre-compressed tablet was produced with direct compression and the production processes included:
(4) Weighing levodopa or carbidopa, or both levodopa and carbidopa, and or a bioadhesive polymer composition, and pharmaceutically acceptable excipients.
(5) Blending the weighed ingredients from step (4) excluding a lubricant, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(6) Blending the dry mix from step (5) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.
(7) Compressing the lubricated mix from step (6) using, e.g., a single-station manual tablet press, GlobePharma Manual Tablet Compaction Machine MTCM-I, equipped with adequate die and punch set. Tablets were prepared, e.g., at a pressure ranging from 250 to 500 pounds per square inch (psi) and a compression time of, e.g., 1 second.

The pre-compressed tablet was compression-coated, e.g., by the coating layers using a single-station manual tablet press, GlobePharma Manual Tablet Compaction Machine MTCM-I, equipped with adequate small and large dies and punch sets. The compression process included:
(8) Adding about half of the first lubricated coating layer blend into the small die cavity.
(9) Placing the pre-compressed tablet on the first half-layer blend in the center of the die.
(10) Adding the second half-layer blend into the die cavity.
(11) Compressing the pre-compressed tablet and the first coating layer together, e.g., at a pressure ranging from 500 to 1000 pounds per square inch (psi) and a compression time of, e.g., 1 second.
(12) Ejecting the double pressed tablet of step (11) from the die.
(13) Adding about half of the second lubricated coating layer blend into the large die cavity.
(14) Placing the double pressed tablet on the first half-layer blend in the center of the die.
(15) Adding the second half-layer blend into the die cavity.
(16) Compressing the double pressed tablet and the second coating layer together, e.g., at a pressure ranging from 2000 to 4000 pounds per square inch (psi) and a compression time of, e.g., 1 to 4 seconds.

Alternatively, the tablets were prepared by first granulating the pre-compressed tablet ingredients blend and coating layer blends followed by mixing the granulations with a lubricant in accordance with the method of Example 4, preparing the pre-compressed tablet, and successive compressing of the pre-compressed tablet with lubricated layer granulations into the final tablet.

### Example 13

### Production of Levodopa-Carbidopa Pellets with Granulation-Extrusion-Spheronization

This example provides exemplary levodopa-carbidopa pellets produced with granulation-extrusion-spheronization. The following steps (or minor variations thereof) may be used:
(1) Weighing levodopa and carbidopa, optionally a bioadhesive polymer composition, and pharmaceutically acceptable excipients.
(2) Blending of the weighed ingredients of step (1) in a planetary type mixer, e.g., Hobart Mixer with a 5-qt mixing bowl, operating at the speed setting #1, for 5-15 min, forming a dry mix.
(3) Granulating the dry mix from step (2) under low shear with a granulation fluid, forming a wet granulation. The granulation fluids were mainly selected from, e.g., purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone.
(4) Extruding the wet granulation from step (3) through the screen of a screen-type extruder, e.g., Caleva Model 20 (or Model 25) Extruder, operating at, e.g., 10-20 rpm, and forming breakable wet strands, the extrudate. The screen aperture was 0.8, 1, or 1.5 mm.
(5) Spheronizing the extrudate from step (4) in a spheronizer, e.g., Caleva Model 250, equipped with a 2.5-mm spheronization plate, operating at, e.g., 1000-2000 rpm for 5-10 min, and forming spheronized pellets.
(6) Drying the spheronized pellets from step (5) in a fluidized bed drier, e.g., Vector MFL.01 Micro Batch Fluid Bed System, operating at an inlet air flow rate of, e.g., 100-300 lpm (liters per minute) and an inlet air temperature of, e.g., 50° C.
(7) Screening and classifying the dried pellets from step (6) through a stack of stainless steel sieves, U.S. standard mesh sizes 8, 10, 12, 14, 16, 18, 20, 25, 30, 40, 45, and 60 using a mechanical sieve shaker, e.g., W.S. Tyler Sieve Shaker Ro-Tap Rx-29, operated for 5 min. Particle size and distribution of pellet formulations were analyzed, and classified pellets ranging from 0.25 mm (mesh # 60) to 2 mm (mesh # 10) were selected for future film coating or other experimentation.

### Example 14

### Film Coating of Levodopa-Carbidopa Pellets

Levodopa-carbidopa pellets were film-coated with a sub-layer of release rate controlling polymer(s) such as EUDRAGIT® RL 100, EUDRAGIT® RS 100, or mixtures thereof, and with a top-layer of a bioadhesive polymer such as SPHEROMER™ I [p(FASA)], SPHEROMER™ III, SPHEROMER™ IV, or mixtures thereof. Optionally, pellets were film-coated with an additional layer of a non-functional polymer such as hydroxypropylmethyl cellulose, hydroxypropyl cellulose, and polyvinyl alcohol. Polymers were dissolved in different solvent systems depending on their solubility characteristics. The film coating was performed in a fluidized bed coater, Vector MFL.01 Micro Batch Fluid Bed System, equipped with a Wurster insert, operating at an inlet air flow rate of 100-300 lpm (liter per minute) and an inlet air temperature of 30° C.-35° C. The pellets were pre-warmed at 35° C. for 2-5 min and after film-coating were post-dried at 30° C. for 15-30 min.

### Example 15

### Production of Levodopa-Carbidopa Rapidly Disintegrating Pelletized Tablets

Rapidly disintegrating pelletized tablets were produced by compression of film-coated spheronized pellets within a carrier matrix. Production processes included the following steps:
(7) Production of levodopa-carbidopa with granulation-extrusion-spheronization in accordance with the method described in Example 13.
(7) Film-coating of a levodopa-carbidopa pellets from step (1) with EUDRAGIT® RL 100, EUDRAGIT® RS 100, or mixtures thereof, in accordance with the method described in Example 14.
(7) Blending of the pre-weighed ingredients of the rapidly disintegrating matrix. The ingredients included levodopa, carbidopa, a superdisintegrant, and pharmaceutically acceptable excipients, excluding a lubricant. Depending on the batch size, blending was carried out, e.g., using an end-over-end ATR rotator, model RKVS, or in a planetary type mixer, Hobart Mixer with a 5-qt mixing bowl, operating at the speed setting #1, for 5-15 min, forming a uniform dry mix.
(7) Blending the dry mix from step (3) with a lubricant, e.g., using an end-over-end ATR rotator, model RKVS, or in a planetary type mixer, Hobart Mixer with a 5-qt mixing bowl, operating at the speed setting #1, for 5-15 min, forming a uniformly lubricated dry mix.
(7) Blending of the pre-weighed amounts of film-coated pellets from step (2) and lubricated dry mix from step (4), adequate to prepare a single tablet.
(7) Compressing the mixture from step (5) into a tablet, e.g., using a single-station manual tablet press, GlobePharma Manual Tablet Compaction Machine MTCM-I, equipped with adequate die and punch set. Tablets were pre-compressed, e.g., at a pressure ranging from 250 to 500 pounds per square inch (psi) for, e.g., 1 to 2 seconds, and subsequently compressed, e.g., at a pressure of 2000 to 4000 psi for, e.g., 1 to 4 seconds.

### Example 16

### Production of Levodopa-Carbidopa Slowly Eroding Pelletized Trilayer Tablets

Slowly eroding pelletized multilayer tablets were produced by compression of film-coated spheronized pellets along with three laminated layers; the bottom layer functioning as a passive supporting and optionally bioadhesive layer—the middle layer carrying the film-coated active pellets and eroding slowly, releasing the pellets—and the top layer disintegrating rapidly and releasing its active contents when exposed to aqueous environments. Production processes included the following steps:
(1) Production of levodopa-carbidopa with granulation-extrusion-spheronization in accordance with the method described in Example 13.
(2) Film-coating of a levodopa-carbidopa pellets from step (1) with EUDRAGIT® RL 100, EUDRAGIT® RS 100, or mixtures thereof, in accordance with the method described in Example 14.
(3) Blending of the pre-weighed ingredients of the supporting layer. The ingredients included pharmaceutically acceptable excipients, excluding a lubricant, and optionally a bioadhesive polymer. Depending on the batch size, blending was carried out, e.g., using an end-over-end ATR rotator, model RKVS, or in a planetary type mixer, e.g., Hobart Mixer with a 5-qt mixing bowl, operating at the speed setting #1, for 5-15 min, forming a uniform dry mix.
(4) Blending of the pre-weighed ingredients of the immediate release layer. The ingredients included levodopa, carbidopa, a superdisintegrant, and pharmaceutically acceptable excipients, excluding a lubricant. Depending on the batch size, blending was carried out, e.g., using an end-over-end ATR rotator, model RKVS, or in a planetary type mixer, e.g., Hobart Mixer with a 5-qt mixing bowl, operating at the speed setting #1, for 5-15 min, forming a uniform dry mix.
(5) Blending of the pre-weighed ingredients of the slow-eroding matrix. The ingredients included pharmaceutically acceptable excipients, excluding a lubricant. Depending on the batch size, blending was carried out, e.g., using an end-over-end ATR rotator, model RKVS, or in a planetary type mixer, e.g., Hobart Mixer with a 5-qt mixing bowl, operating at the speed setting #1, for 5-15 min, forming a uniform dry mix.
(6) Blending the dry blend from steps (3), (4) and (5) with a lubricant, e.g., using an end-over-end ATR rotator, model RKVS, or in a planetary type mixer, e.g., Hobart Mixer with a 5-qt mixing bowl, operating at the speed setting #1, for 5-15 min, forming a uniformly lubricated dry mix.
(7) Blending of the pre-weighed amounts of film-coated pellets from step (2) and lubricated slow-eroding layer mix from step (6), adequate to prepare a single tablet.

Trilayer tablets were manufactured using a single-station manual tablet press, GlobePharma Manual Tablet Compaction Machine MTCM-I, equipped with adequate die and punch set. The compression process included:
(8) Adding the lubricated supporting layer blend from step (6) into the die cavity, optionally followed by manually tapping it using a stainless steel spatula.
(9) Adding the slow eroding layer blend from step (7) into the die cavity, optionally followed by manually tapping it together with the first layer using a stainless steel spatula.
(10) Adding the lubricated immediate release layer blend from step (6) into the die cavity.
(11) Pre-compressing the three layers together, e.g., at a pressure ranging from 250 to 500 pounds per square inch (psi) and a compression time of, e.g., 1 second.
(12) Compressing the pre-compacted layers together, e.g., at a pressure ranging from 1000 to 4000 pounds per square inch (psi) and a compression time of, e.g., 1 to 4 seconds.

Alternatively, trilayer tablets were prepared by first granulating the layer blends followed by blending the granulations with a lubricant, and finally compressing the lubricated layer granulations together with film-coated pellets into a tablet.

### Example 17

### Film Coating of Levodopa, Carbidopa, and Levodopa-Carbidopa Tablets with Bioadhesive Polymer, SPHEROMER™ III

Levodopa, carbidopa, and levodopa-carbidopa tablets were film coated with a bioadhesive polymeric composition, SPHEROMER™ III. Bioadhesive SPHEROMER™ III and optionally a functional polymer, or a non-functional polymer, were dissolved in methanol. The film coating was performed in a laboratory pan coater, O'Hara Labcoat, operating at an inlet air flow rate of 60 cfm (cubic foot per min.) and an inlet air temperature of 35° C. The tablets were pre-warmed at 35° C. for 5-10 min and after film coating were post-dried at 30° C. for 15-30 min.

### Example 18

### Film Coating of Levodopa, Carbidopa, and Levodopa-Carbidopa Tablets with Bioadhesive Polymer, SPHEROMER™ IV

Levodopa, carbidopa, and levodopa-carbidopa tablets were film coated with a bioadhesive polymeric composition, SPHEROMER™ IV. Bioadhesive SPHEROMER™ IV and optionally a functional polymer, or a non-functional polymer, were dissolved in methanol or a mixture of ethanol and water (3:1 v/v). The film coating was performed in a laboratory pan coater, O'Hara Labcoat, operating at an inlet air flow rate of 60 cfm (cubic foot per minute) and an inlet air temperature of 35° C. The tablets were pre-warmed at 35° C. for 5-10 min and after film coating were post-dried at 30° C. for 15-30 min.

### Example 19

### Film Coating of Levodopa, Carbidopa, and Levodopa-Carbidopa Tablets with a Functional or a Non-functional Polymer

Levodopa, carbidopa, and levodopa-carbidopa pellets were film coated with a functional, or with a non-functional polymer. The polymer was dissolved in either of methanol, ethanol, or isopropanol, or their mixture with acetone. The film coating was performed in a laboratory pan coater, O'Hara Labcoat, operating at an inlet air flow rate of 60 cfm (cubic foot per minute) and an inlet air temperature of 30° C. to 40° C. The tablets were pre-warmed at 30° C. to 40° C. for 2-5 min and after film coating were post-dried at 30° C. to 40° C. for 15-30 min.

### Example 20

### In Vitro Dissolution of Tablet Formulations of Levodopa, Carbidopa, and Levodopa-Carbidopa

The in vitro dissolution profile of levodopa, carbidopa, and levodopa-carbidopa tablet formulations were obtained under simulated gastric conditions. The dissolution tests were performed in 900 mL of either of 0.1 N HCl-pH 1.2, phosphate buffer saline (PBS)-pH 4.5, or sodium acetate buffer-pH 4.5 solutions in a USP II apparatus at a temperature of 37° C. The paddle speed was set at 50 rpm. Samples of dissolution media were collected at predetermined intervals and analyzed by either HPLC or UV spectrophotometry.

### Example 21

### In Vitro Dissolution of SINEMET® 10-100 Tablets, Containing 10 mg Carbidopa and 100 mg Levodopa, Lot # 00067

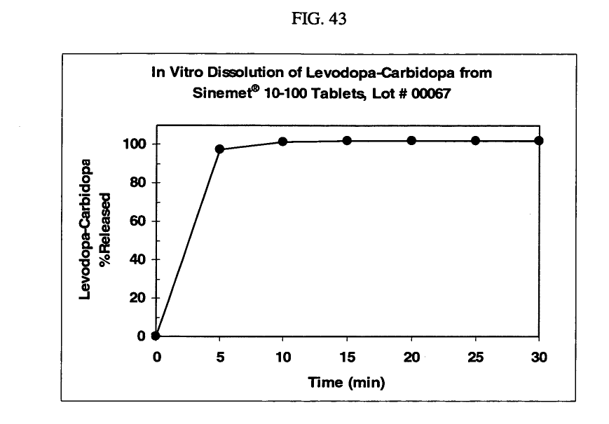

The in vitro dissolution profile of SINEMET® 10-100 tablets, containing 10 mg carbidopa and 100 mg levodopa was obtained under simulated gastric conditions. The dissolution tests were performed in 900 mL of either of 0.1 N HCl-pH 1.2, phosphate buffer saline (PBS)-pH 4.5, or sodium acetate buffer-pH 4.5 solutions, in a USP II apparatus at a temperature of 37° C. The paddle speed was set at 50 rpm. Samples of dissolution media were collected at predetermined intervals and analyzed by UV spectrophotometry. The combined dissolution profile of levodopa-carbidopa obtained from UV spectrophotometry analysis is shown in FIG. 43.

### Example 22

### In Vitro Dissolution of SINEMET® CR 50-200 Tablets, Containing 50 mg Carbidopa and 200 mg Levodopa, Lot # N4682

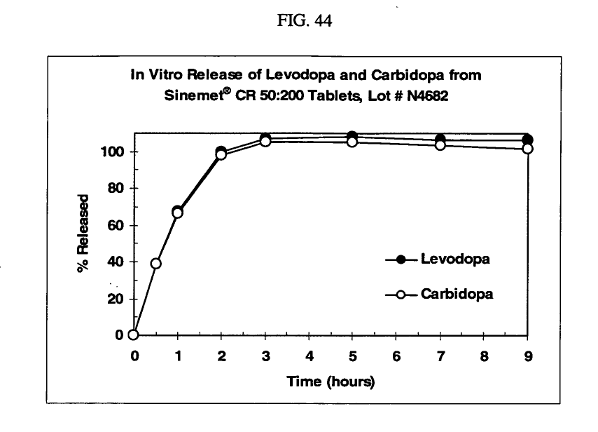

The in vitro dissolution profile of SINEMET® CR 50-200 tablets, containing 50 mg carbidopa and 200 mg levodopa were obtained under simulated gastric conditions. The dissolution tests were performed in 900 mL of 0.1 N HCl-pH 1.2 solution, in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 44.

### Example 23

### In Vivo Pharmacokinetic Performance of SINEMET® 10-100 Tablets in Fed Beagle Dogs, Lot # 00067

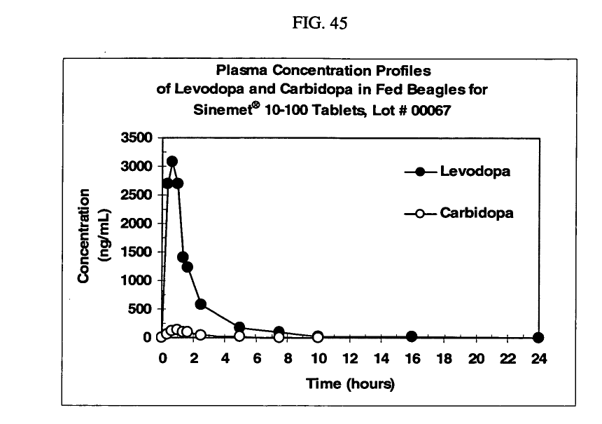

The in vivo performance of SINEMET® 10-100 tablets was evaluated in beagle dogs. SINEMET® tablets were administered to cohorts of six beagle dogs in the fed state and plasma levels of levodopa and carbidopa were measured using LC/MS/MS analysis. FIG. 45 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 1.

TABLE 1
Pharmacokinetic Data for SINEMET ® 10-100 Tablets,
Lot # 00067, in Fed Beagle Dogs; the area under the plasma
levodopa vs. time curve (AUC), maximum concentration
(Cₘₐₓ), and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® 10-100 Tablets | 5,956 | 3,400 | 0.66 |

### Example 24

### In Vivo Pharmacokinetic Performance of SINEMET® CR 50-200 Tablets in Fed Beagle Dogs, Lot # N4682

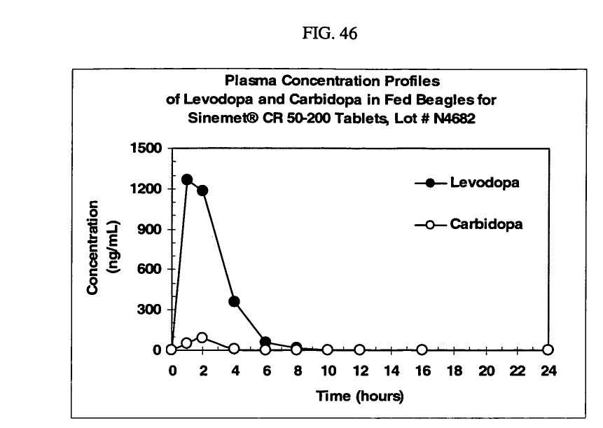

The in vivo performance of SINEMET® CR 50-200 tablets was evaluated in beagle dogs. SINEMET® CR tablets were administered to cohorts of six beagle dogs in the fed state and plasma levels of levodopa and carbidopa were measured using HPLC analysis. FIG. 46 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 2.

TABLE 2
Pharmacokinetic Data for SINEMET ® CR 50-200 Tablets,
Lot # N4682, in Fed Beagle Dogs; the area under the plasma
levodopa vs. time curve (AUC), maximum concentration (Cₘₐₓ),
and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® CR 50-200 Tablets | 3,903 | 1,663 | 2 |

### Example 25

### In Vivo Pharmacokinetic Performance of SINEMET® CR 50-200 Tablets in Fasted Beagle Dogs, Lot # N4682

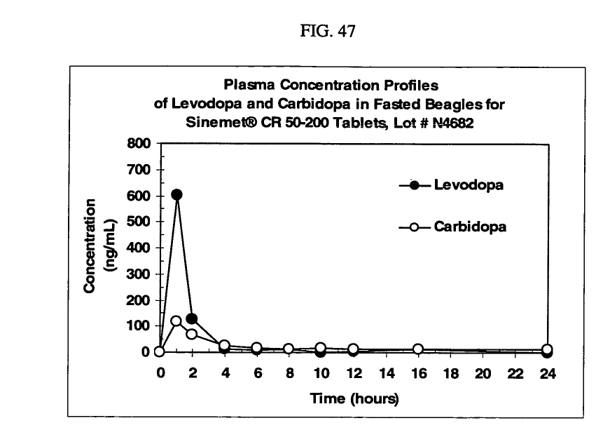

The in vivo performance of SINEMET® CR 50-200 tablets was evaluated in beagle dogs. SINEMET® CR tablets were administered to cohorts of twelve beagle dogs in the fasted state and plasma levels of levodopa and carbidopa were measured using HPLC analysis. FIG. 47 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 3.

TABLE 3
Pharmacokinetic Data for SINEMET ® CR 50-200 Tablets,
Lot # N4682, in Fasted Beagle Dogs; the area under the plasma
levodopa vs. time curve (AUC), maximum concentration
(Cₘₐₓ), and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® CR 50-200 Tablets | 936 | 604 | 1 |

### Example 26

### Bioadhesive Levodopa-Carbidopa 200 mg/50 mg Trilayer Tablets, Lot # 505-065

Bioadhesive levodopa-carbidopa trilayer tablets were produced with direct compression in accordance with the method described in Example 8. Tablets comprised an active controlled release (CR) layer laminated between two passive bioadhesive layers. The weight and composition of the CR and bioadhesive layers are given in Table 4.

TABLE 4
Weight and Composition of Controlled Release and Bioadhesive
Layers of Levodopa-Carbidopa 200 mg/50 mg Trilayer Tablet,
Lot # 505-065

| | | |
|---|---|---|
| Ingredients | Weight % | Weight (mg) |

Controlled Release Layer

| | | |
|---|---|---|
| Levodopa, USP | 47.0 | 200.0 |
| Carbidopa monohydrate, USP | 12.7 | 54.0 |
| Hypromellose 2208, 100 cps, USP | 19.7 | 83.6 |
| Hypromellose 2910, 5 cps, USP | 9.8 | 41.8 |
| L-Glutamic acid, FCC | 4.9 | 20.8 |
| Corn Starch, NF | 4.9 | 20.8 |
| Magnesium Stearate, NF | 1.0 | 4.2 |
| Total | 100.0 | 425.2 |

Bioadhesive Layer

| | | |
|---|---|---|
| SPHEROMER ™ III | 98.0 | 245.0 |
| Ethylcellulose (ETHOCEL ™ Std 100 FP), NF | 1.0 | 2.5 |
| Magnesium Stearate, NF | 1.0 | 2.5 |
| Total | 100.0 | 250.0 |

The ingredients of the CR and bioadhesive layers excluding magnesium stearate were blended on the ATR rotator end-over-end for 5 min. Magnesium stearate was added to the ingredients blend of each layer and the materials were blended for an additional 5 min. A 0.3287″×0.8937″ capsule-shaped die and punch set was installed on GlobePharma Manual Tablet Compaction Machine MTCM-I. The CR and two bioadhesive layers were pre-compressed together at a pressure of 200 psi (pound per square inch) for 5 seconds and then pressed at 3000 psi for 1 sec.

### Example 27

### In Vitro Dissolution and In Vivo Pharmacokinetic Performance of Bioadhesive Levodopa-Carbidopa 200 mg/50 mg Trilayer Tablets, Lot # 505-065

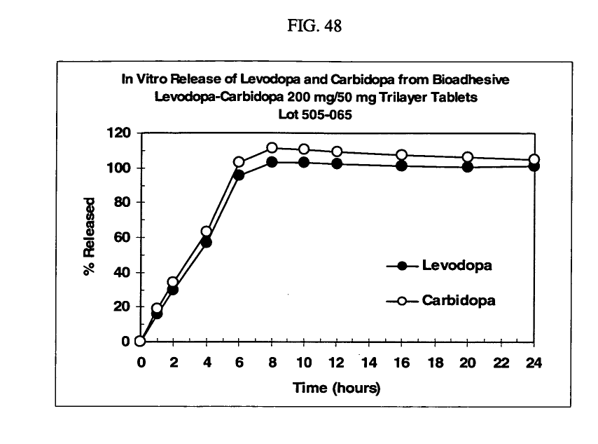

The in vitro dissolution profile of bioadhesive levodopa-carbidopa trilayer tablets, containing 50 mg carbidopa and 200 mg levodopa was obtained under simulated gastric conditions. The dissolution tests were performed in 900 mL of 0.1 NHCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 48.

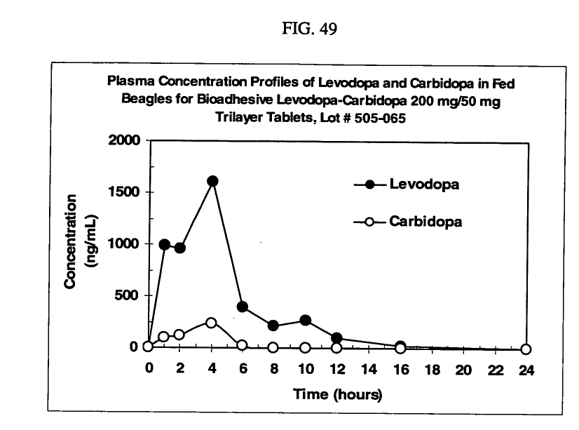

The in vivo performance of bioadhesive levodopa-carbidopa trilayer tablets was evaluated in beagle dogs. The tablets were administered to separate cohorts of six beagle dogs in the fed state. Plasma levels of levodopa and carbidopa were measured using LC/MS/MS analysis. FIG. 49 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 5.

TABLE 5
Pharmacokinetic Data for Bioadhesive Levodopa-Carbidopa Trilayer
Tablets, Lot # 505-065, in Fed Beagle Dogs; the area under
the plasma levodopa vs. time curve (AUC), maximum
concentration (Cₘₐₓ), and time required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 7,782 | 1,918 | 4.2 |

### Example 28

### Bioadhesive Levodopa-Carbidopa 200 mg/50 mg Trilayer Tablets, Lot # 505-066

Bioadhesive levodopa-carbidopa trilayer tablets were produced with direct compression in accordance with the method described in Example 8. Tablets comprised an active controlled release (CR) layer laminated between two passive bioadhesive layers. The weight and composition of the CR and bioadhesive layers are given in Table 6.

TABLE 6
Weight and Composition of Controlled Release and Bioadhesive
Layers of Levodopa-Carbidopa 200 mg/50 mg Trilayer
Tablet, Lot # 505-066

| | | |
|---|---|---|
| Ingredients | Weight % | Weight (mg) |

Controlled Release Layer

| | | |
|---|---|---|
| Levodopa, USP | 42.9 | 200.0 |
| Carbidopa monohydrate, USP | 11.6 | 54.0 |
| Hypromellose 2208, 100 cps, USP | 35.8 | 167.2 |
| Hypromellose 2910, 5 cps, USP | 4.5 | 20.9 |
| L-Glutamic acid, FCC | 2.2 | 10.4 |
| Corn Starch, NF | 2.2 | 10.4 |
| Magnesium Stearate, NF | 0.8 | 3.8 |
| Total | 100.0 | 466.7 |

Bioadhesive Layer

| | | |
|---|---|---|
| SPHEROMER ™ III | 98.0 | 245.0 |
| Ethylcellulose (ETHOCEL ™ Std 100 FP), NF | 1.0 | 2.5 |
| Magnesium Stearate, NF | 1.0 | 2.5 |
| Total | 100.0 | 250.0 |

The ingredients of the CR and bioadhesive layers excluding magnesium stearate were blended on the ATR rotator end-over-end for 5 min. Magnesium stearate was added to the ingredients blend of each layer and the materials were blended for an additional 5 min. A 0.3287″×0.8937″ capsule-shaped die and punch set was installed on GlobePharma Manual Tablet Compaction Machine MTCM-I. The CR and two bioadhesive layers were pre-compressed together at a pressure of 200 psi (pound per square inch) for 5 seconds and then pressed at 3000 psi for 1 sec.

### Example 29

### In Vitro Dissolution and In Vivo Pharmacokinetic Performance of Bioadhesive Levodopa-Carbidopa 200 mg/50 mg Trilayer Tablets, Lot # 505-066

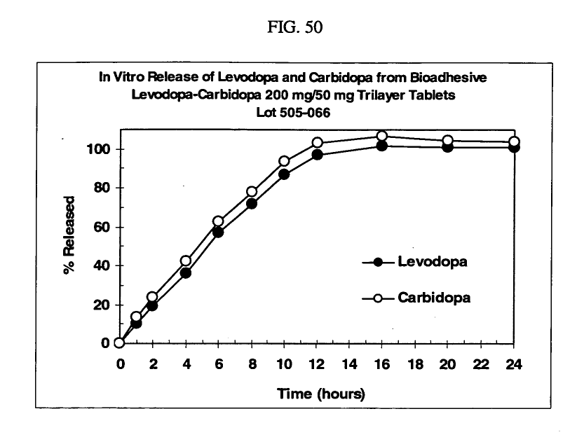

The in vitro dissolution profile of bioadhesive levodopa-carbidopa trilayer tablets, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 50.

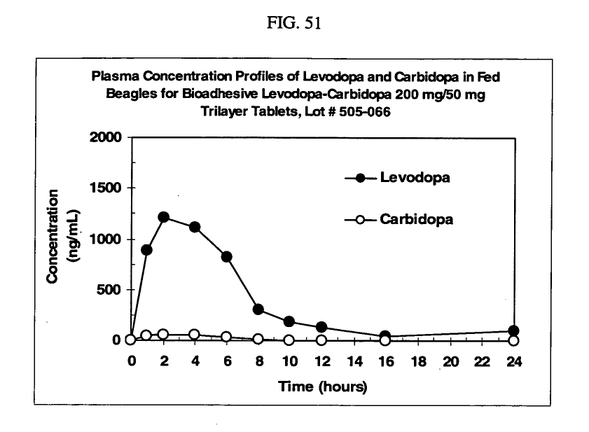

The in vivo performance of bioadhesive levodopa-carbidopa trilayer tablets was evaluated in beagle dogs. The tablets were administered to separate cohorts of six beagle dogs in the fed state. Plasma levels of levodopa and carbidopa were measured using LC/MS/MS analysis. FIG. 51 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 7.

TABLE 7
Pharmacokinetic Data for Bioadhesive Levodopa-Carbidopa Trilayer
Tablets, Lot # 505-066, in Fed Beagle Dogs; the area under the
plasma levodopa vs. time curve (AUC), maximum concentration
(Cₘₐₓ), and time required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 8,537 | 1,584 | 3.5 |

### Example 39

### Bioadhesive Levodopa-Carbidopa 200 mg/50 mg Trilayer Tablets, Lot #506-063

Bioadhesive levodopa-carbidopa trilayer tablets were produced with direct compression in accordance with the method described in Example 8. Tablets comprised an active controlled release (CR) layer laminated between a passive bioadhesive layer and an active immediate release (IR) layer. The weight and composition of the IR, CR and bioadhesive layers are given in Table 8.

TABLE 8
Weight and Composition of Immediate Release, Controlled
Release, and Bioadhesive Layers of Levodopa-Carbidopa
200 mg/50 mg Trilayer Tablet, Lot # 506-063

| | | |
|---|---|---|
| Ingredients | Weight % | Weight (mg) |

Immediate Release Layer

| | | |
|---|---|---|
| Levodopa, USP | 26.67 | 40.00 |
| Carbidopa monohydrate, USP | 7.20 | 10.80 |
| LUDIPRESS ® | 65.63 | 98.45 |
| Magnesium Stearate, NF | 0.50 | 0.75 |
| Total | 100.00 | 150.00 |

Controlled Release Layer

| | | |
|---|---|---|
| Levodopa, USP | 40.00 | 160.00 |
| Carbidopa monohydrate, USP | 10.79 | 43.16 |
| Hypromellose 2208, 100 cps, USP | 38.00 | 152.00 |
| Hypromellose 2910, 5 cps, USP | 4.71 | 18.84 |
| L-Glutamic acid, FCC | 3.00 | 12.00 |
| Corn Starch, NF | 3.00 | 12.00 |
| Magnesium Stearate, NF | 0.50 | 2.00 |
| Total | 100.00 | 400.00 |

Bioadhesive Layer

| | | |
|---|---|---|
| SPHEROMER ™ III | 98.0 | 147.00 |
| Ethylcellulose (ETHOCEL ™ Std 100 FP), NF | 1.50 | 2.25 |
| Magnesium Stearate, NF | 0.50 | 0.75 |
| Total | 100.00 | 150.00 |

The ingredients of the IR, CR and bioadhesive layers excluding magnesium stearate were blended on the ATR rotator end-over-end for 5 min. Magnesium stearate was added to the ingredients blend of each layer and the materials were blended for an additional 5 min. A 0.4375″ standard convex-shaped die and punch set was installed on GlobePharma Manual Tablet Compaction Machine MTCM-I. The trilayer tablet was prepared by compression at 2000 psi for 1 second.

### Example 31

### In Vitro Dissolution and In Vivo Pharmacokinetic Performance of Bioadhesive Levodopa-Carbidopa 200 mg/50 mg Trilayer Tablets, Lot # 506-063

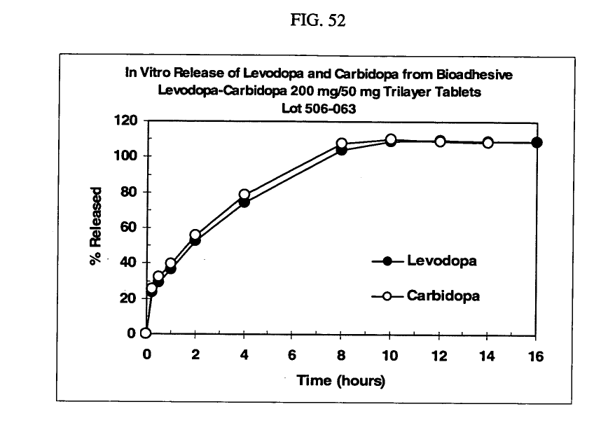

The in vitro dissolution profile of bioadhesive levodopa-carbidopa trilayer tablets, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 52.

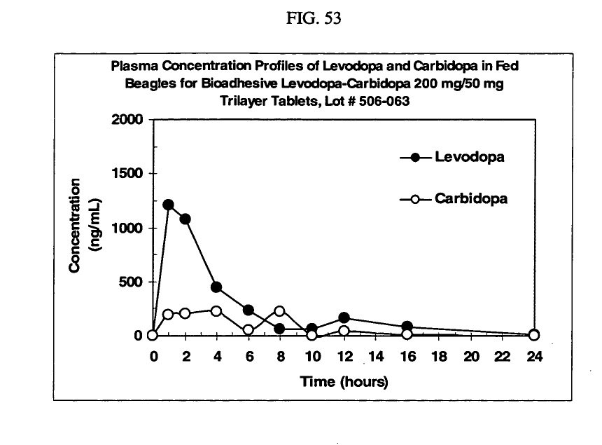

The in vivo performance of bioadhesive levodopa-carbidopa trilayer tablets was evaluated in beagle dogs. The tablets were administered to separate cohorts of six beagle dogs in the fed state. Plasma levels of levodopa and carbidopa were measured using LC/MS/MS analysis. FIG. 53 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 9.

TABLE 9
Pharmacokinetic Data for Bioadhesive Levodopa-Carbidopa Trilayer
Tablets, Lot # 506-063, in Fed Beagle Dogs; the area under the
plasma levodopa vs. time curve (AUC), maximum concentration
(Cₘₐₓ), and time required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 5,445 | 1,473 | 5.7 |

### Example 32

### Bioadhesive Levodopa-Carbidopa 200 Mg/50 Mg Trilayer Tablets with Pre-Compressed Insert, Lot # 506-024

Bioadhesive levodopa-carbidopa trilayer tablets with pre-compressed insert were produced with direct compression in accordance with the method described in Example 10. Tablets comprised a rapidly disintegrating pre-compressed insert embedded in an active controlled release (CR) layer laminated between a passive bioadhesive layer and an active immediate release (IR) layer. The weight and composition of the pre-compressed insert, and CR, IR and bioadhesive layers are given in Table 10.

TABLE 10
Weight and Composition of Pre-compressed Insert, and Controlled
Release, Immediate Release and Bioadhesive Layers of Levodopa-
Carbidopa 200 mg/50 mg Trilayer Tablet, Lot #
506-024

| | | |
|---|---|---|
| Ingredients | Weight % | Weight (mg) |

Pre-compressed Insert

| | | |
|---|---|---|
| Levodopa, USP | 3.53 | 40.00 |
| Carbidopa monohydrate, USP | 6.35 | 10.80 |
| LUDIPRESS ® | 69.68 | 118.45 |
| Magnesium Stearate, NF | 0.44 | 0.75 |
| Total | 100.00 | 170.00 |

Controlled Release Layer

| | | |
|---|---|---|
| Levodopa, USP | 42.86 | 120.00 |
| Carbidopa monohydrate, USP | 11.57 | 32.40 |
| Hypromellose 2208, 100 cps, USP | 38.04 | 106.50 |
| Hypromellose 2910, 5 cps, USP | 2.29 | 6.40 |
| L-Glutamic acid, FCC | 2.21 | 6.20 |
| Corn Starch, NF | 2.21 | 6.20 |
| Magnesium Stearate, NF | 0.82 | 2.30 |
| Total | 100.00 | 280.00 |

Immediate Release Layer

| | | |
|---|---|---|
| Levodopa, USP | 26.67 | 40.00 |
| Carbidopa monohydrate, USP | 7.20 | 10.80 |
| LUDIPRESS ® | 65.63 | 98.45 |
| Magnesium Stearate, NF | 0.50 | 0.75 |
| Total | 100.00 | 150.00 |

Bioadhesive Layer

| | | |
|---|---|---|
| SPHEROMER ™ III | 98.0 | 147.00 |
| Ethylcellulose (ETHOCEL ™ Std 100 FP), NF | 1.50 | 2.25 |
| Magnesium Stearate, NF | 0.50 | 0.75 |
| Total | 100.00 | 150.00 |

The ingredients of the insert, and CR, IR and bioadhesive layers excluding magnesium stearate were blended on the ATR rotator end-over-end for 5 min. Magnesium stearate was added to the ingredients blend of each layer and the materials were blended for an additional 5 min. A 0.2618″ standard convex-shaped die and punch set was installed on GlobePharma Manual Tablet Compaction Machine MTCM-I. The ingredients blend of the insert was compressed into a tablet at 500 psi (pound per square inch) for 1 second. A 0.4375″ standard convex-shaped die and punch set was installed on the same tablet compactor. The trilayer tablet was prepared by compression at 3000 psi for 1 second.

### Example 33

### In Vitro Dissolution and In Vivo Pharmacokinetic Performance of Bioadhesive Levodopa-Carbidopa 200 mg/50 mg Trilayer Tablets with Pre-Compressed Insert, Lot # 506-024

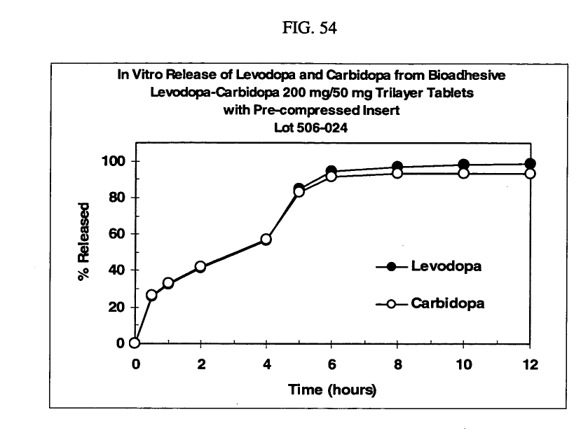

The in vitro dissolution profile of bioadhesive levodopa-carbidopa trilayer tablets with pre-compressed insert, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 54.

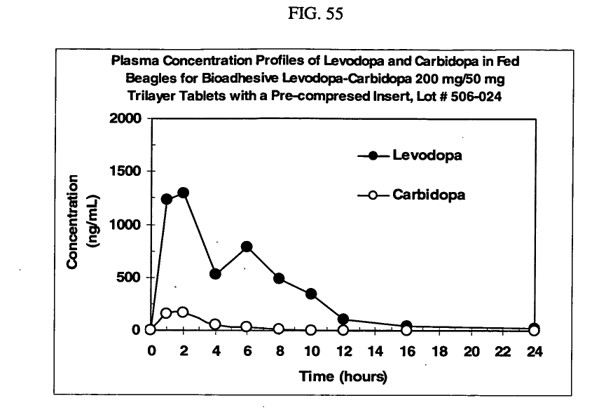

The in vivo performance of bioadhesive levodopa-carbidopa trilayer tablets with pre-compressed insert was evaluated in beagle dogs. The tablets were administered to separate cohorts of six beagle dogs in the fed state. Plasma levels of levodopa and carbidopa were measured using LC/MS/MS analysis. FIG. 55 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 11.

TABLE 11
Pharmacokinetic Data for Bioadhesive Levodopa-Carbidopa
Trilayer Tablets with Pre-compressed Insert, Lot # 506-024,
in Fed Beagle Dogs; the area under the plasma levodopa vs. time
curve (AUC), maximum concentration (Cₘₐₓ), and time required
to achieve C_{max (T}ₘₐₓ)

| | AUC | Cₘₐₓ | Tₘₐₓ |
|---|---|---|---|
| Fasting Period | (ng/ml · hr) | (ng/ml) | (hr) |
| Fed | 8,104 | 1,782 | 2.3 |

### Example 34

### Bioadhesive Levodopa-Carbidopa 200 mg/50 mg Trilayer Tablets with Pre-Compressed Insert, Lot # 507-027

Bioadhesive levodopa-carbidopa trilayer tablets with pre-compressed insert were produced with direct compression in accordance with the method described in Example 10. Tablets comprised a rapidly disintegrating pre-compressed insert embedded in an active controlled release (CR) layer laminated between a passive bioadhesive layer and an active immediate release (IR) layer. The weight and composition of the pre-compressed insert, and CR, IR and bioadhesive layers are given in Table 12.

TABLE 12
Weight and Composition of Pre-compressed Insert, and
Controlled Release, Immediate Release and Bioadhesive
Layers of Levodopa-Carbidopa 200 mg/50 mg Trilayer
Tablet, Lot # 507-027

| | | |
|---|---|---|
| Ingredients | Weight % | Weight (mg) |

Pre-compressed Insert

| | | |
|---|---|---|
| Levodopa, USP | 40.00 | 40.00 |
| Carbidopa monohydrate, USP | 10.80 | 10.80 |
| LUDIPRESS ® LCE | 48.60 | 48.60 |
| Magnesium Stearate, NF | 0.50 | 0.50 |
| Total | 100.00 | 100.00 |

Controlled Release Layer

| | | |
|---|---|---|
| Levodopa, USP | 34.29 | 120.00 |
| Carbidopa monohydrate, USP | 9.26 | 32.40 |
| Hypromellose 2208, 100 cps, USP | 42.14 | 147.50 |
| Hypromellose 2208, 4000 cps, USP | 7.71 | 27.00 |
| L-Glutamic acid, FCC | 3.00 | 10.50 |
| Corn Starch, NF | 3.00 | 10.50 |
| Magnesium Stearate, NF | 0.60 | 2.10 |
| Total | 100.00 | 350.00 |

Immediate Release Layer

| | | |
|---|---|---|
| Levodopa, USP | 26.67 | 40.00 |
| Carbidopa monohydrate, USP | 7.20 | 10.80 |
| LUDIPRESS ® | 65.63 | 98.45 |
| Magnesium Stearate, NF | 0.50 | 0.75 |
| Total | 100.00 | 150.00 |

Bioadhesive Layer

| | | |
|---|---|---|
| SPHEROMER ™ III | 98.00 | 147.00 |
| Ethylcellulose (ETHOCEL ™ Std 100 FP), NF | 1.50 | 2.25 |
| Magnesium Stearate, NF | 0.50 | 0.75 |
| Total | 100.00 | 150.00 |

The ingredients of the insert, and CR, IR and bioadhesive layers excluding magnesium stearate were blended on the ATR rotator end-over-end for 5 min. Magnesium stearate was added to the ingredients blend of each layer and the materials were blended for an additional 5 min. A 0.2618″ standard convex-shaped die and punch set was installed on GlobePharma Manual Tablet Compaction Machine MTCM-I. The ingredients blend of the insert was compressed into a tablet at 500 psi (pound per square inch) for 1 second. A 0.4375″ standard convex-shaped die and punch set was installed on the same tablet compactor. The trilayer tablet was prepared by compression at 3500 psi for 1 second.

### Example 35

### In Vitro Dissolution and In Vivo Pharmacokinetic Performance of Bioadhesive Levodopa-Carbidopa 200 mg/50 mg Trilayer Tablets with Pre-Compressed Insert, Lot # 507-027

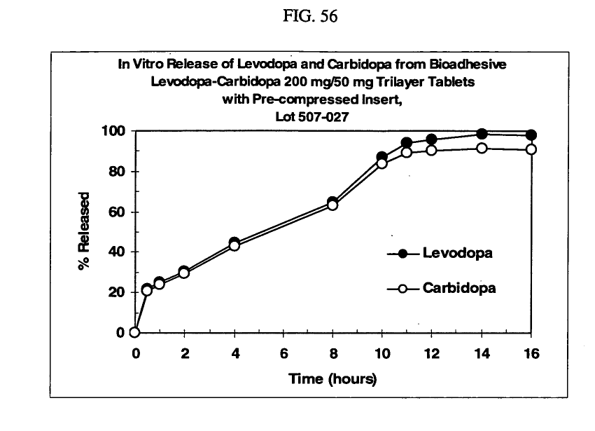

The in vitro dissolution profile of bioadhesive levodopa-carbidopa trilayer tablets with pre-compressed insert, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 56.

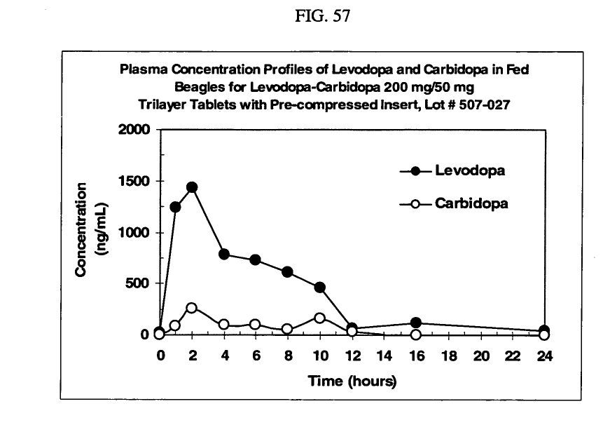

The in vivo performance of bioadhesive levodopa-carbidopa trilayer tablets with pre-compressed insert was evaluated in beagle dogs. The tablets were administered to separate cohorts of six beagle dogs in the fed state. Plasma levels of levodopa and carbidopa were measured using LC/MS/MS analysis. FIG. 57 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 13.

TABLE 13
Pharmacokinetic Data for Bioadhesive Levodopa-Carbidopa
Trilayer Tablets with Pre-compressed Insert, Lot # 507-027,
in Fed Beagle Dogs; the area under the plasma levodopa vs.
time curve (AUC), maximum concentration (Cₘₐₓ), and
time required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 9,597 | 1,742 | 4.2 |

### Example 36

### Bioadhesive Levodopa-Carbidopa 200 mg/50 mg Triple Pressed Tablets, Lot # 507-047

Bioadhesive levodopa-carbidopa triple pressed tablets were produced with direct compression in accordance with the method described in Example 12. Tablets comprised a pre-compressed active inner core, press-coated with an erodible bioadhesive controlled-release (CR) layer overlaid by an immediate-release (IR) layer. The weight and composition of the IR and CR layers, and the inner core are given in Table 14.

TABLE 14
Weight and Composition of Immediate Release and
Bioadhesive Controlled Release Layers, and the
Inner Core of Levodopa-Carbidopa 200 mg/50 mg
Triple Pressed Tablet, Lot # 507-047

| | | |
|---|---|---|
| Ingredients | Weight % | Weight (mg) |

Immediate Release Layer (Outer Layer)

| | | |
|---|---|---|
| Levodopa, USP | 16.00 | 40.00 |
| Carbidopa monohydrate, USP | 4.32 | 10.80 |
| LUDIPRESS ® | 79.18 | 247.95 |
| Magnesium Stearate, NF | 0.50 | 1.25 |
| Total | 100.00 | 300.00 |

Controlled Release Layer (Middle Layer)

| | | |
|---|---|---|
| Levodopa, USP | 40.00 | 120.00 |
| Carbidopa monohydrate, USP | 10.80 | 32.39 |
| Polyethylene Oxide (Polyox ™ WSR-301), NF | 47.70 | 143.11 |
| L-Glutamic acid, FCC | 1.00 | 3.00 |
| Magnesium Stearate, NF | 0.50 | 1.50 |
| Total | 100.00 | 300.00 |

Fast Disintegrating Core (Inner Core)

| | | |
|---|---|---|
| Levodopa, USP | 40.00 | 40.00 |
| Carbidopa monohydrate, USP | 10.80 | 10.80 |
| LUDIPRESS ® LCE | 48.70 | 48.70 |
| Magnesium Stearate, NF | 0.50 | 0.50 |
| Total | 100.00 | 100.00 |

The ingredients of the IR and CR layers and the inner core excluding magnesium stearate were blended on the ATR rotator end-over-end for 5 min. Magnesium stearate was added to the ingredients blend of each layer and the materials were blended for an additional 5 min. A 0.2400″ standard convex-shaped die and punch set was installed on GlobePharma Manual Tablet Compaction Machine MTCM-I. The ingredients blend of the inner core was compressed into a tablet at 250 psi (pound per square inch) for 1 second. A 0.3228″ standard convex-shaped die and punch set was installed on the same tablet compactor. The inner core was press-coated with the CR layer blend by compression at 500 psi for 1 second. A 0.4375″ standard convex-shaped die and punch set was installed on the same tablet compactor. The double pressed tablet prepared above, was press-coated with the IR layer blend by compression at 4000 psi for 1 second.

### Example 37

### In Vitro Dissolution and In Vivo Pharmacokinetic Performance of Bioadhesive Levodopa-Carbidopa 200 mg/50 mg Triple Pressed Tablets, Lot # 507-047

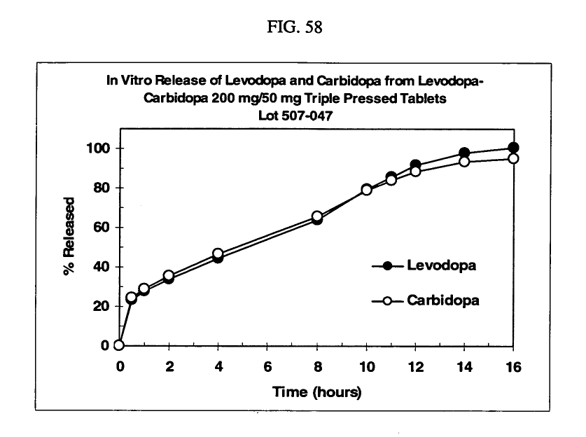

The in vitro dissolution profile of levodopa-carbidopa triple pressed tablets, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 58.

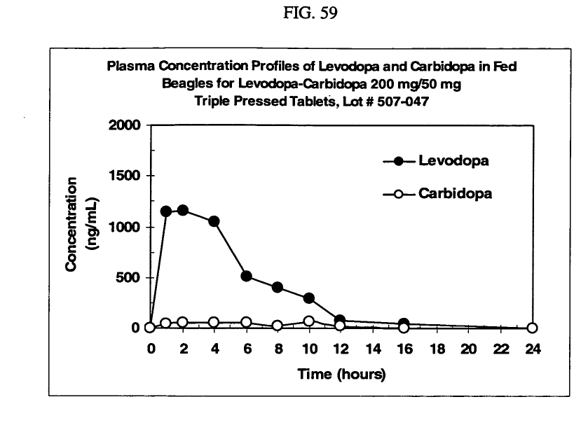

The in vivo performance of levodopa-carbidopa triple pressed tablets was evaluated in beagle dogs. The tablets were administered to separate cohorts of six beagle dogs in the fed state. Plasma levels of levodopa and carbidopa were measured using LC/MS/MS analysis. FIG. 59 shows the plasma concentration profiles of levodopa and carbidopa in the fed state. The pharmacokinetic data including the area under the plasma levodopa vs. time curve (AUC), maximum concentration (Cₘₐₓ) and time required to achieve Cₘₐₓ (Tₘₐₓ) are provided in Table 15.

TABLE 15
Pharmacokinetic Data for Levodopa-Carbidopa Triple Pressed
Tablets, Lot # 507-047, in Fed Beagle Dogs; the area under
the plasma levodopa vs. time curve (AUC), maximum
concentration (Cₘₐₓ), and time required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 7,826 | 1,574 | 2.4 |

### Example 38

### Bioadhesive Levodopa-Carbidopa 200 mg/50 mg Quadrilayer Tablets, Lot # 603-242

Bioadhesive levodopa-carbidopa quadrilayer tablets were produced with wet granulation and compression in accordance with the method described in Example 9. Tablets comprised an active controlled release (CR) layer laminated between two passive bioadhesive layers, and an immediate release (IR) layer overlying one of the bioadhesive layers. The weight and composition of the IR, CR and bioadhesive layers are given in Table 16.

TABLE 16
Weight and Composition of Immediate Release, Controlled
Release, and Bioadhesive Layers of Levodopa-Carbidopa
200 mg/50 mg Quadrilayer Tablet, Lot # 603-242

| | | |
|---|---|---|
| | | Weight |
| Ingredients | Weight % | (mg) |

Immediate Release Layer

| | | |
|---|---|---|
| Levodopa, USP | 33.3 | 40.0 |
| Carbidopa monohydrate, USP | 9.0 | 10.8 |
| LUDIPRESS ® | 49.3 | 59.2 |
| citric acid, anhydrous, USP | 8.0 | 9.6 |
| Magnesium Stearate, NF | 0.3 | 0.3 |
| Butylated hydroxytoluene, NF | 0.1 | 0.1 |
| Total | 100.0 | 120.0 |

Controlled Release Layer

| | | |
|---|---|---|
| Levodopa, USP | 43.5 | 159.6 |
| Carbidopa monohydrate, USP | 11.8 | 43.3 |
| Succinic Acid, FCC | 17.7 | 65.0 |
| Hypromellose 2910, 5 cps, USP | 14.3 | 52.5 |
| Hypromellose 2208, 100 cps, USP | 9.6 | 35.2 |
| Corn Starch, NF | 2.6 | 9.5 |
| Magnesium Stearate, NF | 0.4 | 1.5 |
| Butylated hydroxytoluene, NF | 0.1 | 0.4 |
| Total | 100.0 | 367.0 |

Bioadhesive Layer

| | | |
|---|---|---|
| SPHEROMER ™ III | 62.2 | 155.6 |
| SPHEROMER ™ I [p (FASA)] | 21.6 | 54.0 |
| Hydroxypropyl cellulose (KLUCEL ® EF Pharm), | 13.0 | 32.4 |
| citric acid, anhydrous, USP | 3.0 | 7.5 |
| Magnesium Stearate, NF | 0.2 | 0.5 |
| Total | 100.0 | 250.0 |

The ingredients of the IR layer excluding magnesium stearate were blended in a GlobePharma Maxiblend V-shell blender equipped with a 0.5-qt V-shell, for 10 min. Magnesium stearate was added to the mixed ingredients and the materials were blended for an additional 5 min.

The CR layer was prepared with the granulation method described in Example 4. The ingredients of the CR layer excluding Hypromellose 2910 and magnesium stearate were blended in a Hobart Mixer for 5 min. The dry blend was granulated by using a 5% (w/v) solution of Hypromellose 2910 in methyl alcohol. The wet granulation was dried in a Vector MFL.01 Micro Batch Fluid Bed System, operating at an inlet air flow rate of 200 lpm (liters per minute) and an inlet air temperature of 50° C. The dried granulation was passed through a U.S. Std. mesh # 60 sieve. The screened granulation was mixed with magnesium stearate in the GlobePharma Maxiblend V-shell blender equipped with a 0.5-qt V-shell for 5 min.

SPHEROMER™ III was granulated along with hydroxypropyl cellulose (HPC) and citric acid by using a 3% (w/v) solution of HPC in methylene chloride in a Hobart Mixer in accordance with the method described in Example 5. The wet granulation was dried in the Vector MFL.01 Micro Batch Fluid Bed System, operating at an inlet air flow rate of 290 lpm (liters per minute) and an inlet air temperature of 55° C. The dried granulation was passed through a U.S. Std. mesh # 40 sieve.

SPHEROMER™ I was granulated along with hydroxypropyl cellulose (HPC) by using a 2% (w/v) solution of HPC in dehydrated alcohol in a Hobart Mixer in accordance with the method described in Example 5. The wet granulation was dried in the Vector MFL.01 Micro Batch Fluid Bed System, operating at an inlet air flow rate of 200 lpm (liters per minute) and an inlet air temperature of 60° C. The dried granulation was passed through a U.S. Std. mesh # 40 sieve.

The screened SPHEROMER™ III and SPHEROMER™ I granulations were blended in a GlobePharma Maxiblend V-shell blended equipped with a 2-qt V-shell, for 10 min. Magnesium stearate was added to the blend and the materials were mixed for an additional 5 min. The final blend was passed through a U.S. Std. mesh # 40 sieve.

A 0.3286″×0.8937″ standard capsule-shaped die and punch set was installed on GlobePharma Manual Tablet Compaction Machine MTCM-I. The quadrilayer tablet was prepared by pre-compression of the four layers at 500 psi (pound per square inch) for 2 seconds and final compression at 4000 psi for 2 s.

### Example 39

### In Vitro Dissolution and In Vivo Pharmacokinetic Performance of Bioadhesive Levodopa-Carbidopa 200 mg/50 mg Quadrilayer Tablets, Lot # 603-242

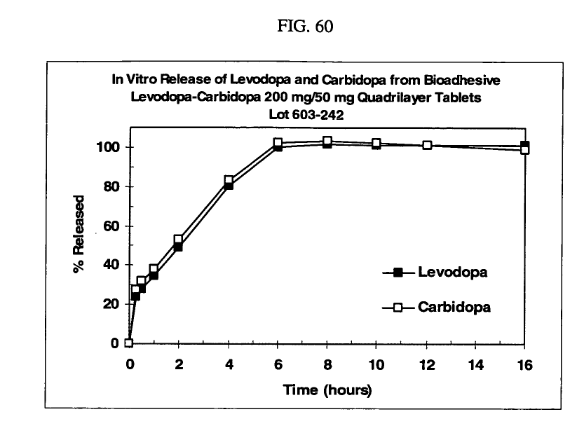

The in vitro dissolution profile of bioadhesive levodopa-carbidopa quadrilayer tablets, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 60.

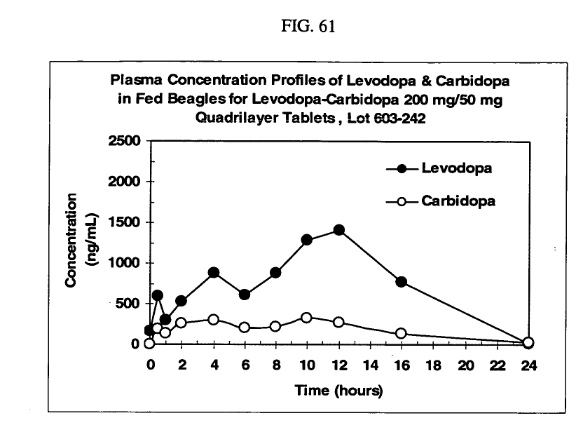

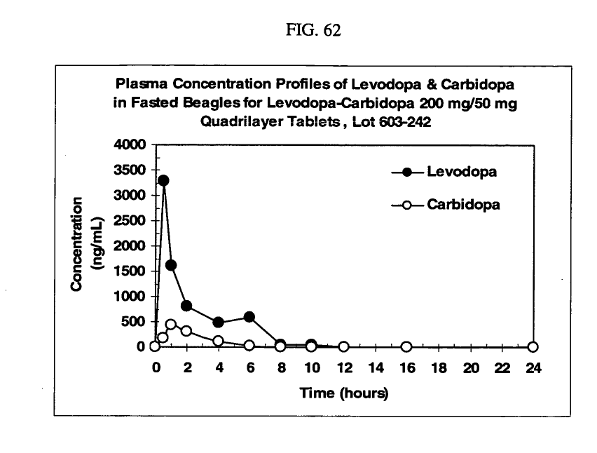

The in vivo performance of bioadhesive levodopa-carbidopa quadrilayer tablets was evaluated in beagle dogs. The tablets were administered to separate cohorts of six beagle dogs in the fed and fasted states. Plasma levels of levodopa and carbidopa were measured using LC/MS/MS analysis. FIGS. 61 and 62 show the plasma concentration profiles of levodopa and carbidopa in the fed and fasted states, respectively. The pharmacokinetic data including the area under the plasma levodopa vs. time curve (AUC), maximum concentration (Cₘₐₓ) and time required to achieve Cₘₐₓ (Tₘₐₓ) are provided in Table 17.

TABLE 17
Pharmacokinetic Data for Bioadhesive Levodopa-Carbidopa
Quadrilayer Tablets, Lot # 603-242, in Fed and Fasted
Beagle Dogs; the area under the plasma levodopa vs. time curve
(AUC), maximum concentration (Cₘₐₓ), and time
required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 16,558 | 1,798 | 10.3 |
| Fasted | 6,375 | 3,277 | 0.5 |

### Example 40

### Bioadhesive Levodopa-Carbidopa 200 mg/50 mg Quadrilayer Tablets, Lot # 603-243

Bioadhesive levodopa-carbidopa quadrilayer tablets were produced with wet granulation and compression in accordance with the method described in Example 9. Tablets comprised an active controlled release (CR) layer laminated between two passive bioadhesive layers, and an immediate release (IR) layer overlying one of the bioadhesive layers. The weight and composition of the IR, CR and bioadhesive layers are given in Table 18.

TABLE 18
Weight and Composition of Immediate Release, Controlled
Release, and Bioadhesive Layers of Levodopa-Carbidopa
200 mg/50 mg Quadrilayer Tablet, Lot # 603-243

| | | |
|---|---|---|
| | | Weight |
| Ingredients | Weight % | (mg) |

Immediate Release Layer

| | | |
|---|---|---|
| Levodopa, USP | 33.3 | 40.0 |
| Carbidopa monohydrate, USP | 9.0 | 10.8 |
| LUDIPRESS ® | 49.3 | 59.2 |
| citric acid, anhydrous, USP | 8.0 | 9.6 |
| Magnesium Stearate, NF | 0.3 | 0.3 |
| Butylated hydroxytoluene, NF | 0.1 | 0.1 |
| Total | 100.0 | 120.0 |

Controlled Release Layer

| | | |
|---|---|---|
| Levodopa, USP | 43.5 | 160.0 |
| Carbidopa monohydrate, USP | 11.7 | 42.9 |
| Hypromellose 2208, 100 cps, USP | 19.1 | 70.1 |
| Succinic Acid, FCC | 17.7 | 65.0 |
| Hypromellose 2910, 5 cps, USP | 4.8 | 17.6 |
| Corn Starch, NF | 1.7 | 6.2 |
| Hypromellose 2208, 4000 cps, USP | 1.0 | 3.7 |
| Magnesium Stearate, NF | 0.4 | 1.5 |
| Butylated hydroxytoluene, NF | 0.1 | 0.4 |
| Total | 100.0 | 367.0 |

Bioadhesive Layer

| | | |
|---|---|---|
| SPHEROMER ™ III | 62.2 | 155.6 |
| SPHEROMER ™ I [p (FASA)] | 21.6 | 54.0 |
| Hydroxypropyl cellulose (KLUCEL ® EF Pharm), | 13.0 | 32.4 |
| citric acid, anhydrous, USP | 3.0 | 7.5 |
| Magnesium Stearate, NF | 0.2 | 0.5 |
| Total | 100.0 | 250.0 |

The ingredients of the IR layer excluding magnesium stearate were blended in a GlobePharma Maxiblend V-shell blender equipped with a 0.5-qt V-shell, for 10 min. Magnesium stearate was added to the mixed ingredients and the materials were blended for an additional 5 min.

The CR layer was prepared with the granulation method described in Example 4. The ingredients of the CR layer excluding Hypromellose 2910 and magnesium stearate were blended in a Hobart Mixer for 5 min. The dry blend was granulated by using a 5% (w/v) solution of Hypromellose 2910 in methyl alcohol. The wet granulation was dried in a Vector MFL.01 Micro Batch Fluid Bed System, operating at an inlet air flow rate of 200 lpm (liters per minute) and an inlet air temperature of 50° C. The dried granulation was passed through a U.S. Std. mesh # 60 sieve. The screened granulation was mixed with magnesium stearate in the GlobePharma Maxiblend V-shell blender equipped with a 0.5-qt V-shell for 5 min.

SPHEROMER™ III was granulated along with hydroxypropyl cellulose (HPC) and citric acid by using a 3% (w/v) solution of HPC in methylene chloride in a Hobart Mixer in accordance with the method described in Example 5. The wet granulation was dried in the Vector MFL.01 Micro Batch Fluid Bed System, operating at an inlet air flow rate of 290 lpm (liters per minute) and an inlet air temperature of 55° C. The dried granulation was passed through a U.S. Std. mesh # 40 sieve.

SPHEROMER™ I was granulated along with hydroxypropyl cellulose (HPC) by using a 2% (w/v) solution of HPC in dehydrated alcohol in a Hobart Mixer in accordance with the method described in Example 5. The wet granulation was dried in the Vector MFL.01 Micro Batch Fluid Bed System, operating at an inlet air flow rate of 200 lpm (liters per minute) and an inlet air temperature of 60° C. The dried granulation was passed through a U.S. Std. mesh # 40 sieve.

The screened SPHEROMER™ III and SPHEROMER™ I granulations were blended in a GlobePharma Maxiblend V-shell blended equipped with a 2-qt V-shell, for 10 min. Magnesium stearate was added to the blend and the materials were mixed for an additional 5 min. The final blend was passed through a U.S. Std. mesh # 40 sieve.

A 0.3286″×0.8937″ standard capsule-shaped die and punch set was installed on GlobePharma Manual Tablet Compaction Machine MTCM-I. The quadrilayer tablet was prepared by pre-compression of the four layers at 500 psi (pound per square inch) for 2 seconds and final compression at 4000 psi for 2 s.

### Example 41

### In Vitro Dissolution and In Vivo Pharmacokinetic Performance of Bioadhesive Levodopa-Carbidopa 200 mg/50 mg Quadrilayer Tablets, Lot # 603-243

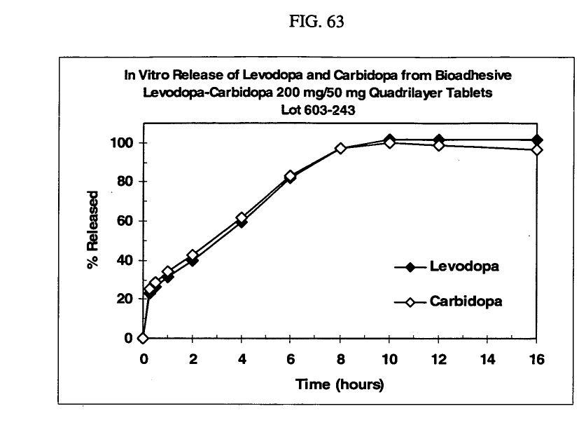

The in vitro dissolution profile of bioadhesive levodopa-carbidopa quadrilayer tablets, containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 63.

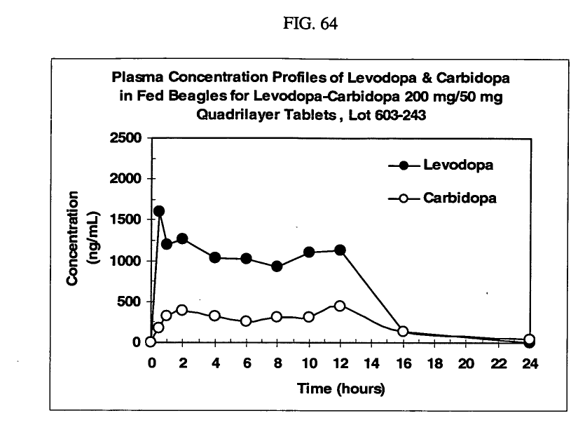

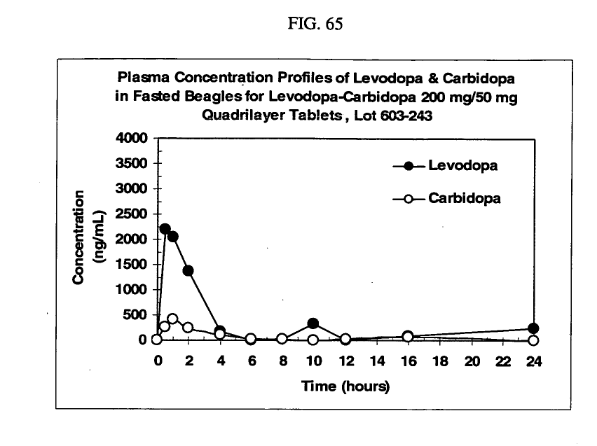

The in vivo performance of bioadhesive levodopa-carbidopa quadrilayer tablets was evaluated in beagle dogs. The tablets were administered to separate cohorts of six beagle dogs in the fed and fasted states. Plasma levels of levodopa and carbidopa were measured using LC/MS/MS analysis. FIGS. 64 and 65 show the plasma concentration profiles of levodopa and carbidopa in the fed and fasted states, respectively. The pharmacokinetic data including the area under the plasma levodopa vs. time curve (AUC), maximum concentration (Cₘₐₓ) and time required to achieve Cₘₐₓ (Tₘₐₓ) are provided in Table 19.

TABLE 19
Pharmacokinetic Data for Bioadhesive Levodopa-Carbidopa
Quadrilayer Tablets, Lot # 603-243, in Fed and Fasted
Beagle Dogs; the area under the plasma levodopa vs. time curve
(AUC), maximum concentration (Cₘₐₓ), and time
required to achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 15,927 | 2,326 | 4.5 |
| Fasted | 7,175 | 3,073 | 0.9 |

### Example 42

### Manufacturing of Levodopa-Carbidopa (4:1) Core Pellets with Granulation-Extrusion-Spheronization, Lot # 606-027

Levodopa-carbidopa core pellets (lot # 606-027) were prepared with granulation-extrusion-spheronization in accordance with the method described in Example 13. The dry weight and composition of pellets are given in Table 20. Micronized levodopa and carbidopa were blended with inactive excipients in a planetary mixer for 15 min. The levodopa-carbidopa-excipients blend was then granulated by spraying a 1.9% (w/v) aqueous solution of polyethylene oxide while mixing at low shear. The granulation was blended for an additional 5 min and then extruded through a 0.8 mm screen of a Caleva Extruder, model 25, operating at 10 rpm. The extrudate was spheronized in a Caleva Spheronizer, model 250, operating at 1250 rpm for 5 min. The spheronized pellets were dried in a Vector MFL.01 Micro Batch Fluid Bed System at 50° C. for 90 minutes. The dried pellets were screened using # 16 and 35 mesh screens and particles with diameters ranging from 0.5 mm to 1.2 mm were selected for future experimentation.

TABLE 20
Dry Weight and Composition of Levodopa-Carbidopa
Pellets, Lot # 606-027

| | Weight | Weight |
|---|---|---|
| Components | % | (g) |
| Levodopa, Micronized | 33.71 | 202.28 |
| Carbidopa, Monohydrate, USP | 9.20 | 55.22 |
| Fumaric Acid, NF | 33.71 | 202.28 |
| Microcrystalline Cellulose (EMCOCEL ® 90 M), NF | 15.31 | 91.83 |
| Croscarmellose Sodium (AC-DI-SOL ®), NF | 4.80 | 28.81 |
| Poloxamer 188 (LUTROL ® F68), NF | 2.70 | 16.21 |
| Polyethylene Oxide (POLYOX ™ WSR N10), NF | 0.50 | 3.00 |
| Butylated Hydroxytoluene, NF | 0.06 | 0.38 |
| Total | 100.00 | 600.00 |

### Example 43

### Manufacturing and In Vitro Dissolution of Levodopa-Carbidopa Bioadhesive Extended Release Pellets, Lot# 606-034

Two hundred fifty grams of levodopa-carbidopa core pellets of lot # 606-027 retained on mesh # 35 (from Example 42) were subsequently coated in a Vector MFL.01 Micro Batch Fluid Bed System with a release rate-controlling composition containing EUDRAGIT® RS 100, EUDRAGIT® RL 100, ACRYL-EZE® and triethyl citrate (65:15:5:15) dissolved in methanol to achieve a weight gain of 4.4% (w/w). These pellets were subsequently film-coated with a bioadhesive polymeric composition comprising SPHEROMER™ III, succinic acid and citric acid (45:50:5) dissolved in methanol to achieve a weight gain of 6.9% (w/w). Finally they were top-coated with a hypromellose and OPADRY® Clear coating mixture (45:55) dissolved in a methanol and water solution (90:10 v/v) to achieve a weight gain of 2.4% (w/w). The top coating was added to keep pellets monodispersed upon release in the stomach.

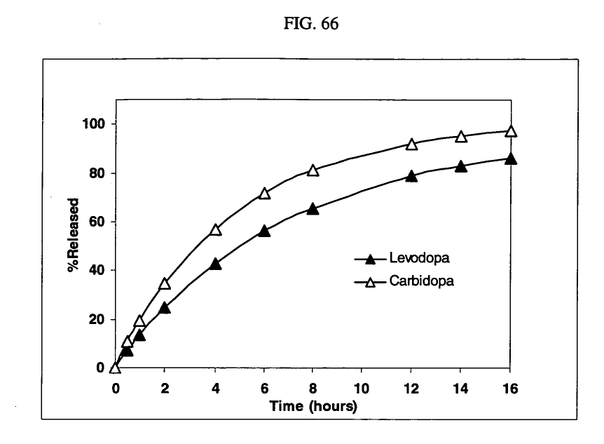

Various film coatings were performed in the Vector MFL.01 Micro Batch Fluid Bed System, equipped with a Wurster insert, operating at an inlet air flow rate of 100-300 lpm (liter per minute) and an inlet air temperature of 35° C.±5° C. The pellets were pre-warmed at 35° C. for 2-5 min and after film-coating were post-dried at 30° C. for 15-30 min. The weight and composition of coated pellets (lot # 606-034) are given in Table 21. FIG. 66 shows the in vitro dissolution profiles of levodopa and carbidopa obtained from HPLC analysis in phosphate buffered saline, pH 4.5.

TABLE 21
Weight and Composition of Levodopa-Carbidopa
Bioadhesive Extended Release Pellets, Lot# 606-034

| | | Weight |
|---|---|---|
| Components | Weight % | (g) |
| Levodopa-Carbidopa Core Pellets (Lot # 606-027) | 76.71 | 250.0 |
| Succinic Acid, FCC | 5.37 | 17.5 |
| EUDRAGIT ® RS 100, NF | 5.00 | 16.3 |
| SPHEROMER ™ III | 4.85 | 15.8 |
| OPADRY ® Clear | 2.64 | 8.6 |
| Hypromellose 2910, 5 cps (METHOCEL ™ E5 | 2.15 | 7.0 |
| Premium LV), USP | | |
| EUDRAGIT ® RL 100, NF | 1.17 | 3.8 |
| Triethyl Citrate, NF | 1.17 | 3.8 |
| Anhydrous Citric Acid, USP | 0.55 | 1.8 |
| ACRYL-EZE ® White (93O18509) | 0.40 | 1.3 |
| Total | 100.00 | 325.9 |

### Example 44

### Manufacturing of Levodopa-Carbidopa Immediate Release Layer Blend, Lot # 606-052

To manufacture a rapidly disintegrating matrix tablet of levodopa and carbidopa, a dry blend of these actives with inactive ingredients was prepared using a V-shell blender. The weight and composition of the blend are given in Table 22. All ingredients excluding magnesium stearate were blended in a GlobePharma Maxiblend V-shell blender equipped with a 2-qt V-shell, for 10 min. Magnesium stearate was added to the mixed ingredients and the materials were blended for an additional 5 min.

TABLE 22
Weight and Composition of Levodopa-Carbidopa
Immediate Release Layer Blend, Lot# 606-052

| | Weight | Weight |
|---|---|---|
| Components | % | (g) |
| Levodopa, Micronized | 4.80 | 24.00 |
| Carbidopa, Monohydrate, USP | 1.30 | 6.50 |
| LUDIPRESS ® | 50.80 | 254.00 |
| Microcrystalline Cellulose (AVICEL ® PH-105), NF | 26.00 | 130.00 |
| Croscarmellose Sodium (AC-DI-SOL ®), NF | 15.00 | 75.00 |
| Anhydrous Citric Acid, USP | 1.10 | 5.50 |
| Poloxamer 188 (LUTROL ® F68), NF | 0.59 | 2.95 |
| Magnesium Stearate, NF | 0.40 | 2.00 |
| Butylated Hydroxytoluene, NF | 0.01 | 0.05 |
| Total | 100.00 | 500.00 |

### Example 45

### Manufacturing and In Vitro Dissolution of Levodopa-Carbidopa 200 mg/50 mg Rapidly Disintegrating Pelletized Extended Release Tablets, Lot # 606-058

This formulation approach is a monolithic tablet system comprising a rapidly disintegrating levodopa-carbidopa matrix component and levodopa-carbidopa bioadhesive extended release multiparticulate pellets. The tablet disintegrates rapidly in the stomach releasing an initial dose of levodopa-carbidopa and the multiparticulate pellets into the gastric environment. This allows an initial dose of levodopa to be immediately absorbed. The multipar-ticulate pellets adhere to the gastric mucosal lining and release levodopa in a regulated manner.

Levodopa-carbidopa rapidly disintegrating pelletized tablets were prepared using direct compression. The manufacturing processes comprised:
(1) Weighing levodopa-carbidopa bioadhesive extended release pellets (lot # 606-034) prepared in accordance with Example 43.
(2) Weighing levodopa-carbidopa rapidly disintegrating layer blend (lot # 606-052) prepared in accordance with Example 44.
(3) Mixing the weighed ingredients from steps 1 and 2 in a small container.

The tablets were produced using a single-station manual tablet press, GlobePharma Manual Tablet Compaction Machine MTCM-I, equipped with a 0.3287″×0.8937″ capsule-shaped die and punch set. The compression process comprised:
(4) Adding the mix from step 3 into the die cavity.
(5) Pre-compressing the mix at a pressure ranging from 200 psi (pounds per square inch) and a compression time of 5 seconds.
(6) Compressing the mix together at a pressure ranging from 1900 psi and a compression time of 5 seconds.

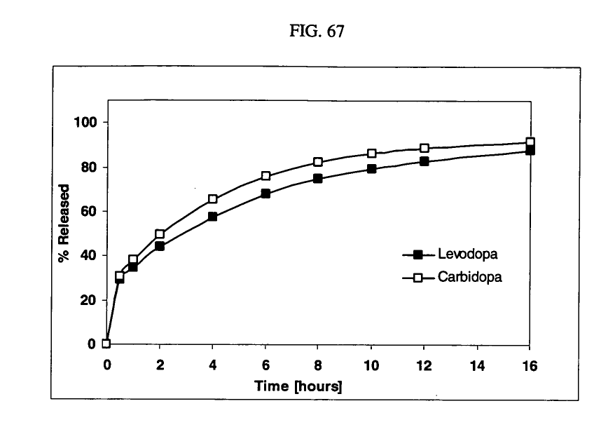

FIG. 67 shows the in vitro dissolution profile of the levodopa-carbidopa rapidly disintegrating pelletized tablets obtained from HPLC analysis in phosphate buffered saline, pH 4.5.

### Example 46

### Manufacturing and In Vitro Dissolution Testing of Levodopa-Carbidopa Bioadhesive Extended Release Pellets, Lot# 606-068

Eighty grams of levodopa-carbidopa core pellets of lot # 606-027 retained on mesh # 35 (from Example 42) were subsequently coated in a Vector MFL.01 Micro Batch Fluid Bed System with a release rate-controlling composition containing EUDRAGIT® RS 100, EUDRAGIT® RL 100, ACRYL-EZE® and triethyl citrate to achieve a weight gain of 4.3% (w/w). These pellets were subsequently film-coated with a bioadhesive polymeric composition comprising SPHEROMER™ III, succinic Acid and citric acid (40:50:5) to achieve a weight gain of 4.2% (w/w). Finally they were top-coated with a SPHEROMER™ I and triethyl citrate (80:20) coating mixture to achieve a weight gain of 1% (w/w). The top coating was added to keep pellets monodispersed upon release in the stomach.

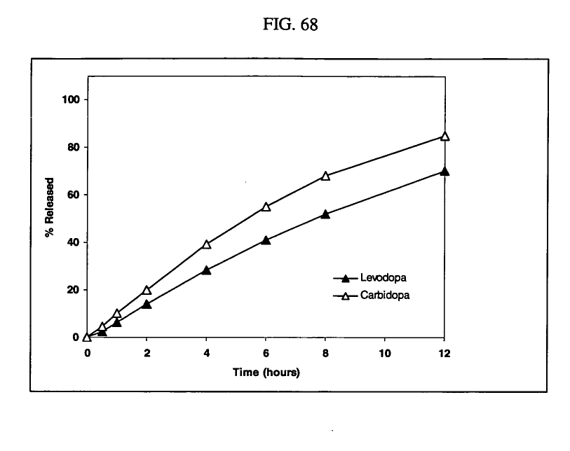

Various film coatings were performed in a fluidized bed coater, Vector MFL.01 Micro Batch Fluid Bed System, equipped with a Wurster insert, operating at an inlet air flow rate of 100±50 lpm (liter per minute) and an inlet air temperature of 35° C.±5° C. The pellets were pre-warmed at 35° C. for 5 min and after film-coating were post-dried at 30° C. for 10 min. The weight and composition of coated pellets (lot # 606-068) are given in Table 23. FIG. 68 shows the dissolution profiles of levodopa and carbidopa obtained from HPLC analysis in phosphate buffered saline, pH 4.5.

TABLE 23
Weight and Composition of Levodopa-Carbidopa
Bioadhesive Extended Release Pellets, Lot# 606-068

| | | Weight |
|---|---|---|
| Components | Weight (%) | (g) |
| Levodopa-Carbidopa Core Pellets | 91.11 | 80 |
| EUDRAGIT ® RS 100, NF | 1.66 | 1.46 |
| EUDRAGIT ® RL 100, NF | 1.66 | 1.46 |
| Triethyl Citrate, NF | 0.58 | 0.51 |
| ACRYL-EZE ® White (93O18509) | 0.19 | 0.17 |
| Succinic Acid, FCC | 2.20 | 1.93 |
| SPHEROMER ™ III | 1.59 | 1.4 |
| Anhydrous Citric Acid, USP | 0.20 | 0.18 |
| SPHEROMER ™ I | 0.80 | 0.7 |
| Total | 100 | 87.81 |

### Example 47

### Manufacturing of Levodopa-Carbidopa Immediate Release Granules, Lot # 606-050

Levodopa-carbidopa immediate-release (IR) granules were prepared by mixing the ingredients of the IR granules (Table 24) excluding hypromellose were blended in a low shear mixer followed by granulation using methanolic solution of hypromellose. The granules were dried using a fluidized bed dryer, Vector MFL.01 Micro Batch Fluid Bed System, operating at an inlet air flow rate of 100-115 lpm (liter per minute) and an inlet air temperature of 50° C. for 2 hours. The granulation was then passed through a U.S. Std mesh #35 sieve.

TABLE 24
Weight and Composition of Levodopa-Carbidopa
Immediate Release Granules, Lot# 606-050

| | | Weight |
|---|---|---|
| Components | Weight (%) | (g) |
| Levodopa, Micronized | 32.30 | 38.96 |
| Carbidopa, Monohydrate, USP | 8.73 | 10.53 |
| AVICEL ® PH-105 (Microcrystalline Cellulose) | 34.05 | 41.07 |
| Croscarmellose Sodium (AC-DI-SOL ®), NF | 9.70 | 11.7 |
| Poloxamer 188 (LUTROL ® F68), NF | 4.37 | 5.27 |
| Butylated Hydroxytoluene, NF | 0.10 | 0.12 |
| Anhydrous Citric Acid, USP | 7.76 | 9.36 |
| Hypromellose 2910, 5 cps (METHOCEL ™ E5 | 2.98 | 3.6 |
| Premium LV), USP | | |
| Total | 100.00 | 120.61 |

### Example 48

### Manufacturing of Bioadhesive Blend, Lot # 603-228

Bioadhesive blend was prepared using SPHEROMER™ III and SPHEROMER™ I granulation. The composition is listed in Table 25. SPHEROMER™ III was granulated along with hydroxypropyl cellulose (HPC) and citric acid by using a 3% (w/v) solution of HPC in methylene chloride in a Hobart Mixer in accordance with the method described in Example 5. The wet granulation was dried in a Vector MFL.01 Micro Batch Fluid Bed System, operating at an inlet air flow rate of 290 lpm (liter per minute) and an inlet air temperature of 55° C. The dried granulation was passed through a U.S. Std. mesh # 40 sieve.

SPHEROMER™ I was granulated along with hydroxypropyl cellulose (HPC) by using a 2% (w/v) solution of HPC in dehydrated alcohol in a Hobart Mixer in accordance with the method described in Example 5. The wet granulation was dried in the Vector MFL.01 Micro Batch Fluid Bed System, operating at an inlet air flow rate of 200 lpm (liter per minute) and an inlet air temperature of 60° C. The dried granulation was passed through a U.S. Std. mesh # 40 sieve.

The screened SPHEROMER™ III and SPHEROMER™ I granulations were blended in a GlobePharma Maxiblend V-shell blender equipped with a 2-qt V-shell, for 10 min. Magnesium stearate was added to the blend and the materials were mixed for an additional 5 min. The final blend was passed through a U.S. Std. mesh # 40 sieve.

TABLE 25
Weight and Composition of Bioadhesive Blend, Lot# 603-228

| | | Weight |
|---|---|---|
| Ingredients | Weight | (mg) |
| SPHEROMER ™ III | 62.2 | 155.6 |
| SPHEROMER ™ I [p (FASA)] | 21.6 | 54.0 |
| Hydroxypropyl cellulose (KLUCEL ® EF Pharm), NF | 13.0 | 32.4 |
| Citric Acid, anhydrous, USP | 3.0 | 7.5 |
| Magnesium Stearate, NF | 0.2 | 0.5 |
| Total | 100.0 | 250.0 |

### Example 49

### Manufacturing of Slowly Eroding Matrix Blend, Lot # 606-072

Slowly eroding matrix blend was prepared using a V-shell blender. The ingredients of the matrix blend (Table 26) excluding magnesium stearate were blended in a GlobePharma Maxiblend V-shell blender equipped with a 2-qt V-shell, for 10 min. Magnesium stearate was added to the mixed ingredients and the materials were blended for an additional 5 min.

TABLE 26
Weight and Composition of Bioadhesive Blend, Lot# 603-228

| | | Weight |
|---|---|---|
| Ingredients | Weight % | (mg) |
| Ethylcellulose (ETHOCEL ™ Std 10 FP Premium), | 32.5 | 243.75 |
| Compressable Sugar, NF | 45.2 | 339.00 |
| Succinic Acid, FCC | 7.0 | 52.50 |
| Talc, USP | 15.0 | 112.50 |
| Magnesium Stearate, NF | 0.3 | 2.25 |
| Total | 100.0 | 750.00 |

### Example 50

### Manufacturing and In Vitro Dissolution of Levodopa-Carbidopa 200 mg/50 mg Slowly Eroding Pelletized Extended-Release Tablets, Lot # 606-072

This formulation approach is a multilayer tablet system is a multilayer tablet system consisting of an immediate release (IR) levodopa-carbidopa component, a bioadhesive or optionally a non-bioadhesive backing layer and levodopa-carbidopa bioadhesive extended release multiparticulate beads embedded in an inner slowly eroding matrix. The bioadhesive layer adheres to the gastric mucosa and further reduces variability by increasing the gastric residence time. The IR layer disintegrates rapidly releasing an initial dose of levodopa-carbidopa in the stomach. This allows an initial dose of levodopa and carbidopa to be immediately absorbed. The inner matrix layer of the tablet erodes slowly and evenly for 3 to 4 hours and releases the multiparticulate beads slowly. The multiparticulate beads adhere to the gastric mucosal lining and release levodopa and carbidopa in a regulated manner.

Levodopa-carbidopa slowly eroding pelletized extended-release tablets were prepared using direct compression. The manufacturing processes comprised:
(1) Weighing levodopa-carbidopa bioadhesive extended-release pellets (lot # 606-068) prepared in accordance with Example 46.
(2) Weighing levodopa-carbidopa immediate-release granules (lot # 606-050) prepared in accordance with Example 47.
(3) Weighing bioadhesive blend (lot # 603-228) prepared in accordance with Example 48.
(4) Weighing slowly eroding matrix blend (lot # 606-072) prepared in accordance with Example 49.
(5) Mixing the weighed ingredients from step (1) and step (4) in a small container.

The tablets were produced using a single-station manual tablet press, GlobePharma Manual Tablet Compaction Machine MTCM-I, equipped with a 0.3287″×0.8937″ capsule-shaped die and punch set. The compression process comprised:
(6) Adding the material from step (3), followed by step (5), and finally step (2) into the die cavity.
(7) Compressing the mix together at a pressure of 2500 psi (pounds per square inch) and a compression time of 5 seconds.

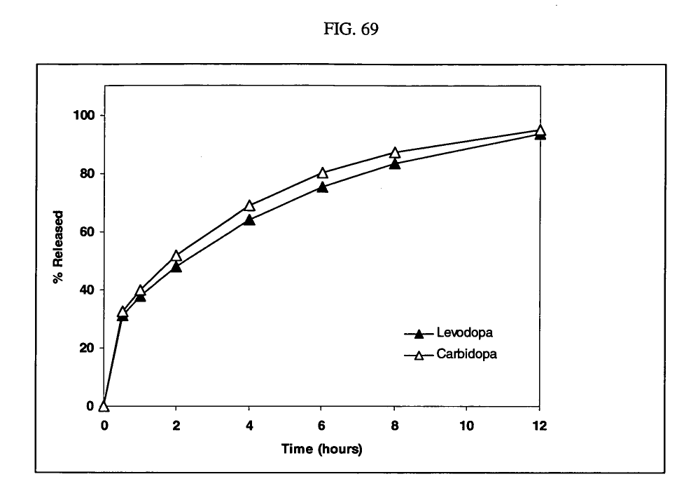

FIG. 69 shows the in vitro dissolution profile of the levodopa-carbidopa slowly eroding pelletized extended-release tablets (lot # 606-072) in phosphate buffered saline, pH 4.5.

The following examples relates to the multiparticulate formulation.

### Example 51

### Production of Levodopa, Carbidopa, and Levodopa-Carbidopa Pellets with Granulation-Extrusion-Spheronization and Fluid Bed Drying

Levodopa, carbidopa, and levodopa-carbidopa pellets were produced with granulation-extrusion-spheronization and fluid bed drying. The following steps (or minor variations thereof) may be followed to produce the pellets:
(1) Weighing levodopa or carbidopa, or both levodopa and carbidopa, optionally a bioadhesive polymer composition, and pharmaceutically acceptable excipients.
(2) Blending levodopa or carbidopa, or both levodopa and carbidopa, and optionally a bioadhesive polymer composition, with pharmaceutically acceptable excipients in a planetary type mixer, e.g., Hobart Mixer with a 5-qt mixing bowl, operating at the speed setting #1, for 5-15 min, forming a dry mix.
(3) Granulating the dry mix from step (2) under low shear with a granulation fluid, forming a wet granulation. The granulation fluids were mainly selected from, e.g., purified water, an aqueous solution of a mineral or organic acid, an aqueous solution of a polymeric composition, a pharmaceutically acceptable alcohol, a ketone or a chlorinated solvent, a hydro-alcoholic mixture, an alcoholic or hydro-alcoholic solution of a polymeric composition, a solution of a polymeric composition in a chlorinated solvent or in a ketone.
(4) Extruding the wet granulation from step (3) through the screen of a screen-type extruder, e.g., Caleva Model 20 (or Model 25) Extruder, operating at 10-20 rpm, and forming breakable wet strands, the extrudate. The screen aperture was 0.8, 1, or 1.5 mm.
(5) Spheronizing the extrudate from step (4) in a spheronizer, e.g., Caleva Model 250, equipped with a 2.5-mm spheronization plate, operating at 1000-2000 rpm for 5-10 min, and forming spheronized pellets.
(6) Drying the spheronized pellets from step (5) in a fluidized bed drier, e.g., Vector MFL.01 Micro Batch Fluid Bed System, operating at an inlet air flow rate of 100-300 lpm (liters per minute) and an inlet air temperature of 50° C. Alternatively, pellets were dried either in an ACT (Applied Chemical Technology) fluidized bed drier or in a conventional Precision oven. The ACT fluidized bed drier was operated at an inlet air flow rate of 140-150 fpm (foot per minute) and an inlet air temperature of 104° F. The oven was set at 50° C.
(7) Screening and classifying the dried pellets from step (6) through a stack of stainless steel sieves, U.S. standard mesh sizes 8, 10, 12, 14, 16, 18, 20, 25, 30, 40, 45, and 60 using a mechanical sieve shaker, W.S. Tyler Sieve Shaker Ro-Tap Rx-29, operated for 5 min. Particle size and distribution of pellet formulations were analyzed, and classified pellets ranging from 0.25 mm (mesh # 60) to 2 mm (mesh # 10) were selected for future film coating or other experimentation.

### Example 52

### Production of Levodopa, Carbidopa, and Levodopa-Carbidopa Pellets with Granulation-Extrusion-Spheronization and Oven Drying

Levodopa, carbidopa, and levodopa-carbidopa pellets were produced with granulation-extrusion-spheronization and oven drying. The production processes included the steps 1 to 5 and 7 of Example 51 but the spheronized pellets were dried in a Precision gravity oven, operating at 50° C., for 8-24 h.

### Example 53

### Film Coating of Levodopa, Carbidopa, and Levodopa-Carbidopa Pellets with Bioadhesive Polymer, SPHEROMER™ I [poly(FASA)]

Levodopa, carbidopa, and levodopa-carbidopa pellets were film-coated with a bioadhesive polymeric composition, SPHEROMER™ I [poly(FASA)]. Bioadhesive SPHEROMER™ I and optionally a functional polymer, or a non-functional polymer, and optionally pharmaceutically acceptable excipients, were dissolved in methylene chloride. The film coating was performed in a fluidized bed coater, Vector MFL.01 Micro Batch Fluid Bed System, equipped with a Wurster insert, operating at an inlet air flow rate of 100-300 lpm (liters per minute) and an inlet air temperature of 25° C. to 30° C. The pellets were pre-warmed at 35° C. for 2-5 min and after film-coating were post-dried at 30° C. for 15-30 min.

### Example 54

### Film Coating of Levodopa, Carbidopa, and Levodopa-Carbidopa Pellets with Bioadhesive Polymer, SPHEROMER™ III

Levodopa, carbidopa, and levodopa-carbidopa pellets were film-coated with a bioadhesive polymeric composition, SPHEROMER™ III. Bioadhesive SPHEROMER™ III and optionally a functional polymer, or a non-functional polymer, and optionally pharmaceutically acceptable excipients, were dissolved in methanol. The film coating was performed in a fluidized bed coater, Vector MFL.01 Micro Batch Fluid Bed System, equipped with a Wurster insert, operating at an inlet air flow rate of 100-300 lpm (liter per minute) and an inlet air temperature of 35° C.±2° C. The pellets were pre-warmed at 35° C. for 2-5 min and after film-coating were post-dried at 30° C. for 15-30 min.

Alternatively, pellets were coated in a Fluid Air Model 5 fluid bed processor, equipped with a Wurster insert, operating at an inlet air flow rate of 70 cfm (cubic foot per minute) and an inlet air temperature of 35° C. The pellets were pre-warmed at 40° C. for 5-7 min and after film-coating were post-dried at 35° C. for 30 min.

### Example 55

### Film Coating of Levodopa, Carbidopa, and Levodopa-Carbidopa Pellets with Bioadhesive Polymeric Composition Comprising SPHEROMER™ I [poly(FASA)] and SPHEROMER™ III

Levodopa, carbidopa, and levodopa-carbidopa pellets were film-coated with a bioadhesive polymeric composition of SPHEROMER™ I [poly(FASA)] and SPHEROMER™ III. Bioadhesive SPHEROMER™ I [poly(FASA)] and SPHEROMER™ III polymers, and optionally a functional polymer, or a non-functional polymer, and optionally pharmaceutically acceptable excipients, were dissolved in a binary mixture of methanol and methylene chloride. The film coating was performed in a fluidized bed coater, Vector MFL.01 Micro Batch Fluid Bed System, equipped with a Wurster insert, operating at an inlet air flow rate of 100-300 lpm (liter per minute) and an inlet air temperature of 25° C. to 35° C. The pellets were pre-warmed at 35° C. for 2-5 min and after film-coating were post-dried at 30° C. for 15-30 min.

### Example 56

### Film Coating of Levodopa, Carbidopa, and Levodopa-Carbidopa Pellets with Bioadhesive Polymer, SPHEROMER™ IV

Levodopa, carbidopa, and levodopa-carbidopa pellets were film-coated with a bioadhesive polymeric composition, SPHEROMER™ IV. Bioadhesive SPHEROMER™ IV and optionally a functional polymer, or a non-functional polymer, were dissolved in methanol or a binary mixture of ethanol and water (3:1 v/v). The film coating was performed in a fluidized bed coater, Vector MFL.01 Micro Batch Fluid Bed System, equipped with a Wurster insert, operating at an inlet air flow rate of 100-300 lpm (liter per minute) and an inlet air temperature of 35° C. The pellets were pre-warmed at 35° C. for 2-5 min and after film coating were post-dried at 30° C. for 15-30 min.

### Example 57

### Film Coating of Levodopa, Carbidopa, and Levodopa-Carbidopa Pellets with a Functional or a Non-Functional Polymer

Levodopa, carbidopa, and levodopa-carbidopa pellets were film coated with a functional or a non-functional polymer. The polymer was dissolved in either of methanol, ethanol, or isopropanol, or their binary mixture with acetone. The film coating was performed in a fluidized bed coater, Vector MFL.01 Micro Batch Fluid Bed System, equipped with a Wurster insert, operating at an inlet air flow rate of 100-300 lpm (liter per minute) and an inlet air temperature of 30° C. to 40° C. The pellets were pre-warmed at 30° C. to 40° C. for 2-5 min and after film coating were post-dried at 30° C. to 40° C. for 15-30 min.

### Example 58

### Production of Carbidopa Granules with Low Shear Granulation and Fluid Bed Drying

Carbidopa granules were produced with low shear granulation method comprising the following processes:
(1) Weighing carbidopa, optionally a bioadhesive polymer composition, and pharmaceutically acceptable excipients.
(2) Blending carbidopa, and optionally a bioadhesive polymer composition, with pharmaceutically acceptable excipients in a planetary type mixer, e.g., Hobart Mixer, operating at the speed setting #1, for 5-15 min, forming a dry mix.
(3) Granulating the dry mix from step (2) under low shear with a granulation fluid, forming a wet granulation. The granulation fluid were mainly selected from purified water, an aqueous solution of a mineral or organic acid, an aqueous solution of a polymeric composition, an alcohol, a hydro-alcoholic mixture, or an alcoholic or hydro-alcoholic solution of a polymeric composition.
(4) Drying the granulation from step (3) in a fluidized bed drier, e.g., Vector MFL.01 Micro Batch Fluid Bed System, operating at an inlet air flow rate of 100-300 lpm (liters per minute) and an inlet air temperature of 50° C. Alternatively, the granulation from step (3) was dried in a Precision gravity oven, operating at 50° C., for 8-24 h.
(5) Screening and classifying the dried granules from step (4) through a stack of stainless steel sieves, U.S. standard mesh sizes 20 and 60, using a mechanical sieve shaker, W.S. Tyler Sieve Shaker Ro-Tap Rx-29, operated for 5 min. Particle size and distribution of granular formulations were analyzed, and classified granules ranging from 0.25 mm (mesh # 60) to 0.85 mm (mesh # 20) were selected for future experimentation.

### Example 59

### Production of Carbidopa Granules with Low Shear Granulation and Oven Drying

Carbidopa granules were produced with low shear granulation and oven drying. The production processes included the steps 1 to 3 and 5 of Example 58 but the granulation was dried in a Precision gravity oven, operating at 50° C., for 8-48 h.

### Example 60

### In Vitro Dissolution of Multiparticulate Formulations of Levodopa, Carbidopa, and Levodopa-Carbidopa

The in vitro dissolution profile of levodopa, carbidopa, and levodopa-carbidopa multiparticulate formulations were obtained under simulated gastric conditions. The dissolution tests were performed in 900 mL of either of 0.1 N HCl-pH 1.2, phosphate buffer saline (PBS) pH 4.5, or sodium acetate buffer pH 4.5 solutions, in a USP II apparatus at a temperature of 37° C. The paddle speed was set at 50 rpm. Samples of dissolution media were collected at predetermined intervals and analyzed by either HPLC or UV spectrophotometry.

### Example 61

### In Vitro Dissolution of SINEMET® 10-100 Tablets, Containing 10 mg Carbidopa and 100 mg Levodopa, Lot # 00067

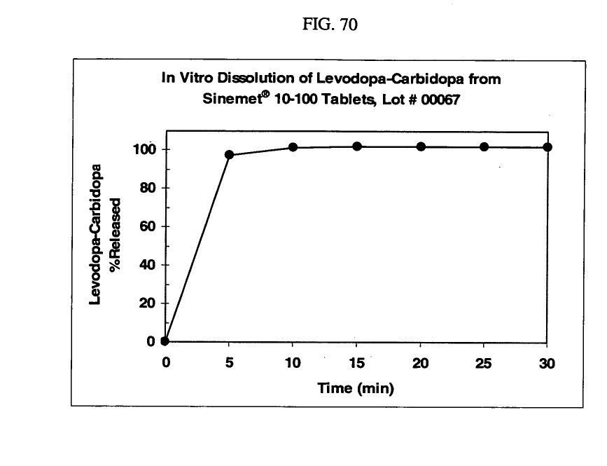

The in vitro dissolution profile of SINEMET® 10-100 tablets, containing 10 mg carbidopa and 100 mg levodopa was obtained under simulated gastric conditions. The dissolution tests were performed in 900 mL of either of 0.1N HCl-pH 1.2, phosphate buffer saline (PBS)-pH 4.5, or sodium acetate buffer-pH 4.5 solutions, in a USP II apparatus at a temperature of 37° C. The paddle speed was set at 50 rpm. Samples of dissolution media were collected at predetermined intervals and analyzed by UV spectrophotometry. The combined dissolution profile of levodopa-carbidopa obtained from UV spectrophotometry analysis is shown in FIG. 70.

### Example 62

### In Vitro Dissolution of SINEMET® CR 50-200 Tablets, Containing 50 mg Carbidopa and 200 mg Levodopa, Lot # N4682

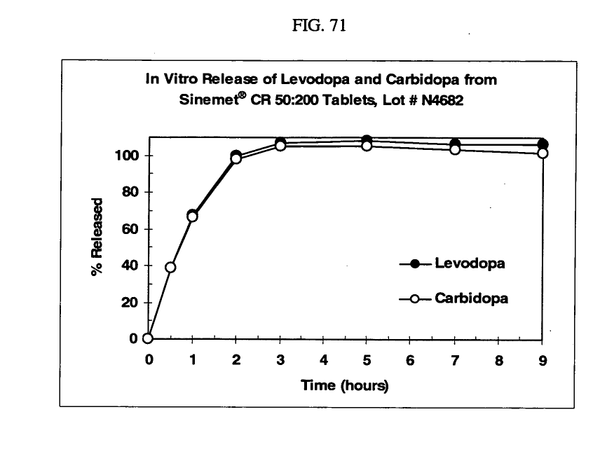

The in vitro dissolution profile of SINEMET® CR 50-200 tablets, containing 50 mg carbidopa and 200 mg levodopa were obtained under simulated gastric conditions. The dissolution tests were performed in 900 mL of 0.1N HCl-pH 1.2 solution, in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 71.

### Example 63

### In Vivo Pharmacokinetic Performance of SINEMET® 10-100 Tablets in Fed Beagle Dogs, Lot # 00067

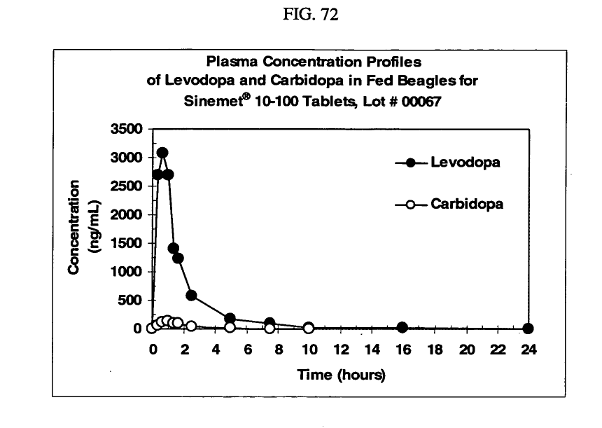

The in vivo performance of SINEMET® 10-100 tablets was evaluated in beagle dogs. SINEMET® tablets were administered to cohorts of six beagle dogs in the fed state and plasma levels of levodopa and carbidopa were measured using LC/MS/MS analysis. FIG. 72 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 1A.

TABLE 1A
Pharmacokinetic Data for SINEMET ® 10-100 Tablets,
Lot # 00067, in Fed Beagle Dogs; the area under the plasma
levodopa vs. time curve (AUC), maximum concentration (Cₘₐₓ),
and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® 10-100 | 5,956 | 3,400 | 0.66 |
| Tablets | | | |

### Example 64

### In vivo Pharmacokinetic Performance of SINEMET® CR 50-200 Tablets in Fed Beagle Dogs, Lot # N4682

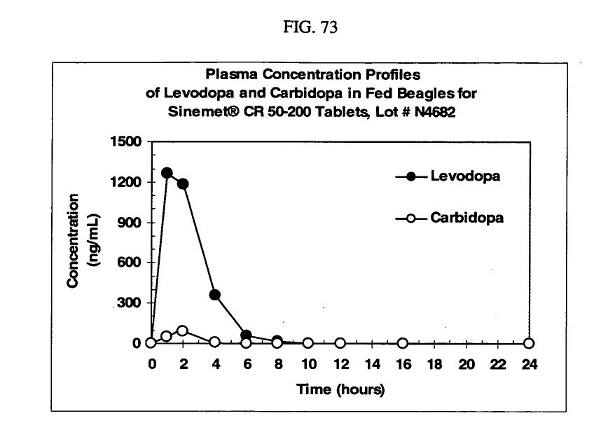

The in vivo performance of SINEMET® CR 50-200 tablets was evaluated in beagle dogs. SINEMET® CR tablets were administered to cohorts of six beagle dogs in the fed state and plasma levels of levodopa and carbidopa were measured using HPLC analysis. FIG. 73 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 2A.

TABLE 2A
Pharmacokinetic Data for SINEMET ® CR 50-200 Tablets,
Lot # N4682, in Fed Beagle Dogs; the area under the plasma
levodopa vs. time curve (AUC), maximum concentration (Cₘₐₓ),
and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® CR | 3,903 | 1,663 | 2 |
| 50-200 Tablets | | | |

### Example 65

### In Vivo Pharmacokinetic Performance of SINEMET® CR 50-200 Tablets in Fasted Beagle Dogs, Lot # N4682

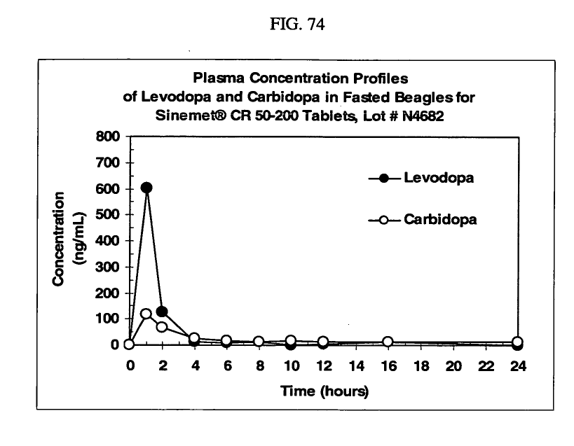

The in vivo performance of SINEMET® CR 50-200 tablets was evaluated in beagle dogs. SINEMET® CR tablets were administered to cohorts of twelve beagle dogs in the fasted state and plasma levels of levodopa and carbidopa were measured using HPLC analysis. FIG. 74 shows the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 3A.

TABLE 3A
Pharmacokinetic Data for SINEMET ® CR 50-200 Tablets,
Lot # N4682, in Fasted Beagle Dogs; the area under the plasma
levodopa vs. time curve (AUC), maximum concentration
(Cₘₐₓ), and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® CR | 936 | 604 | 1 |
| 50-200 Tablets | | | |

### Example 66

### Production of Levodopa Pellets with Granulation-Extrusion-Spheronization, Lot # 510-095

Three identical sub-lots of levodopa pellets (sub-lots # 511-068, 511-069, and 511-070) were prepared in accordance with the method described in Example 51. The weight and composition of pellets of the sub-lot # 511-068 are given in Table 4A. Levodopa was blended with inactive excipients for 5 min. The levodopa-excipients blend was then granulated by spraying purified water while mixing at low shear. The granulation was blended for an additional 5 min and then extruded through a 1.5 mm screen of a Caleva extruder, model 25, operating at 15 rpm. The extrudate was spheronized in a Caleva spheronizer, model 250, operating at 1000 rpm for 5 min. The spheronized pellets were dried in an ACT (Applied Chemical Technology) fluidized bed drier at 104° F.±4° F. for 75 min. The dried pellets were screened and particles with diameters ranging from 1 mm to 2 mm were selected for future experimentation. The screened pellets of the three sub-lots were blended in a GlobePharma Maxiblend Blender equipped with an 8-qt stainless steel V-shell.

TABLE 4A
Weight and Composition of Levodopa Pellets, Sub-lot # 511-068

| Ingredients | Weight % | Weight (g) |
|---|---|---|
| Levodopa, USP | 50.0 | 300 |
| Microcrystalline cellulose | 25.0 | 150 |
| (EMCOCEL ® 90 M), NF | | |
| Mannitol (MANNOGEM ™ Powdered), USP | 14.0 | 84 |
| Hydroxypropylcellulose (HPC-SSL), NF | 5.0 | 30 |
| Croscarmellose sodium (AC-DI-SOL ®), NF | 5.0 | 30 |
| Citric acid, anhydrous, USP | 1.0 | 6 |
| Total | 100.0 | 600 |

### Example 67

### Production of Levodopa-Carbidopa (4:1) Pellets with Granulation-Extrusion-Spheronization, Lot # 510-096

Three identical sub-lots of levodopa-carbidopa pellets (sub-lots # 510-094, 511-043, and 511-055) were prepared in accordance with the method described in Example 51. The weight and composition of the sub-lot # 510-094 are given in Table SA. Levodopa and carbidopa were blended with inactive excipients for 5 min. The levodopa-carbidopa-excipients blend was then granulated by spraying purified water while mixing at low shear. The granulation was blended for an additional 5 min and then extruded through a 1.5 mm screen of a Caleva extruder, model 25, operating at 15 rpm. The extrudate was spheronized in a Caleva spheronizer, model 250, operating at 1000 rpm for 5 min. The spheronized pellets were dried in an ACT (Applied Chemical Technology) fluidized bed drier at 104° F.±4° F. for 25 min. The dried pellets were screened and particles with diameters ranging from 1 mm to 2 mm were selected for future experimentation. The screened pellets of the three sub-lots were blended in a GlobePharma Maxiblend Blender equipped with an 8-qt stainless steel V-shell.

TABLE 5A
Weight and Composition of Levodopa-Carbidopa Pellets,
Sub-lot # 510-094

| Ingredients | Weight % | Weight (g) |
|---|---|---|
| Levodopa, USP | 50.0 | 300 |
| Carbidopa monohydrate, USP | 13.5 | 81 |
| Microcrystalline cellulose (EMCOCEL ® 90 M), | 25.0 | 150 |
| Hydroxypropylcellulose (HPC-SSL), NF | 5.5 | 33 |
| Lactose monohydrate (FASTFLO ® 316), NF | 5.0 | 30 |
| Citric acid, anhydrous, USP | 1.0 | 6 |
| Total | 100.0 | 600 |

### Example 68

### Production of Carbidopa Granules with Low Shear Granulation, Lot #511-101

Carbidopa granules were prepared in accordance with the method described in Example 59. The weight and composition of granules are given in Table 6A. Carbidopa was blended with inactive excipients for 5 min. The carbidopa-excipients blend was then granulated by spraying purified water while mixing at low shear. The granulation was blended for an additional 5 min and then dried in a Precision gravity oven at 50° C. for 41.5 hours. The dried granules were screened and particles smaller than 0.85 mm were selected for future experimentation.

TABLE 6A
Weight and Composition of Carbidopa Granules, Lot # 511-101

| Ingredients | Weight % | Weight (g) |
|---|---|---|
| Carbidopa monohydrate, USP | 52.0 | 104 |
| Microcrystalline cellulose (EMCOCEL ® 90 M), | 23.5 | 47 |
| Mannitol (MANNOGEM ™ Powdered), USP | 13.5 | 27 |
| Hydroxypropylcellulose (HPC-SSL), NF | 5.0 | 10 |
| Croscarmellose sodium (AC-DI-SOL ®), NF | 5.0 | 10 |
| Citric acid, anhydrous, USP | 1.0 | 2 |
| Total | 100.0 | 200 |

### Example 69

### Film Coating of Levodopa Pellets with Bioadhesive Polymer, SPHEROMER™ III, and Hydroxypropylcellulose (HPC-SSL), Lot # 511-092

One thousand grams of levodopa pellets, lot # 510-095, were film-coated in a Fluid Air Model 5 fluid bed processor, equipped with a Wurster insert, in accordance with the method described in Example 54. The composition of coating solution is given in Table 7A. SPHEROMER™ III and Hydroxypropylcellulose (HPC-SSL) were dissolved in methanol and sprayed onto the fluidized pellets to obtain a 12% weight gain on pellets.

TABLE 7A
Composition of SPHEROMER ™ III/Hydroxypropylcellulose
(HPC-SSL) Coating Solution, Lot # 511-092

| Ingredients | Weight % | Weight (g) |
|---|---|---|
| SPHEOROMER ™ III | 80.0 | 120 |
| Hydroxypropylcellulose (HPC SSL), NF | 20.0 | 30 |
| Methyl alcohol, NF | * | (3,000 mL) |
| Total | 100.0 | 150 |

*Methyl alcohol is removed during the coating/drying process.

### Example 70

### Film Coating of Levodopa-Carbidopa Pellets with Bioadhesive Polymer, SPHEROMER™ III, Lot # 510-098

One thousand grams of levodopa-carbidopa pellets, lot # 510-096, were film-coated in a Fluid Air Model 5 fluid bed processor, equipped with a Wurster insert, in accordance with the method described in Example 54. The composition of coating solution is given in Table 8A. SPHEROMER™ III and Poloxamer 188 (Lutrol® F68) were dissolved in methanol and sprayed onto the fluidized pellets to obtain a 6% weight gain on pellets.

TABLE 8A
Composition of SPHEROMER ™ III Coating Solution, Lot # 511-098

| Ingredients | Weight % | Weight (g) |
|---|---|---|
| SPHEOROMER ™ III | 94.7 | 71 |
| Poloxamer 188 (LUTROL ® F68), NF | 5.3 | 4 |
| Methyl alcohol, NF | * | (1,500 mL) |
| Total | 100.0 | 150 |

*Methyl alcohol is removed during the coating/drying process.

### Example 71

### Preparation of Levodopa-Carbidopa 200 mg/50 mg Multiparticulate Capsules, Lots # 510-099 & 510-100

Levodopa pellets (lot # 510-095), SPHEROMER™ III-coated levodopa-carbidopa pellets (lot # 510-098), HPC-SSL/SPHEROMER™ III-coated levodopa pellets (lot # 511-092), and carbidopa granules (lot # 511-101) were encapsulated in 00-size hard gelatin capsules. Each capsule contained 200 mg levodopa and 50 mg carbidopa anhydrous. The composition of multiparticulates in each capsule formulation is given in Table 9A.

TABLE 9A
Composition (mg) of Multiparticulate Capsule Formulations,
Lot # 510-099 & 510-100

| Components | Lot # | 510-099 | 510-100 |
|---|---|---|---|
| Levodopa Pellets | 510-095 | 80 | 80 |
| SPHEROMER III-coated Levodopa- | 510-098 | 340 | 255 |
| Carbidopa Pellets | | | |
| HPC-SSL/SPHEROMER ™ III-coated | 511-092 | — | 90 |
| Levodopa | | | |
| Carbidopa Granules | 511-101 | 20 | 40 |
| Total (mg per capsule) | — | 440 | |

### Example 72

### In Vitro Dissolution and In Vivo Pharmacokinetic Performance of Levodopa-Carbidopa 200 mg/50 mg Multiparticulate Capsules, Lot # 510-099

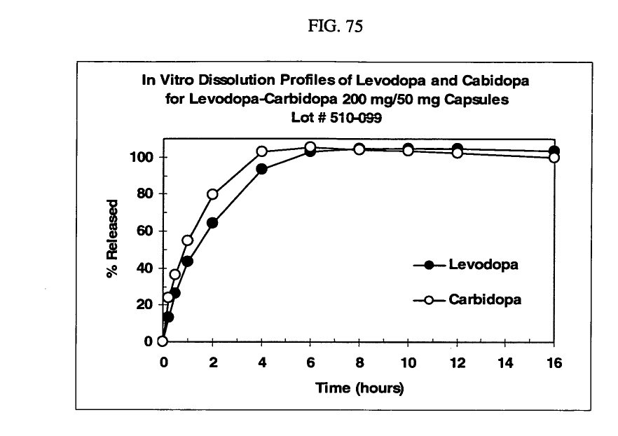

The in vitro dissolution profile of levodopa-carbidopa capsules (Lot # 510-099), containing 50 mg carbidopa and 200 mg levodopa was obtained under simulated gastric conditions. The dissolution tests were performed in 900 mL of 0.1 N HCl-pH 1.2 solution in a USP II apparatus at a temperature of 37° C. The paddle speed was set at 50 rpm. Samples of dissolution media were collected at predetermined intervals and analyzed by HPLC. The dissolu-tion profiles of levodopa and carbidopa obtained from HPLC analysis are shown in FIG. 75.

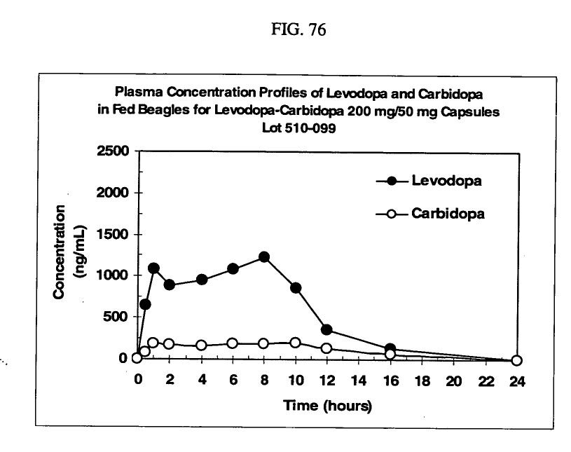

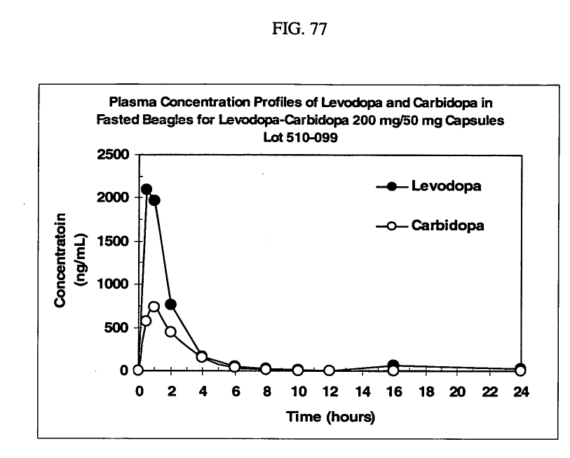

The in vivo performance of levodopa-carbidopa capsules was evaluated in beagle dogs. The capsules were administered to separate cohorts of six beagle dogs in the fed and the fasted states. Plasma levels of levodopa and carbidopa were measured using LC/MS/MS analysis. FIGS. 76 and 77 show the plasma concentration profiles of levodopa and carbidopa in the fed and fasted states, respectively. The pharmacokinetic data including the area under the plasma levodopa vs. time curve (AUC), maximum concentration (Cₘₐₓ) and time required to achieve Cₘₐₓ (Tₘₐₓ) are provided in Table 10A.

It is apparent that, compared to the in vivo pharmacokinetic performance of SINEMET® CR 50-200 Tablets in similarly fed beagle dogs, the AUC of the subject formulation is more than 3 times that of the SINEMET® CR 50-200 Tablets, while the Cₘₐₓ is about the same. Furthermore, the Tₘₐₓ of the subject formulation is more than twice that of the SINEMET® CR 50-200 Tablets (e.g., 4.3 hrs compared to 2 hrs).

The results are even more pronounced in similarly fasted beagle dogs.

TABLE 10A
Pharmacokinetic Data for levodopa-carbidopa capsules, Lot # 510-099,
in Fed and Fasted Beagle Dogs; the area under the plasma levodopa vs.
time curve (AUC), maximum concentration (Cₘₐₓ), and time required to
achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed State | 12,581 | 1,705 | 4.3 |
| Fasted State | 4,678 | 2,743 | 1 |

### Example 73

### In Vitro Dissolution and In Vivo Pharmacokinetic Performance of Levodopa-Carbidopa 200 mg/50 mg Multiparticulate Capsules, Lot # 510-100

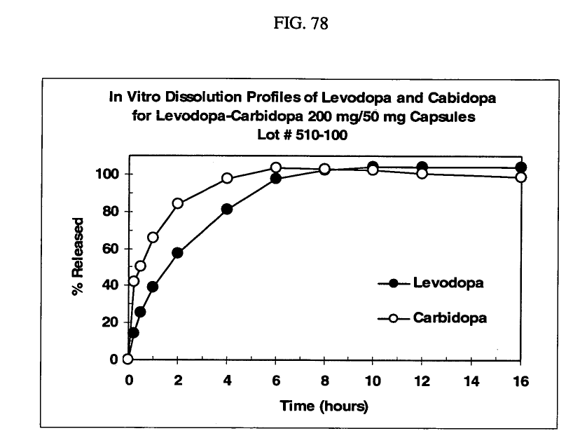

The in vitro dissolution profile of levodopa-carbidopa capsules (Lot # 510-100), containing 50 mg carbidopa and 200 mg levodopa was obtained under simulated gastric conditions. The dissolution tests were performed in 900 mL of 0.1N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 78.

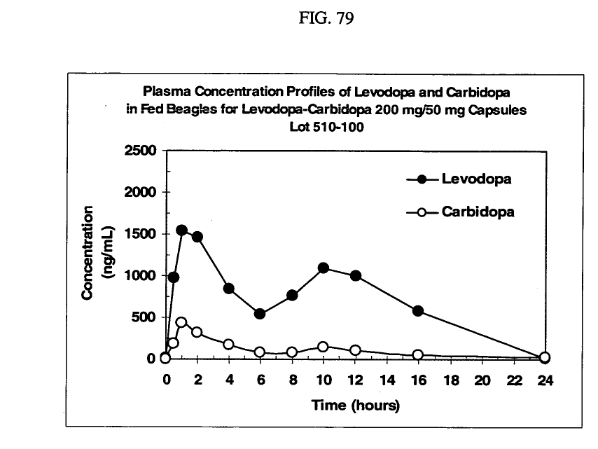

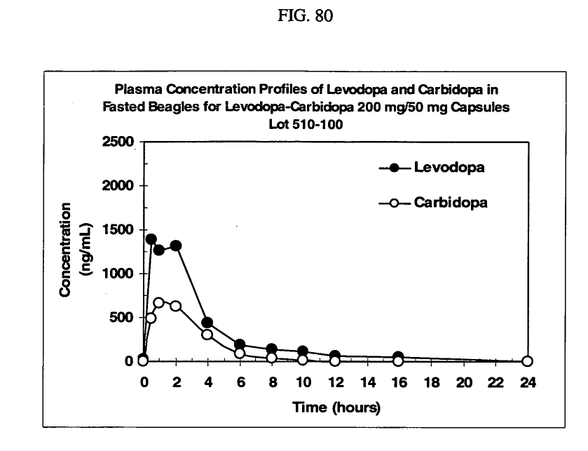

The in vivo performance of levodopa-carbidopa capsules was evaluated in beagle dogs. The capsules were administered to separate cohorts of six beagle dogs in the fed and the fasted states. Plasma levels of levodopa and carbidopa were measured using LC/MS/MS analysis. FIGS. 79 and 80 show the plasma concentration profiles of levodopa and carbidopa in the fed and fasted states, respectively. The pharmacokinetic data including the area under the plasma levodopa vs. time curve (AUC), maximum concentration (Cₘₐₓ) and time required to achieve Cₘₐₓ (Tₘₐₓ) are provided in Table 11A.

Under this formulation, it is apparent that, compared to the in vivo pharmacokinetic performance of SINEMET® CR 50-200 Tablets in similarly fed beagle dogs, the AUC of the subject formulation is more than 4 times that of the SINEMET® CR 50-200 Tablets, while the C. is about 50% higher. Furthermore, the T. of the subject formulation is about 2.5 times that of the SINEMET® CR 50-200 Tablets (e.g., 5 hrs compared to 2 hrs).

The results are even more pronounced in similarly fasted beagle dogs.

TABLE 11A
Pharmacokinetic Data for levodopa-carbidopa capsules, Lot # 510-100,
in Fed and Fasted Beagle Dogs; the area under the plasma levodopa vs.
time curve (AUC), maximum concentration (Cₘₐₓ), and time required to
achieve Cₘₐₓ (Tₘₐₓ)

| | AUC | Cₘₐₓ | Tₘₐₓ |
|---|---|---|---|
| Fasting Period | (ng/ml · hr) | (ng/ml) | (hr) |
| Fed | 16,811 | 2,518 | 5 |
| Fasted | 5,872 | 2,113 | 1.2 |

### Example 74

### Production and In Vitro Dissolution of Levodopa-Carbidopa (4:1) Pellets, Sub-Lots # 602-042 & 602-043

Two identical sub-lots of levodopa-carbidopa pellets (sub-lots # 602-042 and 602-043) were prepared in accordance with the method described in Example 51. The weight and composition of pellets of the sub-lot # 602-042 are given in Table 12A. Levodopa and carbidopa were blended with inactive excipients for 15 min. The levodopa-carbidopa-excipients blend was then granulated by spraying a 1% (w/v) aqueous solution of polyethylene oxide (Polyox™ WSR N10) while mixing at low shear. The granulation was blended for an additional 5 min and then extruded through a 1.5 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 190 min. The dried pellets were screened and particles with diameters ranging from 1 mm to 2 mm were selected for future experimentation.

TABLE 12A
Weight and Composition of Levodopa-Carbidopa Pellets,
Sub-lot # 602-042

| Ingredients | Weight % | Weight (g) |
|---|---|---|
| Levodopa, USP | 59.11 | 90.00 |
| Carbidopa monohydrate, USP | 15.95 | 24.29 |
| Microcrystalline cellulose | 14.78 | 22.50 |
| (EMCOCEL ® 90 M), NF | | |
| L-Glutamic acid, FCC | 9.75 | 14.85 |
| Polyethylene oxide (POLYOX ™ WSR N10), NF | 0.31 | 0.47 |
| Butylated hydroxytoluene (BHT), NF | 0.10 | 0.15 |
| Total | 100.00 | 152.26 |

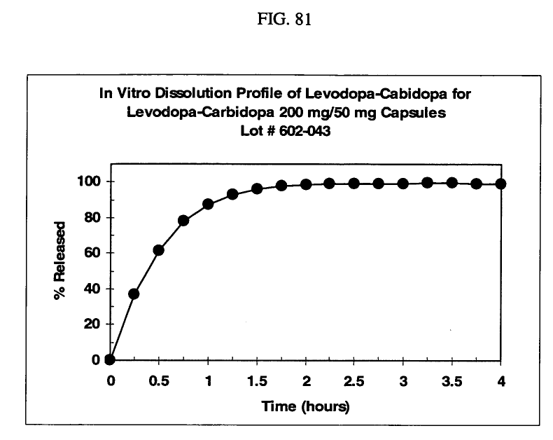

Aliquots of screened pellets, lot # 602-043, were encapsulated in 00-size hard gelatin capsules. The in vitro dissolution profile of levodopa-carbidopa capsules, containing 200 mg levodopa and 50 mg carbidopa anhydrous was obtained under simulated gastric conditions. The dissolution test was performed in 900 mL of 0.1 N HCl-pH 1.2 solution in a USP II apparatus at a temperature of 37° C. The paddle speed was set at 50 rpm. Samples of dissolution medium were collected at predetermined intervals and analyzed by online UV spectrophotometry. The combined dissolution profile of levodopa-carbidopa obtained from UV analysis is shown in FIG. 81.

### Example 75

### Production and In Vitro Dissolution of SPHEROMER™ III/EUDRAGIT® RS 100-Coated Levodopa-Carbidopa Pellets, Lot # 603-139

Fifty grams of levodopa-carbidopa pellets, a blend of sub-lots # 602-042 and 602-043, were film-coated in a Vector MFL.01 Micro Batch Fluid Bed System, equipped with a Wurster insert, in accordance with the method described in Example 54. The screened pellets of the two sub-lots were blended in the fluid bed system by fluidization prior to film-coating. The composition of coating solution is given in Table 13A. SPHEROMER™ III, EUDRAGIT® RS 100, and anhydrous citric acid were dissolved in methanol and sprayed onto the fluidized pellets to obtain a 12% weight gain on pellets.

TABLE 13A
Composition of SPHEROMER ™ III/EUDRAGIT ® RS 100
Coating Solution, Lot # 603-139

| Ingredients | Weight % | Weight (g) |
|---|---|---|
| Spheoromer ™ III | 85 | 5.1 |
| EUDRAGIT ® RS 100 | 10 | 0.6 |
| Anhydrous citric acid, USP | 5 | 0.3 |
| Methyl alcohol, NF | * | (150 mL) |
| Total | 100.0 | 6.0 |

*Methyl alcohol is removed during the coating/drying process.

Aliquots of coated pellets were encapsulated in 00-size hard gelatin capsules.

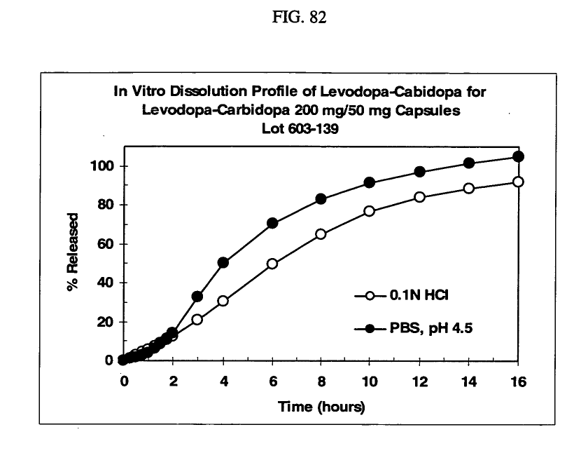

The in vitro dissolution profile of levodopa-carbidopa capsules, containing 200 mg levodopa and 50 mg carbidopa anhydrous was obtained under simulated gastric conditions. The dissolution test was performed in 900 mL of either of 0.1N HCl-pH 1.2 or phosphate buffer saline (PBS)-pH 4.5 solutions in a USP II apparatus at a temperature of 37° C. The paddle speed was set at 50 rpm. Samples of dissolution medium were collected at predetermined intervals and analyzed by online UV spectrophotometry. The combined dissolution profiles of levodopa-carbidopa obtained from UV analysis are shown in FIG. 82.

### Example 76

### Production and In Vitro Dissolution of Immediate Release Levodopa-Carbidopa (4:1) Pellets, Lot # 603-069

Immediate release levodopa-carbidopa pellets (4:1) were prepared in accordance with the method described in Example 51. The weight and composition of pellets are given in Table 14A. Levodopa and carbidopa were blended with inactive excipients for 15 min. The levodopa-carbidopa-excipients blend was then granulated by spraying purified water while mixing at low shear. The granulation was blended for an additional 5 min and then extruded through a 1.0 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 120 min. The dried pellets were screened and particles with diameters ranging from 0.7 mm to 1.4 mm were selected for future experimentation.

TABLE 14A
Weight and Composition of Immediate Release Levodopa-Carbidopa
Pellets, Lot # 603-069

| Ingredients | Weight % | Weight (g) |
|---|---|---|
| Levodopa, USP | 49.44 | 75.00 |
| Carbidopa monohydrate, USP | 13.49 | 20.46 |
| Microcrystalline cellulose | 14.83 | 22.50 |
| (EMCOCEL ® 90 M), NF | | |
| Croscarmellose sodium (AC-DI-SOL ®), NF | 9.88 | 15.00 |
| Anhydrous citric acid, USP | 7.91 | 12.00 |
| Polyethylene oxide (POLYOX ™ WSR N10), NF | 4.35 | 6.60 |
| Butylated hydroxytoluene (BHT), NF | 0.10 | 0.15 |
| Total | 100.00 | 151.71 |

Aliquots of screened pellets were encapsulated in 00-size hard gelatin capsules.

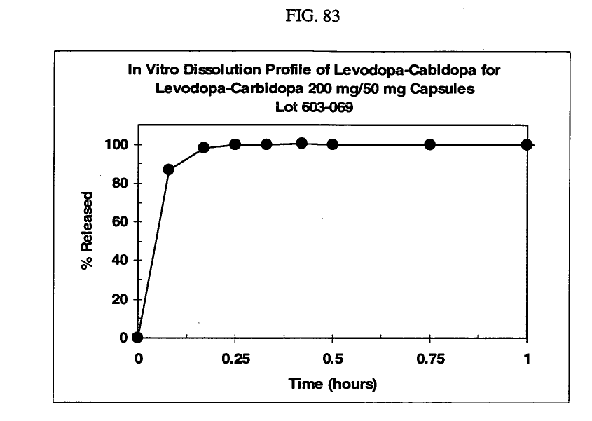

The in vitro dissolution profile of levodopa-carbidopa capsules, containing 200 mg levodopa and 50 mg carbidopa anhydrous was obtained under simulated gastric conditions. The dissolution test was performed in 900 mL of 0.1 N HCl-pH 1.2 solution in a USP II apparatus at a temperature of 37° C. The paddle speed was set at 50 rpm. Samples of dissolution medium were collected at predetermined intervals and analyzed by online UV spectrophotometry. The combined dissolution profile of levodopa-carbidopa obtained from UV analysis is shown in FIG. 83.

### Example 77

### Production and In Vitro Dissolution of Immediate Release Levodopa-Carbidopa (10:1) Pellets, Lot # 602-033

Immediate release levodopa-carbidopa pellets (10:1) were prepared in accordance with the method described in Example 51. The weight and composition of pellets are given in Table 15A. Levodopa and carbidopa were blended with inactive excipients for 15 min. The levodopa-carbidopa-excipients blend was then granulated by spraying purified water while mixing at low shear. The granulation was blended for an additional 5 min and then extruded through a 1.5 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 60 min. The dried pellets were screened and particles with diameters ranging from 1.0 mm to 2.0 mm were selected for future experimentation.

TABLE 15A
Weight and Composition of Immediate Release Levodopa-Carbidopa
Pellets, Lot # 602-033

| Ingredients | Weight % | Weight (g) |
|---|---|---|
| Levodopa, USP | 54.73 | 82.50 |
| Carbidopa monohydrate, USP | 5.97 | 9.00 |
| Microcrystalline cellulose (EMCOCEL ® 90 M), | 14.92 | 22.50 |
| L-Glutamic acid, FCC | 9.95 | 15.00 |
| Croscarmellose sodium (AC-DI-SOL ®), NF | 9.95 | 15.00 |
| Polyethylene oxide (POLYOX ™ WSR N10), NF | 4.38 | 6.60 |
| Butylated hydroxytoluene (BHT), NF | 0.10 | 0.15 |
| Total | 100.00 | 150.75 |

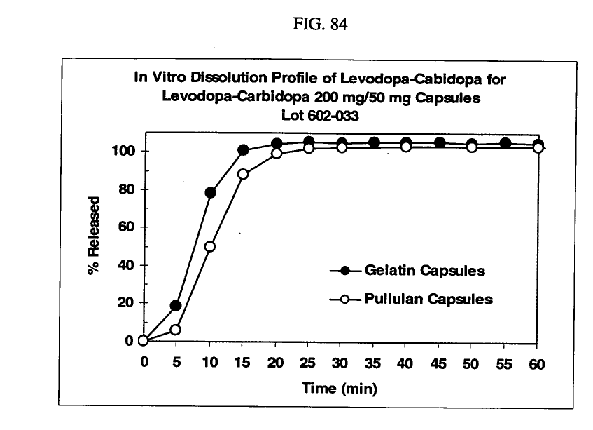

Aliquots of screened pellets were encapsulated in 000-size hard gelatin capsules. The in vitro dissolution profile of levodopa-carbidopa capsules, containing 200 mg levodopa and 50 mg carbidopa anhydrous was obtained under simulated gastric conditions. The dissolution test was performed in 900 mL of 0.1 N HCl-pH 1.2 solution in a USP II apparatus at a temperature of 37° C. The paddle speed was set at 50 rpm. Samples of dissolution medium were collected at predetermined intervals and analyzed by online UV spectrophotometry. The combined dissolution profile of levodopa-carbidopa obtained from UV analysis is shown in FIG. 84.

### Example 78

### Production and In Vitro Dissolution of Levodopa Pellets Prepared with Granulation-Extrusion-Spheronization and Low Concentration of Microcrystalline Cellulose, Lot # 510-048

Levodopa pellets were prepared in accordance with the method described in Example 51. The weight and composition of pellets are given in Table 16A. Levodopa was blended with inactive excipients for 5 min. The levodopa-excipients blend was then granulated by spraying purified water while mixing at low shear. The granulation was extruded through a 1.5 mm screen of a Caleva extruder, model 25, operating at 15 rpm. The extrudate was spheronized in a Caleva spheronizer, model 250, operating at 1000 rpm for 5 min. The spheronized pellets were dried in a Precision oven at 50° C. overnight. The dried pellets were screened and particles with diameters ranging from 1.0 mm to 2.0 mm were selected for future experimentation.

TABLE 16A
Weight and Composition of Levodopa Pellets, Lot # 510-048

| Ingredients | Weight % | Weight (g) |
|---|---|---|
| Mannitol (MANNOGEM ™ Powdered), USP | 59.0 | 118 |
| Levodopa, USP | 30.0 | 60 |
| Microcrystalline cellulose | 10.0 | 20 |
| (EMCOCEL ® 90 M), NF | | |
| Anhydrous citric acid, USP | 1.0 | 2 |
| Total | 100.0 | 200 |

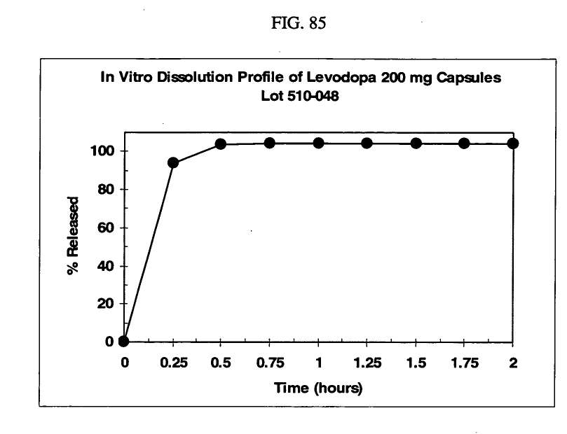

Aliquots of screened pellets were encapsulated in 000-size hard gelatin capsules. The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 85.

### Example 79

### Production and In Vitro Dissolution of Levodopa Pellets Prepared with Granulation-Extrusion-Spheronization and Spheromer™ III, Lot # 511-045

Levodopa pellets were prepared in accordance with the method described in Example 51. The weight and composition of pellets are given in Table 17A. Levodopa was blended with inactive excipients for 5 min. The levodopa-excipients blend was then granulated by spraying purified water while mixing at low shear. The granulation was extruded through a 1.5 mm screen of a Caleva extruder, model 25, operating at 15 rpm. The extrudate was spheronized in a Caleva spheronizer, model 250, operating at 1250 rpm for 5 min. The spheronized pellets were dried in a Precision oven at 50° C. overnight. The dried pellets were screened and particles with diameters ranging from 1.0 mm to 2.0 mm were selected for future experimentation.

TABLE 17A
Weight and Composition of Levodopa Pellets, Lot # 511-045

| Ingredients | Weight % | Weight (g) |
|---|---|---|
| Levodopa, USP | 40.0 | 60.0 |
| SPHEROMER ™ III | 30.0 | 45.0 |
| Microcrystalline cellulose | 28.0 | 42.0 |
| (EMCOCEL ® 90 M), NF | | |
| Anhydrous citric acid, USP | 2.0 | 3.0 |
| Total | 100.0 | 150.0 |

Aliquots of screened pellets were encapsulated in 00-size hard gelatin capsules.

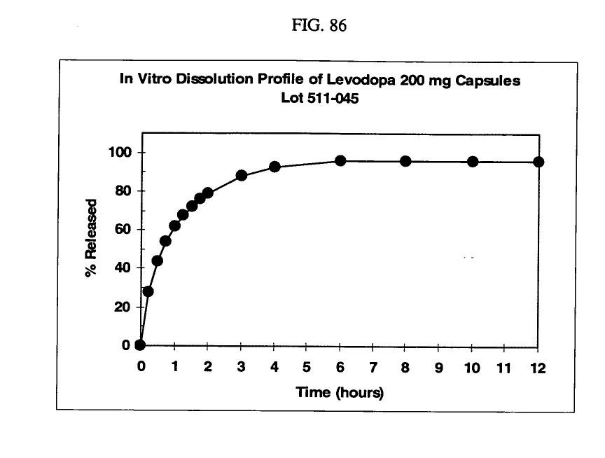

The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 86.

### Example 80

### Production and In Vitro Dissolution of Levodopa Pellets Prepared with Granulation-Extrusion-Spheronization and Spheromer™ III, Lot # 512-085

Levodopa pellets were prepared in accordance with the method described in Example 51. The weight and composition of pellets are given in Table 18A. Levodopa was blended with inactive excipients for 5 min. The levodopa-excipients blend was then granulated by spraying a 5% (w/v) calcium chloride solution in water while mixing at low shear. About two-third of the granulation was extruded through a 1.5 mm screen of a Caleva extruder, model 25, operating at 15 rpm. The remaining part of the granulation was extruded through a 2.0 mm screen. The extrudate was spheronized in a Caleva spheronizer, model 250, operating at 1250 rpm for 5 min. The spheronized pellets were dried in a Precision oven at 50° C. overnight. The dried pellets were screened and particles with diameters ranging from 1.0 mm to 2.0 mm were selected for future experimentation.

TABLE 18A
Weight and Composition of Levodopa Pellets, Lot # 512-085

| Ingredients | Weight % | Weight (g) |
|---|---|---|
| Levodopa, USP | 38.86 | 60.0 |
| SPHEROMER ™ III | 29.15 | 45.0 |
| Microcrystalline cellulose (EMCOCEL ® 90 M), | 27.20 | 42.0 |
| Calcium chloride, anhydrous, FCC | 2.85 | 4.4 |
| Anhydrous Citric Acid, USP | 1.94 | 3.0 |
| Total | 100.00 | 154.4 |

Aliquots of screened pellets were encapsulated in 00-size hard gelatin capsules.

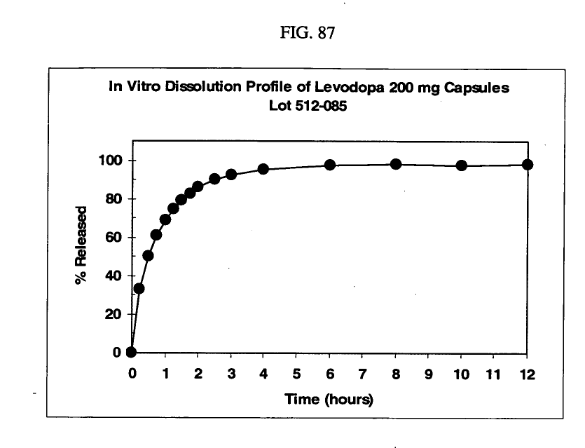

The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 87.

### Example 81

### Production and In Vitro Dissolution of Levodopa Pellets Prepared with Granulation-Extrusion-Spheronization and Glyceryl Monostearate, Lot # 601-002

Levodopa pellets were prepared in accordance with the method described in Example 51. The weight and composition of pellets are given in Table 19A. Levodopa was blended with inactive excipients for 5 min. The levodopa-excipients blend was then granulated by spraying purified water while mixing at low shear. The granulation was extruded through a 1.0 mm screen of a Caleva extruder, model 25, operating at 15 rpm. The extrudate was spheronized in a Caleva spheronizer, model 250, operating at 1000 rpm for 2.5 min, 1250 rpm for 1.5 min, and 1450 rpm for 1 min. The spheronized pellets were dried in a Precision oven at 50° C. overnight. The dried pellets were screened and particles with diameters ranging from 1.0 mm to 2.0 mm were selected for future experimentation.

TABLE 19A
Weight and Composition of Levodopa Pellets, Lot # 601-002

| Ingredients | Weight % | Weight (g) |
|---|---|---|
| Levodopa, USP | 50.0 | 75.0 |
| Glyceryl monostearate, Powder, Food Grade | 40.0 | 60.0 |
| Anhydrous citric acid, USP | 10.0 | 15.0 |
| Total | 100.0 | 150.0 |

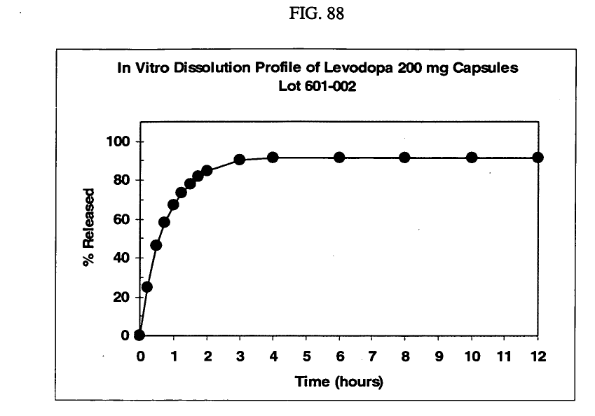

Aliquots of screened pellets were encapsulated in 00-size hard gelatin capsules. The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 88.

### Example 82

### Production and In Vitro Dissolution of Immediate Release Carbidopa Pellets, Lot # 601-012

Immediate release carbidopa pellets were prepared in accordance with the method described in Example 51. The weight and composition of pellets are given in Table 20A. Carbidopa was blended with inactive excipients for 10 min. The carbidopa-excipients blend was then granulated by spraying absolute ethanol while mixing at low shear. The granulation was blended for an additional 5 min and then extruded through a 1.5 mm screen of a Caleva extruder, model 25, operating at 15 rpm. The extrudate was spheronized in a Caleva spheronizer, model 250, operating at 1000 rpm for 5 min. The spheronized pellets were dried in a Precision oven at 50° C. for 60 min. The dried pellets were screened and particles with diameters ranging from 1.0 mm to 2.0 mm were selected for future experimentation.

TABLE 20A
Weight and Composition of Immediate Release
Carbidopa Pellets, Lot # 601-012

| Ingredients | Weight % | Weight (g) |
|---|---|---|
| Carbidopa monohydrate, USP | 51.92 | 81.00 |
| Microcrystalline cellulose (EMCOCEL ® 90 M), | 19.23 | 30.00 |
| NF | | |
| Anhydrous citric acid, USP | 19.23 | 30.00 |
| Croscarmellose sodium (AC-DI-SOL ®), NF | 5.77 | 9.00 |
| Hydroxypropylcellulose (KLUCEL ® | 2.89 | 4.50 |
| EXF Pharm), NF | | |
| Ethylenediamine tetracetic acid | 0.48 | 0.75 |
| Sodium meta-bisulfite | 0.48 | 0.75 |
| Total | 100.00 | 156.00 |

Aliquots of screened pellets were encapsulated in 00-size hard gelatin capsules.

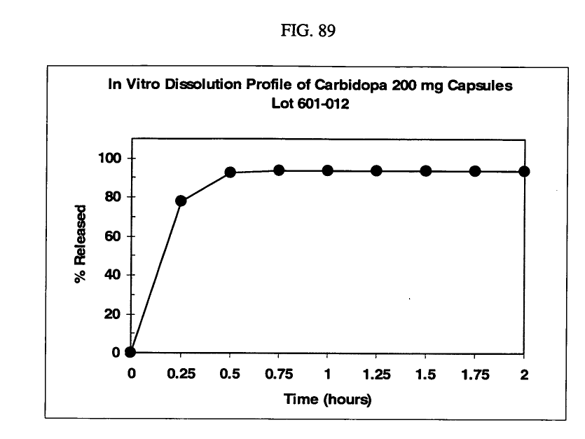

The in vitro dissolution profile of carbidopa capsules, containing 200 mg carbidopa anhydrous was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of carbidopa obtained from UV analysis is shown in FIG. 89.

### Example 83

### Production and In Vitro Dissolution of Levodopa Pellets Prepared with Granulation-Extrusion-Spheronization and Low Concentration of Microcrystalline Cellulose, Lot # 602-001

Levodopa pellets were prepared in accordance with the method described in Example 51. The weight and composition of pellets are given in Table 21A. Levodopa was blended with inactive excipients for 10 min. The levodopa-excipients blend was then granulated by spraying purified water while mixing at low shear. The granulation was extruded through a 1.5 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 60 min. The dried pellets were screened and particles with diameters ranging from 1.0 mm to 2.0 mm were selected for future experimentation.

TABLE 21A
Weight and Composition of Levodopa Pellets, Lot # 602-001

| Ingredients | Weight % | Weight (g) |
|---|---|---|
| Levodopa, USP | 78.95 | 120.0 |
| L-Glutamic acid hydrochloride, FCC | 9.87 | 15.0 |
| Microcrystalline cellulose (EMCOCEL ® 90 M), | 6.25 | 9.5 |
| NF | | |
| Hydroxypropylcellulose (L-HPC LH-31) | 4.93 | 7.5 |
| Total | 100.0 | 152.0 |

Aliquots of screened pellets were encapsulated in 00-size hard gelatin capsules.

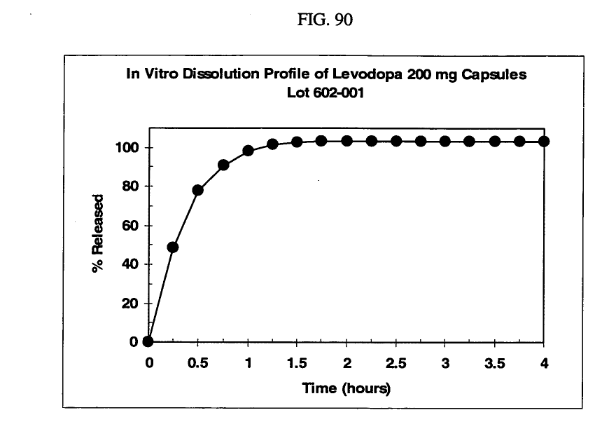

The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 90.

### Example 84

### Production and In Vitro Dissolution of Immediate Release Levodopa Pellets Prepared with Granulation-Extrusion-Spheronization and Low Concentration of Microcrystalline Cellulose, Lot # 602-024

Levodopa pellets were prepared in accordance with the method described in Example 51. The weight and composition of pellets are given in Table 22A. Levodopa was blended with inactive excipients for 10 min. The levodopa-excipients blend was then granulated by spraying purified water while mixing at low shear. The granulation was extruded through a 1.5 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 150 min. The dried pellets were screened and particles with diameters ranging from 1.0 mm to 2.0 mm were selected for future experimentation.

TABLE 22A
Weight and Composition of Levodopa Pellets, Lot # 602-024

| Ingredients | Weight % | Weight (g) |
|---|---|---|
| Levodopa, USP | 54.28 | 82.5 |
| Co-processed Starch (STARCAP 1500 ™)* | 24.67 | 37.5 |
| Microcrystalline cellulose (EMCOCEL ® 90 M), | 11.18 | 17.0 |
| NF | | |
| L-Glutamic acid hydrochloride, FCC | 9.87 | 15.0 |
| Total | 100.0 | 152.0 |

*STARCAP 1500 ™ is a co-processed mixture of corn starch and pregelatinized starch.

Aliquots of screened pellets were encapsulated in 00-size hard gelatin capsules.

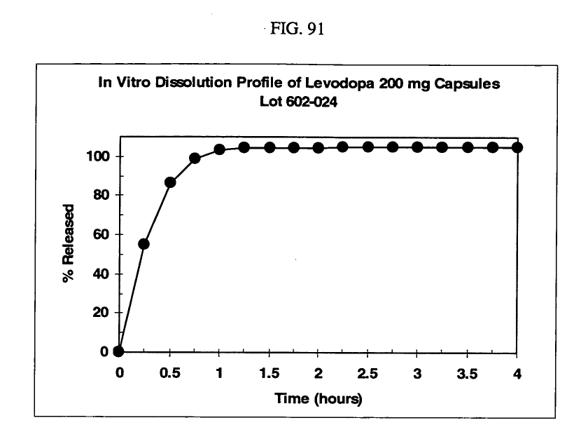

The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 91.

### Example 85

### Production and In Vitro Dissolution of Immediate Release Levodopa Pellets Prepared with Granulation-Extrusion-Spheronization and Co-Processed Starch, Lot # 602-028

Levodopa pellets were prepared in accordance with the method described in Example 51. The weight and composition of pellets are given in Table 23A. Levodopa was blended with inactive excipients for 10 min. The levodopa-excipients blend was then granulated by spraying purified water while mixing at low shear. The granulation was extruded through a 1.5 mm screen of a Caleva extruder, model 25, operating at 10 rpm. The extrudate was spheronized in a Caleva spheronizer, model 250, operating at 1000 rpm for 5 min. The spheronized pellets were dried in a Vector MFL.01 Micro Batch Fluid Bed System at 50° C. for 95 min. The dried pellets were screened and particles with diameters ranging from 1.0 mm to 2.0 mm were selected for future experimentation.

TABLE 23A
Weight and Composition of Levodopa Pellets, Lot # 602-024

| Ingredients | Weight % | Weight (g) |
|---|---|---|
| Levodopa, USP | 53.22 | 82.5 |
| Co-processed Starch (STARCAP 1500 ™) | 37.10 | 57.5 |
| L-Glutamic acid hydrochloride, FCC | 9.68 | 15.0 |
| Total | 100.0 | 155.0 |

*STARCAP 1500 ™ is a co-processed mixture of corn starch and pregelatinized starch.

Aliquots of screened pellets were encapsulated in 00-size hard gelatin capsules.

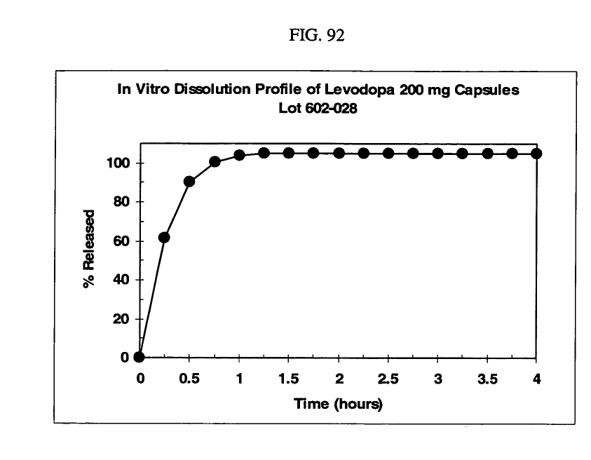

The in vitro dissolution profile of levodopa capsules, containing 200 mg levodopa was obtained under simulated gastric conditions. The dissolution test was performed in 900 mL of 0.1N HCl-pH 1.2 solution in a USP II apparatus at a temperature of 37° C. The paddle speed was set at 50 rpm. Samples of dissolution medium were collected at predetermined intervals and analyzed by online UV spectrophotometry. The dissolution profile of levodopa obtained from UV analysis is shown in FIG. 92.

### Example 86

### Preparation of Pramipexole Extended-Release Pellet Formulation, Lot # 601-048

An extended-release pellet formulation of pramipexole was prepared to be combined with an immediate- and controlled-release multiparticulate formulation of levodopa-carbidopa. Pramipexole was initially layered on placebo core pellets (1.1-1.4 mm dia.) with OPADRY® Clear as a binder using a Vector MFL.01 Micro Batch Fluid Bed System, equipped with a Wurster insert.

The placebo core pellets were prepared using low-shear granulation, extrusion and spheronization technique. Table 24A provides the weight and composition of placebo core pellets.

TABLE 24A
Weight and Composition of Placebo Core Pellets

| Ingredients | Weight (%) | Weight (g) |
|---|---|---|
| Microcrystalline Cellulose (Emcocel ® 90M), | 30.0 | 60.0 |
| NF | | |
| Mannitol (Mannogem ™ Powdered), USP | 65.0 | 130.0 |
| Hydroxypropylcellulose (HPC-SSL), NF | 5.0 | 10.0 |
| Purified Water, USP | * | * |
| Total | 100.0 | 200.0 |

*Evaporated during drying process.

The placebo pellets were dried in an oven at 50° C. to achieve a desired moisture level of 1% (w/w). These pellets were then screened through size 10, 12, 14, 16 and 18 mesh sieves and the particles retained on screen size 14 and 16 were used for subsequent pramipexole layering process.

Pramipexole layered pellets were subsequently coated in the Vector MFL.01 Micro Batch Fluid Bed System, equipped with a Wurster insert, with a release rate-controlling polymer composition containing ethylcellulose to achieve a weight gain of 8.3% (w/w), and then coated with bioadhesive SPHEROMER™ III polymer to a weight gain of 5.3% (w/w).

The unit dose composition of a pramipexole 0.375 mg extended-release pellet formulation is given in Table 25A.

TABLE 25A
Unit Dose Composition of Pramipexole 0.375 mg
Extended-Release Pellet Formulation

| | Weight | Weight |
|---|---|---|
| Components | (%) | (mg) |
| Pramipexole Dihydrochloride Monohydrate, USP | 0.33 | 0.375 |
| Mannitol (MANNOGEM ™ Powdered), USP | 53.44 | 60.93 |
| Microcrystalline Cellulose (EMCOCEL ® 90M), NF | 24.67 | 28.13 |
| Ethylcellulose (ETHOCEL ™ Std 10 FP Premium), | 7.26 | 8.28 |
| NF | | |
| OPADRY ® Clear (YS-1-19025-A) | 4.94 | 5.63 |
| SPHEROMER ™ III | 4.78 | 5.45 |
| Hydroxypropyl Cellulose (HPC-SSL), NF | 4.11 | 4.69 |
| Poloxamer 188 (LUTROL ® F 68), NF | 0.25 | 0.29 |
| Dibutyl Sebacate, NF | 0.22 | 0.25 |
| Total | 100.00 | 114.025 |

The bioadhesive pramipexole pellets may be optionally top-coated with bioadhesive SPHEROMER™ I polymer, a hyprolmellose polymer, a hydroxypropylcellulose polymer, or a polyvinyl alcohol polymer to a weight gain of 2-5% (w/w).

### Example 87

### Preparation of Combined Pramipexole 0.375 mg Extended-Release Pellets and Levodopa-Carbidopa 200 mg/50 mg Immediate/Controlled-Release Multiparticulates as a Delayed-Release Capsule Formulation

Pramipexole extended-release pellets, lot # 601-048 (from Example 86), containing 0.375 mg pramipexole, and levodopa-carbidopa immediate/controlled-release multiparticulates, lot # 510-099 (from Example 71), containing 200 mg levodopa and 50 mg carbidopa, were co-encapsulated in two-piece hard gelatin capsules. These capsules were sealed at the junction of cap and body using an aqueous gelatin solution and then coated with 1.6% (w/w) OPADRY® Clear (YS-1-19025-A). The Opadry-coated capsules were top-coated with an enteric coating composition, ACRYL-EZE™ White, in a pan coater (O'Hara Technologies Labcoat System). The capsules were sprayed with a 10% (w/v) solution of ACRYL-EZE™ White in ethanol and water mixture (90:10 v/v) so as to achieve a final weight gain of 5-12% (w/w).

The bioadhesive pramipexole and levodopa-carbidopa pellets may be optionally top-coated with bioadhesive SPHEROMER™ I polymer, a hyprolmellose polymer, a hydroxy-propylcellulose polymer, or a polyvinyl alcohol polymer to a weight gain of 2-5% (w/w).

### Example 88

### Preparation of Pramipexole 0.375 mg Delayed/Extended-Release Capsule Formulation, Lot # 601-056

Pramipexole extended-release pellets, lot # 601-048 (from Example 86), containing 0.375 mg pramipexole were encapsulated in a size 2 hard shell gelatin capsule. These capsules were sealed at the junction of cap and body using an aqueous gelatin solution and coated with 1.6% OPADRY® Clear (YS-1-19025-A). The OPADRY-coated capsules were then coated with an enteric coating composition, ACRYL-EZE™ White, in a pan coater (O'Hara Technologies Labcoat System). The capsules were sprayed with a 10% (w/v) solution of ACRYL-EZE™ White in ethanol and water mixture (90:10 v/v) so as to achieve a final weight gain of 5-12% (w/w).

The unit dose composition of a pramipexole 0.375 mg delayed/extended-release capsule formulation is given in Table 26A.

TABLE 26A
Unit Dose Composition of Pramipexole 0.375 mg
Delayed/Extended-Release Capsule Formulation

| | Weight | Weight |
|---|---|---|
| Components | (%) | (mg) |
| Pramipexole Dihydrochloride Monohydrate, USP | 0.13 | 0.375 |
| Mannitol (MANNOGEM ™ Powdered), USP | 21.45 | 60.93 |
| Microcrystalline Cellulose (EMCOCEL ® 90M), NF | 9.90 | 28.13 |
| ACRYL-EZE ™ White (93O18509) | 8.14 | 23.12 |
| OPADRY ® Clear (YS-1-19025-A) | 3.13 | 8.90 |
| Ethylcellulose (ETHOCEL ™ Std 10 FP Premium), | 2.91 | 8.28 |
| NF | | |
| SPHEROMER ™ III | 1.81 | 5.14 |
| Hydroxypropyl Cellulose (HPC-SSL), NF | 1.65 | 4.69 |
| Poloxamer 188 (LUTROL ® F 68), NF | 0.10 | 0.27 |
| Dibutyl Sebacate, NF | 0.09 | 0.25 |
| Gelatin Capsule, Size 2 | 50.69 | 144.00 |
| Total | 100.00 | 284.085 |

### Example 89

### Preparation of Combined Pramipexole 0.375 mg Delayed/Extended-Release Pellets and Levodopa-Carbidopa 200 mg/50 mg Immediate/Controlled-Release Multiparticulates as a Capsule Formulation

Pramipexole delayed/extended-release pellets, lot # 601-056 (from Example 88), containing 0.375 mg pramipexole, and levodopa-carbidopa immediate-controlled-release multiparticulates, lot # 510-099 (from Example 71), containing 200 mg levodopa and 50 mg carbidopa, were co-encapsulated in two-piece hard gelatin capsules.

The bioadhesive pramipexole and levodopa-carbidopa pellets may be optionally top-coated with bioadhesive SPHEROMER™ I polymer, a hyprolmellose polymer, a hydroxy-propylcellulose polymer, or a polyvinyl alcohol polymer to a weight gain of 2-5% (w/w).

A multiparticulate capsule formulation of levodopa-carbidopa 200 mg/50 mg comprising carbidopa granules, levodopa pellets, and bioadhesive SPHEROMER™ IV-coated levodopa-carbidopa pellets was prepared, and its in vivo pharmacokinetic performance was compared with that of a marketed controlled-release formulation, SINEMET® CR 50-200, in fed beagle dogs.

In the following, Examples 90 to 94 describe the methods of preparation of levodopa-carbidopa multiparticulate capsules. Examples 95 and 96 present the in vitro dissolution and in vivo pharmacokinetic performance of levodopa-carbidopa multiparticulate capsules and SINEMET® CR 50-200 tablets, respectively.

### Example 90

### Production of Carbidopa Granules with Low Shear Granulation, Lot # 508-081

Carbidopa granules were produced with low shear granulation method comprising the following processes:
(1) Weighing carbidopa and LUDIPRESS®, a co-processed mixture of povidone (3.5%), crospovidone (3.5%), and lactose monohydrate (93.0%).
(2) Blending carbidopa and LUDIPRESS® from step (1) in a planetary type mixer, Hobart Mixer with a 5-qt mixing bowl, operating at the speed setting #1, for 5 min, forming a dry mix.
(3) Granulating the dry mix from step (2) under low shear with a 5% (w/v) aqueous solution of povidone, forming a wet granulation.
(4) Drying the granulation from step (3) to the moisture content of 0.8% in a conventional Precision oven at 50° C.
(5) Screening and classifying the dried granules from step (4) through a stack of stainless steel sieves, U.S. standard mesh sizes 20 and 60, using a mechanical sieve shaker, W.S. Tyler Sieve Shaker Ro-Tap Rx-29, operated for 5 min. Particle size and distribution of granular formulations were analyzed, and classified granules ranging from 0.25 mm (mesh # 60) to 0.85 mm (mesh # 20) were selected for future experimentation.

The dry weight and composition of granules are given in Table 90-1.

TABLE 90-1
Dry Weight and Composition of Carbidopa Granules, Lot # 508-081

| Ingredients | Weight % | Weight (g) |
|---|---|---|
| Carbidopa monohydrate, USP | 24.7 | 50.0 |
| LUDIPRESS | | |
| Povidone, USP | 2.6 | 5.3 |
| Crospovidone, USP | 2.6 | 5.3 |
| Lactose Monohydrate, Ph Eur | 68.9 | 139.4 |
| Povidone (PLASDONE ® K-25), USP | 1.2 | 2.4 |
| Total | 100.0 | 202.4 |

### Example 91

### Production of Levodopa Pellets with Granulation-Extrusion-Spheronization, Lot #509-053

Levodopa pellets were produced with granulation-extrusion-spheronization method comprising the following processes:
(1) Weighing levodopa and pharmaceutically acceptable excipients.
(2) Blending levodopa and excipients from step (1) in a planetary type mixer, Hobart Mixer with a 5-qt mixing bowl, operating at the speed setting #1, for 5 min, forming a dry mix.
(3) Granulating the dry mix from step (2) under low shear with purified water, forming a wet granulation.
(4) Extruding the wet granulation from step (3) through the screen of a screen-type extruder, Caleva Model 25 Extruder, operating at 15 rpm, and forming breakable wet strands, the extrudate. The screen aperture was 1.5 mm.
(5) Spheronizing the extrudate from step (4) in a spheronizer, Caleva Model 250, equipped with a 2.5-mm spheronization plate, operating at 1250 rpm for 5 min, and forming spheronized pellets.
(6) Drying the spheronized pellets from step (5) to the moisture content of 0.4% in a conventional Precision oven at 50° C.
(7) Screening and classifying the dried pellets from step (6) through a stack of stainless steel sieves, U.S. standard mesh sizes 10, 12, 14, 16, and 18 using a mechanical sieve shaker, W.S. Tyler Sieve Shaker Ro-Tap Rx-29, operated for 5 min. Particle size and distribution of pellet formulations were analyzed, and classified pellets ranging from 1.0 mm (mesh # 18) to 2.0 mm (mesh #0.10) were selected for future experimentation.

The dry weight and composition of pellets are given in Table 91-1.

TABLE 91-1
Dry Weight and Composition of Levodopa Pellets, Lot # 509-053

| Ingredients | Weight % | Weight (g) |
|---|---|---|
| Levodopa, USP | 50.0 | 200.0 |
| Microcrystalline cellulose (EMCOCEL ® 90 M), | 25.0 | 100.0 |
| NF | | |
| Mannitol (MANNOGEM ™ Powdered), USP | 14.0 | 56.0 |
| Hydroxypropylcellulose (HPC-SSL), NF | 5.0 | 20.0 |
| Croscarmellose sodium (AC-DI-SOL ®), NF | 5.0 | 20.0 |
| Citric acid, anhydrous, USP | 1.0 | 4.0 |
| Total | 100.0 | 400.0 |

### Example 92

### Production of Levodopa-Carbidopa (4:1) Pellets with Granulation-Extrusion-Spheronization, Lot # 512-062

Levodopa-carbidopa pellets were produced with granulation-extrusion-spheronization method comprising the following processes:
(1) Weighing levodopa, carbidopa, and pharmaceutically acceptable excipients.
(2) Blending levodopa, carbidopa, and excipients from step (1) in a planetary type mixer, Hobart Mixer with a 5-qt mixing bowl, operating at the speed setting #1, for 5 min, forming a dry mix.
(3) Granulating the dry mix from step (2) under low shear with purified water, forming a wet granulation.
(4) Extruding the wet granulation from step (3) through the screen of a screen-type extruder, Caleva Model 25 Extruder, operating at 15 rpm, and forming breakable wet strands, the extrudate. The screen aperture was 1.5 mm.
(5) Spheronizing the extrudate from step (4) in a spheronizer, Caleva Model 250, equipped with a 2.5-mm spheronization plate, operating at 1000 rpm for 5 min, and forming spheronized pellets.
(6) Drying the spheronized pellets from step (5) to the moisture content of 1% in a conventional Precision oven at 50° C.
(7) Screening and classifying the dried pellets from step (6) through a stack of stainless steel sieves, U.S. standard mesh sizes 10, 12, 14, 16, and 18 using a mechanical sieve shaker, W.S. Tyler Sieve Shaker Ro-Tap Rx-29, operated for 5 min. Particle size and distribution of pellet formulations were analyzed, and classified pellets ranging from 1.0 mm (mesh # 18) to 2.0 mm (mesh # 10) were selected for future experimentation.

The dry weight and composition of pellets are given in Table 92-1.

TABLE 92-1
Dry Weight and Composition of Levodopa-Carbidopa
Pellets, Lot # 512-062

| Ingredients | Weight % | Weight (g) |
|---|---|---|
| Levodopa, USP | 48.3 | 100.0 |
| Carbidopa monohydrate, USP | 13.0 | 27.0 |
| Microcrystalline cellulose (EMCOCEL ® 90 M), | 26.6 | 55.0 |
| NF | | |
| Crospovidone (POLYPLASDONE ® XL), USP | 5.8 | 12.0 |
| Povidone (PLASDONE ® K-25), USP | 3.4 | 7.0 |
| Citric acid, anhydrous, USP | 2.9 | 6.0 |
| Total | 100.0 | 207.0 |

### Example 93

### Film Coating of Levodopa-Carbidopa Pellets with Bioadhesive Polymer, SPHEROMER™ IV, Lot # 601-004

Fifty grams of levodopa-carbidopa pellets, lot # 512-062, were film-coated with bioadhesive SPHEROMER™ IV polymer in a Vector MFL.01 Micro Batch Fluid Bed System, equipped with a Wurster insert. The fluid bed system was operated at inlet air flow rate of 100 lpm (liter per minute) and temperature of 35-40° C. The composition of the coating solution is given in Table 80-1. SPHEROMER™ IV and Poloxamer 188 (LUTROL® F68) were dissolved in a mixture of methyl alcohol and water and sprayed onto the fluidized pellets to obtain a 6% weight gain on pellets.

TABLE 93-1
Composition of Spheromer ™ IV Coating Solution, Lot # 601-004

| Ingredients | Weight % | Weight (g) |
|---|---|---|
| SPHEOROMER ™ IV | 95 | 2.90 |
| Poloxamer 188 (LUTROL ® F68), NF | 5 | 0.15 |
| Methyl alcohol, NF | * | (75 mL) |
| Purified Water, HPLC Grade | * | (25 mL) |
| Total Solids | 100 | 3.05 |

*Methyl alcohol and water were removed during the coating/drying process.

### Example 94

### Preparation of Levodopa-Carbidopa 200 mg/50 mg Multiparticulate Capsules, Lot # 601-038

Carbidopa granules (lot # 508-081), levodopa pellets (lot # 509-053), and SPHEROMER™ IV-coated levodopa-carbidopa pellets (lot # 601-004) were encapsulated in 00-size hard gelatin capsules. Each capsule contained 200 mg levodopa and 50 mg carbidopa anhydrous. The composition of encapsulated multiparticulates is given in Table 94-1.

TABLE 94-1
Composition (mg) of Multiparticulate Capsule Formulations,
Lot # 601-038

| Components | Lot # | Wt. (mg) |
|---|---|---|
| Carbidopa Granules | 508-081 | 40 |
| Levodopa Pellets | 509-053 | 80 |
| SPHEROMER ™ IV-coated Levodopa-Carbidopa | 510-098 | 348 |
| Pellets | | |
| Total (mg per capsule) | — | 468 |

### Example 95

### In Vitro Dissolution and In Vivo Pharmacokinetic Performance of Levodopa-Carbidopa 200 mg/50 mg Multiparticulate Capsules, Lot # 601-038

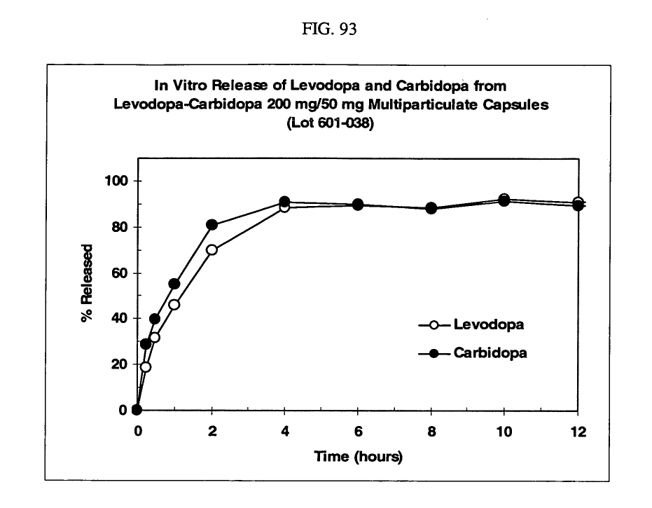

The in vitro dissolution profile of levodopa-carbidopa capsules, containing 200 mg levodopa and 50 mg carbidopa was obtained under simulated gastric conditions. The dissolution tests were performed in 900 mL of 0.1N HCl-pH 1.2 solution in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 93.

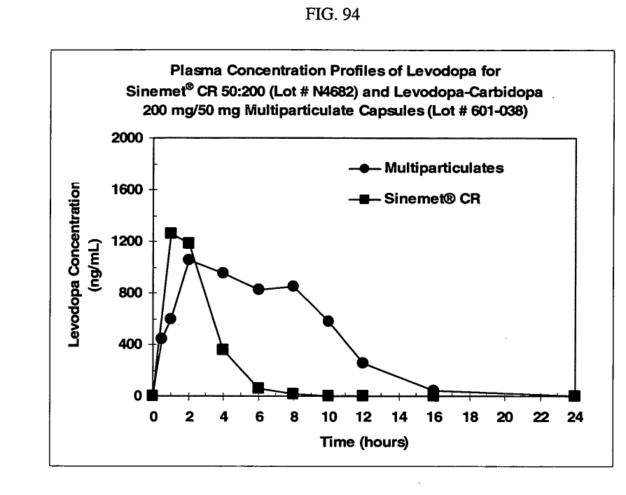

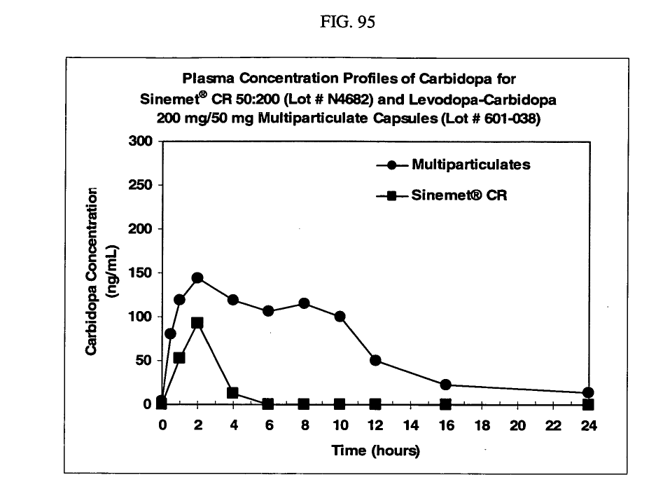

The in vivo performance of levodopa-carbidopa capsules was evaluated in beagle dogs. The capsules were administered to separate cohorts of twelve beagle dogs in the fed state. Plasma levels of levodopa and carbidopa were measured using LC/MS/MS analysis. FIGS. 94 and 95 show the plasma concentration profiles of levodopa and carbidopa in the fed state, respectively. The pharmacokinetic data including the area under the plasma levodopa vs. time curve (AUC), max. concentration (Cₘₐₓ) and time required to achieve Cₘₐₓ (Tₘₐₓ) are provided in Table 95-1.

TABLE 95-1
Pharmacokinetic Data for Levodopa-Carbidopa Multiparticulate
Capsules, Lot # 601-038, in Fed Beagle Dogs; the area under the
plasma levodopa vs. time curve (AUC), maximum concentration
(Cₘₐₓ), and time required to achieve Cₘₐₓ (Tₘₐₓ)

| | Fasting Period | | |
|---|---|---|---|
| | AUC | Cₘₐₓ | Tₘₐₓ |
| | (ng/ml · hr) | (ng/ml) | (hr) |
| Fed State | 9,649 | 1,615 | 3.8 |

### Example 96

### In Vitro Dissolution and In Vivo Pharmacokinetic Performance of SINEMET® CR 50-200 Tablets, containing 50 mg Carbidopa and 200 mg Levodopa, Lot # N4682

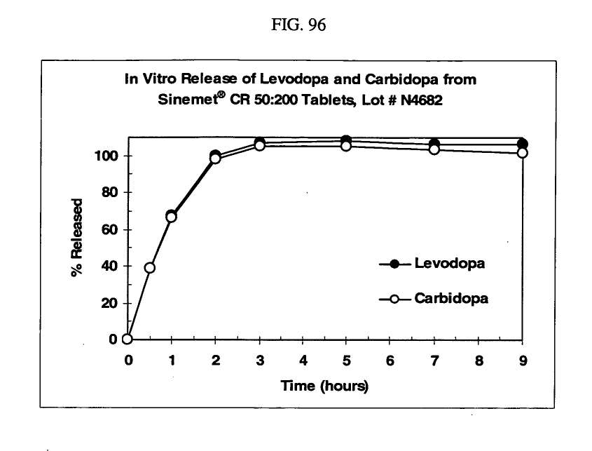

The in vitro dissolution profile of SINEMET® CR 50-200 tablets, containing 50 mg carbidopa and 200 mg levodopa were obtained under simulated gastric conditions. The dissolution tests were performed in 900 mL of 0.1N HCl-pH 1.2 solution, in a USP II apparatus at a temperature of 37° C. The paddle speed was set at 50 rpm. Samples of dissolution media were collected at predetermined intervals and analyzed by HPLC. The dissolution profiles of levodopa and carbidopa obtained from HPLC analysis are shown in FIG. 96.

The in vivo pharmacokinetic performance of SINEMET® CR 50-200 tablets was evaluated in beagle dogs. SINEMET® CR tablets were administered to cohorts of six beagle dogs in the fed state and plasma levels of levodopa and carbidopa were measured using HPLC analysis. FIGS. 94 and 95 show the plasma concentration profiles of levodopa and carbidopa. The pharmacokinetic data including the area under the plasma levodopa vs. time curve (AUC), maximum concentration (Cₘₐₓ) and time required to achieve Cₘₐₓ (Tₘₐₓ) are provided in Table 96-1.

TABLE 96-1
Pharmacokinetic Data for SINEMET ® CR 50-200 Tablets,
Lot # N4682, in Fed Beagle Dogs; the area under the plasma
levodopa vs. time curve (AUC), maximum concentration (Cₘₐₓ),
and time required to achieve Cₘₐₓ (Tₘₐₓ)

| | AUC | Cₘₐₓ | Tₘₐₓ |
|---|---|---|---|
| Formulation | (ng/ml · hr) | (ng/ml) | (hr) |
| SINEMET ® CR 50-200 Tablets | 3,903 | 1,663 | 2 |

### EQUIVALENTS

Those skilled in the art will recognize, or be able to ascertain using no more than routine experimentation, many equivalents to the specific embodiments of the invention described herein. Such equivalents are intended to be encompassed by the following claims.

All patents, publications, and other references cited above are hereby incorporated by reference in their entirety.

1. A pharmaceutical composition for the treatment of a patient suffering from Parkinson's disease and/or another movement disorder, comprising:
(1) a first immediate-release (IR) portion comprising levodopa or a metabolic precursor thereof, formulated to provide a therapeutically effective concentration of levodopa or the precursor in the patient within about 2 hours of administration to the patient; and
(2) a second portion comprising levodopa or a metabolic precursor thereof, formulated to release levodopa or the precursor at a substantially zero-order release rate over a sustained treatment period to maintain the therapeutically effective concentration of levodopa or the precursor in the patient;
wherein at least the first IR portion further comprises a decarboxylase enzyme inhibitor, and the ratio of the inhibitor to levodopa or the precursor in at least the first IR portion is greater than 1:4.

2. The pharmaceutical composition of claim 1, further comprising:
(3) a substantially ascending release portion comprising levodopa or a metabolic precursor thereof, formulated to effect a rapid drop of levodopa concentration in the patient to below the therapeutically effective concentration at the end of the treatment period.

3. The pharmaceutical composition of claim 1, further comprising:
(3) a substantially elevating release portion comprising levodopa or a metabolic precursor thereof, formulated to elevate the substantially zero-order release rate to a higher level beginning around a predetermined time point.

4. The pharmaceutical composition of claim 3, wherein the predetermined time point is about four to seven hours after administration to the patient.

5. The pharmaceutical composition of claim 3, wherein the substantially elevating release portion is formulated to effect a rapid drop of levodopa concentration in the patient to below the therapeutically effective concentration at the end of the treatment period.

6. The pharmaceutical composition of claim 3, wherein the substantially elevating release portion comprises a decarboxylase enzyme inhibitor.

7. A pharmaceutical composition for the treatment of a patient suffering from Parkinson's disease and/or another movement disorder, comprising:
(1) a first immediate-release (IR) portion comprising levodopa or a metabolic precursor thereof, formulated to provide a therapeutically effective concentration of levodopa in the patient within about 2 hours of administration to the patient;
(2) a second portion comprising levodopa or a metabolic precursor thereof, formulated to release levodopa or the precursor at a substantially zero-order release rate over a sustained treatment period to maintain the therapeutically effective concentration of levodopa in the patient; and
(3) a substantially ascending release portion comprising levodopa or a metabolic precursor thereof, formulated to effect a rapid drop of levodopa concentration in the patient to below the therapeutically effective concentration at the end of the treatment period.

8. A pharmaceutical composition for the treatment of a patient suffering from Parkinson's disease and/or another movement disorder, comprising:
(1) a first immediate-release (IR) portion comprising levodopa or a metabolic precursor thereof, formulated to provide a therapeutically effective concentration of levodopa in the patient within about 2 hours of administration to the patient;
(2) a second portion comprising levodopa or a metabolic precursor thereof, formulated to release levodopa or the precursor at a substantially zero-order release rate over a sustained treatment period to maintain the therapeutically effective concentration of levodopa in the patient; and
(3) a substantially elevating release portion comprising levodopa or a metabolic precursor thereof, formulated to elevate the substantially zero-order release rate to a higher level beginning at a predetermined time point.

9. A pharmaceutical composition for the treatment of a patient suffering from Parkinson's disease and/or another movement disorder, comprising:
(1) a sleep-inducing agent; and,
(2) a decarboxylase enzyme inhibitor formulated to provide an effective plasma concentration at a predetermined time after the administration of the pharmaceutical composition to the patient.

10. The pharmaceutical composition of claim 9, further comprising:
(3) a first delayed immediate-release (DIR) portion comprising levodopa or a metabolic precursor thereof, formulated to provide a therapeutically effective concentration of levodopa in the patient within about 2 hours of the predetermined time.

11. The pharmaceutical composition of claim 10, further comprising:
(4) a second delayed controlled release (DCR) portion comprising levodopa or a metabolic precursor thereof, formulated to release levodopa or the precursor at a substantially zero-order release rate over a sustained treatment period after the predetermined time, to maintain the therapeutically effective concentration of levodopa in the patient.

12. The pharmaceutical composition of claim 10, wherein the predetermined time after administration is 6 to 9 hours after administration.

13. The pharmaceutical composition of claim 2, 5, or 7, where the rapid drop of levodopa takes place in less than two hours.

14. The pharmaceutical composition of any of claims 1-13, wherein the formulation further comprises one or more of: a dopaminergic and anti-cholinergic agent selected from: amantadine; an anti-cholinergic agent selected from: trihexyphenidyl, benztropine, ethoproprazine, or procyclidine; a dopamine agonist selected from: apomorphine, bromocriptine, cabergoline, lisuride, pergolide, pramipexole, or ropinirole; a MAO-B (monoamine oxidase B) inhibitor selected from: selegiline or deprenyl; a COMT inhibitor selected from: CGP-28014, entacapone, or tolcapone; a muscle relaxant baclofen; a sedative Clonazepam; an anticonvulsant agent carbamazepine; a dopamine reuptake inhibitor tetrabenazine; a dopamine blocker haloperidol; a β-blocker selected from: propranolol; a carbonic anhydrase inhibitor selected from: acetalzolamide or methazolamide; a narcotic agent codeine; a GABAergic agent gabapentin; or an alpha antagonist clonidine.

15. The pharmaceutical composition of any of claims 1-14, wherein the formulation further comprises a stool softener selected from: bran or psyllium, methylcellulose, polycarbophil, docusate, docusate sodium and casanthranol combination, magnesium hydroxide, magnesium citrate, sorbitol, polyethylene glycol solution, lactulose, lubiprostone or other osmotic or stimulant laxatives, and a natural stool softener.

16. The pharmaceutical composition of any of claims 1-15, wherein the metabolic precursor is a methyl, ethyl, or propyl ester of levodopa, or a combination thereof.

17. The pharmaceutical composition of any of claims 1-15, wherein the metabolic precursor is (−)-L-α-amino-β-(3,4-dihydroxybenzene)propanoic acid, 3-hydroxy-L-tyrosine ethyl ester, phenylglycine, or a mixture thereof.

18. The pharmaceutical composition of any of claims 1-17, wherein the ratio of the decarboxylase inhibitor to levodopa or the precursor in the first IR portion is about 1:3 or greater.

19. The pharmaceutical composition of any of claims 1-17, wherein the ratio of the decarboxylase inhibitor to levodopa or its precursor varies between the start and the end of the release of the second portion.

20. The pharmaceutical composition of claim 19, wherein the ratio changes substantially continuously over the release period of the second portion.

21. The pharmaceutical composition of claim 19, wherein the ratio is substantially constant during all or a part of the release period of the second portion.

22. The pharmaceutical composition of claim 2, 3, 7, or 8, wherein the substantially ascending release portion or the substantially elevating release portion comprises a decarboxylase enzyme inhibitor.

23. The pharmaceutical composition of claim 2, 3, 7, or 8, wherein the substantially ascending release portion or the substantially elevating release portion is a second immediate release portion.

24. The pharmaceutical composition of any of claims 2-23, wherein the ratio of the inhibitor to levodopa or the precursor in the substantially ascending release portion or the substantially elevating release portion is less than 1:4.

25. The pharmaceutical composition of any of the preceding claims, wherein the decarboxylase enzyme inhibitor is carbidopa, a carbidopa prodrug, benserazide, methylphenidate, or a combination thereof.

26. The pharmaceutical composition of any of the preceding claims, wherein the total dose of the decarboxylase enzyme inhibitor per day per human patient is in the range of about 75-600 mg.

27. The pharmaceutical composition of any of the preceding claims, wherein the total dose of levodopa or metabolic precursor thereof per day per human patient is between about 50 mg and about 300 mg.

28. The pharmaceutical composition of any of the preceding claims, wherein at least one of the first IR portion, the second substantially zero order release portion, the substantially elevating release portion, and/or the substantially ascending release portion further comprises at least one dopamine transport inhibitor.

29. The pharmaceutical composition of claim 28, wherein the dopamine transport inhibitor is methylphenidate.

30. The pharmaceutical composition of claim 28, wherein the dopamine transport inhibitor is present in an amount of about 3 mg to about 60 mg.

31. The pharmaceutical composition of claim 28, wherein the dopamine transport inhibitor is released starting after a delay of about 2 hours to about 7 hours.

32. The pharmaceutical composition of claim 31, wherein the dopamine transport inhibitor is released over a period of time of about 1 hour to about 6 hours.

33. The pharmaceutical composition of any one of the claims 1-32, wherein the first IR portion, the second substantially zero order release portion, the substantially elevating release portion (if present), and the substantially ascending release portion (if present), are formulated to provide a sustained dose over at least 4 hours when administered to the patient.

34. The pharmaceutical composition of claim 33, wherein the first IR portion, the second substantially zero order release portion, the substantially elevating release portion, and the substantially ascending release portion (if present), are formulated to provide a sustained dose over at least 8 hours when administered to the patient.

35. The pharmaceutical composition of claim 34, wherein the first IR portion, the second substantially zero order release portion, the substantially elevating release portion (if present), and the substantially ascending release portion (if present), are formulated to provide a sustained dose over at least 12 hours when administered to the patient.

36. The pharmaceutical composition of claim 35, wherein the first IR portion, the second substantially zero order release portion, the substantially elevating release portion (if present), and the substantially ascending release portion (if present), are formulated to provide a sustained dose over at least 14 hours when administered to the patient.

37. The pharmaceutical composition of any one of the claims 1-36, wherein the first IR portion, the second substantially zero order release portion, the substantially elevating release portion (if present), and the substantially ascending release portion (if present), are formulated into a stack of compressed inserts encased inside a shell, each portion having an independent dissolution profile, wherein drug is released only from an exposed surface at a predetermined face of the stack.

38. The pharmaceutical composition of any one of the claims 1-36, wherein the first IR portion, the second substantially zero order release portion, the substantially elevating release portion (if present), and the substantially ascending release portion (if present), are each formulated as a plurality of individual beads or pellets, each of the portions having an independent dissolution profile.

39. The pharmaceutical composition of claim 38, wherein the ratio of beads corresponding to the first IR portion, the second substantially zero order release portion, and the substantially ascending release portion (if present), are customized for the patient to provide a predetermined release profile selected from: a predetermined duration of release, a predetermined rate of reaching a therapeutic plasma concentration of levodopa, or a predetermined maximum release rate customized for the size, sensitivity, or clearance rate of the particular patient.

40. The pharmaceutical composition of claim 39, wherein some or all of the beads are fully or partially coated by a bioadhesive material.

41. The pharmaceutical composition of claim 40, wherein the beads of the second substantially zero order release and third substantially elevating or substantially ascending release portions (if present), are coated by a bioadhesive material.

42. The pharmaceutical composition of any of claims 38-41, wherein some or all of the beads are coated by a dispersion-promoting coating.

43. The pharmaceutical composition of any of claims 38-42, wherein the beads are less than about 1 mm in diameter.

44. The pharmaceutical composition of any of claims 38-43, wherein the beads are dispersed in a matrix that disintegrates in less than about 5 minutes.

45. The pharmaceutical composition of any of claims 38-43, wherein the beads are dispersed in an eroding tablet that gradually erodes over the treatment period.

46. The pharmaceutical composition of claim 45, wherein the tablet is at least partially coated by a bioadhesive material and/or an immediate release portion.

47. The pharmaceutical composition of claim 46, wherein the bioadhesive material, if present, is exposed upon dissolution of the immediate release portion.

48. The pharmaceutical composition of claim 37, wherein the shell is fully or partially coated by a bioadhesive material.

49. The pharmaceutical composition of any of the preceding claims, wherein at least the substantially zero-order release rate second portion is coated or partially coated by a bioadhesive material.

50. The pharmaceutical composition of any of claims 40-49, wherein the bioadhesive material is selected from polyamides, polyalkylene glycols, polyalkylene oxides, polyvinyl alcohols, polyvinylpyrrolidone, polyglycolides, polyurethanes, polymers of acrylic and methacrylic esters, polylactides, poly(butyric acid), polyanhydrides, polyorthoesters, poly(fumaric) anhydride, blends and copolymers thereof.

51. The pharmaceutical composition of any of claims 40-49, wherein the bioadhesive material is poly(fumaric-co-sebacic) anhydride.

52. The pharmaceutical composition of any of claims 40-49, wherein the bioadhesive material comprises a catechol moiety.

53. The pharmaceutical composition of claim 52, wherein the bioadhesive material comprises a mixture of a material and a compound comprising a catechol moiety selected from L-Dopa, D-dopa, dopamine, or carbidopa.

54. The pharmaceutical composition of claim 52 or 53, wherein the bioadhesive material is selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric) anhydride, blends, and/or copolymers thereof.

55. The pharmaceutical composition of any of claims 40-49, wherein the bioadhesive material is covalently functionalized with a catechol moiety.

56. The pharmaceutical composition of claim 55, wherein the catechol moiety is derived from L-dopa, D-dopa, dopamine, or carbidopa.

57. The pharmaceutical composition of any of the preceding claims, formulated for oral administration.

58. The pharmaceutical composition of any of claims 1-56, formulated for parenteral administration.

59. The pharmaceutical composition of any of the preceding claims, suitable for human treatment, or for veterinary treatment of a non-human mammal.

60. The pharmaceutical composition of any of the preceding claims, wherein the first IR portion is liquid.

61. The pharmaceutical composition of any of the preceding claims, wherein the first IR portion, the second substantially zero order release portion, the substantially elevating release portion (if present), and the substantially ascending release portion (if present), are suspended in a pharmaceutically acceptable liquid carrier.

62. A method of making a pharmaceutical composition for the treatment of a patient suffering from Parkinson's disease and/or another movement disorder, comprising combining the first IR portion, the second portion, the substantially elevating release portion (if present), and the substantially ascending release portion (if present), of any of the pharmaceutical composition of claims 1-60 into a single dosage form.

63. A method of treating a patient suffering from Parkinson's disease and/or another movement disorder, comprising administering to the patient a pharmaceutical composition of any of claims 1-61.

64. A method of treating a patient suffering from Parkinson's disease and/or another movement disorder, comprising administering to the patient the pharmaceutical composition of claim 1, conjointly with a substantially ascending release portion comprising levodopa or precursor thereof formulated to effect a rapid drop of levodopa concentration in the patient to below the therapeutically effective concentration at the end of the treatment period, when the release of the pharmaceutical composition of claim 1 is or is about to be completed in the patient.

65. A pharmaceutical preparation of any of claims 1-60 formulated as a transdermal patch, a buccal patch, or a buccal tablet, and formulated for sustained release of the pharmaceutical composition in order to administer an amount sufficient to treat a patient suffering from Parkinson's disease and/or another movement disorder, wherein said pharmaceutical composition is formulated to provide a sustained substantial zero-order release over at least 8 hours after applying to the patient.

66. A single dosage formulation for treatment of a movement disorder comprising levodopa or a metabolic precursor thereof, wherein the dosage formulation produces and maintains a therapeutically effective concentration of levodopa or precursor thereof over a period of at least about 7 hours.

67. The single dosage formulation of claim 66, further comprising a decarboxylase enzyme inhibitor.

68. The single dosage formulation of claim 66 or 67, wherein the dosage formulation produces and maintains a therapeutically effective concentration of levodopa or precursor thereof over a period of at least about 9 hours.

69. The single dosage formulation of claim 68, wherein the dosage formulation produces and maintains a therapeutically effective concentration of levodopa or precursor thereof over a period of at least about 12 hours.

70. The single dosage formulation of any of claims 66-69, wherein the dosage formulation includes a first immediate-release (IR) portion to attain a therapeutically effective concentration of said levodopa in less than about 2 hours after administration to a patient.

71. The single dosage formulation of claim 70, which further comprises:
(1) a sustained zero-order release portion to maintain the therapeutically effective concentration of levodopa over a first period of hours; and
(2) an ascending-release portion to maintain the therapeutically effective concentration of levodopa at the end of said sustained zero-order release portion;
wherein the single dosage formulation, upon administration to the patient, produces a therapeutically effective concentration of said levodopa or precursor in less than about 2 hours of administration to a patient.

72. The single dosage formulation of claim 71, wherein the ascending release portion provides for a rate of decrease of levodopa in the patient from a therapeutically effective concentration to a sub-therapeutically effective concentration (e.g., <75% or less) in a second period of time less than about 2 hours.

73. The single dosage formulation of any of claims 67-72, wherein the ratio of the inhibitor to levodopa or the precursor in at least the first IR portion is greater than 1:4.

74. The single dosage formulation of any of claims 70-73, wherein the decarboxylase enzyme inhibitor is present in only the first IR portion and the sustained zero-order release portion.

75. The single dosage formulation of any of claims 70-73, wherein the decarboxylase enzyme inhibitor is present in each of the portions, wherein the ratio of decarboxylase enzyme inhibitor to levodopa in each portion is different.

76. The single dosage formulation of any of claims 71-75, wherein the sustained zero-order release portion comprises two or more sub-portions differing in the ratio of decarboxylase enzyme inhibitor to levodopa or the precursor.

77. The single dosage formulation of any of claims 71-75, wherein at least one of the first IR portion, the second substantially zero order release portion, and the substantially ascending release portion further comprises at least one dopamine transport inhibitor.

78. The single dosage formulation of any of claims 71-75, wherein the second substantially zero order release portion and/or the substantially ascending release portion further comprises a bioadhesive material.

79. The pharmaceutical composition of claim 40, wherein the bioadhesive material comprises an additive that stabilizes the material from erosion, dissolution or both, wherein at least 50% by weight of a 1 mm thick film of the bioadhesive material remains after 12 hours in a buffered pH 4.5 dissolution bath.

80. The pharmaceutical composition of claim 40, wherein the bioadhesive material comprises an additive selected from one or more of a polyanhydride, an acidic component, a metal compound, a stabilizing polymer and a hydrophobic component.

81. A packaged pharmaceutical composition for the treatment of a patient suffering from Parkinson's disease and/or another movement disorder, comprising:
(1) a first pharmaceutical composition comprising the pharmaceutical composition of claim 1, 2, or 3;
(2) a second pharmaceutical composition comprising the pharmaceutical composition of claim 9, 10, or 11.

82-86. (canceled)
